(19) United States
(12) Patent Application Publication
(10) Pub. No.: US 2019/0000850 A1
(43) Pub. Date: Jan. 3, 2019

(54) COMBINATION CANCER THERAPY

(71) Applicant: The Regents of the University of California, Oakland, CA (US)

(72) Inventors: Caius Gabriel Radu, Los Angeles, CA (US); Soumya Poddar, Los Angeles, CA (US); Johannes Czernin, Los Angeles, CA (US); David Nathanson, Los Angeles, CA (US); Thuc Le, Los Angeles, CA (US)

(21) Appl. No.: 15/549,609

(22) PCT Filed: Feb. 9, 2016
(86) PCT No.: PCT/US2016/017199

§ 371 Date: Aug. 8, 2017

Related U.S. Application Data
(60) Provisional application No. 62/113,871, filed on Feb. 9, 2015.

(51) Int. Cl. A61K 31/519; A61K 31/497; A61K 31/506; A61P 35/00

(52) U.S. Cl. CPC A61K 31/519; A61P 35/00; A61K 31/506; A61K 31/497

(57) ABSTRACT

Described herein, inter alia, are anti-cancer agents, anti-cancer pharmaceutical compositions, combinations of the agents and/or pharmaceutical compositions, and methods of using the same.

### CROSS-REFERENCES TO RELATED APPLICATIONS

This application claims the benefit of U.S. Provisional Application No. 62/113,871, filed Feb. 9, 2015, and U.S. Provisional Application No. 62/276,546, filed Jan. 8, 2016, each of which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. CA086306 awarded by the National Institutes of Health. The government has certain rights in the invention.

### BACKGROUND

Cancer cells are more susceptible than normal cells to perturbations in the quantity, balance and quality of the deoxyribonucleotide triphosphate (dNTP) pools. Ribonucleotide reductase (RNR) controls the rate-limiting step in de novo dNTP production and is capable of generating all four diphosphate deoxyribonucleotide (dNDP) precursors of DNA. RNR is an important therapeutic target in cancer. However, in multiple clinical trials RNR inhibitors have shown modest therapeutic efficacy and significant off-target toxicity (possibly due to overdosing). Disclosed herein are solutions to these and other problems in the art.

### BRIEF SUMMARY

A pharmaceutical composition including a pharmaceutically acceptable excipient, a de novo nucleotide biosynthesis pathway inhibitor, a nucleoside salvage pathway inhibitor, and a replication stress response pathway inhibitor.

A pharmaceutical composition described herein for use in treating cancer in a patient in need of such treatment, the use including administering an effective amount of the pharmaceutical composition to the patient.

A pharmaceutical composition described herein for use in inhibiting the growth of a cancer cell including contacting the cancer cell with the pharmaceutical composition.

### BRIEF DESCRIPTION OF THE DRAWINGS

FIGS. 1A-1C. (FIG. 1A) Schematic representation of the mechanisms of action for various RNR inhibitors. dT is converted to thymidine triphosphate (dTTP) which binds to the allosteric specificity site in the R¹ subunit and switches RNR to reduce GDP in preference to CDP and UDP. GaM releases Ga³⁺ which mimics Fe³⁺ and disrupts the iron complex in the R² subunit. HU scavenges the R² tyrosyl radical. 3-AP forms a complex with Fe²⁺ which reduces the R² tyrosyl radical (Aye et al., 2012); (FIGS. 1B-1C) Effects of various RNR inhibitors on leukemia cell growth and IC₅₀ values; 3-AP (3-aminopyridine-2-carboxyaldehyde-thiosemicarbazone), dT—thymidine; GaM—gallium maltolate; HU—hydroxyurea; 3-AP-3-aminopyridine-2-carboxyaldehyde-thiosemicarbazone.

FIG. 2. The 3-AP/dCKi combination therapy is effective against the thymidine (dT) resistant cell lines MV4-11 (mixed lineage leukemia) and THP-1 (acute monocytic leukemia).

FIGS. 3A-3D. 3-AP synergizes with dCK inhibition by DI-82 in CEM T-Acute lymphoblastic leukemia cells. (FIG. 3A) Induction of DNA damage as measured by pH2A.X staining in CEM cells treated with 3-AP and DI-82 alone or in combination. (FIGS. 3B-3C) Induction of cell death as measured by Annexin V/PI staining in CEM cells treated with 3-AP and DI-82 alone or in combination. (FIG. 3D) Replication stress characterization in CEM cells treated with 3-AP and DI-82 alone or in combination.

FIGS. 4A-4E. dCTP pool becomes rate-limiting with 3-AP treatment, possibly due to the CDP pool being the smallest pool amongst the RNR substrates; the dC salvage pathway prevents the pool to be depleted, (FIG. 4A) Schematic representation of the de novo deoxyribonucleotide (dNTP) production by RNR, and of the deoxycytidine kinase (dCK) salvage pathway as a potential alternate pathway to prevent dCTP pool depletion by RNR. (FIG. 4B) NDP (ribonucleoside diphosphate) levels in leukemia cells (Jurkat T-ALL): the CDP pool is the smallest pool amongst the RNR substrates; (FIG. 4C) dNTP (deoxyribonucleotide triphosphate) levels in leukemia cells (Jurkat T-ALL) treated with or without 300 nM 3-AP for 24 hours; the dCTP pool becomes rate-limiting first upon treatment with 3-AP. (FIG. 4D) dCTP levels in leukemia cells (CEM T-ALL) treated with 300 nM or 750 nM 3-AP+/−2.5 μM dC for 24 hours, exogenous dC can replenish the dCTP pool via the dC salvage pathway; (FIG. 4E) dCTP levels in leukemia cells (CEM T-ALL) treated with 300 nM or 750 nM 3-AP+/−1 μM (R) DI-82 (dCKi) in presence of 2.5 μM dC; addition of a dCK inhibitor synergizes with 3-AP to deplete the dCTP pool.

FIG. 5. 3-AP+RSRi treatment of HepG2 and Hep3B; 72 h profiling of the RSR pathway by WB.

FIGS. 6A-6C. Co-targeting dCTP biosynthesis and the RSR pathway improves treatment of BCR-ABL p185⁺/Arf^{-/-} preB leukemia; BCR-ABL p185⁺/Arf^{-/-} preB leukemia cells were treated with 3-AP, dCK inhibitor (dCKi-DI-82 racemic) and VE-822, alone or in combination. 72 h following treatment, cultures were analyzed by flow cytometry for Annexin V/PI staining to determine dead or apoptotic cells. (FIG. 6A) Annexin V/Propidium Iodide FACS plots (FIG. 6B) percentages of Annexin V and/or Propidium Iodide positive cells, as determined by FACS; (FIG. 6C) Total number of Trypan Blue negative cells, as measured by Vi-Cell.

FIGS. 7A-7C. Co-targeting dCTP biosynthesis and the RSR pathway improves treatment of CEM T-Acute lymphoblastic leukemia (T-ALL) cells; (FIG. 7A) CEM cells were treated with 3-AP, dCK inhibitor (dCKi-DI-82 racemic) and VE-822, alone or in combination; Plots of Annexin V and/or Propidium Iodide positive cells, as determined by Flow cytometry; (FIG. 7B) Percentages of Annexin V and/or Propidium Iodide positive cells. (FIG. 7C) Total number of Trypan Blue negative cells, as measured by Vi-Cell.

FIGS. 8A-8C. Co-targeting dCTP biosynthesis and the RSR pathway improves treatment of Jurkat T-ALL cells. (FIG. 8A) Jurkat 3-AP, dCK inhibitor (dCKi-DI-82 racemic) and VE-822, alone or in combination; Plots of Annexin V and/or Propidium Iodide positive cells, as determined by Flow cytometry; (FIG. 8B) Percentages of Annexin V and/or Propidium Iodide positive cells. (FIG. 8C) Total number of Trypan Blue negative cells, as measured by Vi-Cell.

FIGS. 9A-9D. Co-targeting dCTP biosynthesis and the RSR pathway improves treatment of B16 melanoma cells; B16 melanoma cells were treated with 3-AP alone or in combination VE-822. 72 h following treatment, cultures were analyzed by flow cytometry for Annexin V/PI staining to determine dead or apoptotic cells, and Cell TiterGlo to monitor cell proliferation/viability. (FIG. 9A) Annexin V/Propidium Iodide FACS plots (FIG. 9B) percentages of Annexin V and/or Propidium Iodide positive cells, as determined by FACS; (FIG. 9C) Total number of Trypan Blue negative cells, as measured by Vi-Cell. (FIG. 9D) % of control of ATP content after 72 hours determined by Cell Titer Glo assay. Note: the deoxycytidine salvage pathway was rendered inactive by not adding the deoxycytidine substrate; this approach simplifies the experimental setup and is equivalent to using a dCK inhibitor.

FIG. 10. Co-targeting dCTP biosynthesis and the RSR pathway improves treatment of B16 U87-VIII Glioblastoma cells; U87-VIII Glioblastoma multiforme (GBM) cells were treated with 3-AP and VE-822 alone or in combination; 72 hours following treatment, viability was determine by the Cell Titer Glo assay. Note: the deoxycytidine salvage pathway was rendered inactive in this experiment by not-adding the deoxycytidine substrate; this approach simplifies the experimental setup and is equivalent to using a dCK inhibitor.

FIGS. 11A-11E. Co-targeting dCTP biosynthesis and the RSR pathway improves treatment of MiaPaCa pancreatic cancer cells; MiaPaCA pancreatic cancer cells were treated 3-AP alone or in combination with VE-822. (FIG. 11A) 72 hours after treatment, cultures were analyzed by flow cytometry for Annexin V/PI staining to determine dead or apoptotic cells. (FIG. 11B) percentages of Annexin V and/or Propidium Iodide positive cells, as determined by FACS; (FIG. 11C) Total number of Trypan Blue negative cells, as measured by Vi-Cell. (FIG. 11D) Replication stress characterization after 24 hours of treatment. (FIG. 11E) % of control of ATP content after 72 hours determined by Cell Titer Glo assay. Note: the deoxycytidine salvage pathway was rendered inactive in this experiment by not-adding the deoxycytidine substrate; this approach simplifies the experimental setup and is equivalent to using a dCK inhibitor.

FIGS. 12A-12E. Co-targeting dCTP biosynthesis and the RSR pathway improves treatment of 22RV1 prostate cancer cells; the combination therapy is effective against 22RV1 prostate cancer cell lines. (FIG. 12A) Schematic of the experimental setup. (FIG. 12B) Percentages of Annexin V and/or Propidium Iodide positive cells 72 h after treatment. (FIGS. 12C-12E) Percentages of Annexin V/PI positive cells (upper row), Annexin V/PI negative cells (bottom row) at days 3, 6 and 9.

FIG. 13. Co-targeting dCTP biosynthesis and the RSR pathway improves treatment of HEY ovarian carcinoma cells; HEY ovarian cancer cells were treated with 3-AP and VE-822 alone or in combination; 72 hours following treatment, viability was determine by the Cell Titer Glo assay. Note: the deoxycytidine salvage pathway was rendered inactive in this experiment by not-adding the deoxycytidine substrate; this approach simplifies the experimental setup and is equivalent to using a dCK inhibitor.

FIG. 14. Cell Titer Glo assay for detection of cellular ATP content using a luminescence-based assay has a large dynamic range and is easily adaptable to high-throughput screening; Cell Titer Glo HTS Optimization—Cell Number and Reagent Ratio; Day −1: Cells are plated at 200-500 cells/well in opaque white 384 well plates (Thermo Nunc) in 30 μL media. 100 μL of dH₂O is plated in empty wells to prevent evaporation; Day 0: 24 h following seeding 10 μL of 4× drug (in media, final DMSO<0.1%) is added to each well; n=4 for each condition; Days 1/3: 24 h and 72 h after drug treatment; 10 μL of Cell-Titer Glo reagent is added to each well (1:4), mixed by pipetting, shaken at 500 rpm for 1 min at room temperature. Luminescence signal is read 10 min after shaking.

FIG. 15. Cell Titer Glo—Cell Plating Density Optimization; fold change in relative light units (RLU) from 24 to 72 h after plating identifies the optimal plating density for the HTS assay; this range is 100-250 cells/well for B16 melanoma and 150-450 cells/well for HEY ovarian cancer cells, at a 4:1 media: CTG reagent ratio; additional assay could be Caspase 3/7 Glo—Luminescent assay that allows for high-throughput detection of a specific pathway of cell death (Caspase 7 mediated apoptosis).

FIG. 16A-16D. Pharmacokinetic (PK) studies of select combinations. (FIG. A) Graphic representation of the PK profile of DI-39 and DI-82. (FIG. B) Solubility study results for 3-AP in phosphate buffer solution (PEG-Tris). (FIG. C) Solubility study results for 3-AP in phosphate buffer solution (VitE-TGPS). (FIG. D) Solubility study results for 3-AP in phosphate buffer solution (VitE-TGPS). VE-822 3-AP<0.3 mg/mL, DI-82<<0.3 mg/mL. Pharmacokinetic (PK) studies of select combinations; solubility study results for 3-AP and DI-82 in phosphate buffer solution, 3-AP<0.3 mg/mL, DI-82<<0.3 mg/mL.

FIGS. 17A-17F. Efficacy of the 3-AP/DI-82 combination treatment against a primary BCR-ABL p185⁺/Arf^{-/-} preB-ALL systemic model. Pharmacological co-targeting of RNR by 3-AP and dCK by DI-82 is efficacious against primary mouse p185^{BCR-ABL} Arf^{-/-} pre-B ALL cells (FIG. 17A) Schematic illustration of 3-AP/DI-82 combination treatment study. (FIG. 17B) Representative bioluminescence images of mice (n=4) on day 16 (after intravenous injection of pre-B leukemia cells) of the treatment groups, treated with vehicle, 5 mg/kg 3-AP, 50 mg/kg DI-82, or DI-82+3-AP. (FIG. 17C) Whole body bioluminescence quantification of respective images on day 16. (FIG. 17D) Bioluminescence quantification of mice (n=4) till day 21 treated with vehicle, 5 mg/kg 3-AP, 50 mg/kg DI-82, or DI-82+3-AP for 11 days after intravenous injection of 2×10⁵ pre-B leukemia cells/mouse; 3-AP and DI-82 were administered i.p. in PEG-Tris. (FIG. 17E) Body weights of C57Bl6 mice for 25 days, (n=4) treated with vehicle, 5 mg/kg 3-AP. DI-82 or DI-82+3-AP. (FIG. 17F) Kaplan-Meier survival analysis of mice treated with vehicle, 50 mg/kg DI-82, 5 mg/kg 3-AP, or DI-82+3-AP after intravenous injection of 2×10⁵ pre-B leukemia cells/mouse.

FIGS. 18A-18F. Triple combination therapy targeting RNR by 3-AP, dCK by LP-661438 and ATR by VE-822 is efficacious against primary mouse p185^{BCR-ABL} Arf^{-/-} pre-B ALL cells; TX: 3-AP (RNR inhibitor):1/2/3 mg/kg IP 2× Daily, VE-822 (ATR inhibitor): 40 mg/kg PO 1× Daily, LP-661438 (dCK inhibitor): 100 mg/kg PO 1× Daily; (FIG. 18A) Schematic illustration of the triple combination treatment study to determine the therapeutic efficacy in vivo. (FIG. 18B) Representative bioluminescence images of different treatment groups of mice (n=5) on day 0, 2 and 5 post-treatment. (FIG. 18C) Representative bioluminescence images of different treatment groups of mice (n=5) on day 5, 7 and 12 post-treatment. (FIG. 18D) Bioluminescence quantification of mice (n=5) till day 24 (post-treatment) treated with vehicle, 1, 2 or 3 mg/kg 3-AP in combination with 100 mg/kg LP-661438, and 40 mg/kg VE-822 for 12 days; Tx: Treatment, VE-822 and LP-661438 administered orally once daily (P.O.) in TPGS for 12 days; 3-AP administered twice daily (b.i.d) in PEG-Tris for 12 days. (FIG. 18E) Body weights of C57Bl6 mice till 42 days, (n=5) treated with vehicle, 1, 2 or 3 mg/kg 3-AP in combination with 100 mg/kg LP-661438, and 40 mg/kg VE-822. (FIG. 18F) Kaplan-Meier survival analysis of mice till day 42 treated with vehicle, 1, 2 or 3 mg/kg 3-AP in combination with 100 mg/kg LP-661438, and 40 mg/kg VE-822.

FIG. 19. ¹⁸F-clofarabine has higher sensitivity than ¹⁸F-L-FAC for imaging dCK activity in humans; this new probe could be used to determine the degree of pharmacological inhibition of dCK in clinical trials.

FIG. 20. Potential candidate cell line for HTS: MiaPaca PDAC cells; Day −1: Cells are plated at 200-500 cells/well in opaque white 384 well plates (Thermo Nunc) in 30 μL media. 100 μL of dH₂O is plated in empty wells to prevent evaporation; Day 0: 24 h following seeding 10 μL of 4× drug (in media, final DMSO<0.1%) is added to each well; n=4 for each condition; Days 1/3: 24 h and 72 h after drug treatment; 10 μL of Cell-Titer Glo reagent is added to each well (1:4), mixed by pipetting, shaken at 500 rpm for 1 min at room temperature. Luminescence signal is read 10 min after shaking.

FIGS. 21A-21F. Effects on 3-AP on the deoxycytidine salvage pathway in cell culture and in vivo. (FIG. 21A) dNTP levels in Jurkat cells treated for 18 h with vehicle and 300 nM 3-AP+/−1 μM dCKi. (FIG. 21B) CEM cell cycle analysis after treatment with 500 nM 3-AP+/−2.5 μM dC. (FIG. 21C) 3H-deoxycytidine (dC) uptake as percent change relative to control in CEM T-ALL cells treated with 500, 750 and 1500 nM of 3-AP respectively with and without 1 μM dCK inhibitor (R-DI-82). (FIG. 21D) 18F-D-FAC PET/CT scans of C57/Bl6 mice to assess dCK activity at 1, 3 and 5 h after 3-AP treatment (7.5 mg/kg). (FIG. 21E) Quantification of PET scans in bone marrow (BM), gastrointestinal track (GI) and liver (L) relative to control; n=2 independent experiments with 3 mice/group. (FIG. 21F) LC/MS/MS quantification of dC concentrations in plasma from 3-AP treated mice relative to control.

FIG. 22. Replication stress response is a resistance mechanisms for 3-AP treatment; quantification of pChk1 (S296), pChk1 (S345) and pH2A.X (S139) phosphorylation in the treatment groups.

FIGS. 23A-23D. Triple combination treatment in 22RV1 cells leads to synthetic lethality. (FIG. 23A) Cells were treated with 500 nM 3-AP+/−1 μM (R)-DI-82+/−RSRi (100 nM AZD-7762: Chk1/2 dual inhibitor; 100 nM MK-1775: Wee1 inhibitor) for 72 hours, cells were then washed and released in warm media for 72 hours, followed by treatment of respective drugs again at day 6. Cell death was investigated using Annexin V/Propidium iodide staining analyzed by Flow cytometry. Viable cells were counted using Beckmann Cell counter. Cells were supplemented with 2.5 μM dC daily. (FIG. 23B) Cells were plated 1000 cells at day 0 and allowed to grow over 12 days in presence of respective drug combinations, with dC supplementation every day. Colony growth of cells were visualized using Crystal violet staining. (FIG. 23C) Cells were titrated with combinations of different concentrations of 3-AP and MK-1775, and cell viability was measured using Cell TiterGlo assay. (FIG. 23D) Representative Immunoblot of replication stress response biomarkers treated with respective drugs.

FIGS. 24A-24C. Double combination treatment in MiaPaCa cells leads to synthetic lethality. (FIG. 24A) Cells were treated with 300 nM 3-AP+/−RSRi (100 nM AZD-7762: Chk1/2 dual inhibitor; 100 nM MK-1775: Wee1 inhibitor; 50 nM VE-822: ATR inhibitor) for 72 hours. Cell death was investigated using Annexin V/Propidium iodide staining analyzed by Flow cytometry. Viable cells were counted using Beckmann Cell counter. Cells were supplemented with 2.5 μM dC daily. (FIG. 24B) Cells were plated 1000 cells at day 0 and allowed to grow over 7 days in presence of respective drug combinations, with dC supplementation every day. Colony growth of cells were visualized using Crystal violet staining. (FIG. 24C) Representative Immunoblot of replication stress response biomarkers treated with respective drugs.

FIGS. 25A-25B. Double combination treatment in DU-145 cells leads to synthetic lethality. (FIG. 25A) Cells were treated with 300 nM 3-AP+/−RSRi (100 nM AZD-7762: Chk1/2 dual inhibitor; 100 nM MK-1775: Wee1 inhibitor; 50 nM VE-822: ATR inhibitor) for 72 hours. Cell death was investigated using Annexin V/Propidium iodide staining analyzed by Flow cytometry. Viable cells were counted using Beckmann Cell counter. Cells were supplemented with 2.5 μM dC daily. (FIG. 25B) Cells were plated 1000 cells at day 0 and allowed to grow over 7 days in presence of respective drug combinations, with dC supplementation every day. Colony growth of cells were visualized using Crystal violet staining.

FIGS. 26A-26C. Triple combination treatment in CEM T-ALL cells leads to synthetic lethality. (FIG. 26A) Cells were treated with 300 nM 3-AP+/−dCKi+/−RSRi (100 nM AZD-7762: Chk1/2 dual inhibitor; 100 nM MK-1775: Wee1 inhibitor; 1 μM (R)-DI-82: dCK inhibitor) for 72 hours. Cell death was investigated using Annexin V/Propidium iodide staining analyzed by Flow cytometry. Viable cells were counted using Beckmann Cell counter. Cells were supplemented with 2.5 μM dC daily. FIG. 26B: Representative immunoblot for pChk1 (S345) of CEM T-ALL cells treated with low (300 nM) and high (750 nM) concentration of 3-AP in combination with (R)-DI-82. (FIG. 26C) Representative Immunoblot of replication stress response biomarkers treated with different combination of drugs immunoblot.

FIGS. 27A-27B. CEM: T-ALL cell line: (FIG. 27A) Cells were treated with 300 nM 3-AP+/−1 μM (R)-DI-82+/−RSRi (100 nM AZD-7762: Chk1/2 dual inhibitor; 100 nM MK-1775: Wee1 inhibitor) for 72 hours, cells were then washed and released in warm media for 72 hours, followed by treatment of respective drugs again at day 6. Cell death was investigated using Annexin V/Propidium iodide staining analyzed by Flow cytometry. Viable cells were counted using Beckmann Cell counter. Cells were supplemented with 2.5 μM dC daily. (FIG. 27B) 72 hour cell death results at day 3, drug release results at day 6, and second treatment results at day 9.

FIG. 28. p185: pre-B ALL cell line: 72 hour cell death assay.

FIG. 29. In vitro biological data in CEM cells for compounds S1-S31.

FIG. 30. In vitro biological data in L1210 and CEM cells for compounds 15-18.

FIG. 31. In vitro biological data in CEM cells for compounds 25-37.

FIGS. 32A-32N. Examples of nucleoside salvage pathway inhibitors and dCK inhibitors.

FIG. 33. In vitro biological data in L1210 and CEM cells for compounds 8-14.

FIG. 34 Table of DI compounds.

FIGS. 35A-35C. Co-inhibition of ATR and dCK impairs the G1-S transition in cancer cells. (FIG. 35A) Schematic representation of the roles played by ATR, RNR, and dCK for coordinating dCTP biosynthesis and DNA-C replication. (FIG. 35B) CEM T-ALL cells were synchronized in G1 phase by 24 h treatment with the CDK4/6 inhibitor, pablociclib and were then released in fresh media. *EdU was added 1 hour before cell harvest for FACS analysis of EdU uptake and DNA content per cell. FACS profile show EdU uptake in asynchronized cells, and G1 arrested cells. Cells were also harvested at indicated time points after release from G1 for western blot. Immunoblot showing RRM2, dCK, and actin protein expressions in synchronous cells at indicated time points following release from G1 arrest. (FIG. 35C) CEM T-ALL cells were cultured with Palbociclib for 24 h and released in fresh media with VE-822±DI-82 treatment for the time indicated. *EdU was added 1 hour before cell harvest for FACS analyses. The number indicated in each FACS plot represent percentage of cells in the corresponding gates S1, S2 and S3. Percentage of cells in gate S1, S2 and S3, 6, 12, and 18 hours after release from G1 into VE-822±DI-82 treatment is shown as bar graphs. All data are representative of two independent experiments (N=2 for each experiment). *, P<0.05; **, P<0.005; ***, P<0.0005.

FIGS. 36A-36D. Effects of ATR and dCK inhibition on RSR/DDR signaling pathways. (FIG. 36A) Workflow for the proteomic/phosphoproteomic analyses: Synchronized CEM T-ALL cells were released in drug treated media, collected at 6 and 12 h time points, and lysed. Protein lysates were digested by trypsin and differentially labeled by stable-isotope dimethylation. 100 μg labeled peptides were sub-fractionated using STAGETips. Remaining sample were used for phosphopeptide enrichment using HILIC/IMAC. The samples were analyzed using reverse-phase nLS-MS/MS. (FIG. 36B) Phosphorylation status of Chk1, CLSPN and CDK proteins relative to untreated at respective time point. (FIG. 36C) List of cell cycle and RSR/DDR phosphoproteins that exhibit either more than 50% increase or 50% decrease of the phosphorylation level by the addition of DI-82 under ATR inhibition at 12 h. (FIG. 36D) RRM2 and dCK expression at 6 and 12 h after G1 release.

FIGS. 37A-37E. Mass spectrometric assay to simultaneously measure the differential contribution of de novo and salvage pathways to newly replicated DNA. (FIG. 37A) Workflow of the assay. Cells are incubated for 3-12 h in medium supplemented with stable isotope-labeled nucleotide precursors. dNTP and genomic DNA were extracted, hydrolyzed and analyzed on a triple quadruple mass spectrometer (QQQ) running in the multiple reaction monitoring (MRM) mode. Using dC as an example, the first (Q1) and third (Q3) quadruples act as mass filters, while the second (Q2) quadrupole acts as a collision chamber. The ion chromatogram showed the differential contributions of the de novo and salvage pathways, which are quantified by peak area analysis. (FIG. 37B and FIG. 37C) Measurements of the contributions of the de novo (RNR) and salvage (dCK) pathways to the newly synthesized dCTP (FIG. 37B) and total dCTP pool levels (FIG. 37C) in CEM cells at indicated time points released from G1 synchronization. (FIG. 37D and FIG. 37E) Measurements of contributions of the de novo (RNR) and salvage (dCK) pathways to newly replicated DNA-C (FIG. 37D) and total newly replicated DNA-C (FIG. 37E) in CEM cells released from G1 synchronization. All data are representative of two independent experiments (N=3 for each experiment).

FIGS. 38A-38F. Inhibition of residual RNR activity in VE-822+DI-82 treated cells by low dose 3-AP. (FIG. 38A) Sites of action of the different RNR inhibitors. GaM, gallium maltolate; HU, hydroxyurea; dT, thymidine; 3-AP, triapine. (FIG. 38B) IC₅₀ measurements for the four RNR inhibitors after 72 h treatment in CEM cells. (FIG. 38C and FIG. 38D) Measurements of the contributions of the de novo (RNR) and salvage (dCK) pathways to the newly synthesized dCTP (FIG. 38C) and total dCTP pool levels (FIG. 38D) in CEM cells in indicated treatment groups at 18 h. (FIG. 38E and FIG. 38F) Measurements of contributions of the de novo (RNR) and salvage (dCK) pathways to newly replicated DNA-C (FIG. 38E) and total newly replicated DNA-C (FIG. 38F) in CEM cells in indicated treatment groups at 18 h.

FIGS. 39A-39C. Replication stress overload induced by combined ATR and nucleotide metabolism inhibition. (FIG. 39A) CEM T-ALL cells were treated with VE-822+DI-82±500 nM 3-AP, and ssDNA accumulation and DSB were measured after 0.5, 4 and 18 h after treatment by FACS analyses. The quantification of ssDNA and DSB at different time points are shown in the right panel. (FIG. 39B) Measurement of apoptosis 72 h after respective treatments in CEM T-ALL cells (FIG. 39C) Cell Trace Violet (CTV) dye dilution curves showing the number of cell divisions under indicated treatment conditions. All data are representative of 2 independent experiments. *, P<0.05; **, P<0.005; ***, P<0.0005.

FIGS. 40A-40I. Replication stress overload eradicates cancer cells and is well-tolerated in a mouse model of preB-ALL. (FIG. 40A) A waterfall plot showing the IC₅₀ values of VE-822 following 72 h treatment in a panel of cancer cell lines and primary cancer cells measured by Cell-Titer-Glo assay. (FIG. 40B) Kaplan-Meier survival curves of C57BL/6 mice bearing syngeneic systemic BCR-ABL p185⁺/Arf^{-/-} pre B-ALL cells treated with vehicle or VE-822 for 3 weeks. Treatment was started 7 days after inoculation of leukemia initiating cells. (FIG. 40C) Pharmacokinetic profile of 3-AP, VE-822 and DI-82 co-administered orally in prototype 9′ in C57Bl/6 mice. (FIG. 40D) Schematic representation of the treatment schedule of C57BL/6 mice bearing syngeneic systemic BCR-ABL p185⁺/Arf^{-/-} pre B-ALL cells. q.d. and b.i.d. stands for once/day and twice/day respectively. The treatment dosages were 15 mg/kg 3-AP, 50 mg/kg DI-82 and 40 mg/kg VE-822. (FIG. 40E-FIG. 40H) Representative bioluminescence images (FIG. 40E), the quantification of whole-body radiance values (FIG. 40F), Kaplan-Meier survival curves (FIG. 40G), and body weight measurements (FIG. 40H) of tumor bearing mice treated with vehicle or with the triple combination therapy at indicated days after tumor inoculation. (FIG. 40I) The model summarizing the demand and supply of dNTP for DNA replication and repair coordinated by RNR, DCK and ATR and by pharmacologically targeting them. Results of FIGS. 40A and 40C are representative of 2 independent experiments.

FIGS. 41A-41I. Analysis of the de novo and salvage dATP/DNA-A biosynthetic pathways reveals a significant role of dCK upon the inhibition of ADA-mediated dA catabolism. (FIG. 41A) A detailed schematic representation of the de novo and salvage pathways for dATP and DNA-A biosynthesis illustrating the targets of dCF and DI-82. (FIG. 41B-FIG. 41E) metabolic profiles. Jurkat cells were incubated in culture media containing 11 mM [¹³C₆]glucose and 5 μM [¹⁵N₅]dA and were then treated with 10 μM dCF and/or 1 μM DI-82. (FIG. 41B) [¹⁵N₅]dA levels in the cell culture media (FIG. 41B); profile of de novo biosynthesis of dATP and DNA-A (FIG. 41C); profile of salvage biosynthesis of dATP and DNA-A from [¹⁵N₄]Hx (nucleobase salvage from deamination and phosphorylase of [¹⁵N₅]dA) and [¹⁵N₅]dA (nucleoside salvage) (FIG. 41D); fold change in total dNTP levels in the indicated treatment groups following 18 h incubation under the indicated conditions (FIG. 41E). (FIG. 41F and FIG. 41G) phenotypic assays. Jurkat cells were incubated in culture media containing 11 mM glucose and 5 μM dA and were then treated with 10 μM dCF and/or 1 μM DI-82. DNA histograms showing the cell cycle profiles (FIG. 41F) and quantification of the cells in S phase (FIG. 41G, left panel) and G2/M phase (FIG. 41G, right panel) following 24 h treatment. (FIG. 41H) Western blot analysis of pChk1 (S345), pChk2 (T68), pH2A.X (S139) and actin following 24 h treatment. (FIG. 41I) A schematic summary of the effects of dCF and DI-82 on dATP and DNA-A biosynthesis.*, P<0.05; **, P<0.005; ***, P<0.0005.

FIGS. 42A-42I. Analysis of dGTP/DNA-G biosynthetic pathways reveals a significant role of dCK upon the inhibition of PNP-mediated dG catabolism (FIG. 42A) Schematic representation of the de novo and salvage pathways for dGTP and DNA-G biosynthesis illustrating the targets of BCX-1777 and DI-82. (FIG. 42B-FIG. 42E) [¹⁵N₅]dG levels in the cell culture media (FIG. 42B), profile of de novo biosynthesis of dGTP and DNA-G (FIG. 42C), profile of salvage biosynthesis of dGTP and DNA-G from [¹⁵N₅]G and [¹⁵N₅]dG (FIG. 42D), and changes in total dNTP levels (FIG. 42E) in the indicated treatment groups following 18 h incubation under the indicated conditions. Jurkat cells were incubated in culture media containing 11 mM [¹³C₆]glucose and 5 μM [¹⁵N₅]dG and were then treated with 5 nM BCX-1777 and/or 1 μM DI-82. (FIG. 42F and FIG. 42G) DNA histograms showing the cell cycle profiles (FIG. 42F) and quantification of the cells in S phase (left panel) and G2/M phase (right panel) (FIG. 42G) following 24 h treatment. (FIG. 42H) Western blot analysis of pChk1 (S345), pChk2 (T68), pH2A.X (S139) and actin following 24 h treatment. (FIG. 42I) A schematic summary of the effects of BCX-1777 and DI-82 on dGTP and DNA-G biosynthesis. *, P<0.05; **, P<0.005; ***, P<0.0005.

FIGS. 43A-43B. (FIG. 43A) CEM T-ALL cells were pulsed with 10 μM EdU, and the EdU labeled population was followed in respective treatments. (FIG. 43A) The progression of labeled asynchronous CEM cells was monitored 8 h after release in fresh media with different treatments. (FIG. 43B) Bar graphs show S-phase duration calculated from the data using a mathematical model (middle panel) and % EdU-negative cells in S phase (right panel).

FIGS. 44A-44F. (FIG. 44A) Schematic representation of de novo and salvage pathways for dCTP/DNA-C biosynthesis illustrating the target of 3-AP and DI-82. (FIG. 44B) Evaluating dCK activity by the uptake of radiolabeled substrate of dCK, [³H]FAC, in CEM cells treated with varying concentrations of 3-AP in the presence or absence of DI-82. (FIG. 44C) A profile of the differential contribution of de novo and salvage pathways on dCTP (left panel) and DNA-C (right panel) biosynthesis following 18 h incubation under the indicated treatment conditions. (FIG. 44D) Time-dependent changes in the levels of total dCTP (left panel) and newly replicated DNA-C (right panel) under indicated treatment conditions. (FIG. 44E and FIG. 44F) Representative bioluminescence images (FIG. 44E) and the quantification of whole-body radiance values (FIG. 44F) of tumor bearing mice treated with indicated conditions. 2×10⁵ luciferase expressing p185 cells were injected intravenously into C57BL/6 female mice for leukemia induction. Treatment was started 7 days post-inoculation of cells. 3-AP (5 mg/kg) and DI-82 (50 mg/kg) were administered i.p. solubilized in PEG-400 and 100 mM Tris-HCl (1:1 v/v) formulation. Results are representative of 2 independent experiments, with n=5 mice/group.

FIGS. 45A-45C. (FIG. 45A) Multiplexed analysis of cell cycle kinetics and DNA damage in CEM cells 10 h after EdU pulse (1 h, CONCENTRATION?). G1* cells represent EdU-positive cells that have completed the S-phase and have returned to G1. Formation of double stranded breaks (DSBs) was also monitored by pH2A.X staining under indicated treatment conditions: yellow, orange and red represent the degree of pH2A.X levels respectively in ascending order. (FIG. 45B) Quantification of pH2A.X cells and the degree of pH2A.X levels in CEM cells treated with indicated drugs for 10 h (FIG. 45C) Quantification G1* cells shown in Panel C.

FIGS. 46A-46E (FIG. 46A and FIG. 46B) Representative bioluminescence images (FIG. 46A) and quantification of whole-body radiance values (FIG. 46B) of tumor bearing mice treated as indicated. 2×10⁵ luciferase expressing p185 cells were injected intravenously into C57BL/6 female mice (n=5 mice/group) for leukemia induction. Treatment was started 7 days post-inoculation of cells. 3-AP (30 mg/kg and 15 mg/kg), DI-82 (50 mg/kg) and VE-822 (40 mg/kg) were administered orally as one solution, solubilized in prototype 9′. (FIG. 46C) Schematic representation of the treatment schedule of C57BL/6 mice bearing syngeneic systemic BCR-ABL p185⁺/Arf^{-/-} pre B-ALL cells (top panel) and representative bioluminescence images at indicated days post tumor inoculation (bottom panel). q.d. and b.i.d. stands for once/day and twice/day respectively. (FIG. 46D-FIG. 46F) Quantification of whole body radiance values (FIG. 46D), Kaplan-Meier survival curves (FIG. 46E) and the body weight measurements (FIG. 46F) in the mice in Panel C.

FIGS. 47A-47F. (FIG. 47A) Schematic representation of development of Dasatinib-resistant BCR-ABL p185⁺/Arf^{-/-} cells. p185 pre-B ALL cells inoculated mice were treated with 10 mg/kg q.d. (once/day) for 20 days. (FIG. 47B) Dasatinib resistant cells harbor the T315I gatekeeper mutation (left panel) and are resistant to Dasatinib (1 nM). (FIG. 47C) Schematic representation of the treatment schedule. (FIG. 47D to FIG. 47F) Representative bioluminescence images (FIG. 47D), quantification of whole body radiance values (FIG. 47E), and Kaplan-Meier survival curves (FIG. 47F) in the mice in Panel C (N=4 for control group 20 for treated group. Bioluminescence images for 10 out of 20 mice were displayed). 65% of mice (13 out of 20) had no detectable disease at the end of treatment (42 days post tumor inoculation).

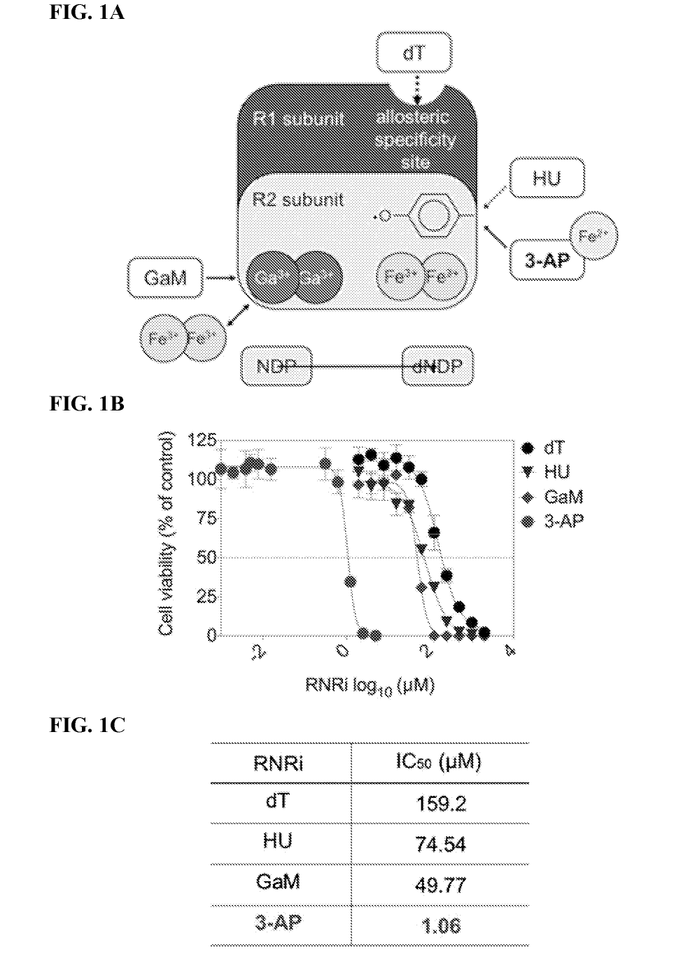

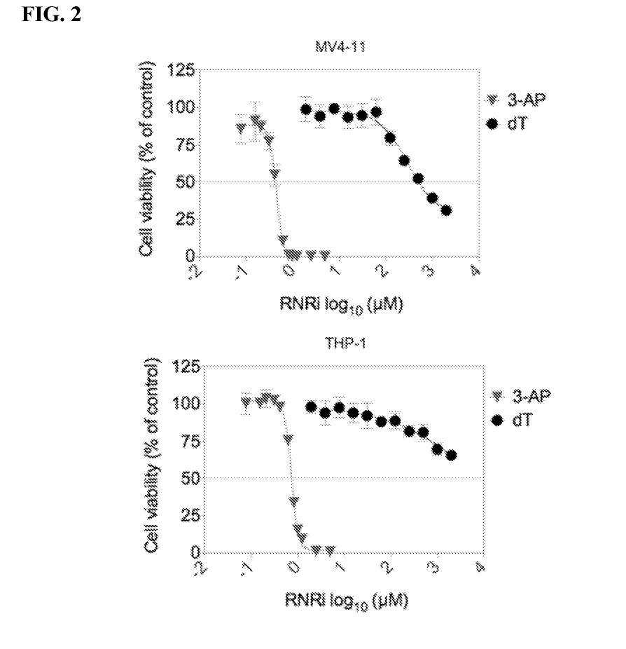

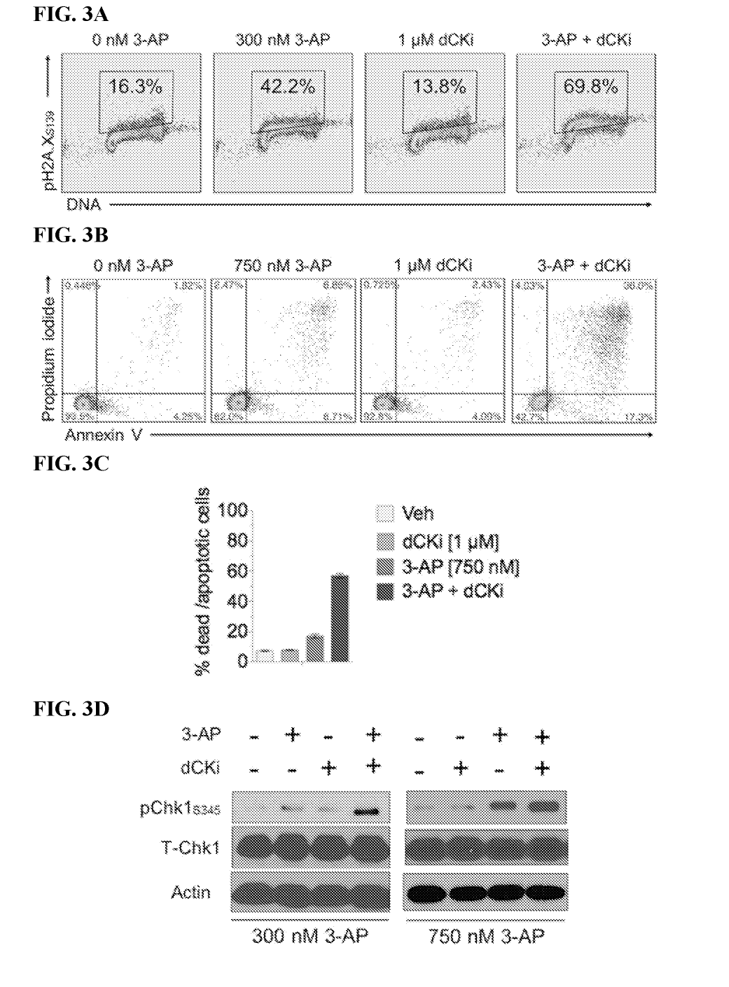

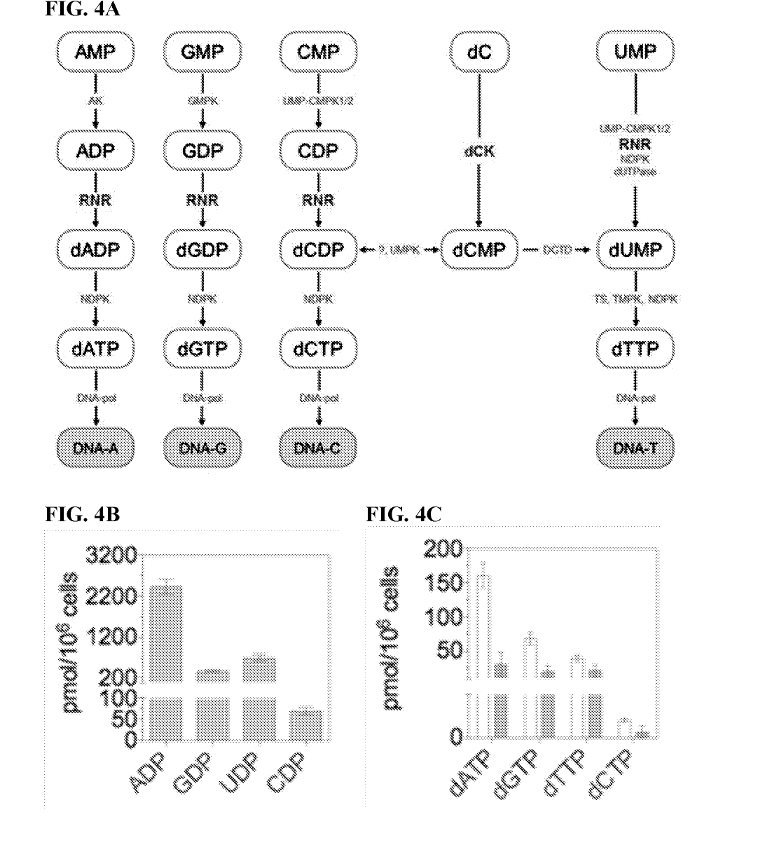

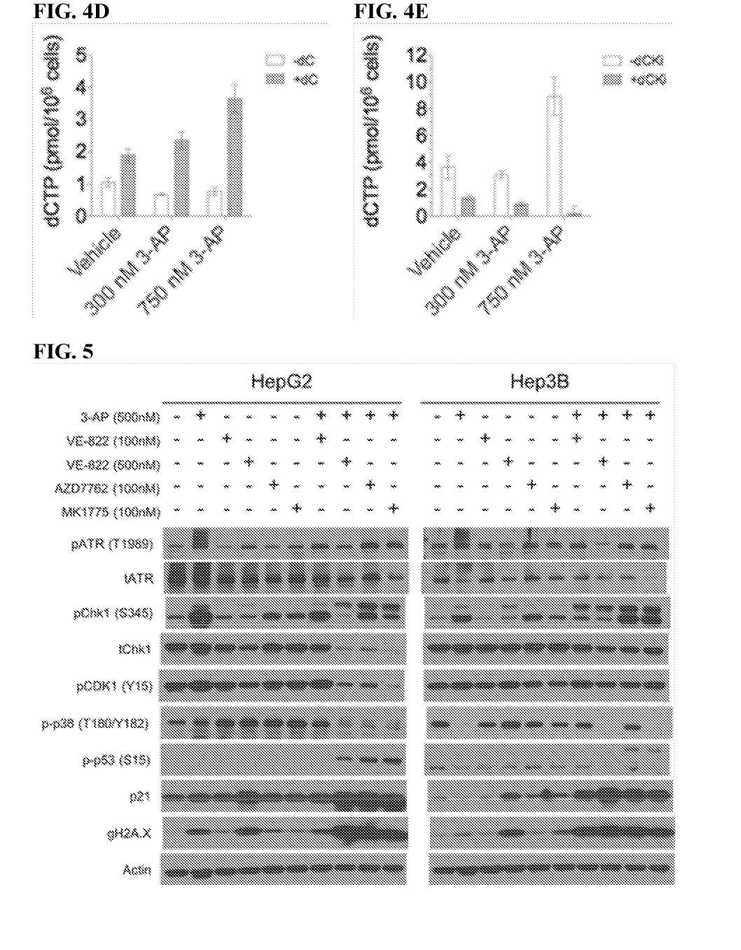

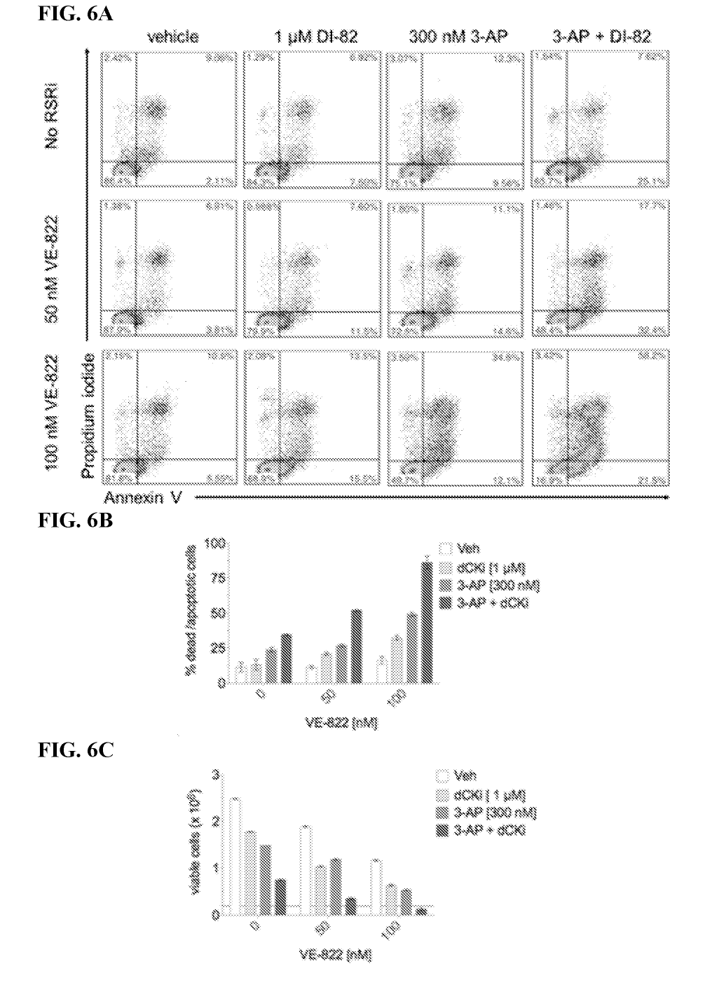

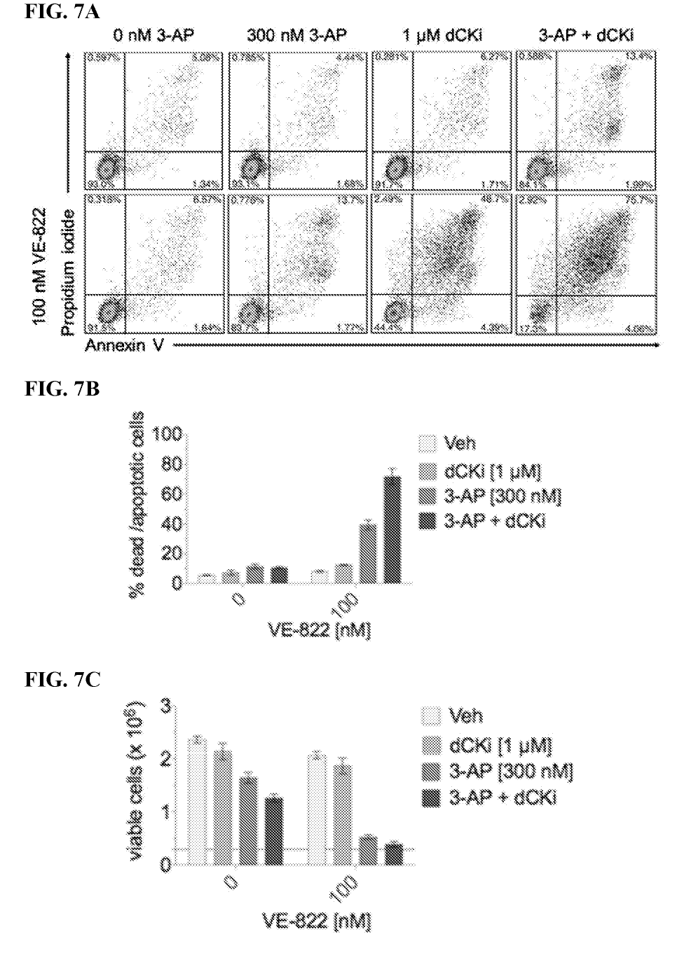

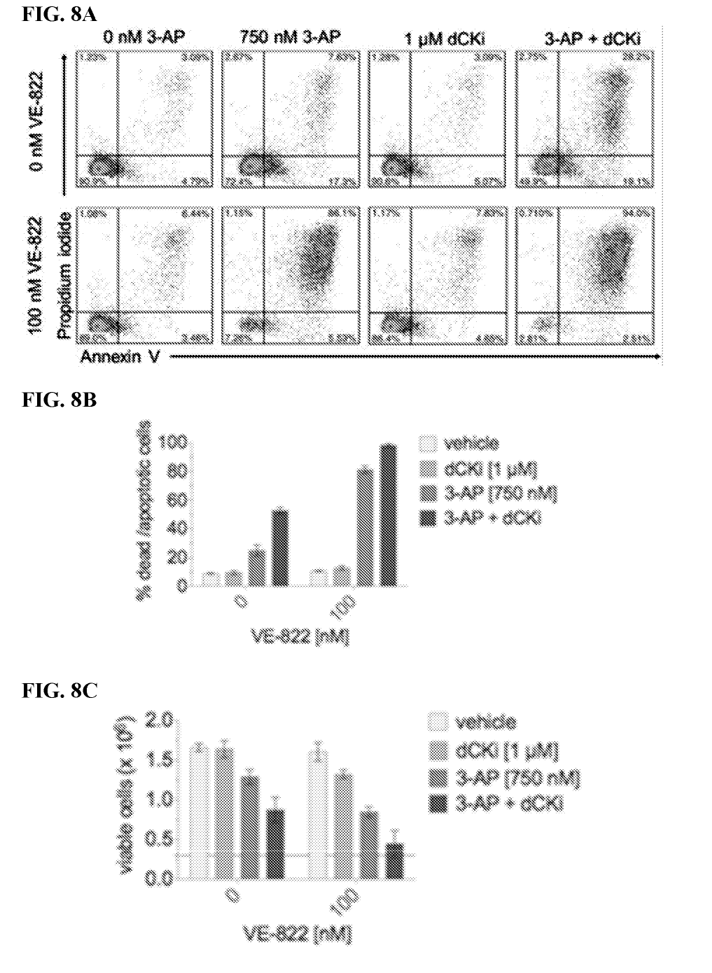

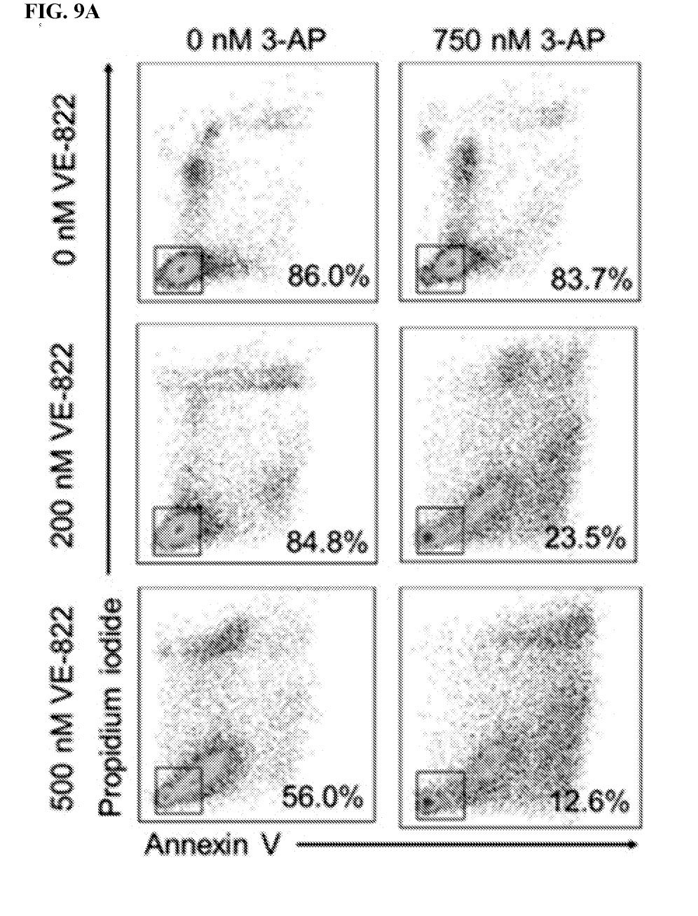

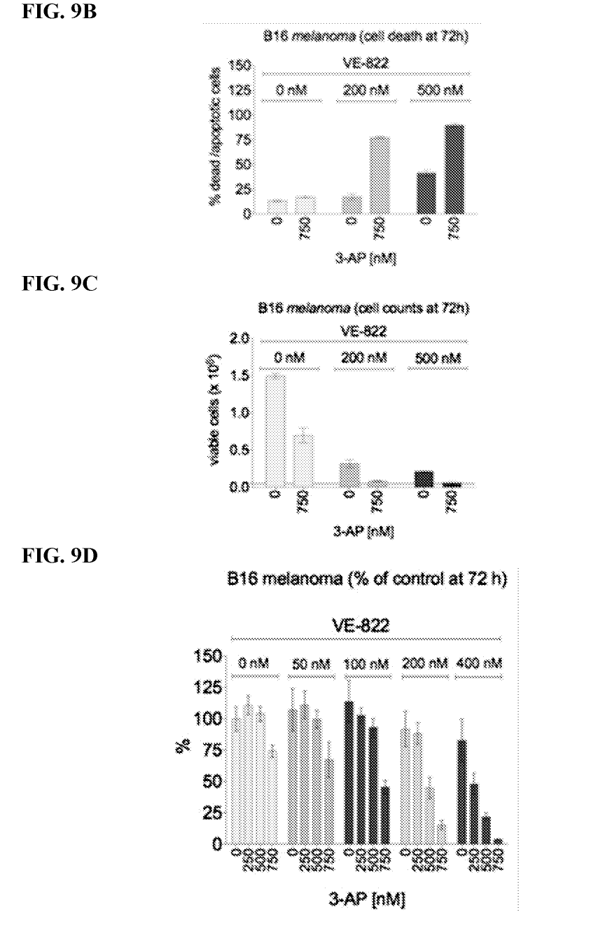

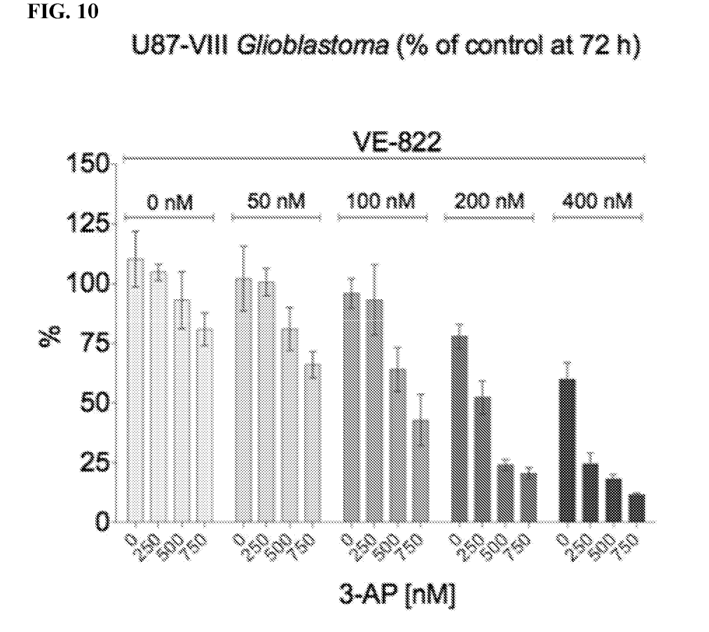

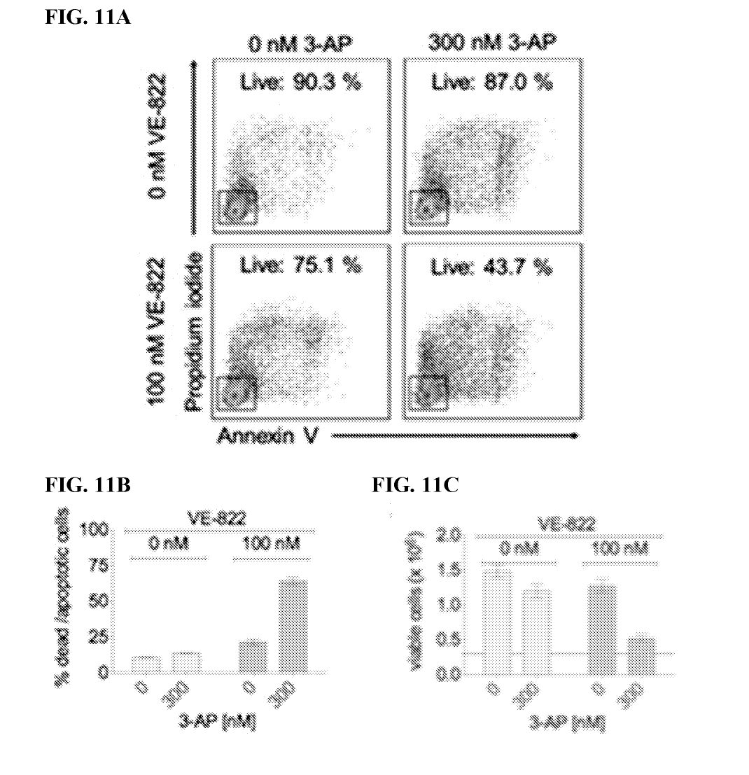

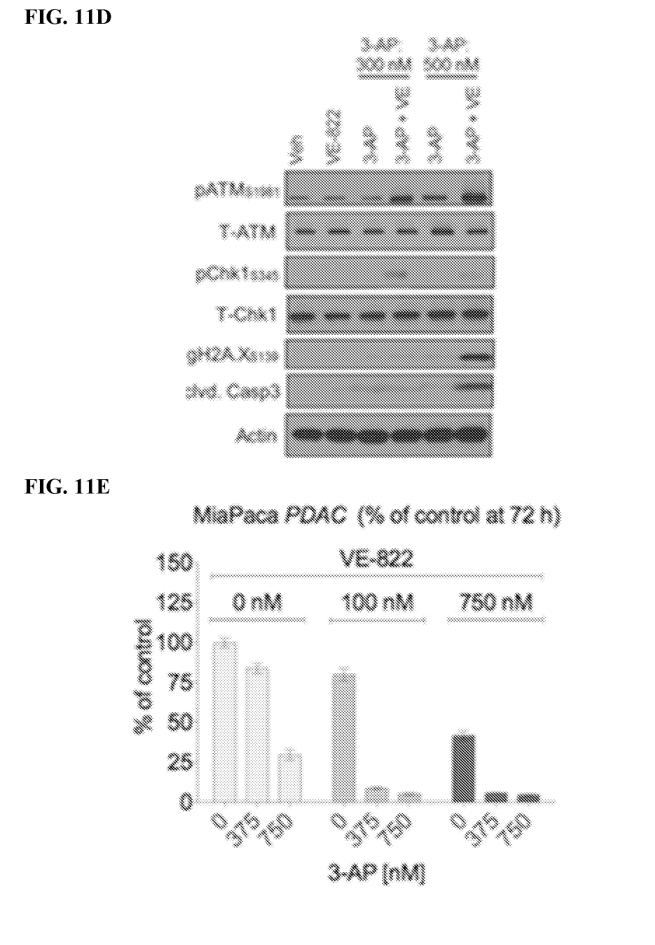

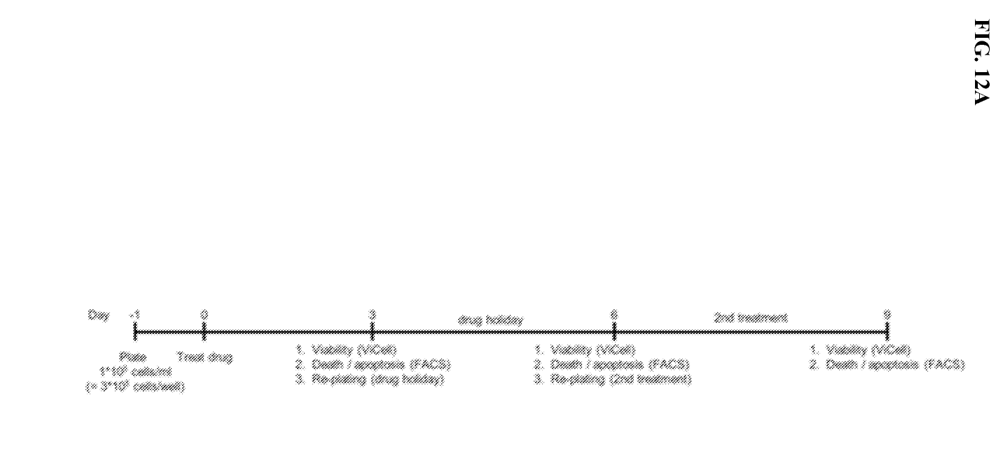

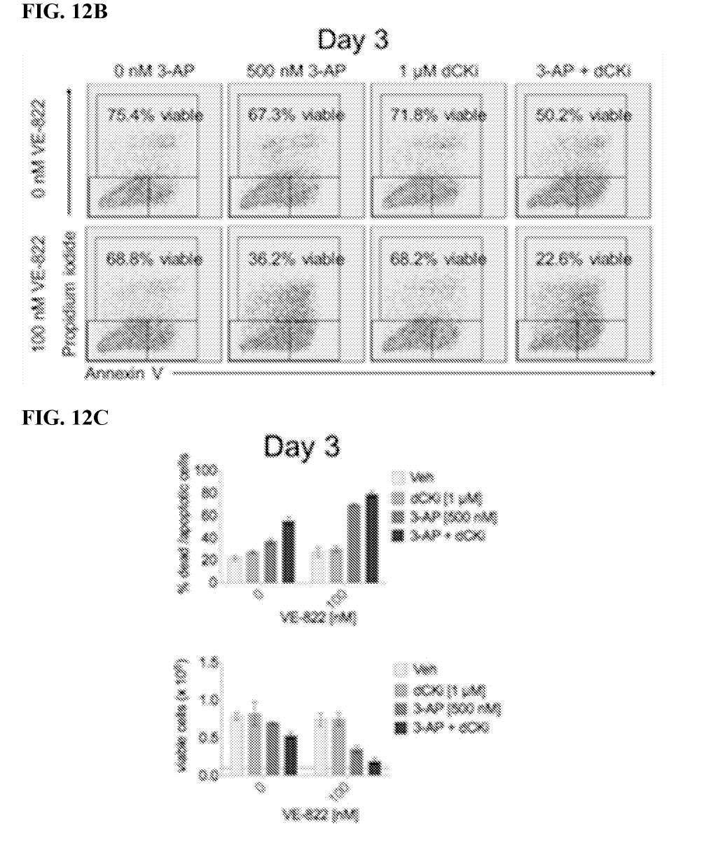

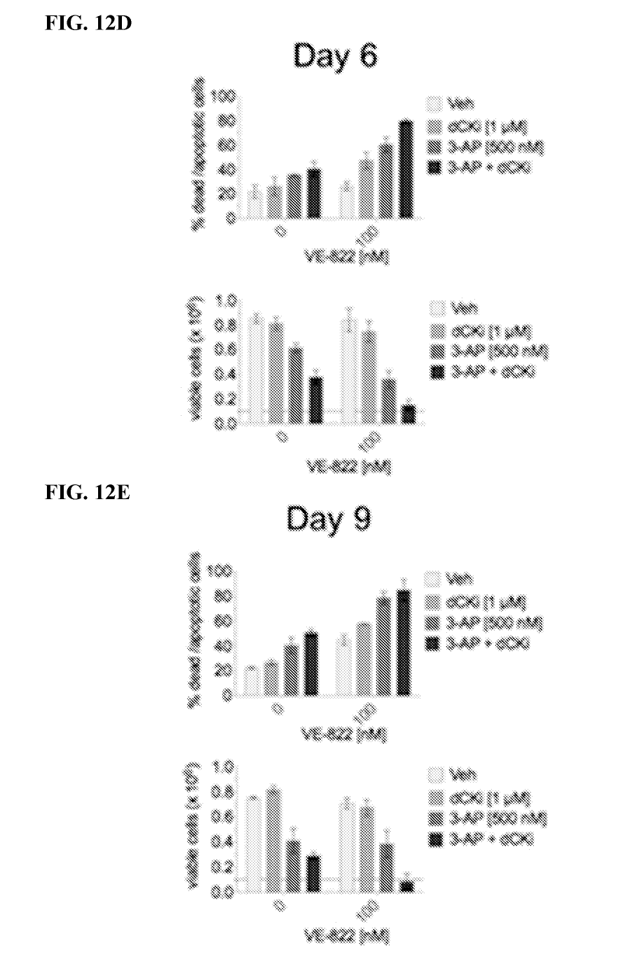

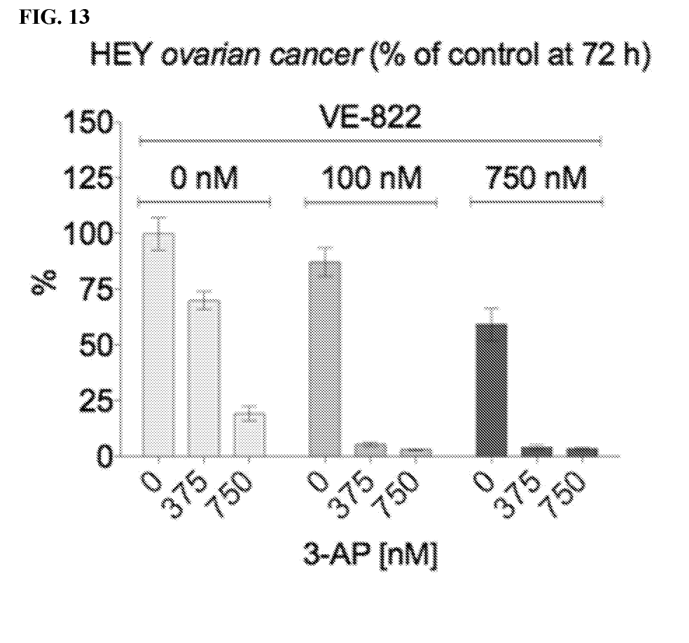

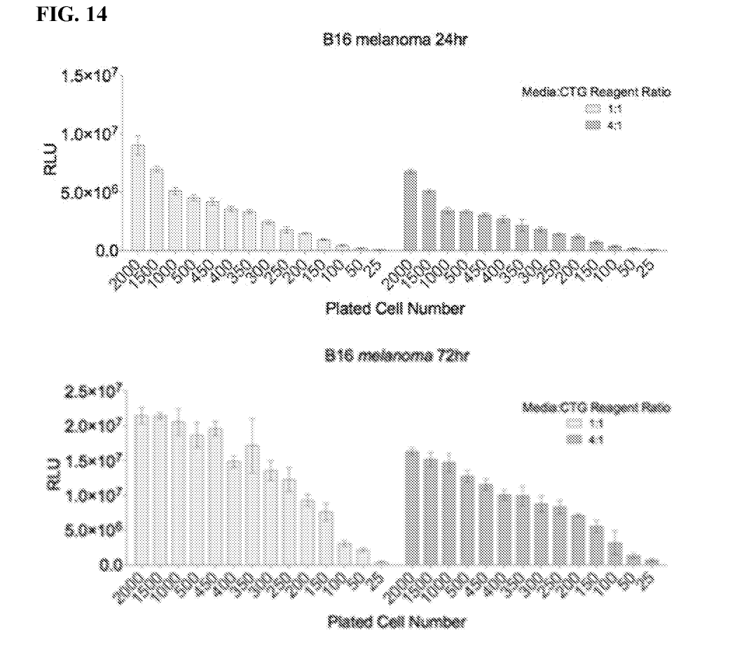

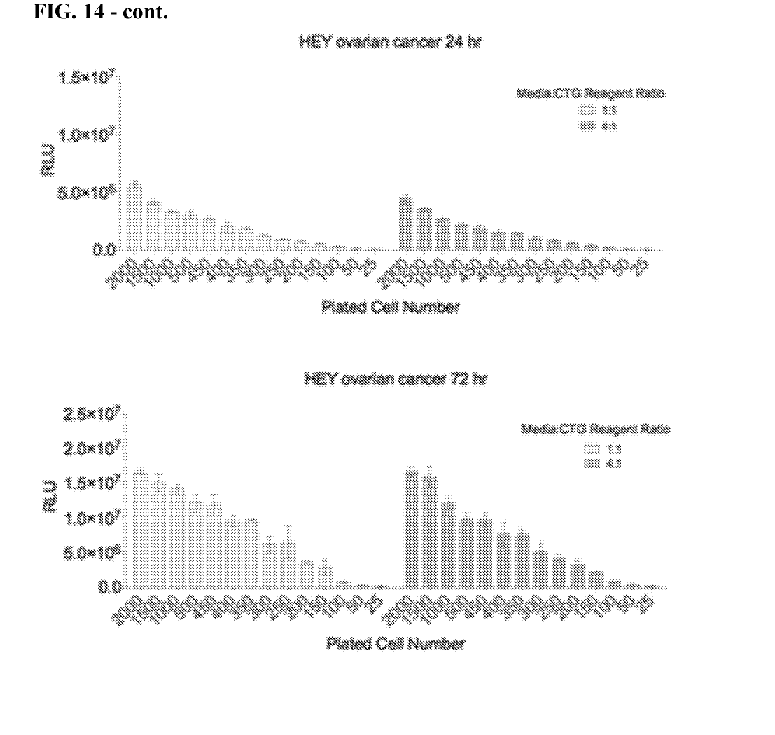

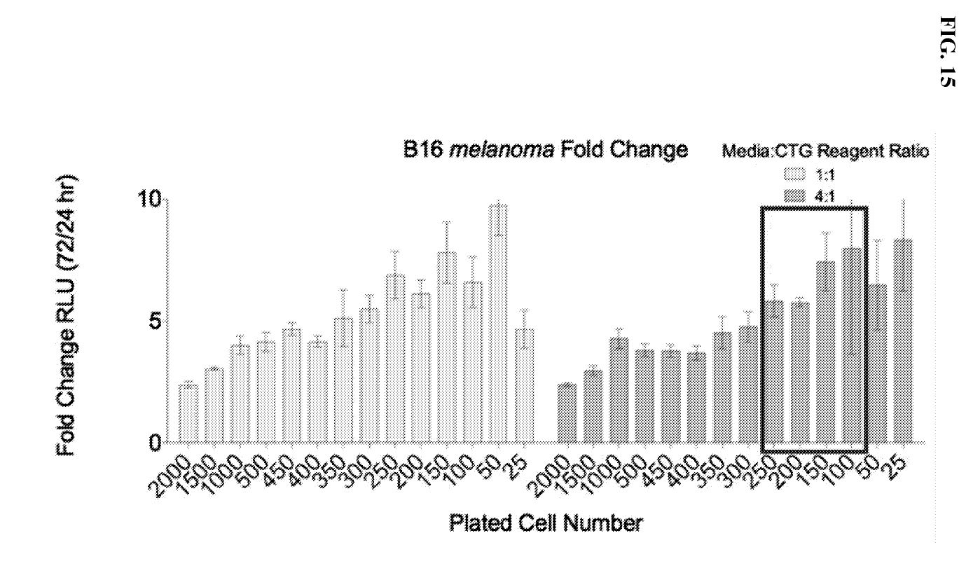

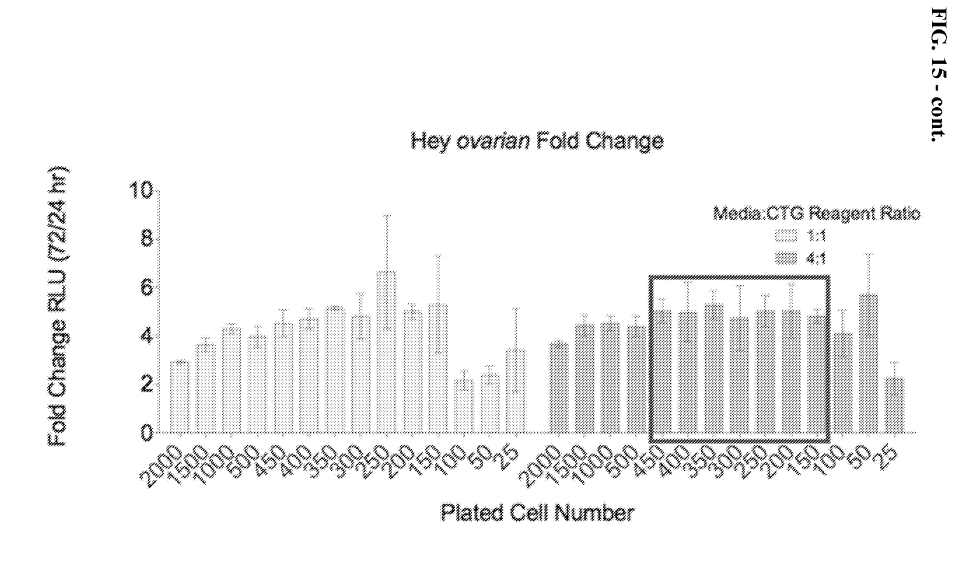

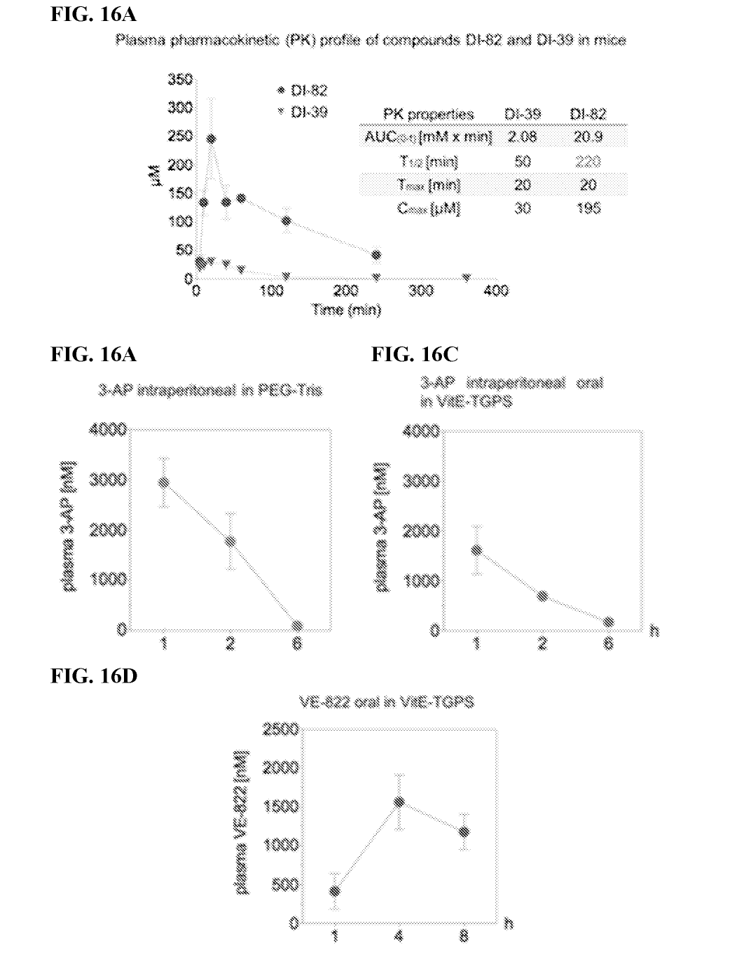

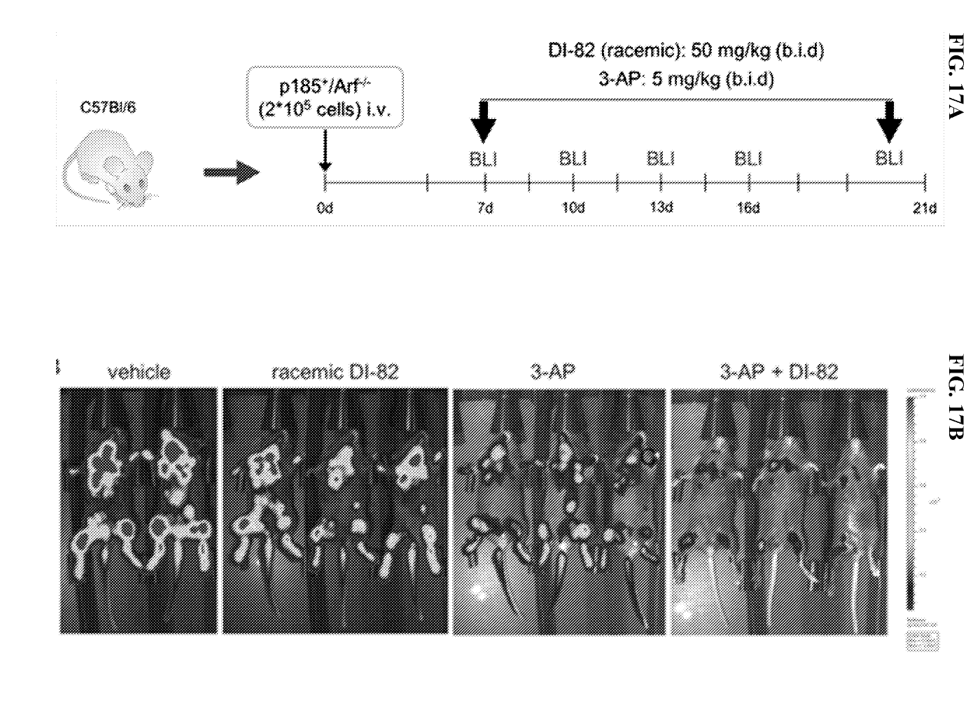

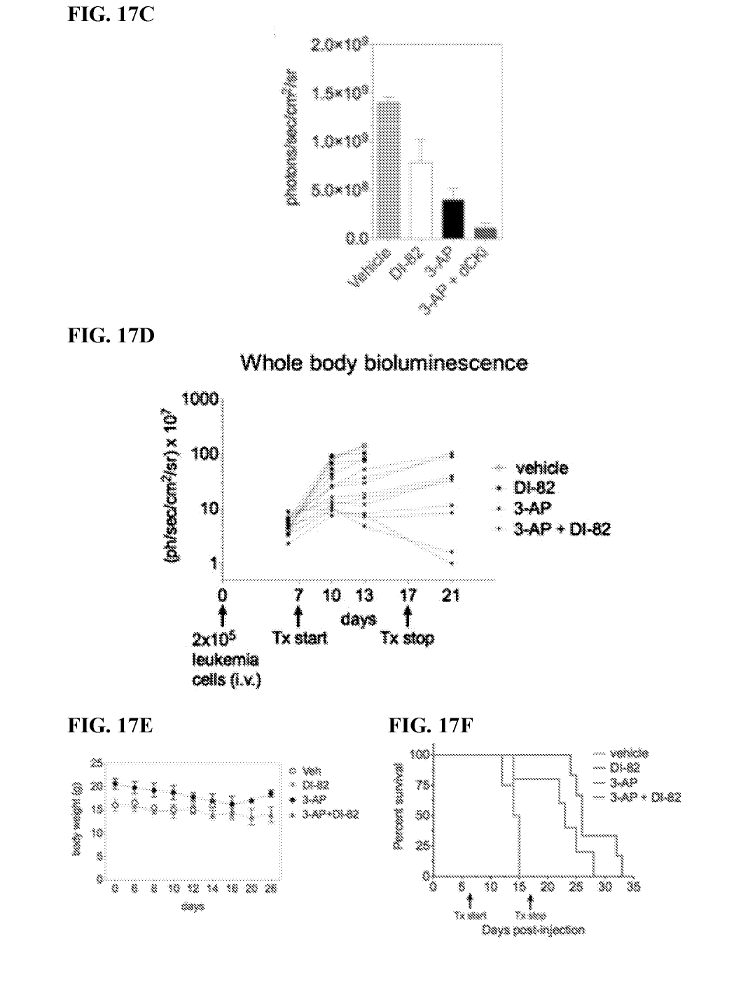

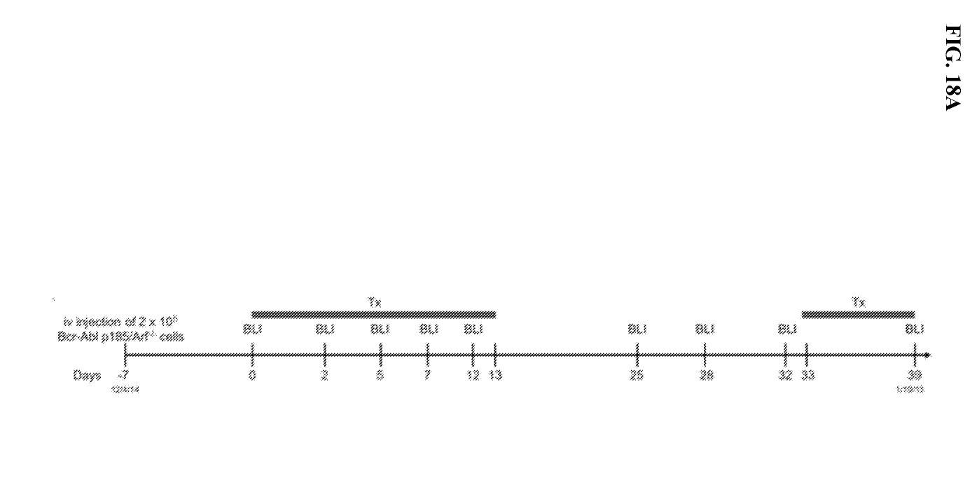

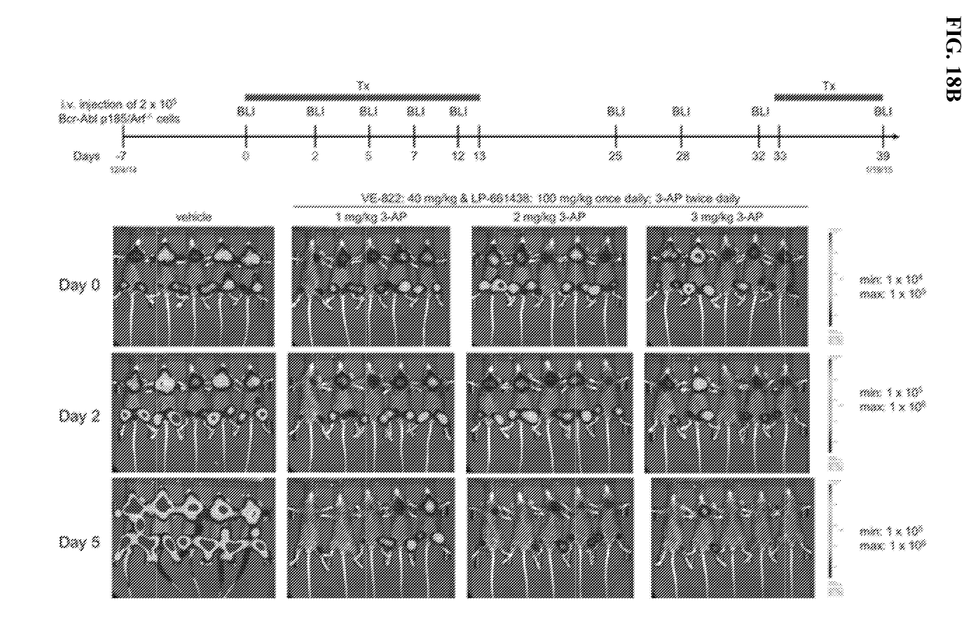

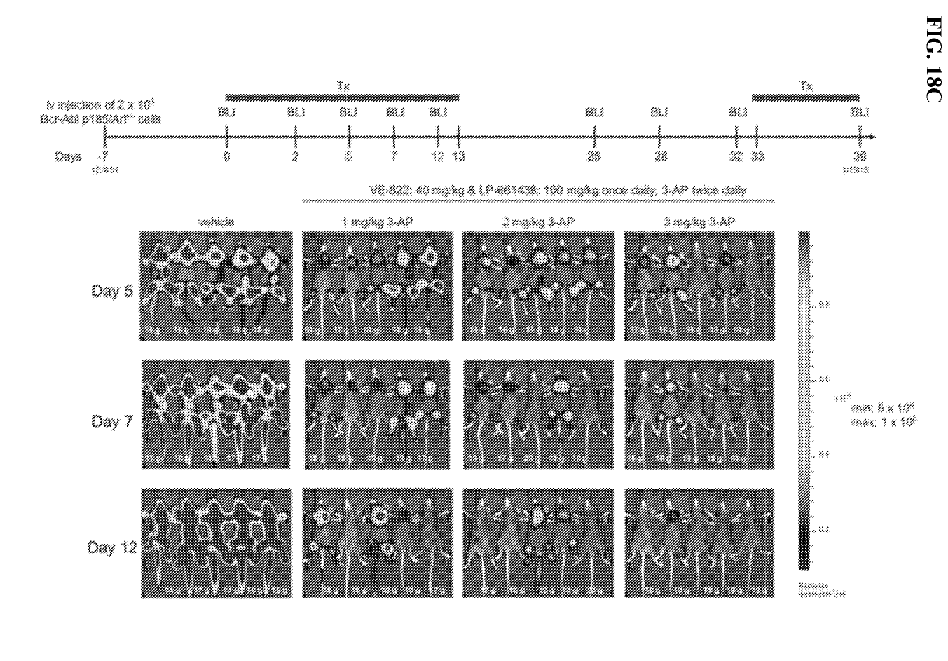

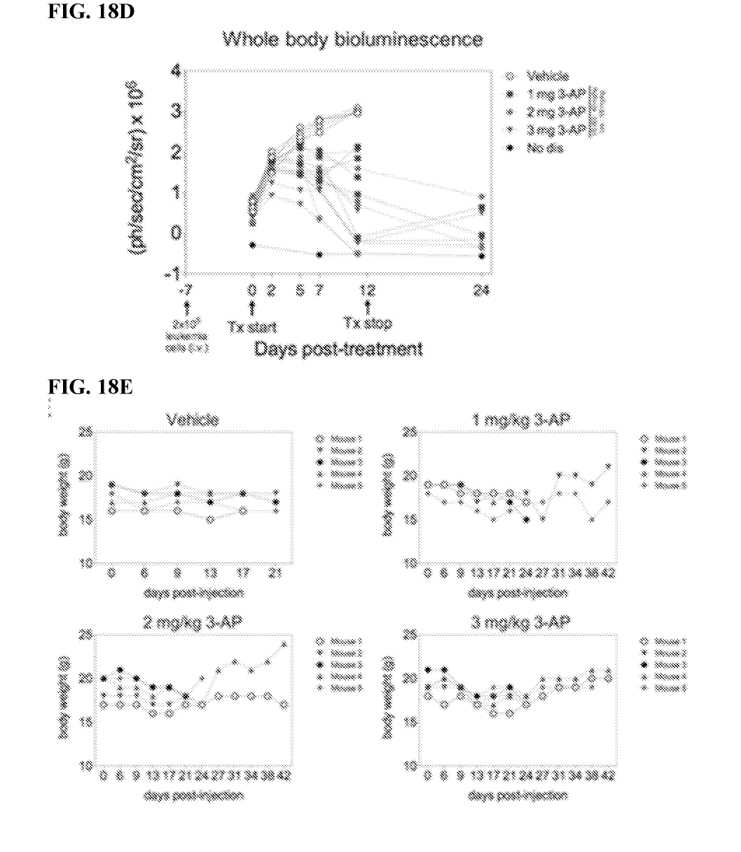

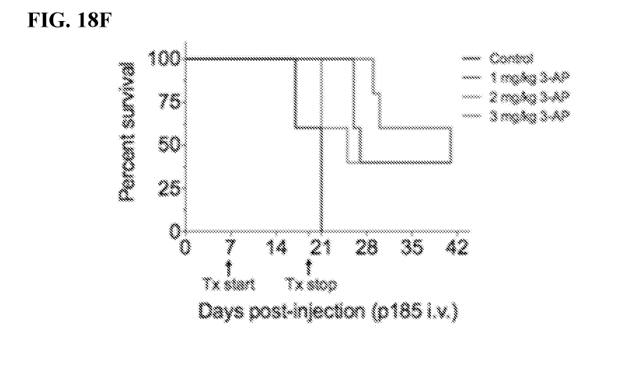

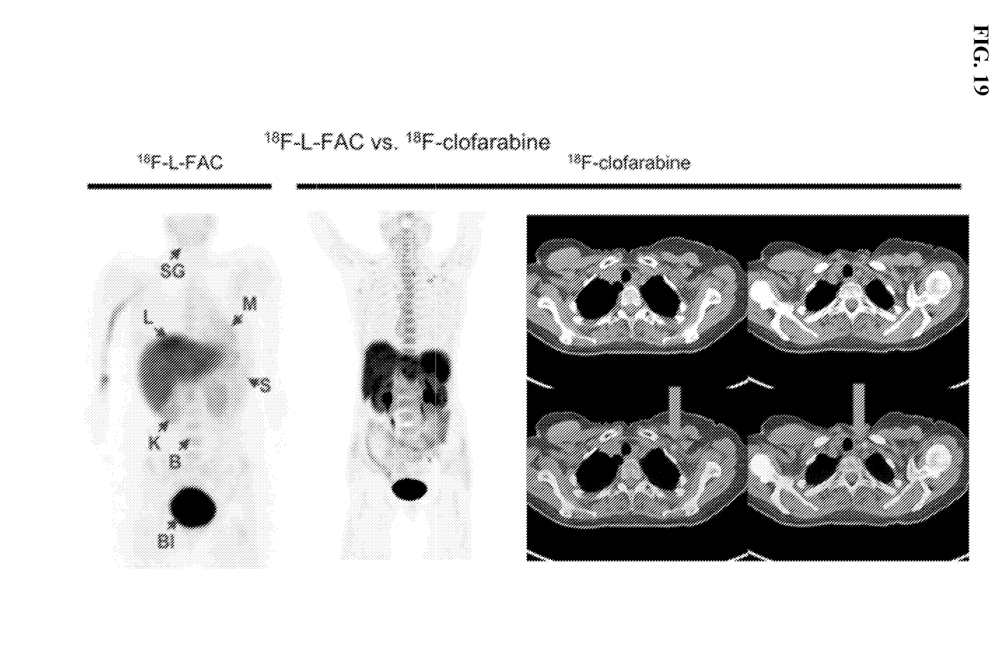

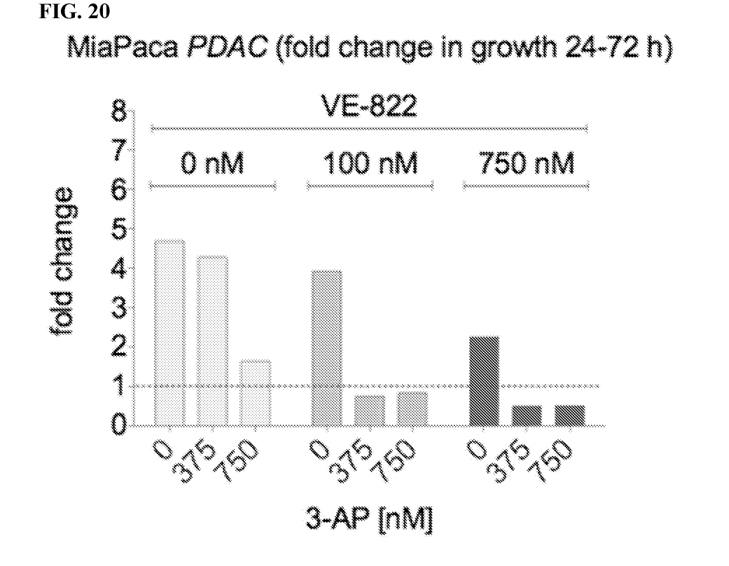

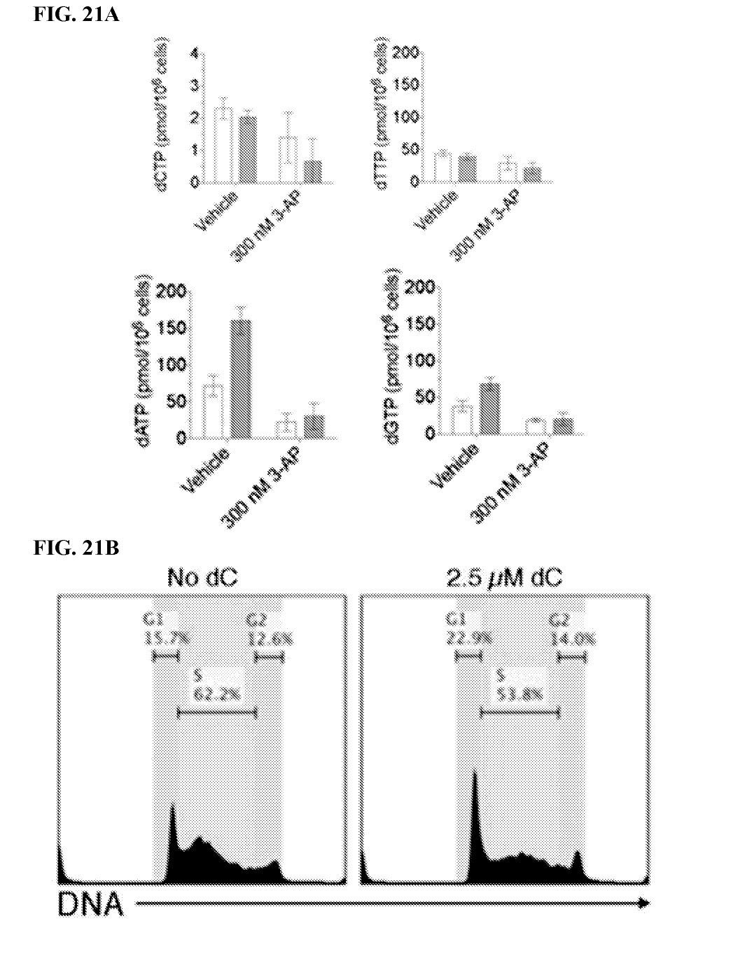

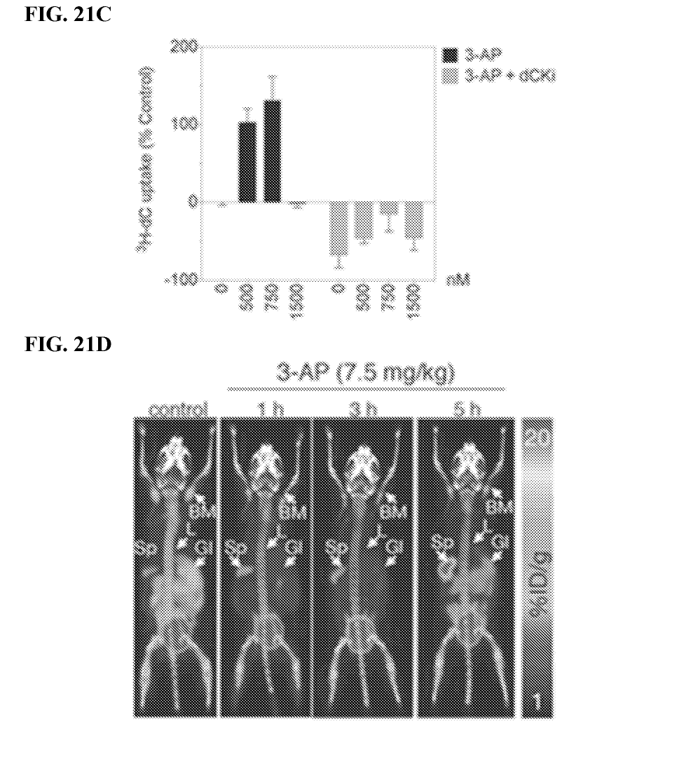

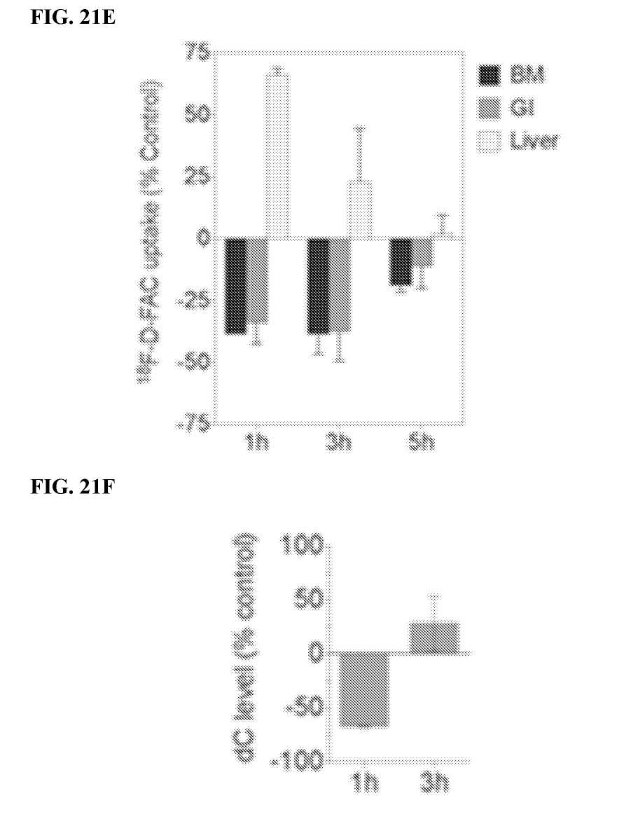

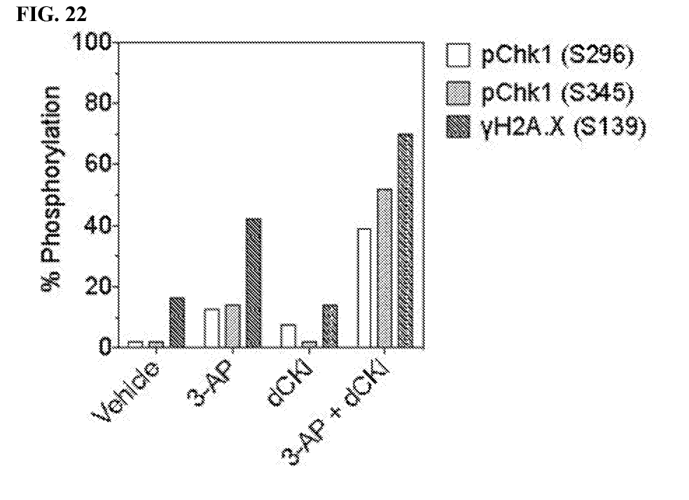

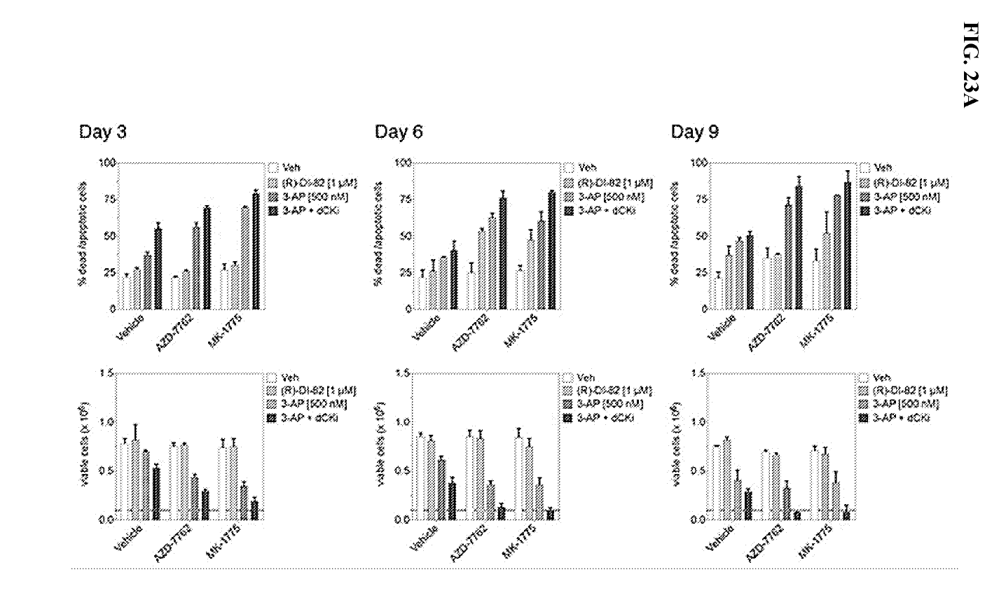

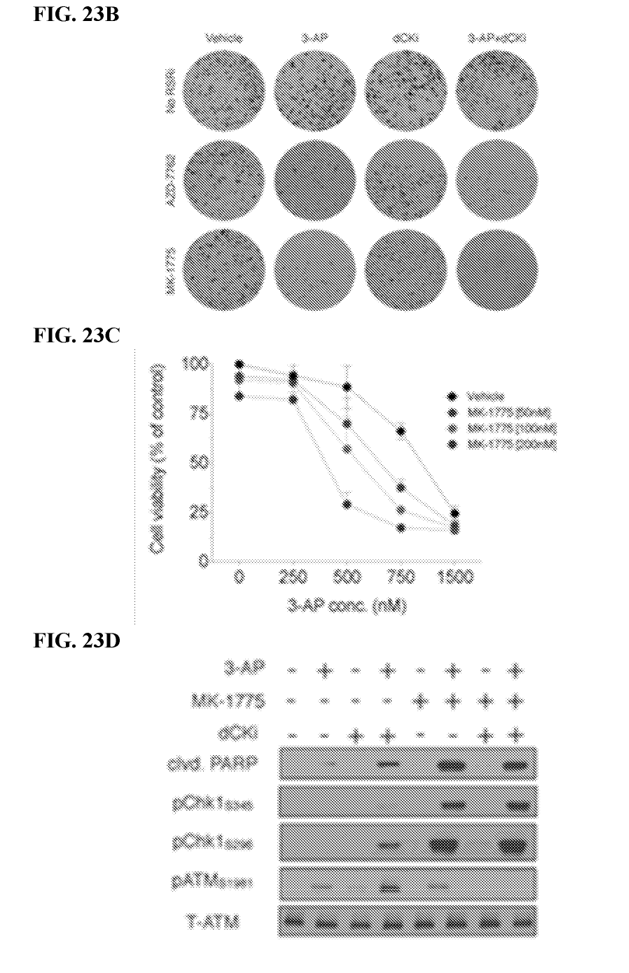

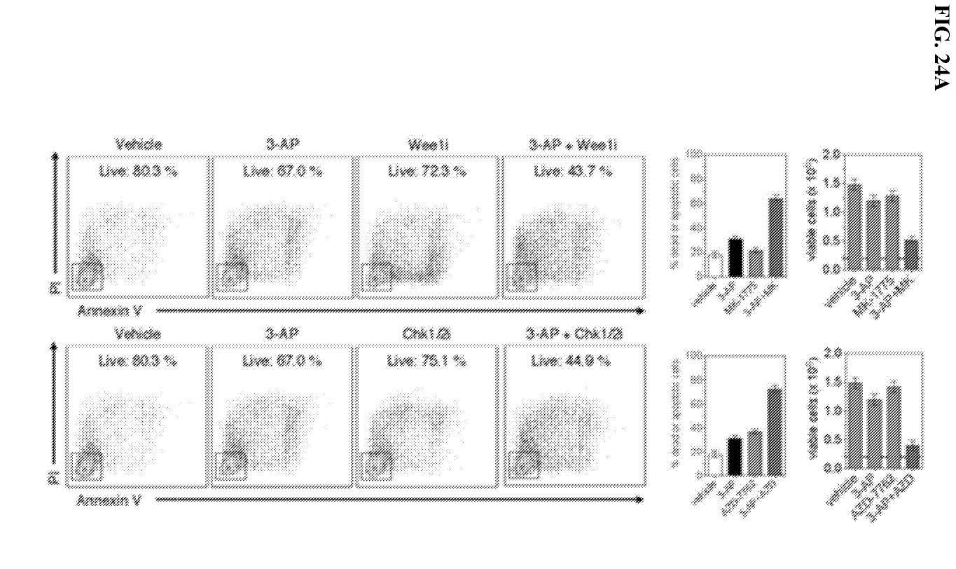

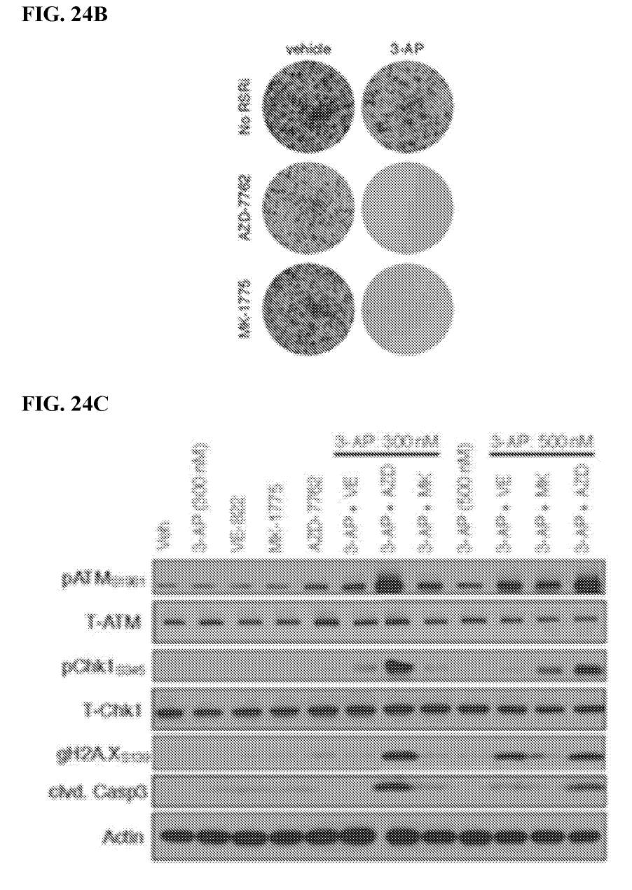

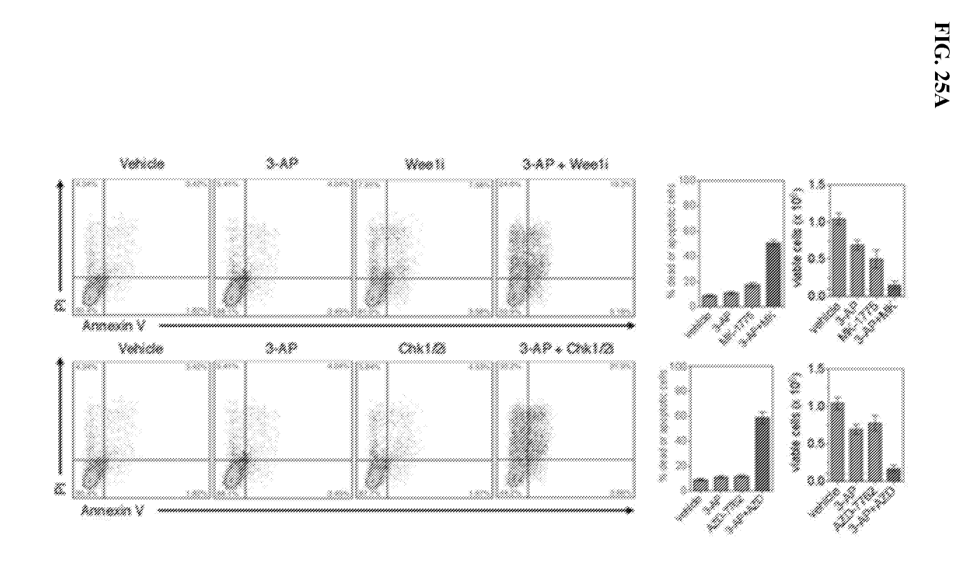

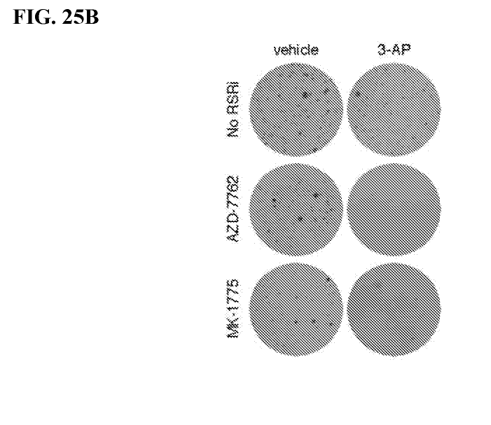

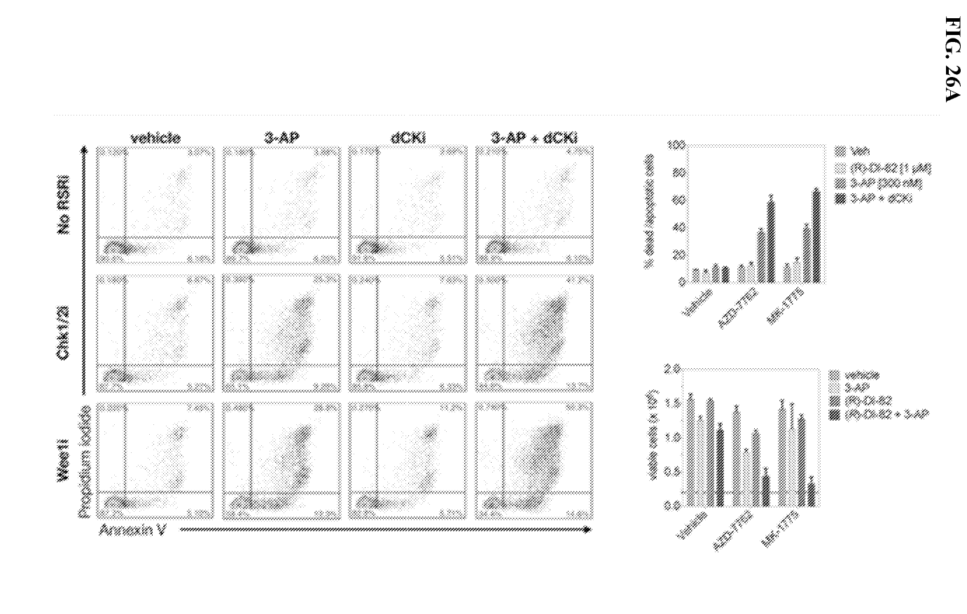

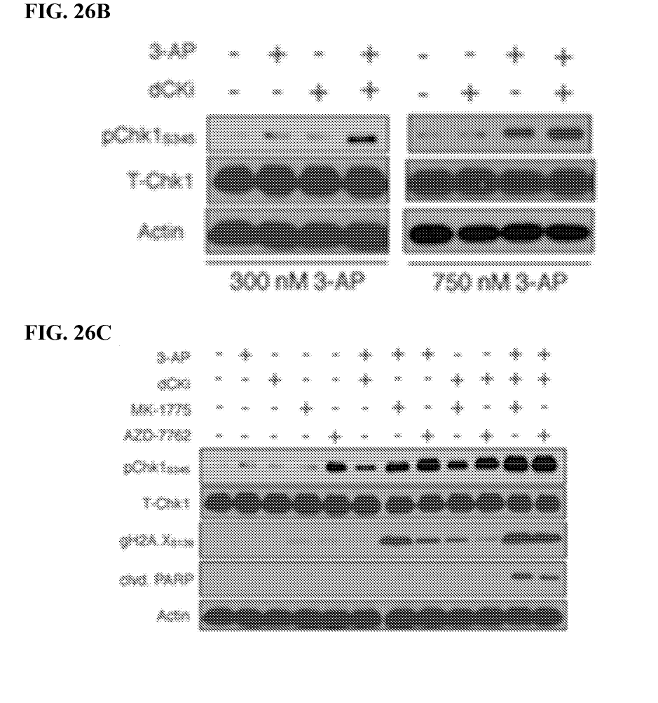

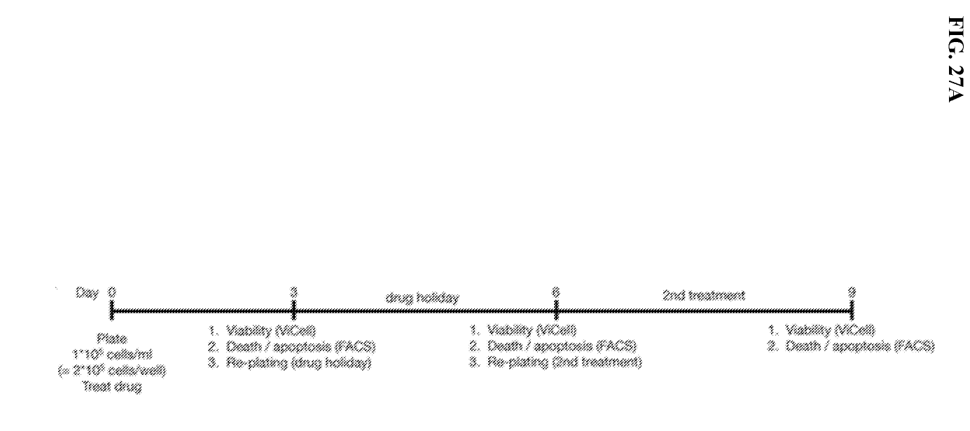

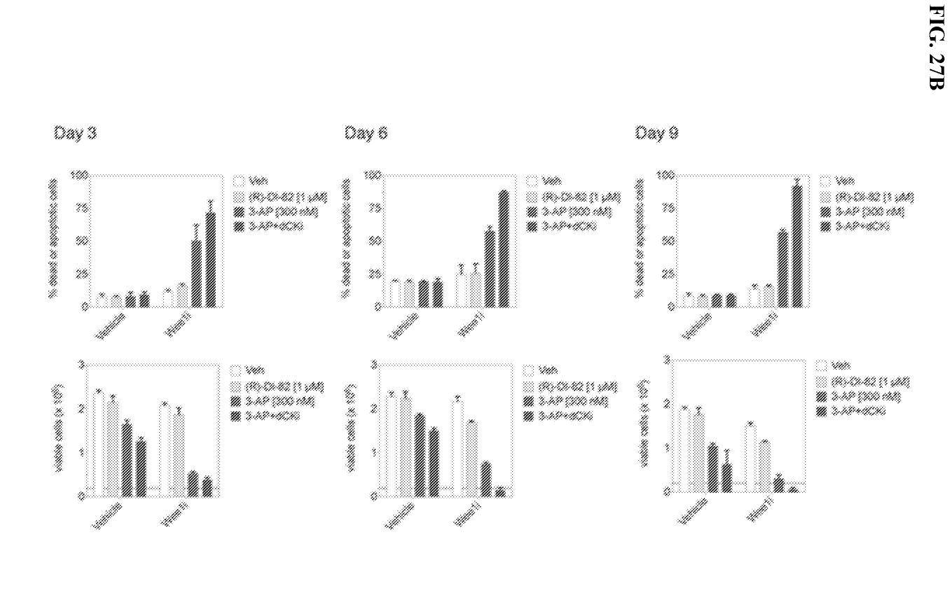

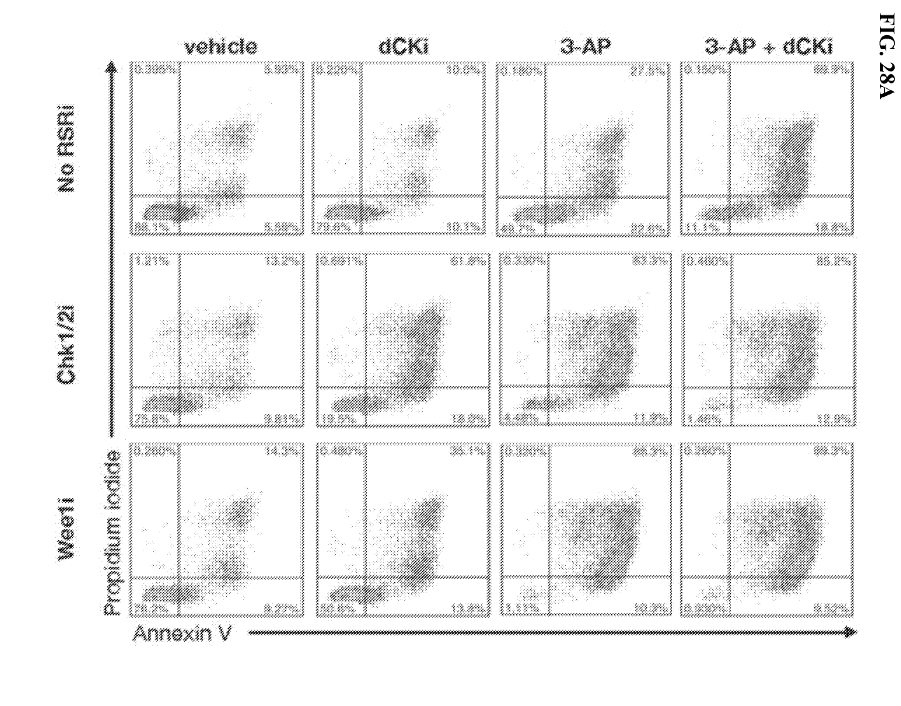

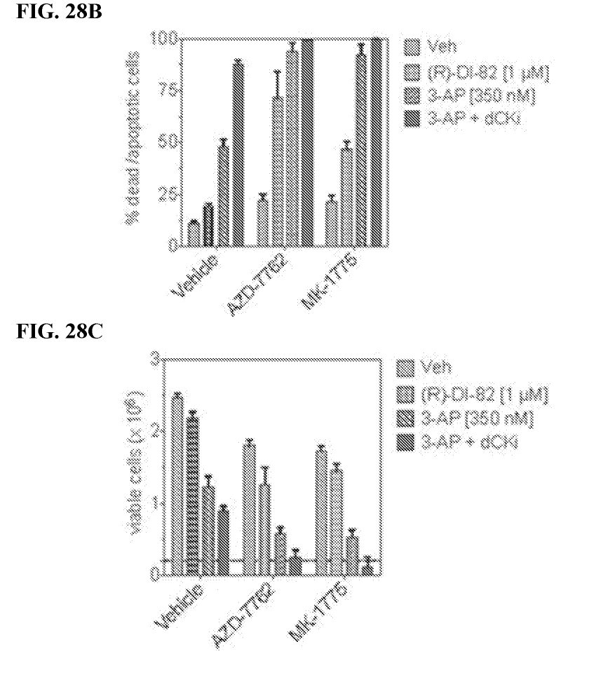

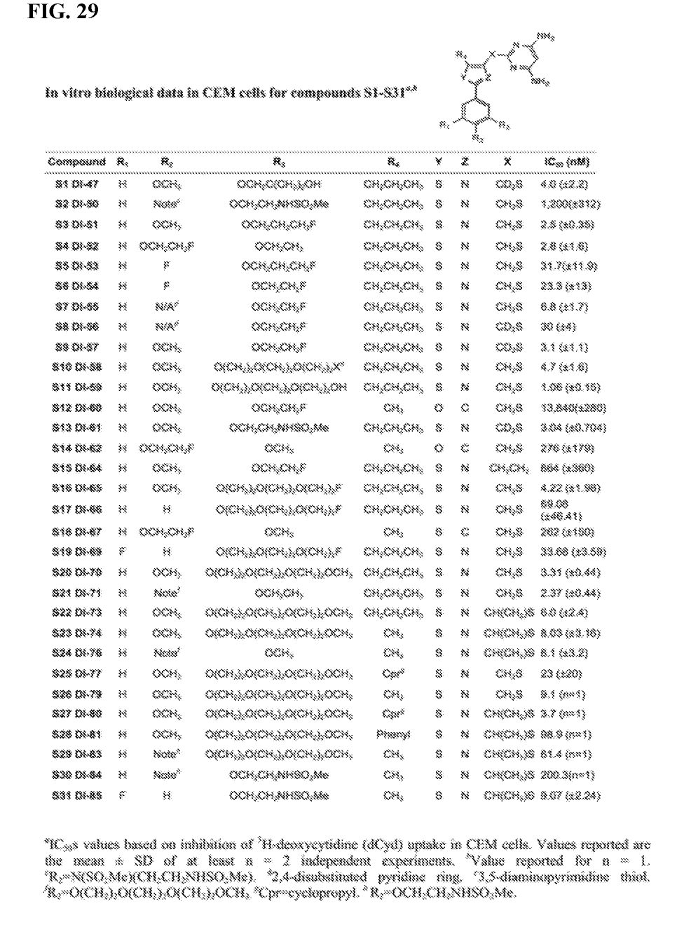

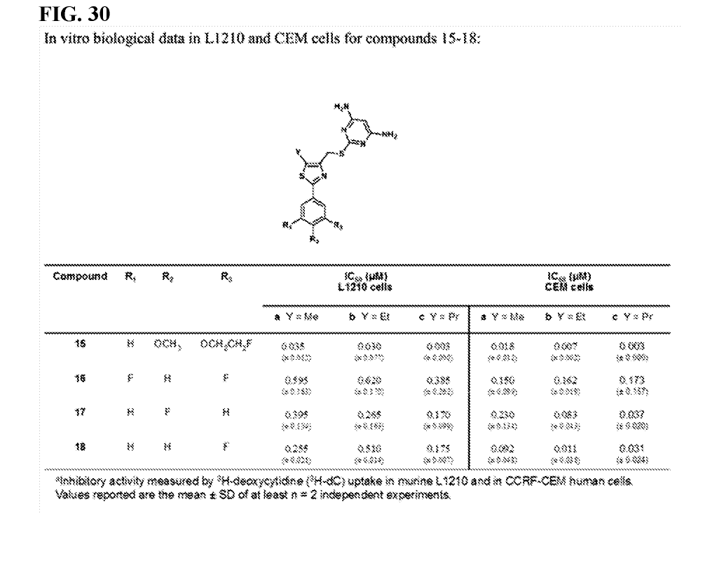

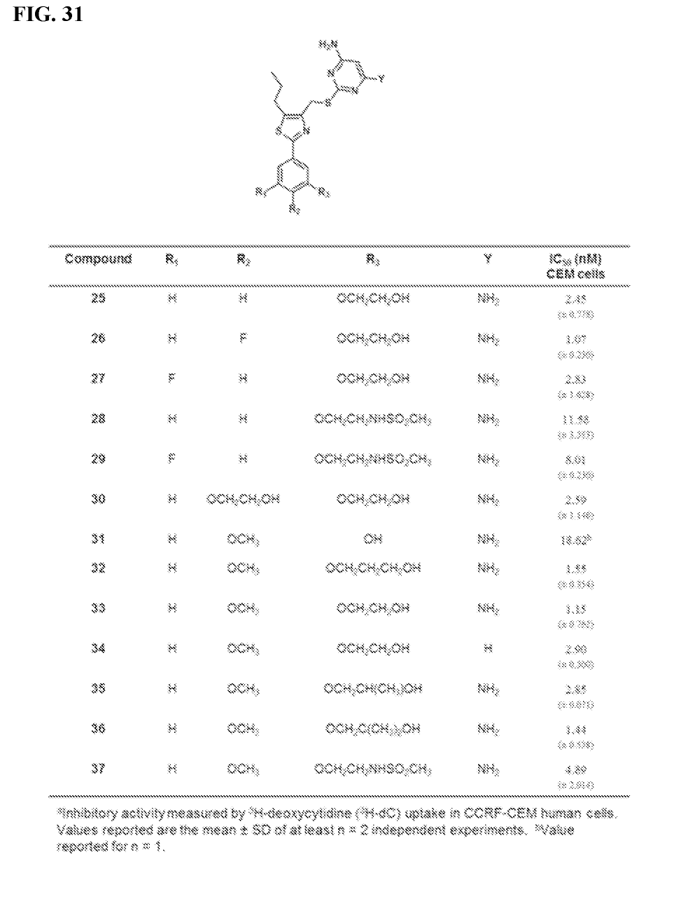

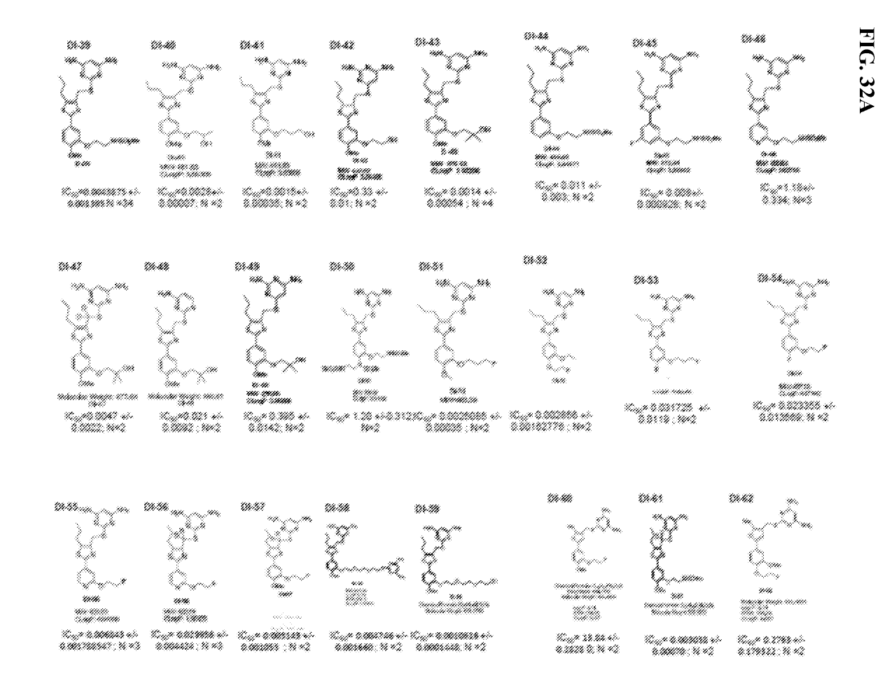

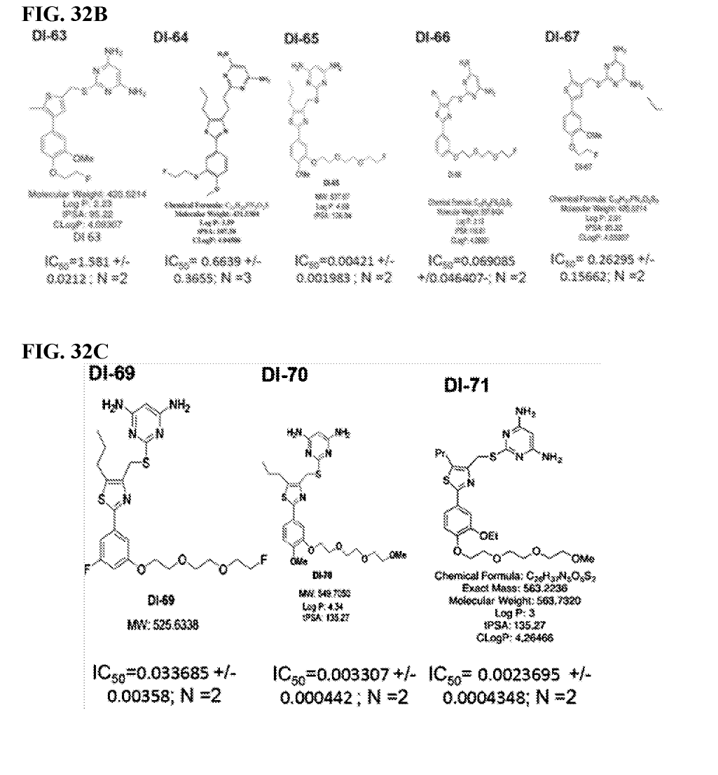

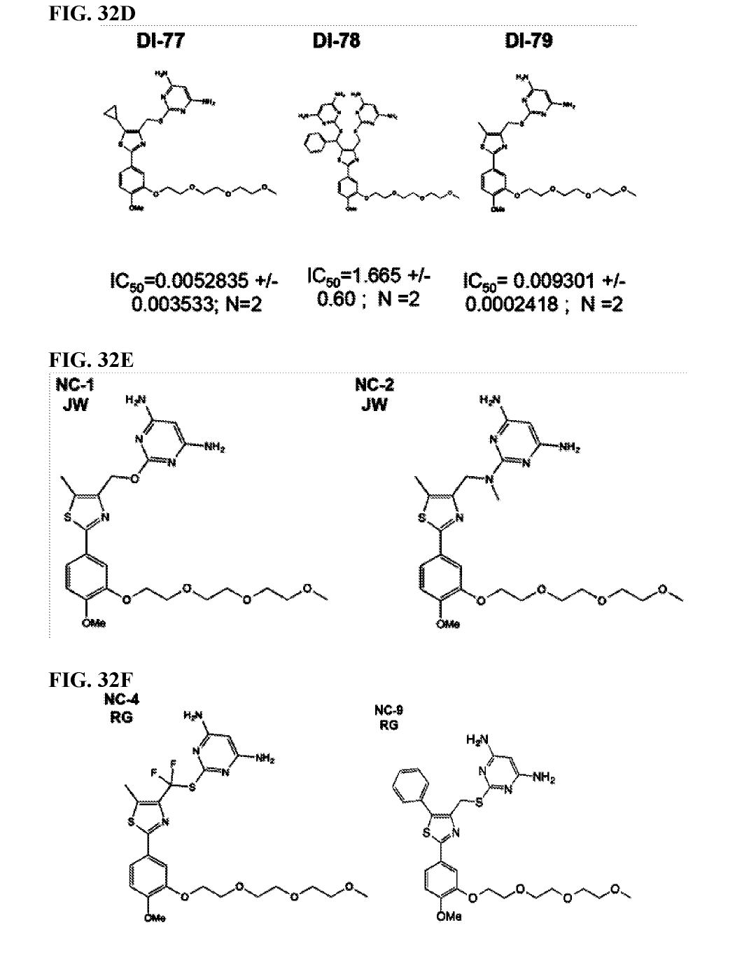

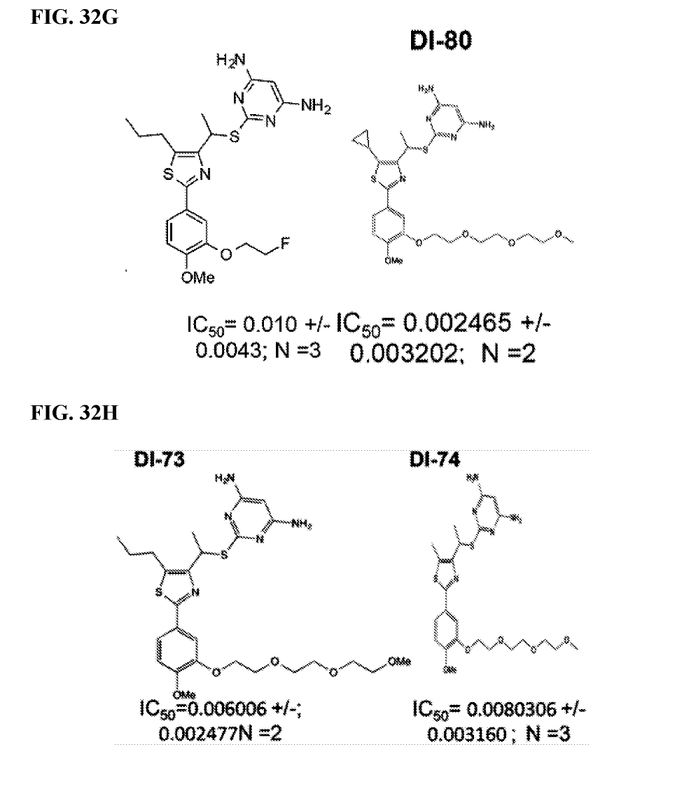

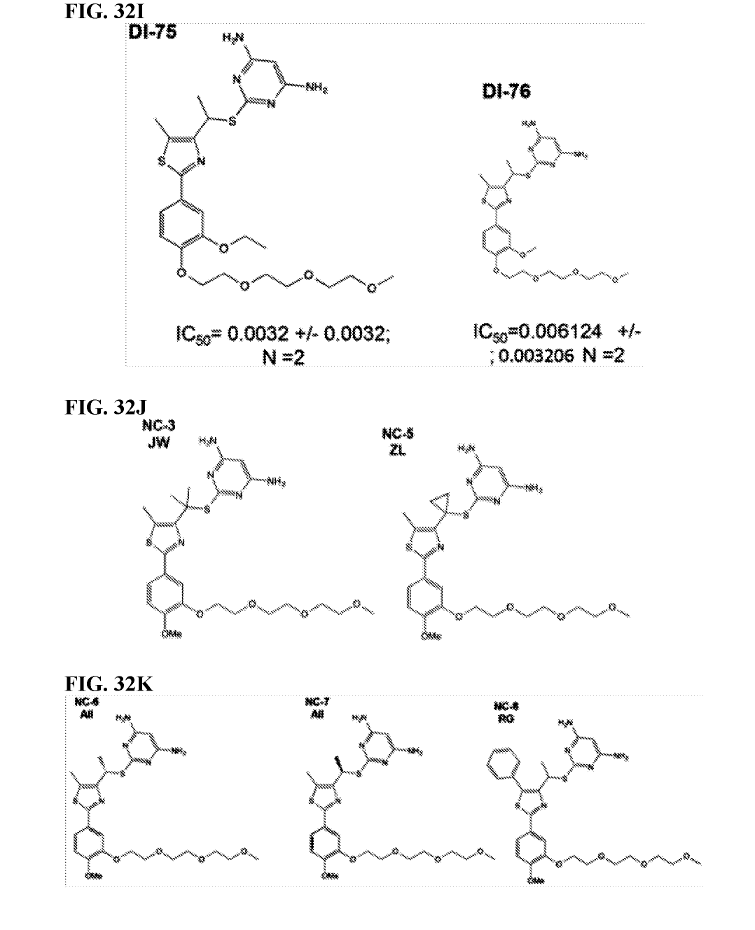

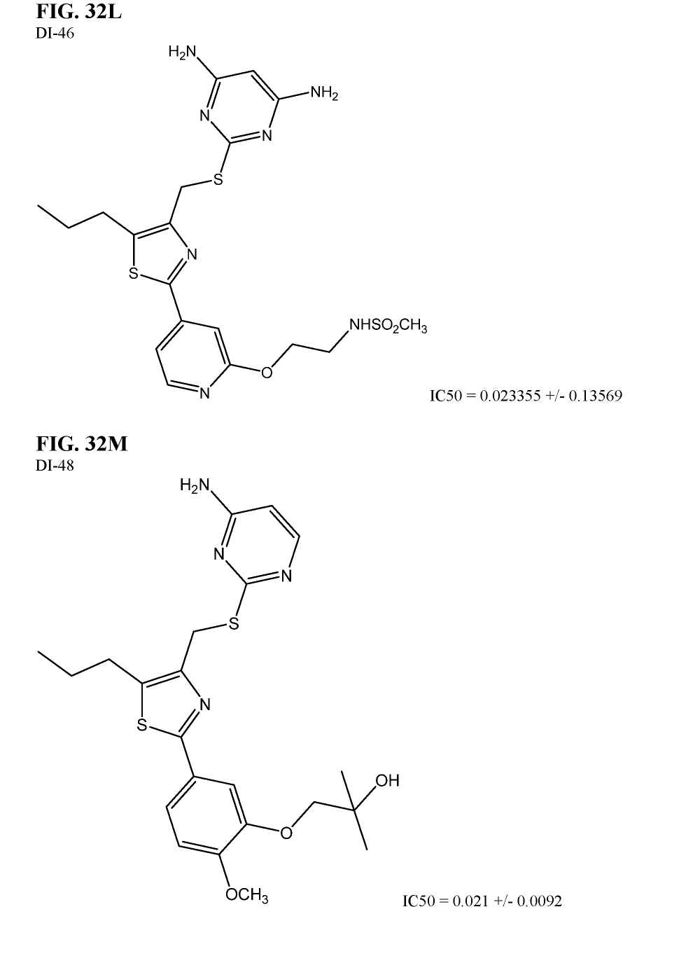

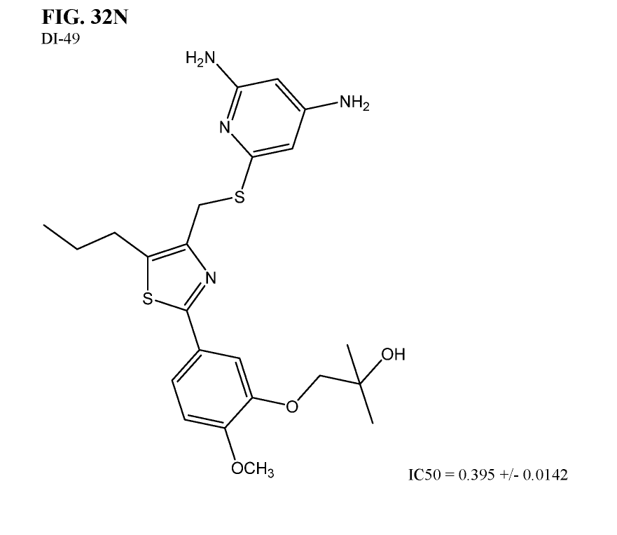

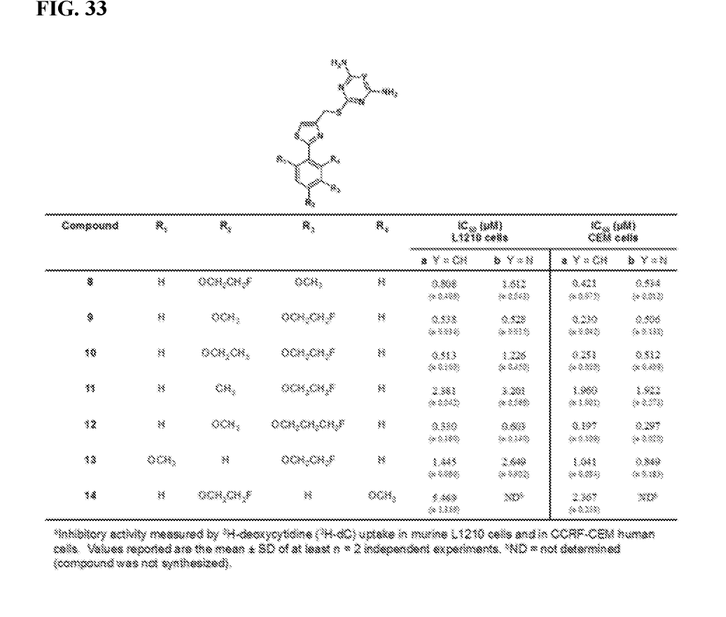

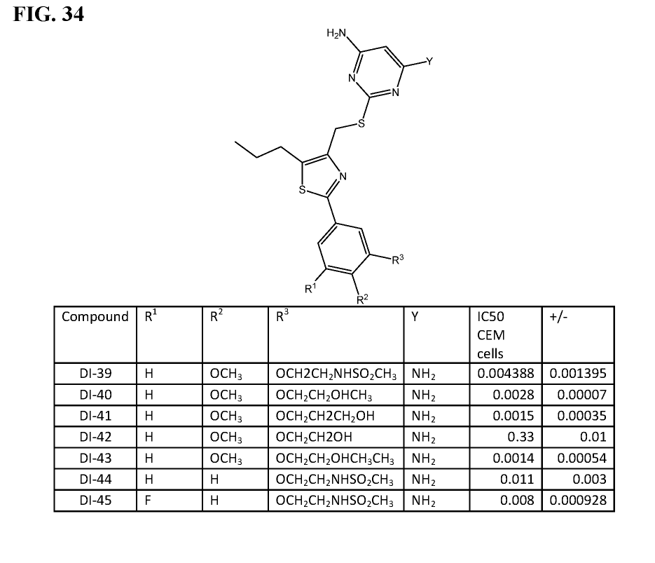

Cancer cells may be more sensitive than normal cells to perturbations of nucleotide metabolism and to inhibition of a signaling pathway termed the Replication Stress Response pathway. Of the four deoxyribonucleotide triphosphates (dNTP) required for DNA replication, deoxycytidine triphosphate (dCTP) is the most susceptible to pharmacological depletion, since it is present in low amounts, and therefore it is the first dNTP to become rate-limiting for DNA replication. The functional redundancy in nucleotide metabolism may be a cause of single target treatment failure. Another possible cause of single treatment failure may be adaptation mechanisms through the Replication Stress Response pathway. Targeting of new or additional targets in the salvage pathway may overcome single treatment failure, for example targeting the dCTP pool, which is rate-limiting for DNA replication. RNR inhibitors (examples in Table 1) failed to show clinical efficacy because of resistance mechanisms involving activation of two stress response pathways: the nucleoside salvage pathway (which may supply dNTPs in cancer cells treated with RNR inhibition) and the replication stress response (RSR) pathway (which enables cancer cells to survive the inhibition of dNTP synthesis via stabilization of stalled replication forks and other protective mechanisms). These two types of resistance mechanisms were targeted pharmacologically with compounds (Tables 2 and 3), with minimal toxicity to normal tissues and significant therapeutic efficacy. Triple combinations therapies include an RNR inhibitor (e.g., as shown in Table 1, 3-AP (Triapine)), a dCK inhibitor (e.g., as shown in Table 2, DI-82), and an inhibitor of the Replication Stress Response (RSR) pathway (e.g., as in Table 3, VE-822).

### DETAILED DESCRIPTION OF THE INVENTION

### Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, B, As, and Si may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heteroalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be a —O— bonded to a ring heteroatom nitrogen.

A “fused ring aryl-heterocycloalkyl” is an aryl fused to a heterocycloalkyl. A “fused ring heteroaryl-heterocycloalkyl” is a heteroaryl fused to a heterocycloalkyl. A “fused ring heterocycloalkyl-cycloalkyl” is a heterocycloalkyl fused to a cycloalkyl. A “fused ring heterocycloalkyl-heterocycloalkyl” is a heterocycloalkyl fused to another heterocycloalkyl. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be unsubstituted or substituted with one or more of the substituents described herein. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be named according to the size of each of the fused rings. Thus, for example, 6,5 aryl-heterocycloalkyl fused ring describes a 6 membered aryl moiety fused to a 5 membered heterocycloalkyl. Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The term “oxo,” as used herein, means an oxygen that is double bonded to a carbon atom.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R, —C(O)R, —CO₂R′, —CONR′R″, —OC(O)NR′R″, —NR″C(O)R, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″, —CN, —NO₂, —NR′SO₂R″, —NR′C═(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R, —C(O)R, —CO₂R′, —CONR′R″, —OC(O)NR′R″, —NR″C(O)R, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C═(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), Boron (B), Arsenic (As), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and/or each substituted or unsubstituted heterocycloalkyl is a substituted or unsubstituted 3 to 8 membered heterocycloalkyl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, and/or each substituted or unsubstituted heterocycloalkylene is a substituted or unsubstituted 3 to 8 membered heterocycloalkylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and/or each substituted or unsubstituted heterocycloalkyl is a substituted or unsubstituted 3 to 7 membered heterocycloalkyl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, and/or each substituted or unsubstituted heterocycloalkylene is a substituted or unsubstituted 3 to 7 membered heterocycloalkylene.

Certain compounds described herein possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the (R) and (S) configurations for each asymmetric center. Therefore, single stereochemical isomers as well as enantiomeric and diastereomeric mixtures of the present compounds, generally recognized as stable by those skilled in the art, are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, replacement of fluoride by ¹⁸F, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), fluroide (¹⁸F), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman decimal symbol may be used to distinguish each appearance of that particular R group. For example, where multiple R¹³ substituents are present, each R¹³ substituent may be distinguished as R^{13.1}, R^{13.2}, R^{13.3}, R^{13.4} etc., wherein each of R^{13.1}, R^{13.2}, R^{13.3}, R^{13.4} etc. is defined within the scope of the definition of R¹³ and optionally differently.

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

“Analog” or “analogue” are used in accordance with plain ordinary meaning within Chemistry and Biology and refer to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analogue is a compound that is similar or comparable in function and appearance but not in structure or origin to a reference compound.

The terms “Deoxycytidine kinase,” “DCK,” and “dCK” are here used interchangeably and according to their common, ordinary meaning and refer to proteins of the same or similar names and functional fragments and homologs thereof. The term includes any recombinant or naturally occurring form of dCK (NP000779.1 GI:4503269), or variants thereof that maintain dCK activity (e.g. within at least 30%, 40%, 50%, 60%, 70%, 80%, 90%, 95%, or 100% activity compared to dCK).

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The terms “a” or “an,” as used in herein means one or more.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat cancer (e.g. prostate cancer, castration-resistant prostate cancer, breast cancer, triple negative breast cancer, glioblastoma, ovarian cancer, lung cancer, squamous cell carcinoma (e.g head, neck, or esophagus), colorectal cancer, leukemia, acute myeloid leukemia, lymphoma, B cell lymphoma, or multiple myeloma). For example certain methods herein treat cancer by decreasing or reducing or preventing the occurrence, growth, metastasis, or progression of cancer; or treat cancer by decreasing a symptom of cancer. Symptoms of cancer (e.g. prostate cancer, castration-resistant prostate cancer, breast cancer, triple negative breast cancer, glioblastoma, ovarian cancer, lung cancer, squamous cell carcinoma (e.g head, neck, or esophagus), colorectal cancer, leukemia, acute myeloid leukemia, lymphoma, B cell lymphoma, or multiple myeloma) would be known or may be determined by a person of ordinary skill in the art. The term “treating” and conjugations thereof, include prevention of an injury, pathology, condition, or disease (e.g. preventing the development of one or more symptoms of cancer (e.g. prostate cancer, castration-resistant prostate cancer, breast cancer, triple negative breast cancer, glioblastoma, ovarian cancer, lung cancer, squamous cell carcinoma (e.g head, neck, or esophagus), colorectal cancer, leukemia, acute myeloid leukemia, lymphoma, B cell lymphoma, or multiple myeloma)).

An “effective amount” is an amount sufficient to accomplish a stated purpose (e.g. achieve the effect for which it is administered, treat a disease, reduce enzyme activity, increase enzyme activity, reduce transcriptional activity, increase transcriptional activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein (e.g. transcription factor) relative to the absence of the antagonist. An “activity increasing amount,” as used herein, refers to an amount of agonist (activator) required to increase the activity of an enzyme or protein (e.g. transcription factor) relative to the absence of the agonist. A “function disrupting amount,” as used herein, refers to the amount of antagonist (inhibitor) required to disrupt the function of an enzyme or protein (e.g. transcription factor) relative to the absence of the antagonist. A “function increasing amount,” as used herein, refers to the amount of agonist (activator) required to increase the function of an enzyme or protein (e.g. transcription factor) relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g., cancer) means that the disease (e.g. cancer) is caused by (in whole or in part), or a symptom of the disease is caused by (in whole or in part) the substance or substance activity or function. For example, a symptom of a disease or condition associated with an increase in nucleoside salvage pathway activity, ribonucleotide reductase (RNR) activity, or replication stress response pathway (RSR) may be a symptom that results (entirely or partially) from an increase in nucleoside salvage pathway activity, ribonucleotide reductase (RNR) activity, or replication stress response pathway (RSR), respectively. As used herein, what is described as being associated with a disease, if a causative agent, could be a target for treatment of the disease. For example, a disease associated with nucleoside salvage pathway activity, ribonucleotide reductase (RNR) activity, or replication stress response pathway (RSR), may be treated with an agent (e.g. compound as described herein) effective for decreasing the level of activity of nucleoside salvage pathway activity, ribonucleotide reductase (RNR) activity, or replication stress response pathway (RSR), respectively. For example, a disease associated with RNR, may be treated with an agent (e.g. compound as described herein) effective for decreasing the level of activity of RNR or a downstream component or effector of RNR. As used herein, what is described as being associated with a disease, if a causative agent, could be a target for treatment of the disease.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme (e.g. a RNR, dCK, ATR, Chk1). In some embodiments contacting includes allowing a compound described herein to interact with a protein or enzyme that is involved in a signaling pathway (e.g. nucleoside salvage pathway, ribonucleotide reductase (RNR) pathway, or replication stress response pathway (RSR)).

As defined herein, the term “inhibition”, “inhibit”, “inhibiting” and the like in reference to a protein-inhibitor (e.g. antagonist) interaction means negatively affecting (e.g. decreasing) the activity or function of the protein (e.g., RNR, dCK, ATR, Chk1) relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway (e.g., nucleoside salvage pathway, ribonucleotide reductase (RNR) pathway, or replication stress response pathway (RSR)). Thus, inhibition includes, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g. agonist) interaction means positively affecting (e.g. increasing) the activity or function of the protein (e.g. RNR, dCK, ATR, Chk1), In some embodiments, activation refers to an increase in the activity of a signal transduction pathway or signaling pathway (e.g. nucleoside salvage pathway, ribonucleotide reductase (RNR) pathway, or replication stress response pathway (RSR)).

The term “modulator” refers to a composition that increases or decreases the level of a target molecule or the function of a target molecule (e.g., RNR, dCK, ATR, Chk1). In some embodiments, modulation refers to an increase or decrease in the activity of a signal transduction pathway or signaling pathway (e.g. nucleoside salvage pathway, ribonucleotide reductase (RNR) pathway, or replication stress response pathway (RSR)).

In some embodiments, a modulator is a compound that reduces the severity of one or more symptoms of a disease associated with a protein (e.g., RNR, dCK, ATR, Chk1) or pathway (e.g. nucleoside salvage pathway, ribonucleotide reductase (RNR) pathway, or replication stress response pathway (RSR)), for example cancer.

“Patient” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a mammal. In some embodiments, a patient is a mouse. In some embodiments, a patient is an experimental animal. In some embodiments, a patient is a rat. In some embodiments, a patient is a test animal.

“Disease” or “condition” refer to a state of being or health status of a patient or subject capable of being treated with a compound, pharmaceutical composition, or method provided herein. In some embodiments, the disease is a disease related to (e.g. caused by) an increase in the level of a protein or activity of a protein (e.g., RNR, dCK, ATR, Chk1) or pathway activity (e.g. nucleoside salvage pathway, ribonucleotide reductase (RNR) pathway, or replication stress response pathway (RSR)). In some embodiments, the disease is cancer.

Examples of diseases, disorders, or conditions include, but are not limited to, cancer (e.g. prostate cancer, castration-resistant prostate cancer, breast cancer, triple negative breast cancer, glioblastoma, ovarian cancer, lung cancer, squamous cell carcinoma (e.g head, neck, or esophagus), colorectal cancer, leukemia, acute myeloid leukemia, lymphoma, B cell lymphoma, or multiple myeloma). In some instances, “disease” or “condition” refers to cancer. In some further instances, “cancer” refers to human cancers and carcinomas, sarcomas, adenocarcinomas, lymphomas, leukemias, melanomas, etc., including solid and lymphoid cancers, kidney, breast, lung, bladder, colon, ovarian, prostate, pancreas, stomach, brain, head and neck, skin, uterine, testicular, glioma, esophagus, liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), and/or multiple myeloma. In some further instances, “cancer” refers to lung cancer, breast cancer, ovarian cancer, leukemia, lymphoma, melanoma, pancreatic cancer, sarcoma, bladder cancer, bone cancer, brain cancer, cervical cancer, colon cancer, esophageal cancer, gastric cancer, liver cancer, head and neck cancer, kidney cancer, myeloma, thyroid cancer, prostate cancer, metastatic cancer, or carcinoma.

As used herein, the term “cancer” refers to all types of cancer, neoplasm or malignant tumors found in mammals, including leukemia, lymphoma, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER² positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

Pharmaceutical compositions may include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

By “co-administer” it is meant that a compound described herein is administered at the same time, just prior to, or just after the administration of one or more additional therapies, for example, an anticancer agent as described herein. The compounds described herein can be administered alone or can be co-administered to the patient. Co-administration is meant to include simultaneous or sequential administration of the compound individually or in combination (more than one compound or agent). Thus, the preparations can also be combined, when desired, with other active substances (e.g. anticancer agents).

Co-administration includes administering one active agent (e.g. a complex described herein) within 0.5, 1, 2, 4, 6, 8, 10, 12, 16, 20, or 24 hours of a second active agent (e.g. anti-cancer agents). Also contemplated herein, are embodiments, where co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. Co-administration can be accomplished by co-formulation, i.e., preparing a single pharmaceutical composition including both active agents. In other embodiments, the active agents can be formulated separately. The active and/or adjunctive agents may be linked or conjugated to one another. The compounds described herein may be combined with treatments for cancer such as chemotherapy or radiation therapy.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). In embodiments, administration includes direct administration to a tumor. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent or chemotherapeutic). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

Pharmaceutical compositions provided by the present invention include compositions wherein the active ingredient (e.g. compounds described herein, including embodiments or examples) is contained in an effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g., RNR, dCK, ATR, Chk1) or pathway (e.g. nucleoside salvage pathway, ribonucleotide reductase (RNR) pathway, or replication stress response pathway (RSR)) and/or reducing, eliminating, or slowing the progression of disease symptoms (e.g. symptoms of cancer. Determination of a therapeutically effective amount of a compound or combination of compounds of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating cancer, or with adjunctive agents that may not be effective alone, but may contribute to the efficacy of the active agent(s).

In some embodiments, co-administration includes administering one or more active agents within 0.5, 1, 2, 4, 6, 8, 10, 12, 16, 20, or 24 hours of another active agent. Co-administration includes administering two or more active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with each other and/or with other treatments for cancer such as surgery.

The term “pharmaceutical composition” also be referred to herein as a “combination product,” or “pharmaceutical formulation” includes a combination of the recited active components (e.g. a de novo nucleotide biosynthesis pathway inhibitor, a nucleoside salvage pathway inhibitor, and a replication stress response pathway inhibitor) in a single unit dose (e.g. dosage form) or in multiple unit doses (e.g. dosage forms). For example, in embodiments where the pharmaceutical composition or combination product includes a de novo nucleotide biosynthesis pathway inhibitor, a nucleoside salvage pathway inhibitor, and a replication stress response pathway inhibitor, the de novo nucleotide biosynthesis pathway inhibitor, the nucleoside salvage pathway inhibitor, and a replication stress response pathway inhibitor may all be in a single unit dose (e.g. a single pill, tablet or iv injection). Alternatively, where the pharmaceutical composition or combination product includes a de novo nucleotide biosynthesis pathway inhibitor, a nucleoside salvage pathway inhibitor, and a replication stress response pathway inhibitor, the de novo nucleotide biosynthesis pathway inhibitor may be present in a first unit dose, the nucleoside salvage pathway inhibitor may be present in a second unit dose, and the replication stress response pathway inhibitor may be present in a third unit dose, wherein the first, second and unit dose are independent and separate dosage forms. In embodiments, the de novo nucleotide biosynthesis pathway inhibitor and the nucleoside salvage pathway inhibitor are present in a first unit dose, and the replication stress response pathway inhibitor is present in a second unit dose. In embodiments, the de novo nucleotide biosynthesis pathway inhibitor and the replication stress response pathway inhibitor is present in a first unit dose, and the nucleoside salvage pathway inhibitor are present in a second unit dose. In embodiments, the nucleoside salvage pathway inhibitor and the replication stress response pathway inhibitor is present in a first unit dose and the de novo nucleotide biosynthesis pathway inhibitor is present in a second unit dose.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, or in separate containers (e.g., capsules and/or intravenous formulations) for each active ingredient. In addition, such administration also encompasses use of each type of therapeutic agent in a sequential manner, either at approximately the same time or at different times. In either case, the treatment regimen will provide beneficial effects of the drug combination in treating the conditions or disorders described herein.

The term “non-fixed combination” means that the active ingredients are administered to a patient as separate entities either simultaneously, concurrently or sequentially with no specific time limits, wherein such administration provides therapeutically effective levels of the two compounds in the body of the warm-blooded animal in need thereof.

The term “unit dose” is used herein to mean administration of a single agent, simultaneous administration of two agents together or simultaneous administration of three agents together, in one dosage form, to the patient being treated. In some embodiments, the unit dose is a single formulation. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, or patches administered to the patient at the same time.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec®), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein kinase C inhibitors, protein tyrosine phosphatase inhibitors; purine nucleoside phosphorylase inhibitors; ras farnesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER², anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™) erlotinib (Tarceva™) cetuximab (Erbitux™), lapatinib (Tykerb™), panitumumab (Vectibix™) vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

Additionally, the compounds described herein can be co-administered with conventional immunotherapeutic agents including, but not limited to, immunostimulants (e.g., Bacillus Calmette-Guérin (BCG), levamisole, interleukin-2, alpha-interferon, etc.), monoclonal antibodies (e.g., anti-CD20, anti-HER², anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y or ¹³¹I, etc.).

In a further embodiment, the compounds described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

As used herein, the term “about” means a range of values including the specified value, which a person of ordinary skill in the art would consider reasonably similar to the specified value. In embodiments, “about” means within a standard deviation using measurements generally acceptable in the art. In embodiments, about means a range extending to +/−10% of the specified value. In embodiments, about means the specified value.

The term “ribonucleotide reductase” or “RNR” refers to a protein that catalyzes the de novo synthesis of deoxyribonucleotides and the formation of deoxyribonucleotides from ribonucleotides, and homologs thereof. Functional RNR is a heterodimeric tetramer of RNR¹ and RNR² subunits. In humans the RNR¹ subunit is encoded by the RRM1 gene associated with Entrez Gene 6240, OMIM 180410, UniProt P23921, and/or RefSeq NM_001033. In humans the RNR² subunit is encoded by the RRM2 gene associated with Entrez Gene 6241, OMIM 180390, UniProt P31350, and/or RefSeq NM_001034 and the RRM2B gene associated with Entrez Gene 50484, OMIM 604712, UniProt Q9NTD8, and/or RefSeq NM_015713. In embodiments, the reference numbers immediately above refer to the protein, and associated nucleic acids, known as of the date of filing of this application.

The term “deoxycytidine kinase” or “dCK” refers to a protein that phosphorylates certain deoxyribonucleosides and select analogs, and homologs thereof. In embodiments, “dCK” refers to the protein associated with Entrez Gene 1633, OMIM 125450, UniProt P27707, and/or RefSeq (protein) NP_000779. In embodiments, the reference numbers immediately above refer to the protein, and associated nucleic acids, known as of the date of filing of this application.

The term “Ataxia telangiectasia and Rad3 related protein” or “ATR” refers to a phosphatidylinositol 3-kinase-related kinase protein and homologs thereof. In embodiments, “ATR” refers to the protein associated with Entrez Gene 545, OMIM 601215, UniProt Q13535, and/or RefSeq (protein) NP_001175. In embodiments, the reference numbers immediately above refer to the protein, and associated nucleic acids, known as of the date of filing of this application.

The term “Checkpoint kinase 1” or “Chk1” or “CHEK1” refers to a serine/threonine-specific protein kinase and homologs thereof. In embodiments, “Chk1” refers to the protein associated with Entrez Gene 1111, OMIM 603078, UniProt O14757, and/or RefSeq (protein) NP_001107593. In embodiments, the reference numbers immediately above refer to the protein, and associated nucleic acids, known as of the date of filing of this application.

The term “WEE1-like protein kinase” or “WEE1” refers to a kinase that acts on Cdk1. In embodiments, WEE1 may refer to one or both of the WEE1-like protein kinases in humans (human WEE1 homolog and/or human WEE1 homolog 2). Human WEE1 homolog is encoded by the WEE1 gene associated with Entrez Gene 7465, OMIM 193525, UniProt P30291, and/or RefSeq NM_003390. Human WEE1 homolog 2 is encoded by the WEE1 gene associated with Entrez Gene 494551, UniProt P0C1S8, and/or RefSeq NM_001105558. In embodiments, WEE1 refers to both of the WEE1-like protein kinases in humans (human WEE1 homolog and human WEE1 homolog 2. In embodiments, WEE1 refers to human WEE1 homolog. In embodiments, WEE1 refers to human WEE1 homolog 2. In embodiments, the reference numbers immediately above refer to the protein, and associated nucleic acids, known as of the date of filing of this application.

The term “ribonucleotide reductase antagonist”, “ribonucleotide reductase inhibitor”, “RNR antagonist”, or “RNR inhibitor” refers to an agent (e.g., compound, antibody, protein, or nucleic acid) capable of reducing the level of RNR protein, RNR mRNA, or RNR activity, relative to a control (e.g., comparison of level in the absence of the RNR antagonist). In embodiments, the RNR inhibitor is a compound (e.g., small molecule). The RNR inhibitor may reduce the level of activity of RNR. The RNR inhibitor may reduce the level of activity of RNR when the RNR inhibitor binds RNR. The RNR inhibitor may reduce the production of a deoxyribonucleotide from a ribonucleotide by RNR. Non-limiting examples of RNR inhibitors are included in Table 1.

TABLE 1
Inhibitors of de novo nucleotide biosynthesis
pathway, (RNR) dCTP biosynthesis

| | | IC₅₀ | |
|---|---|---|---|
| | Compound | (cytotoxicity | |
| Target | (Company) | assay) | Clinical Status |
| RNR | Hydroxyurea | 39 μM | approved |
| RNR | Gallium Maltolate | 30 μM | Phase I/II |
| | (Titan) | | |
| RNR | 3-AP (Triapine) | 1.31 μM (cell type | Phase I/II |
| | (Nanotherapeutics) | independent) | |
| RNR | Thymidine | 50 μM-5 mM | — |
| | | (cell type | |
| | | dependent) | |
| RNR | clofarabine | | |

The RNR inhibitor 3-AP is a thiosemicarbazone. Other examples of thiosemicarbazones include, but are not limited to, di-2-pyridylketone-4,4-dimethyl-3-thiosemicarbazone (Dp44mT), di-2-pyridylketone thiosemicarbazone (DpT), 2-benzoylpyridine thiosemicarbazone (BpT), 2-benzoylpyridine 4-ethyl-3-thiosemicarbazone (Bp4eT), di-2-pyridylketone 4-cyclohexyl-4-methyl-3-thiosemicarbazone hydrochloride (DpC), di-2-pyridylketone-4-ethyl-4-methyl-3-thiosemicarbazone (Dp4e4mT), di-2-pyridylketone-4-phenyl-3-thiosemicarbazone (Dp4pT) and di-2-pyridylketone-2-methyl-3-thiosemicarbazone (Dp2mT).

The term “deoxycytidine kinase antagonist”, “deoxycytidine kinase inhibitor”, “dCK antagonist”, or “dCK inhibitor” refers to an agent (e.g., compound, antibody, protein, or nucleic acid) capable of reducing the level of dCK protein, dCK mRNA, or dCK activity, relative to a control (e.g., comparison of level in the absence of the dCK antagonist). In embodiments, the dCK inhibitor is a compound (e.g., small molecule). The dCK inhibitor may reduce the level of activity of dCK. The dCK inhibitor may reduce the level of activity of dCK when the dCK inhibitor binds dCK. The dCK inhibitor may reduce the production of a phosphorylated deoxyribonucleoside from a deoxyribonucleoside by dCK. Non-limiting examples of dCK inhibitors are included in Table 2.

TABLE 2
Inhibitors of nucleoside salvage
pathway, (dCK) dCTP biosynthesis

| | | IC₅₀ | | |
|---|---|---|---|---|
| | Compound | (cytotoxicity | | |
| Target | (Company) | assay) | | Clinical Status |
| dCK | LP-661438 | 0.45 | μM | — |
| | (Lexicon) | | | |
| dCK | DI-39 | 3.22 | nM | — |
| dCK | (R)-DI-82 | 3.7 | nM | — |

The term “nucleoside salvage pathway antagonist” or “nucleoside salvage pathway inhibitor” refers to an agent (e.g., compound, antibody, protein, or nucleic acid) capable of reducing the level of a protein component of the nucleoside salvage pathway, an mRNA of a protein component of the nucleoside salvage pathway, or the activity of a component of the nucleoside salvage pathway, relative to a control (e.g., comparison of level in the absence of the nucleoside salvage pathway antagonist). In embodiments, the nucleoside salvage pathway inhibitor is a compound (e.g., small molecule). The nucleoside salvage pathway inhibitor may reduce the level of activity of a component of the nucleoside salvage pathway. The nucleoside salvage pathway inhibitor may reduce the level of activity of a component of the nucleoside salvage pathway when the nucleoside salvage pathway inhibitor binds to the component. The nucleoside salvage pathway inhibitor may reduce the production of a deoxyribonucleotide triphosphate from a deoxyribonucleoside. Non-limiting examples of nucleoside salvage pathway inhibitors are included in Table 2. A dCK inhibitor is a nucleoside salvage pathway inhibitor. Non-limiting examples of nucleoside salvage pathway inhibitors are described in WO2012/122368 (PCT/US2012/028259) enstitled “Deoxycytidine kinase binding compounds” by Radu et al., which is incorporated by reference in its entirety for all purposes. Non-limiting examples of dCK inhibitors are described in WO2012/122368 (PCT/US2012/028259). Non-limiting examples of nucleoside salvage pathway inhibitors are described herein. Non-limiting examples of dCK inhibitors are described herein. In embodiments, a nucleoside salvage pathway inhibitor is a compound described in WO2012/122368 (PCT/US2012/028259) or herein. In embodiments, a dCK inhibitor is a compound described in WO2012/122368 (PCT/US2012/028259) or herein. WO2012/122368 (PCT/US2012/028259) and the compounds of the disclosure exemplify nucleoside salvage pathway inhibitors and dCK inhibitors that may be included in the compositions and/or methods described herein.

The term “Ataxia telangiectasia and Rad3 related protein antagonist”, “Ataxia telangiectasia and Rad3 related protein inhibitor”, “ATR antagonist”, or “ATR inhibitor” refers to an agent (e.g., compound, antibody, protein, or nucleic acid) capable of reducing the level of ATR protein, ATR mRNA, or ATR activity, relative to a control (e.g., comparison of level in the absence of the ATR antagonist). In embodiments, the ATR inhibitor is a compound (e.g., small molecule). The ATR inhibitor may reduce the level of activity of ATR. The ATR inhibitor may reduce the level of activity of ATR when the ATR inhibitor binds ATR. The ATR inhibitor may reduce the phosphorylation of Chk1. The ATR inhibitor may reduce the level of cell cycle arrest induced by ATR compared to control (e.g., level of cell cycle arrest in the absence of the ATR inhibitor). The ATR inhibitor may reduce the phosphorylation of a protein by ATR. The ATR inhibitor may reduce the detection of single stranded DNA by ATR. The ATR inhibitor may reduce the level of activity of the replication stress response pathway. Non-limiting examples of ATR inhibitors are included in Table 3.

TABLE 3
Inhibitors of the Replication Stress Response pathway (RSR)

| | | IC₅₀ against | |
|---|---|---|---|
| | | primary target | |
| | Compound | and additional | |
| Target | (Company) | targets | Clinical Status |
| ATR | VE-821, VE-822 | 19 nM vs. 34 μM | Phase I |
| | (Vertex) | for ATM | |
| ATR | AZ20(Astra Zeneca) | 5 nM, 7.6-fold | — |
| | | selectivity over | |
| | | 442 kinases | |
| ATR | AZD6738 (Astra | AZ20 analogue | Phase I |
| | Zeneca) | (better PD, | |
| | | solubility and | |
| | | bioavailable) | |
| ATR | Torin-2 | 35 nM; 28 nM for | |
| | | ATM, 118 nM for | |
| | | DNA-PK | |
| ATR | NVP-BEZ235 | 21 nM, dual | Phase I/II |
| | (Novartis) | mTOR/PI3K | |
| | | inhibitor | |
| Chk1 | CHIR-124(Chiron) | 0.3 nM; 2,000-fold | — |
| | | selectivity against | |
| | | Chk2, and | |
| | | 500-5,000-fold | |
| | | against CDK2/4 | |
| | | and Cdc2 | |
| Chk1 | SB-218078(GSK) | 15 nM, 250 nM for | — |
| | | cdc2, 1000 nM for | |
| | | PKC | |
| Chk1 | SAR-020106 | 13.3 nM for | Phase I |
| | (Institute of Cancer | Chk1, >10 μM | |
| | Research, Sareum) | for Chk2 | |
| Chk1 | SCH900776 (MK- | 3 nM, 1.5 μM for | Phase I/II |
| | 8776, Merck) | Chk2 and 0.16 μM | |
| | | for CDK2, also | |
| | | targets Pim1 | |
| Chk1 | LY2603618 (Eli | 5 nM, Chk1 | Phase I/II |
| | Lilly) | selective | |
| Chk1 | GNE-783; GNE- | 1 nM, Chk1 | — |
| | 900 (Genentech) | selective | |
| Chk1 | PD-321852 | 5 nM, selectivity | Phase I |
| | (Pfizer) | data unavailable | |
| Chk1 | CCT244747 | 29-170 nM, also | — |
| | (Institute of Cancer | targets FLT3, | |
| | Research) | Chk2 and CDK1 | |
| | | (orally | |
| | | bioavailable) | |
| Chk1 | UCN-01 | 11 nM, 1.1 μM for | Phase II |
| | | Chk2, MARK3 | |
| | | (27 nM) and PKC | |
| | | isoenzymes, | |
| | | CDK1 and CDK2 | |
| Chk1 | PF 00477736 | 0.49 nM; ~100- | Phase I |
| | (Pfizer) | fold selectivity for | |
| | | Chk1 over Chk2; | |
| | | also inhibits | |
| | | VEGFR2, Aurora-A, | |
| | | FGFR3, Flt3, | |
| | | Fms (CSF1R), Ret | |
| | | and Yes. | |
| Chk1 | XL844 (Exelixis) | 2.2 nM, 0.07 nM | Phase I |
| | | against Cnk2; | |
| | | inhibits Flt-4, | |
| | | Flt-3, KDR and PDGF | |
| | | at less than 20 nM | |
| Chk1 | AZD7762 | Chk1 and Chk2 | Phase I |
| | (Astra Zeneca) | 5 nM, less potent | |
| | | against CAM, Yes, | |
| | | Fyn, Lyn, Hck and | |
| | | Lck | |
| Chk1 | AR458323 (Array) | 27 nM | — |
| Chk1 | GDC0425, GDC0575 | not disclosed; | Phase I |
| | (ARRY-575) | orally bioavailable | |
| | (Genentech & | | |
| | Array) | | |
| Chk1 | AR323, AR678 | 0.9 nM; 110 nM | — |
| | (Array) | (RSK3), 50 nM | |
| | | (Mylk) | |
| Chk1 | TCS2312 (Tocris | 125-550 nM, | |
| | bioscience) | undisclosed | |
| | | selectivity | |
| Chk1 | V158411 | 3.5 nM; 2.5 nM | — |
| | (Vernalis) | against Chk2 | |
| Chk1 | CEP-3891 | not disclosed | — |
| | (Cephalon) | | |
| Wee1 | MK-1775 (Merck) | 5.2 nM, selective | Phase I |
| | | Wee1 inhibitor | |
| Wee1 | PD-407824 | 97 nM for Wee1 | Phase I |
| | (Pfizer) | and Chk1; 100 fold | |
| | | selectivity over | |
| | | CDKs, c-Src, | |
| | | PDGFR and FDFR | |

The term “Checkpoint kinase 1 antagonist”, “Checkpoint kinase 1 inhibitor”, “Chk1 antagonist”, or “Chk1 inhibitor” refers to an agent (e.g., compound, antibody, protein, or nucleic acid) capable of reducing the level of Chk1 protein, Chk1 mRNA, or Chk1 activity, relative to a control (e.g., comparison of level in the absence of the Chk1 antagonist). In embodiments, the Chk1 inhibitor is a compound (e.g., small molecule). The Chk1 inhibitor may reduce the level of activity of Chk1. The Chk1 inhibitor may reduce the level of activity of Chk1 when the Chk1 inhibitor binds Chk1. The Chk1 inhibitor may reduce the level of cell cycle arrest induced by Chk1 compared to control (e.g., level of cell cycle arrest in the absence of the Chk1 inhibitor). The Chk1 inhibitor may reduce the phosphorylation of a protein by Chk1. The Chk1 inhibitor may reduce the phosphorylation of cdc25 by Chk1. The Chk1 inhibitor may reduce the phosphorylation of WEE1 by Chk1. The Chk1 inhibitor may reduce the level of activity of the replication stress response pathway. Non-limiting examples of Chk1 inhibitors are included in Table 3.

The term “WEE1 antagonist” or “WEE1 inhibitor” refers to an agent (e.g., compound, antibody, protein, or nucleic acid) capable of reducing the level of WEE1 protein, WEE1 mRNA, or WEE1 activity, relative to a control (e.g., comparison of level in the absence of the WEE1 antagonist). In embodiments, the WEE1 inhibitor is a compound (e.g., small molecule). The WEE1 inhibitor may reduce the level of activity of WEE1. The WEE1 inhibitor may reduce the level of activity of WEE1 when the WEE1 inhibitor binds WEE1. The WEE1 inhibitor may reduce the level of cell cycle arrest induced by WEE1 compared to control (e.g., level of cell cycle arrest in the absence of the WEE1 inhibitor). The WEE1 inhibitor may reduce the phosphorylation of a protein by WEE1. The WEE1 inhibitor may reduce the phosphorylation of Cdk1 by WEE1. The WEE1 inhibitor may reduce the level of activity of the replication stress response pathway. Non-limiting examples of WEE1 inhibitors are included in Table 3.

The term “replication stress response pathway antagonist”, “replication stress response pathway inhibitor”, “RSR pathway antagonist”, or “RSR pathway inhibitor” refers to an agent (e.g., compound, antibody, protein, or nucleic acid) capable of reducing the level of a protein component of the replication stress response pathway, an mRNA of a protein component of the replication stress response pathway, or the activity of a component of the replication stress response pathway, relative to a control (e.g., comparison of level in the absence of the replication stress response pathway antagonist). In embodiments, the replication stress response pathway inhibitor is a compound (e.g., small molecule). The replication stress response pathway inhibitor may reduce the level of activity of a component of the replication stress response pathway. The replication stress response pathway inhibitor may reduce the level of activity of a component of the replication stress response pathway when the replication stress response pathway inhibitor binds to the component. The replication stress response pathway inhibitor may reduce the level of cell cycle arrest compared to control (e.g., absence of the replication stress response pathway inhibitor). Non-limiting examples of replication stress response pathway inhibitors are included in Table 3. An ATR inhibitor is a replication stress response pathway inhibitor. A Chk1 inhibitor is a replication stress response pathway inhibitor. A WEE1 inhibitor is a replication stress response pathway inhibitor.

The term “de novo nucleotide biosynthesis pathway antagonist” or “de novo nucleotide biosynthesis pathway inhibitor” refers to an agent (e.g., compound, antibody, protein, or nucleic acid) capable of reducing the level of a protein component of the de novo nucleotide biosynthesis pathway, an mRNA of a protein component of the de novo nucleotide biosynthesis pathway, or the activity of a component of the de novo nucleotide biosynthesis pathway, relative to a control (e.g., comparison of level in the absence of the de novo nucleotide biosynthesis pathway antagonist). In embodiments, the de novo nucleotide biosynthesis pathway inhibitor is a compound (e.g., small molecule). The de novo nucleotide biosynthesis pathway inhibitor may reduce the level of activity of a component of the de novo nucleotide biosynthesis pathway. The de novo nucleotide biosynthesis pathway inhibitor may reduce the level of activity of a component of the de novo nucleotide biosynthesis pathway when the de novo nucleotide biosynthesis pathway inhibitor binds to the component. The de novo nucleotide biosynthesis pathway inhibitor may reduce the level of production of a nucleotide compared to control (e.g., absence of the de novo nucleotide biosynthesis pathway inhibitor). The de novo nucleotide biosynthesis pathway inhibitor may reduce the level of production of a ribonucleotide compared to control (e.g., absence of the de novo nucleotide biosynthesis pathway inhibitor). The de novo nucleotide biosynthesis pathway inhibitor may reduce the level of production of a deoxyribonucleotide compared to control (e.g., absence of the de novo nucleotide biosynthesis pathway inhibitor). The de novo nucleotide biosynthesis pathway inhibitor may reduce the level of production of a ribonucleotide triphosphate compared to control (e.g., absence of the de novo nucleotide biosynthesis pathway inhibitor). The de novo nucleotide biosynthesis pathway inhibitor may reduce the level of production of a deoxyribonucleotide triphosphate compared to control (e.g., absence of the de novo nucleotide biosynthesis pathway inhibitor). The de novo nucleotide biosynthesis pathway inhibitor may reduce the level of production of dCTP compared to control (e.g., absence of the de novo nucleotide biosynthesis pathway inhibitor). The de novo nucleotide biosynthesis pathway inhibitor may reduce the level of production of dATP compared to control (e.g., absence of the de novo nucleotide biosynthesis pathway inhibitor). The de novo nucleotide biosynthesis pathway inhibitor may reduce the level of production of dGTP compared to control (e.g., absence of the de novo nucleotide biosynthesis pathway inhibitor). The de novo nucleotide biosynthesis pathway inhibitor may reduce the level of production of dTTP compared to control (e.g., absence of the de novo nucleotide biosynthesis pathway inhibitor). Non-limiting examples of de novo nucleotide biosynthesis pathway inhibitors are included in Table 3. An RNR inhibitor is a de novo nucleotide biosynthesis pathway inhibitor.

Pharmaceutical Compositions

In an aspect is provided a pharmaceutical composition including a pharmaceutically acceptable excipient and a compound, or pharmaceutically acceptable salt thereof, wherein the compound is a de novo nucleotide biosynthesis pathway inhibitor, a nucleoside salvage pathway inhibitor, or a replication stress response pathway inhibitor or any combination thereof.

In embodiments, the pharmaceutical composition includes two compounds, or pharmaceutically acceptable salts thereof, wherein the compounds are a de novo nucleotide biosynthesis pathway inhibitor, a nucleoside salvage pathway inhibitor, or a replication stress response pathway inhibitor. In embodiments, the pharmaceutical composition includes a de novo nucleotide biosynthesis pathway inhibitor, a nucleoside salvage pathway inhibitor, and a replication stress response pathway inhibitor.

In embodiments, the pharmaceutical composition includes a de novo nucleotide biosynthesis pathway inhibitor and a nucleoside salvage pathway inhibitor. In embodiments, the pharmaceutical composition includes a de novo nucleotide biosynthesis pathway inhibitor and a replication stress response pathway inhibitor. In embodiments, the pharmaceutical composition includes a nucleoside salvage pathway inhibitor and a replication stress response pathway inhibitor. In embodiments, the pharmaceutical composition includes a de novo nucleotide biosynthesis pathway inhibitor, a nucleoside salvage pathway inhibitor, and a replication stress response pathway inhibitor.

In embodiments, the de novo nucleotide biosynthesis pathway inhibitor is an RNR inhibitor. In embodiments, the de novo nucleotide biosynthesis pathway inhibitor is a compound listed in Table 1. In embodiments, the de novo nucleotide biosynthesis pathway inhibitor is 3-AP.

In embodiments, the nucleoside salvage pathway inhibitor is a dCK inhibitor. In embodiments, the nucleoside salvage pathway inhibitor is a compound listed in Table 2. In embodiments, the nucleoside salvage pathway inhibitor is a racemic mixture of the enantiomers of DI-82 or D-87. In embodiments, the nucleoside salvage pathway inhibitor is (R) DI-82. DI-82 is

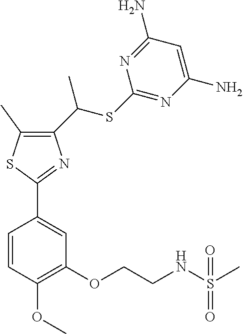

(i.e., N-(2-(5-(4-(1-((4,6-diaminopyrimidin-2-yl)thio)ethyl)-5-methylthiazol-2-yl)-2-methoxyphenoxy)ethyl)methanesulfonamide). (R) DI-82 is

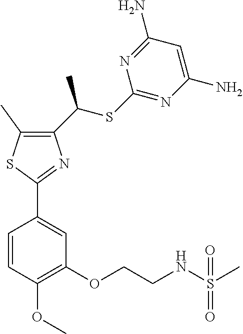

(i.e., (R)—N-(2-(5-(4-(1-((4,6-diaminopyrimidin-2-yl)thio)ethyl)-5-methylthiazol-2-yl)-2-methoxyphenoxy)ethyl)methanesulfonamide). See 12R in Nomme et al., J. Med Chem. 2014 Nov. 26; 57(22):9480-94, incorporated herein by reference in its entirety for all purposes.

In certain embodiments, clofarabine, an RNR inhibitor, is used in combination with ATR inhibitors (small molecule compounds targeting the DNA repair kinases: ataxia-telangiectasia mutated (ATM) and ataxia telangiectasia-mutated and Rad3 related (ATR)). In other embodiments, clofarabine is used in combination with ATR inhibitors as described above and R-DI-87 (TRE-515).

Provided herein are compounds having the formula:

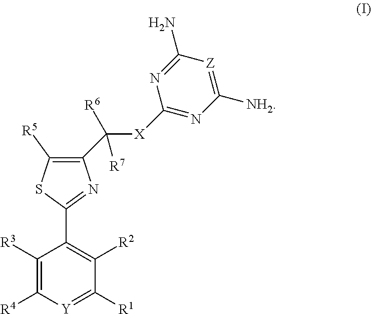

Y is C(R⁸) or N. Z is C(R⁹) or N. X is —CH₂—, —O—, —N(R¹⁰)—, —S—, —S(O)—, or —S(O)₂—. R¹ is hydrogen, halogen, —N₃, —CF₃, —CCl₃, —CBr₃, —CI₃, —CN, —COR^{1A}, —OR^{1A}, —NR^{1A}R^{1B}, —C(O)OR^{1A}, —C(O)NR^{1A}R^{1B}, —NO₂, —SR^{1A}, —S(O)ₙ₁R^{1A}, —S(O)ₙ₁OR^{1A}, —S(O)ₙ₁NR^{1A}R^{1B}, —NHNR^{1A}R^{1B}, —ONR^{1A}R^{1B}, —NHC(O)NHNR^{1A}R^{1B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is hydrogen, halogen, —N₃, —CF₃, —CCl₃, —CBr₃, —CI₃, —CN, —COR^{2A}, —OR^{2A}, —NR^{2A}R^{2B}, —C(O)OR^{2A}, —C(O)NR^{2A}R^{2B}, —NO₂, —SR^{2A}, —S(O)ₙ₂R^{2A}, —S(O)ₙ₂OR^{2A}, —S(O)ₙ₂NR^{2A}R^{2B}, —NHNR^{2A}R^{2B}, —ONR^{2A}R^{2B}, —NHC(O)NHNR^{2A}R^{2B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³ is hydrogen, halogen, —N₃, —CF₃, —CCl₃, —CBr₃, —CI₃, —CN, —COR^{3A}, —OR^{3A}, —NR^{3A}R^{3B}, —C(O)OR^{3A}, —C(O)NR^{3A}R^{3B}, —NO₂, —SR^{3A}, —S(O)ₙ₃R^{3A}, —S(O)ₙ₃OR^{3A}, —S(O)ₙ₃NR^{3A}R^{3B}, —NHNR^{3A}R^{3B}, —ONR^{3A}R^{3B}, —NHC(O)NHNR^{3A}R^{3B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁴ is hydrogen, halogen, —N₃, —CF₃, —CCl₃, —CBr₃, —CI₃, —CN, —COR^{4A}, —OR^{4A}, —NR^{4A}R^{4B}, —C(O)OR^{4A}, —C(O)NR^{4A}R^{4B}, —NO₂, —SR^{4A}, —S(O)ₙ₄R^{4A}, —S(O)ₙ₄OR^{4A}, —S(O)ₙ₄NR^{4A}R^{4B}, —NHNR^{4A}R^{4B}, —ONR^{4A}R^{4B}, —NHC(O)NHNR^{4A}R^{4B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁵ is independently hydrogen, halogen, —N₃, —CF₃, —CCl₃, —CBr₃, —CI₃, —CN, —COR^{5A}, —OR^{5A}, —NR^{5A}R^{5B}, —C(O)OR^{5A}, —C(O)NR^{5A}R^{5B}, —NO₂, —SR^{5A}, —S(O)ₙ₅R^{5A}, —S(O)ₙ₅OR^{5A}, —S(O)ₙ₅NR^{5A}R^{5B}, —NHNR^{5A}R^{5B}, —ONR^{5A}R^{5B}, —NHC(O)NHNR^{5A}R^{5B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R⁵ and R⁶ are optionally combined to form a substituted or unsubstituted cycloalkyl. R⁶ is unsubstituted C₁-C₆ alkyl. R⁷ is H, D, F or —CH₃. R⁸ is hydrogen, halogen, —N₃, —CF₃, —CCl₃, —CBr₃, —CI₃, —CN, —COR^{8A}, —OR^{8A}, —NR^{8A}R^{8B}, —C(O)OR^{8A}, —C(O)NR^{8A}R^{8B}, —NO₂, —SR^{8A}, —S(O)ₙ₈R^{8A}, —S(O)ₙ₈OR^{8A}, —S(O)ₙ₈NR^{8A}R^{8B}, —NHNR^{8A}R^{8B}, —ONR^{8A}R^{8B}, —NHC(O)NHNR^{8A}R^{8B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁹ is hydrogen, halogen, —N₃, —CF₃, —CCl₃, —CBr₃, —CI₃, —CN, —COR^{9A}, —OR^{9A}, —NR^{9A}R^{9B}, —C(O)OR^{9A}, —C(O)NR^{9A}R^{9B}, —NO₂, —SR^{9A}, —S(O)ₙ₉R^{9A}, —S(O)ₙ₉OR^{9A}, —S(O)ₙ₉NR^{9A}R^{9B}, —NHNR^{9A}R^{9B}, —ONR^{9A}R^{9B}, —NHC(O)NHNR^{9A}R^{9B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁰ is H, —CH₃, —C₂H₅, —C₃H₇, —CH₂C₆H₅. R^{1A}, R^{1B}, R^{2A}, R^{2B}, R^{3A}, R^{3B}, R^{4A}, R^{4B}, R^{5A}, R^{5B}, R^{8A}, R^{8B}, R^{9A}, and R^{9B} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbols n1, n2, n3, n4, n5, n8, and n9 are independently 1, 2, or 3.

R¹ may be hydrogen, halogen, —N₃, —CF₃, —CCl₃, —CBr₃, —CI₃, —CN, —COR^{1A}, —OR^{1A}, —NR^{1A}R^{1B}, —C(O)OR^{1A}, —C(O)NR^{1A}R^{1B}, —NO₂, —SR^{1A}, —S(O)ₙ₁R^{1A}, —S(O)ₙ₁OR^{1A}, —S(O)ₙ₁NR^{1A}R^{1B}, —NHNR^{1A}R^{1B}, —ONR^{1A}R^{1B}, or —NHC(O)NHNR^{1A}R^{1B}. R¹ may be hydrogen, halogen, —OR^{1A}. R¹ may be hydrogen. R¹ may be halogen. R¹ may be —OR^{1A}. R^{1A} is as described herein.

R¹ may be hydrogen, halogen, —OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ may be —OR^{1A}, where R^{1A} is as described herein. R¹ may be —OR^{1A}, where R^{1A} is hydrogen, substituted or unsubstituted alkyl, or substituted or unsubstituted heteroalkyl. R¹ may be —OR^{1A}, where R^{1A} is substituted or unsubstituted alkyl, or substituted or unsubstituted heteroalkyl.

R¹ may be substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ may be R^{1A}-substituted or unsubstituted alkyl, R^{1A}-substituted or unsubstituted heteroalkyl, R^{1A}-substituted or unsubstituted cycloalkyl, R^{1A}-substituted or unsubstituted heterocycloalkyl, R^{1A}-substituted or unsubstituted aryl, or R^{1A}-substituted or unsubstituted heteroaryl.

R¹ may be substituted or unsubstituted alkyl. R¹ may be substituted alkyl. R¹ may be unsubstituted alkyl. R¹ may be substituted or unsubstituted C₁-C₂₀ alkyl. R¹ may be substituted C₁-C₂₀ alkyl. R¹ may be unsubstituted C₁-C₂₀ alkyl. R¹ may be substituted or unsubstituted C₁₀ alkyl. R¹ may be substituted C₁-C₁₀ alkyl. R¹ may be unsubstituted C₁-C₁₀ alkyl. R¹ may be substituted or unsubstituted C₁-C₅ alkyl. R¹ may be substituted C₁-C₅ alkyl. R¹ may be unsubstituted C₁-C₅ alkyl. R¹ may be methyl. R¹ may be ethyl. R¹ may be propyl.

R¹ may be R^{1A}-substituted or unsubstituted alkyl. R¹ may be R^{1A}-substituted alkyl. R¹ may be unsubstituted alkyl. R¹ may be R^{1A}-substituted or unsubstituted C₁-C₂₀ alkyl. R¹ may be R^{1A}-substituted C₁-C₂₀ alkyl. R¹ may be unsubstituted C₁-C₂₀ alkyl. R¹ may be R^{1A}-substituted or unsubstituted C₁-C₁₀ alkyl. R¹ may be R^{1A}-substituted C₁-C₁₀ alkyl. R¹ may be unsubstituted C₁₀ alkyl. R¹ may be R^{1A}-substituted or unsubstituted C₁-C₅ alkyl. R¹ may be R^{1A}-substituted C₁-C₅ alkyl. R¹ may be unsubstituted C₁-C₅ alkyl.

R¹ may be substituted or unsubstituted heteroalkyl. R¹ may be substituted heteroalkyl. R¹ may be unsubstituted heteroalkyl. R¹ may be substituted or unsubstituted 2 to 20 membered heteroalkyl. R¹ may be substituted 2 to 20 membered heteroalkyl. R¹ may be unsubstituted 2 to 20 membered heteroalkyl. R¹ may be substituted or unsubstituted 2 to 10 membered heteroalkyl. R¹ may be substituted 2 to 10 membered heteroalkyl. R¹ may be unsubstituted 2 to 10 membered heteroalkyl. R¹ may be substituted or unsubstituted 2 to 6 membered heteroalkyl. R¹ may be substituted 2 to 6 membered heteroalkyl. R¹ may be unsubstituted 2 to 6 membered heteroalkyl.

R¹ may be R^{1A}-substituted or unsubstituted heteroalkyl. R¹ may be R^{1A}-substituted heteroalkyl. R¹ may be unsubstituted heteroalkyl. R¹ may be R^{1A}-substituted or unsubstituted 2 to 20 membered heteroalkyl. R¹ may be R^{1A}-substituted 2 to 20 membered heteroalkyl. R¹ may be unsubstituted 2 to 20 membered heteroalkyl. R¹ may be R^{1A}-substituted or unsubstituted 2 to 10 membered heteroalkyl. R¹ may be R^{1A}-substituted 2 to 10 membered heteroalkyl. R¹ may be unsubstituted 2 to 10 membered heteroalkyl. R¹ may be R^{1A}-substituted or unsubstituted 2 to 6 membered heteroalkyl. R¹ may be R^{1A}-substituted 2 to 6 membered heteroalkyl. R¹ may be unsubstituted 2 to 6 membered heteroalkyl.

R¹ may be substituted or unsubstituted cycloalkyl. R¹ may be substituted cycloalkyl. R¹ may be unsubstituted cycloalkyl. R¹ may be substituted or unsubstituted 3 to 10 membered cycloalkyl. R¹ may be substituted 3 to 10 membered cycloalkyl. R¹ may be unsubstituted 3 to 10 membered cycloalkyl. R¹ may be substituted or unsubstituted 3 to 8 membered cycloalkyl. R¹ may be substituted 3 to 8 membered cycloalkyl. R¹ may be unsubstituted 3 to 8 membered cycloalkyl. R¹ may be substituted or unsubstituted 3 to 6 membered cycloalkyl. R¹ may be substituted 3 to 6 membered cycloalkyl. R¹ may be unsubstituted 3 to 6 membered cycloalkyl. R¹ may be substituted or unsubstituted 3 membered cycloalkyl. R¹ may be substituted or unsubstituted 4 membered cycloalkyl. R¹ may be substituted or unsubstituted 5 membered cycloalkyl. R¹ may be substituted or unsubstituted 6 membered cycloalkyl.

R¹ may be R^{1A}-substituted or unsubstituted cycloalkyl. R¹ may be R^{1A}-substituted cycloalkyl. R¹ may be unsubstituted cycloalkyl. R¹ may be R^{1A}-substituted or unsubstituted 3 to 10 membered cycloalkyl. R¹ may be R^{1A}-substituted 3 to 10 membered cycloalkyl. R¹ may be unsubstituted 3 to 10 membered cycloalkyl. R¹ may be R^{1A}-substituted or unsubstituted 3 to 8 membered cycloalkyl. R¹ may be R^{1A}-substituted 3 to 8 membered cycloalkyl. R¹ may be unsubstituted 3 to 8 membered cycloalkyl. R¹ may be R^{1A}-substituted or unsubstituted 3 to 6 membered cycloalkyl. R¹ may be R^{1A}-substituted 3 to 6 membered cycloalkyl. R¹ may be unsubstituted 3 to 6 membered cycloalkyl. R¹ may be R^{1A}-substituted or unsubstituted 3 membered cycloalkyl. R¹ may be R^{1A}-substituted or unsubstituted 4 membered cycloalkyl. R¹ may be R^{1A}-substituted or unsubstituted 5 membered cycloalkyl. R¹ may be R^{1A}-substituted or unsubstituted 6 membered cycloalkyl.

R¹ may be substituted or unsubstituted heterocycloalkyl. R¹ may be substituted heterocycloalkyl. R¹ may be unsubstituted heterocycloalkyl. R¹ may be substituted or unsubstituted 3 to 10 membered heterocycloalkyl. R¹ may be substituted 3 to 10 membered heterocycloalkyl. R¹ may be unsubstituted 3 to 10 membered heterocycloalkyl. R¹ may be substituted or unsubstituted 3 to 8 membered heterocycloalkyl. R¹ may be substituted 3 to 8 membered heterocycloalkyl. R¹ may be unsubstituted 3 to 8 membered heterocycloalkyl. R¹ may be substituted or unsubstituted 3 to 6 membered heterocycloalkyl. R¹ may be substituted 3 to 6 membered heterocycloalkyl. R¹ may be unsubstituted 3 to 6 membered heterocycloalkyl. R¹ may be substituted or unsubstituted 3 membered heterocycloalkyl. R¹ may be substituted or unsubstituted 4 membered heterocycloalkyl. R¹ may be substituted or unsubstituted 5 membered heterocycloalkyl. R¹ may be substituted or unsubstituted 6 membered heterocycloalkyl.

R¹ may be R^{1A}-substituted or unsubstituted heterocycloalkyl. R¹ may be R^{1A}-substituted heterocycloalkyl. R¹ may be unsubstituted heterocycloalkyl. R¹ may be R^{1A}-substituted or unsubstituted 3 to 10 membered heterocycloalkyl. R¹ may be R^{1A}-substituted 3 to 10 membered heterocycloalkyl. R¹ may be unsubstituted 3 to 10 membered heterocycloalkyl. R¹ may be R^{1A}-substituted or unsubstituted 3 to 8 membered heterocycloalkyl. R¹ may be R^{1A}-substituted 3 to 8 membered heterocycloalkyl. R¹ may be unsubstituted 3 to 8 membered heterocycloalkyl. R¹ may be R^{1A}-substituted or unsubstituted 3 to 6 membered heterocycloalkyl. R¹ may be R^{1A}-substituted 3 to 6 membered heterocycloalkyl. R¹ may be unsubstituted 3 to 6 membered heterocycloalkyl. R¹ may be R^{1A}-substituted or unsubstituted 3 membered heterocycloalkyl. R¹ may be R^{1A}-substituted or unsubstituted 4 membered heterocycloalkyl. R¹ may be R^{1A}-substituted or unsubstituted 5 membered heterocycloalkyl. R¹ may be R^{1A}-substituted or unsubstituted 6 membered heterocycloalkyl.

R¹ may be substituted or unsubstituted aryl. R¹ may be substituted aryl. R¹ may be unsubstituted aryl. R¹ may be substituted or unsubstituted 5 to 10 membered aryl. R¹ may be substituted 5 to 10 membered aryl. R¹ may be unsubstituted 5 to 10 membered aryl. R¹ may be substituted or unsubstituted 5 to 8 membered aryl. R¹ may be substituted 5 to 8 membered aryl. R¹ may be unsubstituted 5 to 8 membered aryl. R¹ may be substituted or unsubstituted 5 or 6 membered aryl. R¹ may be substituted 5 or 6 membered aryl. R¹ may be unsubstituted 5 or 6 membered aryl. R¹ may be substituted or unsubstituted 5 membered aryl. R¹ may be substituted or unsubstituted 6 membered aryl (e.g. phenyl).

R¹ may be R^{1A}-substituted or unsubstituted aryl. R¹ may be R^{1A}-substituted aryl. R¹ may be unsubstituted aryl. R¹ may be R^{1A}-substituted or unsubstituted 5 to 10 membered aryl. R¹ may be R^{1A}-substituted 5 to 10 membered aryl. R¹ may be unsubstituted 5 to 10 membered aryl. R¹ may be R^{1A}-substituted or unsubstituted 5 to 8 membered aryl. R¹ may be R^{1A}-substituted 5 to 8 membered aryl. R¹ may be unsubstituted 5 to 8 membered aryl. R¹ may be R^{1A}-substituted or unsubstituted 5 or 6 membered aryl. R¹ may be R^{1A}-substituted 5 or 6 membered aryl. R¹ may be unsubstituted 5 or 6 membered aryl. R¹ may be R^{1A}-substituted or unsubstituted 5 membered aryl. R¹ may be R^{1A}-substituted or unsubstituted 6 membered aryl (e.g. phenyl).

R¹ may be substituted or unsubstituted heteroaryl. R¹ may be substituted heteroaryl. R¹ may be unsubstituted heteroaryl. R¹ may be substituted or unsubstituted 5 to 10 membered heteroaryl. R¹ may be substituted 5 to 10 membered heteroaryl. R¹ may be unsubstituted 5 to 10 membered heteroaryl. R¹ may be substituted or unsubstituted 5 to 8 membered heteroaryl. R¹ may be substituted 5 to 8 membered heteroaryl. R¹ may be unsubstituted 5 to 8 membered heteroaryl. R¹ may be substituted or unsubstituted 5 or 6 membered heteroaryl. R¹ may be substituted 5 or 6 membered heteroaryl. R¹ may be unsubstituted 5 or 6 membered heteroaryl. R¹ may be substituted or unsubstituted 5 membered heteroaryl. R¹ may be substituted or unsubstituted 6 membered heteroaryl.

R¹ may be R^{1A}-substituted or unsubstituted heteroaryl. R¹ may be R^{1A}-substituted heteroaryl. R¹ may be unsubstituted heteroaryl. R¹ may be R^{1A}-substituted or unsubstituted 5 to 10 membered heteroaryl. R¹ may be R^{1A}-substituted 5 to 10 membered heteroaryl. R¹ may be unsubstituted 5 to 10 membered heteroaryl. R¹ may be R^{1A}-substituted or unsubstituted 5 to 8 membered heteroaryl. R¹ may be R^{1A}-substituted 5 to 8 membered heteroaryl. R¹ may be unsubstituted 5 to 8 membered heteroaryl. R¹ may be R^{1A}-substituted or unsubstituted 5 or 6 membered heteroaryl. R¹ may be R^{1A}-substituted 5 or 6 membered heteroaryl. R¹ may be unsubstituted 5 or 6 membered heteroaryl. R¹ may be R^{1A}-substituted or unsubstituted 5 membered heteroaryl. R¹ may be R^{1A}-substituted or unsubstituted 6 membered heteroaryl.

R¹ may be —O-L^{1A}-R^{1A}. L^{1A} is substituted or unsubstituted alkylene or substituted or unsubstituted heteroalkylene. L^{1A} may be substituted or unsubstituted alkylene. L^{1A} may be substituted or unsubstituted C₁-C₂₀ alkyl alkylene. L^{1A} may be substituted or unsubstituted C₁-C₁₀ alkylene. L^{1A} may be substituted or unsubstituted C₁-C₅ alkylene. L^{1A} may be substituted C₁-C₂₀ alkylene. L^{1A} may be unsubstituted C₁-C₂₀ alkylene. L^{1A} may be substituted C₁-C₁₀ alkylene. L^{1A} may be unsubstituted C₁-C₁₀ alkylene. L^{1A} may be substituted C₁-C₅ alkylene. L^{1A} may be unsubstituted C₁-C₅ alkylene. L^{1A} may be —(CH₂)ₘ—R^{1A}, where m is an integer selected from 1, 2, 3, 4 or 5. The symbol m may be 1. The symbol m may be 2. The symbol m may be 3. The symbol m may be 4. The symbol m may be 5.

L^{1A} may be substituted or unsubstituted heteroalkylene. L^{1A} may be substituted heteroalkylene. L^{1A} may be unsubstituted heteroalkylene. L^{1A} may be substituted or unsubstituted 2 to 20 membered heteroalkylene. L^{1A} may be substituted 2 to 20 membered heteroalkylene. L^{1A} may be substituted or unsubstituted 2 to 10 membered heteroalkylene. L^{1A} may be substituted 2 to 10 membered heteroalkylene. L^{1A} may be unsubstituted 2 to 10 membered heteroalkylene. L^{1A} may be substituted or unsubstituted 2 to 6 membered heteroalkylene. L^{1A} may be substituted 2 to 6 membered heteroalkylene. L^{1A} may be unsubstituted 2 to 6 membered heteroalkylene. L^{1A} may be —(CH₂CH₂O)ₘ₁—R^{1A}, where m1 is an integer of 1, 2, 3, or 4. The symbol m1 may be 1. The symbol m1 may be 2. The symbol m1 may be 3. The symbol m1 may be 4.

R¹ may be —O-L^{1A}-N(R^{1C})—S(O)ₙ₁—R^{1A}. R^{1A} is as described herein. R^{1A} may be hydrogen or substituted or unsubstituted alkyl (e.g. C₁-C₅ alkyl).

R^{1A} is hydrogen, halogen, oxo, —CF₃, —CN, —OR¹², —N(R^{12.1})(R^{12.2}), —COOR¹², —CON(R^{12.1})(R^{12.2}), —NO₂, —S(R¹²), —S(O)₂R¹², —S(O)₃R¹², —S(O)₄R¹², —S(O)₂N(R^{12.1})(R^{12.2}), —NHN(R^{12.1})(R^{12.2}), —ON(R^{12.1})(R^{12.2}), —NHC(O)NHN(R^{12.1})(R^{12.2}), —NHC(O)N(R^{12.1})(R^{12.2}), —NHS(O)₂R¹², —NHC(O)R¹², —NHC(O)—OR¹², —NHOR¹², —OCF₃, —OCHF₂, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl.

R¹¹ is hydrogen, halogen, oxo, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCHF₂, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl, or R¹²-substituted or unsubstituted heteroaryl.

R¹², R^{12.1} and R^{12.2} are independently hydrogen, halogen, oxo, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

R^{1A} may be —CH₃, —C₂H₅, —C₃H₇, —CD₃, —CD₂CD₃, —(CH₂)₂OH, —(CH₂)₃OH, —CH₂CH(OH)CH₃, —(CH₂)₂CH(OH)CH₃, —CH₂C(CH₃)₂OH, —(CH₂)₂C(CH₃)₂OH, —(CH₂)₂F, —(CH₂)₃F, —CH₂CH(F)CH₃, —(CH₂)₂CH(F)CH₃, —(CH₂)₂C(CH₃)₂F, —(CH₂)₂Cl, —(CH₂)₃Cl, —CH₂CH(Cl)CH₃, —(CH₂)₂CH(Cl)CH₃, —CH₂C(CH₃)₂Cl, —(CH₂)₂C(CH₃)₂Cl, —(CH₂)₂NHSO₂CH₃, —(CH₂)₃NHSO₂CH₃, —(CH₂)₂N(CH₂CH₂OH)SO₂CH₃, —(CH₂)₃N(CH₂CH₂OH)SO₂CH₃, —(CH₂)₂N(CH₂CH₂F)SO₂CH₃, —(CH₂)₂N(CH₂CH₂Cl)SO₂CH₃,

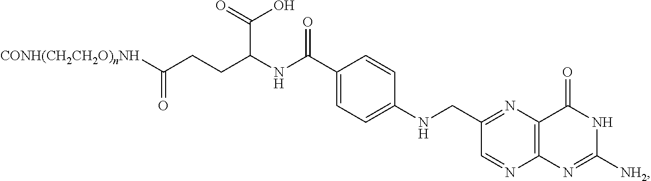

—(CH₂CH₂O)ₙCH₂CH₂-G^{1A} or —COCH₂CH₂COO(CH₂CH₂O)ₙCH₂CH₂-G^{1B}. The symbol n is 2-20. G^{1A} is H, —OH, —NH₂, —OCH₃, —OCF₃, F, Cl, —N₃, —NHCH₂C₆H₄NO₂, —NHCH₂C₆H₄F, —NHCH₂C₆H₄NO₂, —NHCH₂C₆H₄F,

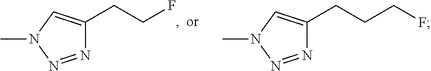

G^{1B} is H, —OH, —NH₂, —OCH₃, F, Cl,

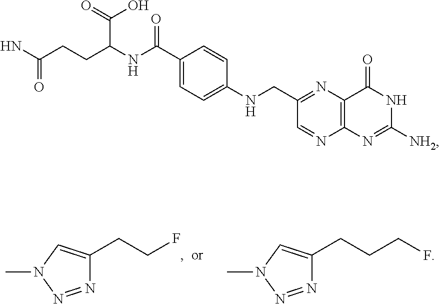

The symbol n may be 2-10. The symbol n may be 2-8. The symbol n may be 2-5. The symbol n may be 2, 3, or 4. The symbol n may be 3.

R^{1A} may be —OCH₃, —OCH₂CH₃, —O(CH₂)₂F, —(CH₂)₂NHSO₂CH₃, —(CH₂CH₂O)ₙF, —(CH₂CH₂O)ₙCH₃, where n is 2 to 5.

R^{1B} and R^{1C} are independently hydrogen, halogen, oxo, —OH, —NH₂, —COOH, —CONH₂, —S(O)₂Cl, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1B} may be hydrogen or substituted or unsubstituted alkyl.

R^{1C} are independently hydrogen, halogen, oxo, —OH, —NH₂, —COOH, —CONH₂, —S(O)₂Cl, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl, or R¹²-substituted or unsubstituted heteroaryl.

The compound of formula (I) may have formula:

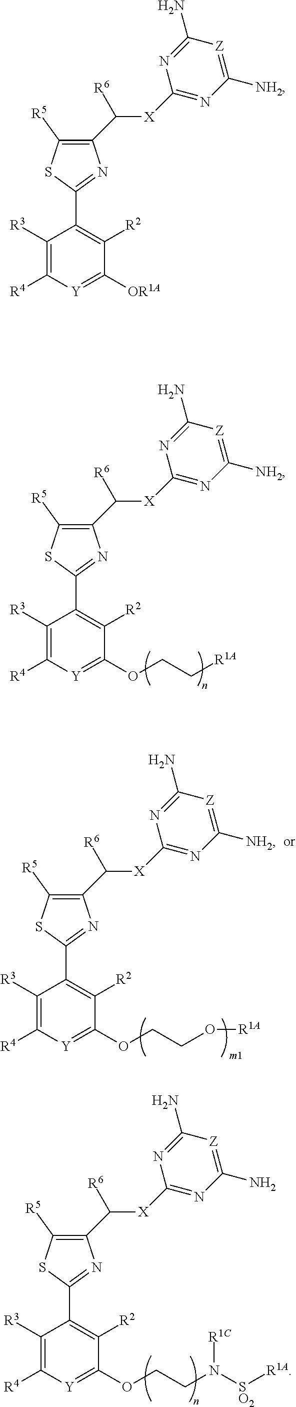

The symbol n is as described herein. The symbol n may be 1, 2, 3, or 4. The symbol n may be 1. The symbol n may be 2. The symbol n may be 3. The symbol n may be 4.

R² may be hydrogen, halogen, —N₃, —CF₃, —CCl₃, —CBr₃, —CI₃, —CN, —COR^{2A}, —OR^{2A}, —NR^{2A}R^{2B}, —C(O)OR^{2A}, —C(O)NR^{2A}R^{2B}, —NO₂, —SR^{2A}, —S(O)ₙ₂R^{2A}, —S(O)ₙ₂OR^{2A}, —S(O)ₙ₂NR^{2A}R^{2B}, —NHNR^{2A}R^{2B}, —ONR^{2A}R^{2B}, or —NHC(O)NHNR^{2A}R^{2B}. R² may be hydrogen, halogen, —CF₃, —OR^{2A}, or —NR^{2A}R^{2B}. R² may hydrogen. R² may be halogen. R² may be —CF₃. R² may be —OR^{2A}. R² may be —NR^{2A}R^{2B}. R² and R³ may be hydrogen.

R² may be substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² may be substituted or unsubstituted alkyl. R² may be unsubstituted alkyl R² may be substituted alkyl. R² may be substituted or unsubstituted C₁-C₂₀ alkyl. R² may be substituted or unsubstituted C₁-C₁₀ alkyl. R² may be substituted C₁-C₁₀ alkyl. R² may be unsubstituted C₁-C₁₀ alkyl. R² may be C₁-C₅ substituted or unsubstituted alkyl. R² may be substituted C₁-C₅ alkyl. R² may be unsubstituted C₁-C₅ alkyl. R² may be substituted or unsubstituted C₁-C₃ alkyl. R² may be unsubstituted C₁-C₃ alkyl. R² may be saturated C₁-C₃ alkyl. R² may be methyl. R² may be ethyl. R² may be propyl.

R² may be substituted or unsubstituted heteroalkyl. R² may be substituted heteroalkyl. R² may be unsubstituted alkyl. R² may be substituted or unsubstituted 2 to 10 membered heteroalkyl. R² may be substituted 2 to 10 membered heteroalkyl. R² may be unsubstituted 2 to 10 membered heteroalkyl. R² may be 2 to 6 membered heteroalkyl. R² may be substituted 2 to 6 membered heteroalkyl. R² may be unsubstituted 2 to 6 membered heteroalkyl.

R² may be substituted or unsubstituted 3 to 8 membered cycloalkyl. R² may be substituted 3 to 8 membered cycloalkyl. R² may be unsubstituted 3 to 8 membered cycloalkyl. R² may be substituted or unsubstituted 3 to 6 membered cycloalkyl. R² may be substituted 3 to 6 membered cycloalkyl. R² may be unsubstituted 3 to 6 membered cycloalkyl. R² may be substituted or unsubstituted 3 membered cycloalkyl. R² may be substituted or unsubstituted 4 membered cycloalkyl. R² may be 5 membered cycloalkyl. R² may be 6 membered cycloalkyl.

R² may be substituted or unsubstituted 3 to 8 membered heterocycloalkyl. R² may be substituted 3 to 8 membered heterocycloalkyl. R² may be unsubstituted 3 to 8 membered heterocycloalkyl. R² may be substituted or unsubstituted 3 to 6 membered heterocycloalkyl. R² may be substituted 3 to 6 membered heterocycloalkyl. R² may be unsubstituted 3 to 6 membered heterocycloalkyl. R² may be substituted or unsubstituted 3 membered heterocycloalkyl. R² may be substituted or unsubstituted 4 membered heterocycloalkyl. R² may be 5 membered heterocycloalkyl. R² may be 6 membered heterocycloalkyl.

R² may be substituted or unsubstituted 5 to 8 membered aryl. R² may be substituted 5 to 8 membered aryl. R² may be unsubstituted 5 to 8 membered aryl. R² may be substituted or unsubstituted 5 membered aryl. R² may be substituted 5 membered aryl. R² may be unsubstituted 5 membered aryl. R² may be substituted 6 membered aryl. R² may be unsubstituted 6 membered aryl (e.g. phenyl).

R² may be substituted or unsubstituted 5 to 8 membered heteroaryl. R² may be substituted 5 to 8 membered heteroaryl. R² may be unsubstituted 5 to 8 membered heteroaryl. R² may be substituted or unsubstituted 5 membered heteroaryl. R² may be substituted 5 membered aryl. R² may be unsubstituted 5 membered heteroaryl. R² may be substituted 6 membered aryl. R² may be unsubstituted 6 membered heteroaryl.

R³ may be hydrogen, halogen, —N₃, —CF₃, —CCl₃, —CBr₃, —CI₃, —CN, —COR^{3A}, —OR^{3A}, —NR^{3A}R^{3B}, —C(O)OR^{3A}, —C(O)NR^{3A}R^{3B}, —NO₂, —SR^{3A}, —S(O)ₙ₃R^{3A}, —S(O)ₙ₃OR^{3A}, —S(O)ₙ₃NR^{3A}R^{3B}, —NHNR^{3A}R^{3B}, —ONR^{3A}R^{3B}, or —NHC(O)NHNR^{3A}R^{3B}. R³ may be hydrogen, halogen, —CF₃, —OR^{3A}, or —NR^{3A}R^{3B}. R³ may hydrogen. R³ may be halogen. R³ may be —CF₃. R³ may be —OR^{3A}. R³ may be —NR^{3A}R^{3B}. R² and R³ may be hydrogen.

R³ may be substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ may be substituted or unsubstituted alkyl. R³ may be unsubstituted alkyl. R³ may be substituted alkyl. R³ may be substituted or unsubstituted C₁-C₂₀ alkyl. R³ may be substituted or unsubstituted C₁-C₁₀ alkyl. R³ may be substituted C₁-C₁₀ alkyl. R³ may be unsubstituted C₁-C₁₀ alkyl. R³ may be C₁-C₅ substituted or unsubstituted alkyl. R³ may be substituted C₁-C₅ alkyl. R³ may be unsubstituted C₁-C₅ alkyl. R³ may be substituted or unsubstituted C₁-C₃ alkyl. R³ may be unsubstituted C₁-C₃ alkyl. R³ may be saturated C₁-C₃ alkyl. R³ may be methyl. R³ may be ethyl. R³ may be propyl.

R³ may be substituted or unsubstituted heteroalkyl. R³ may be substituted heteroalkyl. R³ may be unsubstituted alkyl. R³ may be substituted or unsubstituted 2 to 10 membered heteroalkyl. R³ may be substituted 2 to 10 membered heteroalkyl. R³ may be unsubstituted 2 to 10 membered heteroalkyl. R³ may be 2 to 6 membered heteroalkyl. R³ may be substituted 2 to 6 membered heteroalkyl. R³ may be unsubstituted 2 to 6 membered heteroalkyl.

R³ may be substituted or unsubstituted 3 to 8 membered cycloalkyl. R³ may be substituted 3 to 8 membered cycloalkyl. R³ may be unsubstituted 3 to 8 membered cycloalkyl. R³ may be substituted or unsubstituted 3 to 6 membered cycloalkyl. R³ may be substituted 3 to 6 membered cycloalkyl. R³ may be unsubstituted 3 to 6 membered cycloalkyl. R³ may be substituted or unsubstituted 3 membered cycloalkyl. R³ may be substituted or unsubstituted 4 membered cycloalkyl. R³ may be 5 membered cycloalkyl. R³ may be 6 membered cycloalkyl.

R³ may be substituted or unsubstituted 3 to 8 membered heterocycloalkyl. R³ may be substituted 3 to 8 membered heterocycloalkyl. R³ may be unsubstituted 3 to 8 membered heterocycloalkyl. R³ may be substituted or unsubstituted 3 to 6 membered heterocycloalkyl. R³ may be substituted 3 to 6 membered heterocycloalkyl. R³ may be unsubstituted 3 to 6 membered heterocycloalkyl. R³ may be substituted or unsubstituted 3 membered heterocycloalkyl. R³ may be substituted or unsubstituted 4 membered heterocycloalkyl. R³ may be 5 membered heterocycloalkyl. R³ may be 6 membered heterocycloalkyl.

R³ may be substituted or unsubstituted 5 to 8 membered aryl. R³ may be substituted 5 to 8 membered aryl. R³ may be unsubstituted 5 to 8 membered aryl. R³ may be substituted or unsubstituted 5 membered aryl. R³ may be substituted 5 membered aryl. R³ may be unsubstituted 5 membered aryl. R³ may be substituted 6 membered aryl. R³ may be unsubstituted 6 membered aryl (e.g. phenyl).

R³ may be substituted or unsubstituted 5 to 8 membered heteroaryl. R³ may be substituted 5 to 8 membered heteroaryl. R³ may be unsubstituted 5 to 8 membered heteroaryl. R³ may be substituted or unsubstituted 5 membered heteroaryl. R³ may be substituted 5 membered aryl. R³ may be unsubstituted 5 membered heteroaryl. R³ may be substituted 6 membered aryl. R³ may be unsubstituted 6 membered heteroaryl.

R⁴ may be hydrogen, halogen, —N₃, —CF₃, —CCl₃, —CBr₃, —CI₃, —CN, —COR^{4A}, —OR^{4A}, —NR^{4A}R^{4B}, —C(O)OR^{4A}, —C(O)NR^{4A}R^{4B}, —NO₂, —SR^{4A}, —S(O)ₙ₄R^{4A}, —S(O)ₙ₄OR^{4A}, —S(O)ₙ₄NR^{4A}R^{4B}, —NHNR^{4A}R^{4B}, —ONR^{4A}R^{4B}, or —NHC(O)NHNR^{4A}R^{4B}. R⁴ may be hydrogen or halogen. R⁴ may be hydrogen. R⁴ may be halogen.

R⁴ may be substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁴ may be substituted or unsubstituted alkyl. R⁴ may be unsubstituted alkyl. R⁴ may be substituted alkyl. R⁴ may be substituted or unsubstituted C₁-C₂₀ alkyl. R⁴ may be substituted or unsubstituted C₁-C₁₀ alkyl. R⁴ may be substituted C₁-C₁₀ alkyl. R⁴ may be unsubstituted C₁-C₁₀ alkyl. R⁴ may be C₁-C₅ substituted or unsubstituted alkyl. R⁴ may be substituted C₁-C₅ alkyl. R⁴ may be unsubstituted C₁-C₅ alkyl. R⁴ may be substituted or unsubstituted C₁-C₃ alkyl. R⁴ may be unsubstituted C₁-C₃ alkyl. R⁴ may be saturated C₁-C₃ alkyl. R⁴ may be methyl. R⁴ may be ethyl. R⁴ may be propyl.

R⁴ may be substituted or unsubstituted heteroalkyl. R⁴ may be substituted heteroalkyl. R⁴ may be unsubstituted alkyl. R⁴ may be substituted or unsubstituted 2 to 10 membered heteroalkyl. R⁴ may be substituted 2 to 10 membered heteroalkyl. R⁴ may be unsubstituted 2 to 10 membered heteroalkyl. R⁴ may be 2 to 6 membered heteroalkyl. R⁴ may be substituted 2 to 6 membered heteroalkyl. R⁴ may be unsubstituted 2 to 6 membered heteroalkyl.

R⁴ may be substituted or unsubstituted 3 to 8 membered cycloalkyl. R⁴ may be substituted 3 to 8 membered cycloalkyl. R⁴ may be unsubstituted 3 to 8 membered cycloalkyl. R⁴ may be substituted or unsubstituted 3 to 6 membered cycloalkyl. R⁴ may be substituted 3 to 6 membered cycloalkyl. R⁴ may be unsubstituted 3 to 6 membered cycloalkyl. R⁴ may be substituted or unsubstituted 3 membered cycloalkyl. R⁴ may be substituted or unsubstituted 4 membered cycloalkyl. R⁴ may be 5 membered cycloalkyl. R⁴ may be 6 membered cycloalkyl.

R⁴ may be substituted or unsubstituted 3 to 8 membered heterocycloalkyl. R⁴ may be substituted 3 to 8 membered heterocycloalkyl. R⁴ may be unsubstituted 3 to 8 membered heterocycloalkyl. R⁴ may be substituted or unsubstituted 3 to 6 membered heterocycloalkyl. R⁴ may be substituted 3 to 6 membered heterocycloalkyl. R⁴ may be unsubstituted 3 to 6 membered heterocycloalkyl. R⁴ may be substituted or unsubstituted 3 membered heterocycloalkyl. R⁴ may be substituted or unsubstituted 4 membered heterocycloalkyl. R⁴ may be 5 membered heterocycloalkyl. R⁴ may be 6 membered heterocycloalkyl.

R⁴ may be substituted or unsubstituted 5 to 8 membered aryl. R⁴ may be substituted 5 to 8 membered aryl. R⁴ may be unsubstituted 5 to 8 membered aryl. R⁴ may be substituted or unsubstituted 5 membered aryl. R⁴ may be substituted 5 membered aryl. R⁴ may be unsubstituted 5 membered aryl. R⁴ may be substituted 6 membered aryl. R⁴ may be unsubstituted 6 membered aryl (e.g. phenyl).

R⁴ may be substituted or unsubstituted 5 to 8 membered heteroaryl. R⁴ may be substituted 5 to 8 membered heteroaryl. R⁴ may be unsubstituted 5 to 8 membered heteroaryl. R⁴ may be substituted or unsubstituted 5 membered heteroaryl. R⁴ may be substituted 5 membered aryl. R⁴ may be unsubstituted 5 membered heteroaryl. R⁴ may be substituted 6 membered aryl. R⁴ may be unsubstituted 6 membered heteroaryl.

R⁵ may be hydrogen, halogen, —N₃, —CF₃, —CCl₃, —CBr₃, —CI₃, —CN, —COR^{5A}, —OR^{5A}, —NR^{5A}R^{5B}, —C(O)OR^{5A}, —C(O)NR^{5A}R^{5B}, —NO₂, —SR^{5A}, —S(O)ₙ₅R^{5A}, —S(O)ₙ₅OR^{5A}, —S(O)ₙ₅NR^{5A}R^{5B}, —NHNR^{5A}R^{5B}, —ONR^{5A}R^{5B}, or —NHC(O)NHNR^{5A}R^{5B}.

R⁵ may be substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵ may be substituted or unsubstituted alkyl or substituted or unsubstituted heteroalkyl. R⁵ may be substituted or unsubstituted alkyl. R⁵ may be unsubstituted alkyl. R⁵ may be substituted alkyl. R⁵ may be substituted or unsubstituted C₁-C₂₀ alkyl. R⁵ may be substituted C₁-C₂₀ alkyl. R⁵ may be unsubstituted C₁-C₂₀ alkyl. R⁵ may be substituted or unsubstituted C₁-C₁₀ alkyl. R⁵ may be substituted C₁-C₁₀ alkyl. R⁵ may be unsubstituted C₁-C₁₀ alkyl. R⁵ may be C₁-C₆ substituted or unsubstituted alkyl. R⁴ may be substituted C₁-C₆ alkyl. R⁵ may be unsubstituted C₁-C₆ alkyl. R⁵ may be substituted or unsubstituted C₁-C₃ alkyl. R⁵ may be unsubstituted C₁-C₃ alkyl. R⁵ may be saturated C₁-C₃ alkyl. R⁵ may be methyl. R⁵ may be ethyl. R⁵ may be propyl.

R⁵ may be substituted or unsubstituted heteroalkyl. R⁵ may be substituted heteroalkyl. R⁵ may be unsubstituted alkyl. R⁵ may be substituted or unsubstituted 2 to 10 membered heteroalkyl. R⁵ may be substituted 2 to 10 membered heteroalkyl. R⁵ may be unsubstituted 2 to 10 membered heteroalkyl. R⁵ may be 2 to 6 membered heteroalkyl. R⁵ may be substituted 2 to 6 membered heteroalkyl. R⁵ may be unsubstituted 2 to 6 membered heteroalkyl.

R⁵ may be substituted or unsubstituted 3 to 8 membered cycloalkyl. R⁵ may be substituted 3 to 8 membered cycloalkyl. R⁵ may be unsubstituted 3 to 8 membered cycloalkyl. R⁵ may be substituted or unsubstituted 3 to 6 membered cycloalkyl. R⁵ may be substituted 3 to 6 membered cycloalkyl. R⁵ may be unsubstituted 3 to 6 membered cycloalkyl. R⁵ may be substituted or unsubstituted 3 membered cycloalkyl. R⁵ may be substituted or unsubstituted 4 membered cycloalkyl. R⁵ may be 5 membered cycloalkyl. R⁵ may be 6 membered cycloalkyl.

R⁵ may be substituted or unsubstituted 3 to 8 membered heterocycloalkyl. R⁵ may be substituted 3 to 8 membered heterocycloalkyl. R⁵ may be unsubstituted 3 to 8 membered heterocycloalkyl. R⁵ may be substituted or unsubstituted 3 to 6 membered heterocycloalkyl. R⁵ may be substituted 3 to 6 membered heterocycloalkyl. R⁵ may be unsubstituted 3 to 6 membered heterocycloalkyl. R⁵ may be substituted or unsubstituted 3 membered heterocycloalkyl. R⁵ may be substituted or unsubstituted 4 membered heterocycloalkyl. R⁵ may be 5 membered heterocycloalkyl. R⁵ may be 6 membered heterocycloalkyl.

R⁵ may be substituted or unsubstituted 5 to 8 membered aryl. R⁵ may be substituted 5 to 8 membered aryl. R⁵ may be unsubstituted 5 to 8 membered aryl. R⁵ may be substituted or unsubstituted 5 membered aryl. R⁵ may be substituted 5 membered aryl. R⁵ may be unsubstituted 5 membered aryl. R⁵ may be substituted 6 membered aryl. R⁵ may be unsubstituted 6 membered aryl (e.g. phenyl).

R⁵ may be substituted or unsubstituted 5 to 8 membered heteroaryl. R⁵ may be substituted 5 to 8 membered heteroaryl. R⁵ may be unsubstituted 5 to 8 membered heteroaryl. R⁵ may be substituted or unsubstituted 5 membered heteroaryl. R⁵ may be substituted 5 membered aryl. R⁵ may be unsubstituted 5 membered heteroaryl. R⁵ may be substituted 6 membered aryl. R⁵ may be unsubstituted 6 membered heteroaryl.

R⁵ and R⁶ may optionally be combined to form a substituted or unsubstituted cycloalkyl. R⁵ and R⁶ may optionally be combined to form a substituted cycloalkyl. R⁵ and R⁶ may optionally be combined to form an unsubstituted cycloalkyl. R⁵ and R⁶ may optionally be combined to form a substituted or unsubstituted 3 to 10 membered cycloalkyl. R⁵ and R⁶ may optionally be combined to form a substituted 3 to 10 membered cycloalkyl. R⁵ and R⁶ may optionally be combined to form an unsubstituted 3 to 10 membered cycloalkyl. R⁵ and R⁶ may optionally be combined to form a substituted or unsubstituted 3 to 8 membered cycloalkyl. R⁵ and R⁶ may optionally be combined to form a substituted 3 to 8 membered cycloalkyl. R⁵ and R⁶ may optionally be combined to form an unsubstituted 3 to 8 membered cycloalkyl. R⁵ and R⁶ may optionally be combined to form a substituted or unsubstituted 3 to 6 membered cycloalkyl. R⁵ and R⁶ may optionally be combined to form a substituted 3 to 6 membered cycloalkyl. R⁵ and R⁶ may optionally be combined to form an unsubstituted 3 to 6 membered cycloalkyl. R⁵ and R⁶ may optionally be combined to form a substituted or unsubstituted 3 membered cycloalkyl. R⁵ and R⁶ may optionally be combined to form a substituted or unsubstituted 4 membered cycloalkyl. R⁵ and R⁶ may optionally be combined to form a substituted or unsubstituted 5 membered cycloalkyl. R⁵ and R⁶ may optionally be combined to form a substituted or unsubstituted 6 membered cycloalkyl.

R⁵ and R⁶ may optionally be combined to form a R^{5A}-substituted or unsubstituted cycloalkyl. R⁵ and R⁶ may optionally be combined to form a R^{5A}-substituted R⁵ and R⁶ may optionally be combined to form a R^{5A}-substituted or unsubstituted 3 to 10 membered cycloalkyl. R⁵ and R⁶ may optionally be combined to form a R^{5A}-substituted 3 to 10 membered cycloalkyl. R⁵ and R⁶ may optionally be combined to form a R^{5A}-substituted or unsubstituted 3 to 8 membered cycloalkyl. R⁵ and R⁶ may optionally be combined to form a R^{5A}-substituted 3 to 8 membered cycloalkyl. R⁵ and R⁶ may optionally be combined to form a R^{5A}-substituted or unsubstituted 3 to 6 membered cycloalkyl. R⁵ and R⁶ may optionally be combined to form a R^{5A}-substituted 3 to 6 membered cycloalkyl. R⁵ and R⁶ may optionally be combined to form a R^{5A}-substituted or unsubstituted 3 membered cycloalkyl. R⁵ and R⁶ may optionally be combined to form a R^{5A}-substituted 3 membered cycloalkyl. R⁵ and R⁶ may optionally be combined to form an unsubstituted 3 membered cycloalkyl. R⁵ and R⁶ may optionally be combined to form a R^{5A}-substituted or unsubstituted 4 membered cycloalkyl. R⁵ and R⁶ may optionally be combined to form a R^{5A}-substituted 4 membered cycloalkyl. R⁵ and R⁶ may optionally be combined to form an unsubstituted 4 membered cycloalkyl. R⁵ and R⁶ may optionally be combined to form a R^{5A}-substituted or unsubstituted 5 membered cycloalkyl. R⁵ and R⁶ may optionally be combined to form a R^{5A}-substituted 5 membered cycloalkyl. R⁵ and R⁶ may optionally be combined to form an unsubstituted 5 membered cycloalkyl. R⁵ and R⁶ may optionally be combined to form a R^{5A}-substituted or unsubstituted 6 membered cycloalkyl. R⁵ and R⁶ may optionally be combined to form a R^{5A}-substituted 6 membered cycloalkyl. R⁵ and R⁶ may optionally be combined to form an unsubstituted 6 membered cycloalkyl.

R⁵ and R⁶ may independently be unsubstituted C₁-C₆ alkyl. R⁵ and R⁶ may independently be unsubstituted C₁-C₄ alkyl. R⁵ and R⁶ may independently be methyl, ethyl, or propyl. R⁵ and R⁶ may independently be methyl. When R⁵ is methyl or propyl, R⁶ may be methyl.

R⁶ may be unsubstituted C₁-C₆ alkyl. R⁶ may be unsubstituted C₁-C₅ alkyl. R⁶ may be unsubstituted C₁-C₄ alkyl. R⁶ may be unsubstituted C₁-C₃ alkyl. R⁶ may be methyl, ethyl, or propyl. R⁶ may be methyl. R⁶ may be ethyl. R⁶ may be propyl. R⁶ may be methyl and R⁵ may be methyl, ethyl, or propyl. R⁶ may be methyl and R⁵ may be methyl. R⁶ may be methyl and R⁵ may be ethyl. R⁶ may be methyl and R⁵ may be propyl. R⁶ may be halogen.

R⁶ may be described as herein and attached to a carbon having (R) stereochemistry. R⁶ may be (R)—C₁-C₆ alkyl. R⁶ may be (R)—C₁-C₅ alkyl. R⁶ may be a (R)—C₁-C₄ alkyl. R⁶ may be a (R)—C₁-C₃ alkyl. R⁶ may be (R)-methyl. R⁶ may be (R)-ethyl. R⁶ may be a (R)-propyl.

R⁶ may be as described herein and attached to a carbon having (S) stereochemistry. R⁶ may be (S)—C₁-C₆ alkyl. R⁶ may be (S)—C₁-C₅ alkyl. R⁶ may be a (S)—C₁-C₄ alkyl. R⁶ may be a (S)—C₁-C₃ alkyl. R⁶ may be (S)-methyl. R⁶ may be (S)-ethyl. R⁶ may be a (S)-propyl. When R⁵ is methyl or propyl, R⁶ may be (R)-methyl.

R⁷ may be hydrogen, halogen, —N₃, —CF₃, —CCl₃, —CBr₃, —CI₃, —CN, —COR^{7A}, —OR^{7A}, —NR^{7A}R^{7B}, —C(O)OR^{7A}, —C(O)NR^{7A}R^{7B}, —NO₂, —SR^{7A}, —S(O)ₙ₇R^{7A}, —S(O)ₙ₇OR^{7A}, —S(O)ₙ₇NR^{7A}R^{7B}, —NHNR^{7A}R^{7B}, —ONR^{7A}R^{7B}, or —NHC(O)NHNR^{7A}R^{7B}. R⁷ may be hydrogen, halogen, —CF₃, —OR^{7A}, or —NR^{7A}R^{7B}. R⁷ may hydrogen. R⁷ may be halogen. R⁷ may be —CF₃. R⁷ may be —OR^{7A}. R⁷ may be —NR^{7A}R^{7B}.

R⁷ may be substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁷ may be substituted or unsubstituted alkyl. R⁷ may be unsubstituted alkyl R⁷ may be substituted alkyl. R⁷ may be substituted or unsubstituted C₁-C₂₀ alkyl. R⁷ may be substituted or unsubstituted C₁-C₁₀ alkyl. R⁷ may be substituted C₁-C₁₀ alkyl. R⁷ may be unsubstituted C₁-C₁₀ alkyl. R⁷ may be C₁-C₅ substituted or unsubstituted alkyl. R⁷ may be substituted C₁-C₅ alkyl. R⁷ may be unsubstituted C₁-C₅ alkyl. R⁷ may be substituted or unsubstituted C₁-C₃ alkyl. R⁷ may be unsubstituted C₁-C₃ alkyl. R⁷ may be saturated C₁-C₃ alkyl. R⁷ may be methyl. R⁷ may be ethyl. R⁷ may be propyl.

R⁷ may be substituted or unsubstituted heteroalkyl. R² may be substituted heteroalkyl. R⁷ may be unsubstituted alkyl. R⁷ may be substituted or unsubstituted 2 to 10 membered heteroalkyl. R⁷ may be substituted 2 to 10 membered heteroalkyl. R⁷ may be unsubstituted 2 to 10 membered heteroalkyl. R⁷ may be 2 to 6 membered heteroalkyl. R⁷ may be substituted 2 to 6 membered heteroalkyl. R⁷ may be unsubstituted 2 to 6 membered heteroalkyl.

R⁷ may be substituted or unsubstituted 3 to 8 membered cycloalkyl. R⁷ may be substituted 3 to 8 membered cycloalkyl. R⁷ may be unsubstituted 3 to 8 membered cycloalkyl. R⁷ may be substituted or unsubstituted 3 to 6 membered cycloalkyl. R⁷ may be substituted 3 to 6 membered cycloalkyl. R⁷ may be unsubstituted 3 to 6 membered cycloalkyl. R⁷ may be substituted or unsubstituted 3 membered cycloalkyl. R⁷ may be substituted or unsubstituted 4 membered cycloalkyl. R² may be 5 membered cycloalkyl. R⁷ may be 6 membered cycloalkyl.

R⁷ may be substituted or unsubstituted 3 to 8 membered heterocycloalkyl. R⁷ may be substituted 3 to 8 membered heterocycloalkyl. R⁷ may be unsubstituted 3 to 8 membered heterocycloalkyl. R⁷ may be substituted or unsubstituted 3 to 6 membered heterocycloalkyl. R⁷ may be substituted 3 to 6 membered heterocycloalkyl. R⁷ may be unsubstituted 3 to 6 membered heterocycloalkyl. R⁷ may be substituted or unsubstituted 3 membered heterocycloalkyl. R⁷ may be substituted or unsubstituted 4 membered heterocycloalkyl. R⁷ may be 5 membered heterocycloalkyl. R⁷ may be 6 membered heterocycloalkyl.

R⁷ may be substituted or unsubstituted 5 to 8 membered aryl. R⁷ may be substituted 5 to 8 membered aryl. R⁷ may be unsubstituted 5 to 8 membered aryl. R⁷ may be substituted or unsubstituted 5 membered aryl. R⁷ may be substituted 5 membered aryl. R⁷ may be unsubstituted 5 membered aryl. R⁷ may be substituted 6 membered aryl. R⁷ may be unsubstituted 6 membered aryl (e.g. phenyl).

R⁷ may be substituted or unsubstituted 5 to 8 membered heteroaryl. R⁷ may be substituted 5 to 8 membered heteroaryl. R⁷ may be unsubstituted 5 to 8 membered heteroaryl. R⁷ may be substituted or unsubstituted 5 membered heteroaryl. R⁷ may be substituted 5 membered aryl. R⁷ may be unsubstituted 5 membered heteroaryl. R⁷ may be substituted 6 membered aryl. R⁷ may be unsubstituted 6 membered heteroaryl.

Y may be N. Y may be C(R⁸). Z may be N. Z may be C(R⁹). Y and Z may be N. Y may be C(R⁸), where R⁸ is as described herein and Z may be C(R⁹), where R⁹ is as described herein. Y may be C(R⁸), where R⁸ is as described herein and Z may be C(R⁹), where R⁹ is independently hydrogen. Y may be N and Z may be C(R⁹), where R⁹ is as described herein. Y may be N and Z may be C(R⁹), where R⁹ is independently hydrogen.

X may be —CH₂. X may be O, N(R¹⁰), or S, where R¹⁰ is as described herein. X may be S(O) or S(O)₂. X may be S. X may be O. X may be N(R¹⁰), where R¹⁰ is as described herein.

R¹⁰ may be hydrogen. R¹⁰ may be —CH₃, —C₂H₅, —C₃H₇, —CH₂C₆H₅. R¹⁰ may be hydrogen or methyl. R¹⁰ may be hydrogen or —C₂H₅. R¹⁰ may be hydrogen or —C₃H₇. R¹⁰ may be hydrogen or —CH₂C₆H₅. R¹⁰ may be —CH₃. R¹⁰ may be —C₂H₅. R¹⁰ may be —C₃H₇. R¹⁰ may be —CH₂C₆H₅.

R⁸ may be hydrogen, halogen, —N₃, —CF₃, —CCl₃, —CBr₃, —CI₃, —CN, —COR^{8A}, —OR^{8A}, —O-L^{8A}-R^{8C}, —NR^{8A}R^{8B}, —C(O)OR^{8A}, —C(O)NR^{8A}R^{8B}, —NO₂, —SR^{8A}, —S(O)ₙ⁸R^{8A}, —S(O)ₙ₈OR^{8A}, —S(O)ₙ⁸NR^{8A}R^{8B}, —NHNR^{8A}R^{8B}, —ONR^{8A}R^{8B}, or —NHC(O)NHNR^{8A}R^{8B}. R⁸ may be hydrogen, halogen, —OR^{8A}. R⁸ may be hydrogen. R⁸ may be halogen. R⁸ may be —OR^{8A}. R^{8A} is as described herein.

R⁸ may be hydrogen, halogen, —OR^{8A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁸ may be —OR^{8A}, where R^{8A} is as described herein. R⁸ may be —OR^{8A}, where R^{8A} is hydrogen, substituted or unsubstituted alkyl, or substituted or unsubstituted heteroalkyl. R⁸ may be —OR^{8A}, where R^{8A} is substituted or unsubstituted alkyl, or substituted or unsubstituted heteroalkyl.

R⁸ may be substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁸ may be R^{8A}-substituted or unsubstituted alkyl, R^{8A}-substituted or unsubstituted heteroalkyl, R^{8A}-substituted or unsubstituted cycloalkyl, R^{8A}-substituted or unsubstituted heterocycloalkyl, R^{8A}-substituted or unsubstituted aryl, or R^{8A}-substituted or unsubstituted heteroaryl.

R⁸ may be substituted or unsubstituted alkyl. R⁸ may be substituted alkyl. R⁸ may be unsubstituted alkyl. R⁸ may be substituted or unsubstituted C₁-C₂₀ alkyl. R⁸ may be substituted C₁-C₂₀ alkyl. R⁸ may be unsubstituted C₁-C₂₀ alkyl. R⁸ may be substituted or unsubstituted C₁-C₁₀ alkyl. R⁸ may be substituted C₁-C₁₀ alkyl. R⁸ may be unsubstituted C₁-C₁₀ alkyl. R⁸ may be substituted or unsubstituted C₁-C₅ alkyl. R⁸ may be substituted C₁-C₅ alkyl. R⁸ may be unsubstituted C₁-C₅ alkyl. R⁸ may be methyl. R⁸ may be ethyl. R⁸ may be propyl.

R⁸ may be R^{8A}-substituted or unsubstituted alkyl. R⁸ may be R^{8A}-substituted alkyl. R⁸ may be unsubstituted alkyl. R⁸ may be R^{8A}-substituted or unsubstituted C₁-C₂₀ alkyl. R⁸ may be R^{8A}-substituted C₁-C₂₀ alkyl. R⁸ may be unsubstituted C₁-C₂₀ alkyl. R⁸ may be R^{8A}-substituted or unsubstituted C₁-C₁₀ alkyl. R⁸ may be R^{8A}-substituted C₁-C₁₀ alkyl. R⁸ may be unsubstituted C₁-C₁₀ alkyl. R⁸ may be R^{8A}-substituted or unsubstituted C₁-C₅ alkyl. R⁸ may be R^{8A}-substituted C₁-C₅ alkyl. R⁸ may be unsubstituted C₁-C₅ alkyl.

R⁸ may be substituted or unsubstituted heteroalkyl. R⁸ may be substituted heteroalkyl. R⁸ may be unsubstituted heteroalkyl. R⁸ may be substituted or unsubstituted 2 to 20 membered heteroalkyl. R⁸ may be substituted 2 to 20 membered heteroalkyl. R⁸ may be unsubstituted 2 to 20 membered heteroalkyl. R⁸ may be substituted or unsubstituted 2 to 10 membered heteroalkyl. R⁸ may be substituted 2 to 10 membered heteroalkyl. R⁸ may be unsubstituted 2 to 10 membered heteroalkyl. R⁸ may be substituted or unsubstituted 2 to 6 membered heteroalkyl. R⁸ may be substituted 2 to 6 membered heteroalkyl. R⁸ may be unsubstituted 2 to 6 membered heteroalkyl.

R⁸ may be R^{8A}-substituted or unsubstituted heteroalkyl. R⁸ may be R^{8A}-substituted heteroalkyl. R⁸ may be unsubstituted heteroalkyl. R⁸ may be R^{8A}-substituted or unsubstituted 2 to 20 membered heteroalkyl. R⁸ may be R^{8A}-substituted 2 to 20 membered heteroalkyl. R⁸ may be unsubstituted 2 to 20 membered heteroalkyl. R⁸ may be R^{8A}-substituted or unsubstituted 2 to 10 membered heteroalkyl. R⁸ may be R^{8A}-substituted 2 to 10 membered heteroalkyl. R⁸ may be unsubstituted 2 to 10 membered heteroalkyl. R⁸ may be R^{8A}-substituted or unsubstituted 2 to 6 membered heteroalkyl. R⁸ may be R^{8A}-substituted 2 to 6 membered heteroalkyl. R⁸ may be unsubstituted 2 to 6 membered heteroalkyl.

R⁸ may be substituted or unsubstituted cycloalkyl. R⁸ may be substituted cycloalkyl. R⁸ may be unsubstituted cycloalkyl. R⁸ may be substituted or unsubstituted 3 to 10 membered cycloalkyl. R⁸ may be substituted 3 to 10 membered cycloalkyl. R⁸ may be unsubstituted 3 to 10 membered cycloalkyl. R⁸ may be substituted or unsubstituted 3 to 8 membered cycloalkyl. R⁸ may be substituted 3 to 8 membered cycloalkyl. R⁸ may be unsubstituted 3 to 8 membered cycloalkyl. R⁸ may be substituted or unsubstituted 3 to 6 membered cycloalkyl. R⁸ may be substituted 3 to 6 membered cycloalkyl. R⁸ may be unsubstituted 3 to 6 membered cycloalkyl. R⁸ may be substituted or unsubstituted 3 membered cycloalkyl. R⁸ may be substituted or unsubstituted 4 membered cycloalkyl. R⁸ may be substituted or unsubstituted 5 membered cycloalkyl. R⁸ may be substituted or unsubstituted 6 membered cycloalkyl.

R⁸ may be R^{8A}-substituted or unsubstituted cycloalkyl. R⁸ may be R^{8A}-substituted cycloalkyl. R⁸ may be unsubstituted cycloalkyl. R⁸ may be R^{8A}-substituted or unsubstituted 3 to 10 membered cycloalkyl. R⁸ may be R^{8A}-substituted 3 to 10 membered cycloalkyl. R⁸ may be unsubstituted 3 to 10 membered cycloalkyl. R⁸ may be R^{8A}-substituted or unsubstituted 3 to 8 membered cycloalkyl. R⁸ may be R^{8A}-substituted 3 to 8 membered cycloalkyl. R⁸ may be unsubstituted 3 to 8 membered cycloalkyl. R⁸ may be R^{8A}-substituted or unsubstituted 3 to 6 membered cycloalkyl. R⁸ may be R^{8A}-substituted 3 to 6 membered cycloalkyl. R⁸ may be unsubstituted 3 to 6 membered cycloalkyl. R⁸ may be R^{8A}-substituted or unsubstituted 3 membered cycloalkyl. R⁸ may be R^{8A}-substituted or unsubstituted 4 membered cycloalkyl. R⁸ may be R^{8A}-substituted or unsubstituted 5 membered cycloalkyl. R⁸ may be R^{8A}-substituted or unsubstituted 6 membered cycloalkyl.

R⁸ may be substituted or unsubstituted heterocycloalkyl. R⁸ may be substituted heterocycloalkyl. R⁸ may be unsubstituted heterocycloalkyl. R⁸ may be substituted or unsubstituted 3 to 10 membered heterocycloalkyl. R⁸ may be substituted 3 to 10 membered heterocycloalkyl. R⁸ may be unsubstituted 3 to 10 membered heterocycloalkyl. R⁸ may be substituted or unsubstituted 3 to 8 membered heterocycloalkyl. R⁸ may be substituted 3 to 8 membered heterocycloalkyl. R⁸ may be unsubstituted 3 to 8 membered heterocycloalkyl. R⁸ may be substituted or unsubstituted 3 to 6 membered heterocycloalkyl. R⁸ may be substituted 3 to 6 membered heterocycloalkyl. R⁸ may be unsubstituted 3 to 6 membered heterocycloalkyl. R⁸ may be substituted or unsubstituted 3 membered heterocycloalkyl. R⁸ may be substituted or unsubstituted 4 membered heterocycloalkyl. R⁸ may be substituted or unsubstituted 5 membered heterocycloalkyl. R⁸ may be substituted or unsubstituted 6 membered heterocycloalkyl.

R⁸ may be R^{8A}-substituted or unsubstituted heterocycloalkyl. R⁸ may be R^{8A}-substituted heterocycloalkyl. R⁸ may be unsubstituted heterocycloalkyl. R⁸ may be R^{8A}-substituted or unsubstituted 3 to 10 membered heterocycloalkyl. R⁸ may be R^{8A}-substituted 3 to 10 membered heterocycloalkyl. R⁸ may be unsubstituted 3 to 10 membered heterocycloalkyl. R⁸ may be R^{8A}-substituted or unsubstituted 3 to 8 membered heterocycloalkyl. R⁸ may be R^{8A}-substituted 3 to 8 membered heterocycloalkyl. R⁸ may be unsubstituted 3 to 8 membered heterocycloalkyl. R⁸ may be R^{8A}-substituted or unsubstituted 3 to 6 membered heterocycloalkyl. R⁸ may be R^{8A}-substituted 3 to 6 membered heterocycloalkyl. R⁸ may be unsubstituted 3 to 6 membered heterocycloalkyl. R⁸ may be R^{8A}-substituted or unsubstituted 3 membered heterocycloalkyl. R⁸ may be R^{8A}-substituted or unsubstituted 4 membered heterocycloalkyl. R⁸ may be R^{8A}-substituted or unsubstituted 5 membered heterocycloalkyl. R⁸ may be R^{8A}-substituted or unsubstituted 6 membered heterocycloalkyl.

R⁸ may be substituted or unsubstituted aryl. R⁸ may be substituted aryl. R⁸ may be unsubstituted aryl. R⁸ may be substituted or unsubstituted 5 to 10 membered aryl. R⁸ may be substituted 5 to 10 membered aryl. R⁸ may be unsubstituted 5 to 10 membered aryl. R⁸ may be substituted or unsubstituted 5 to 8 membered aryl. R⁸ may be substituted 5 to 8 membered aryl. R⁸ may be unsubstituted 5 to 8 membered aryl. R⁸ may be substituted or unsubstituted 5 or 6 membered aryl. R⁸ may be substituted 5 or 6 membered aryl. R⁸ may be unsubstituted 5 or 6 membered aryl. R⁸ may be substituted or unsubstituted 5 membered aryl. R⁸ may be substituted or unsubstituted 6 membered aryl (e.g. phenyl).

R⁸ may be R^{8A}-substituted or unsubstituted aryl. R⁸ may be R^{8A}-substituted aryl. R⁸ may be unsubstituted aryl. R⁸ may be R^{8A}-substituted or unsubstituted 5 to 10 membered aryl. R⁸ may be R^{8A}-substituted 5 to 10 membered aryl. R⁸ may be unsubstituted 5 to 10 membered aryl. R⁸ may be R^{8A}-substituted or unsubstituted 5 to 8 membered aryl. R⁸ may be R^{8A}-substituted 5 to 8 membered aryl. R⁸ may be unsubstituted 5 to 8 membered aryl. R⁸ may be R^{8A}-substituted or unsubstituted 5 or 6 membered aryl. R⁸ may be R^{8A}-substituted 5 or 6 membered aryl. R⁸ may be unsubstituted 5 or 6 membered aryl. R⁸ may be R^{8A}-substituted or unsubstituted 5 membered aryl. R⁸ may be R^{8A}-substituted or unsubstituted 6 membered aryl (e.g. phenyl).

R⁸ may be substituted or unsubstituted heteroaryl. R⁸ may be substituted heteroaryl. R⁸ may be unsubstituted heteroaryl. R⁸ may be substituted or unsubstituted 5 to 10 membered heteroaryl. R⁸ may be substituted 5 to 10 membered heteroaryl. R⁸ may be unsubstituted 5 to 10 membered heteroaryl. R⁸ may be substituted or unsubstituted 5 to 8 membered heteroaryl. R⁸ may be substituted 5 to 8 membered heteroaryl. R⁸ may be unsubstituted 5 to 8 membered heteroaryl. R⁸ may be substituted or unsubstituted 5 or 6 membered heteroaryl. R⁸ may be substituted 5 or 6 membered heteroaryl. R⁸ may be unsubstituted 5 or 6 membered heteroaryl. R⁸ may be substituted or unsubstituted 5 membered heteroaryl. R⁸ may be substituted or unsubstituted 6 membered heteroaryl.

R⁸ may be R^{8A}-substituted or unsubstituted heteroaryl. R⁸ may be R^{8A}-substituted heteroaryl. R⁸ may be unsubstituted heteroaryl. R⁸ may be R^{8A}-substituted or unsubstituted 5 to 10 membered heteroaryl. R⁸ may be R^{8A}-substituted 5 to 10 membered heteroaryl. R⁸ may be unsubstituted 5 to 10 membered heteroaryl. R⁸ may be R^{8A}-substituted or unsubstituted 5 to 8 membered heteroaryl. R⁸ may be R^{8A}-substituted 5 to 8 membered heteroaryl. R⁸ may be unsubstituted 5 to 8 membered heteroaryl. R⁸ may be R^{8A}-substituted or unsubstituted 5 or 6 membered heteroaryl. R⁸ may be R^{8A}-substituted 5 or 6 membered heteroaryl. R⁸ may be unsubstituted 5 or 6 membered heteroaryl. R⁸ may be R^{8A}-substituted or unsubstituted 5 membered heteroaryl. R⁸ may be R^{8A}-substituted or unsubstituted 6 membered heteroaryl.

R⁸ may be —O-L^{8A}-R^{8A}. L^{8A} is substituted or unsubstituted alkylene or substituted or unsubstituted heteroalkylene. L^{8A} may be substituted or unsubstituted alkylene. L^{8A} may be substituted or unsubstituted C₁-C₂₀ alkylene. L^{8A} may be substituted or unsubstituted C₁-C₁₀ alkylene. L^{8A} may be substituted or unsubstituted C₁-C₅ alkylene. L^{8A} may be substituted C₁-C₂₀ alkylene. L^{8A} may be unsubstituted C₁-C₂₀ alkylene. L^{8A} may be substituted C₁-C₁₀ alkylene. L^{8A} may be unsubstituted C₁-C₁₀ alkylene. L^{8A} may be substituted C₁-C₅ alkylene. L^{8A} may be unsubstituted C₁-C₅ alkylene. L^{8A} may be —(CH₂)ₘ—R^{8A}, where m is an integer of 1, 2, 3, 4 or 5.

L^{8A} may be substituted or unsubstituted heteroalkylene. L^{8A} may be substituted heteroalkylene. L^{8A} may be unsubstituted heteroalkylene. L^{8A} may be substituted or unsubstituted 2 to 20 membered heteroalkylene. L^{8A} may be substituted 2 to 20 membered heteroalkylene. L^{8A} may be substituted or unsubstituted 2 to 10 membered heteroalkylene. L^{8A} may be substituted 2 to 10 membered heteroalkylene. L^{8A} may be unsubstituted 2 to 10 membered heteroalkylene. L^{8A} may be substituted or unsubstituted 2 to 6 membered heteroalkylene. L^{8A} may be substituted 2 to 6 membered heteroalkylene. L^{8A} may be unsubstituted 2 to 6 membered heteroalkylene. L^{8A} may be —(CH₂CH₂O)ₘ₁—R^{8A}, where m1 is an integer selected from 1, 2, 3, or 4.

R⁸ may be —O-L^{8A}-N(R^{8C})—S(O)ₙ₈—R^{8A}, where R^{8A} is as described herein. R⁸ may be —O-L^{8A}-N(R^{8C})—S(O)ₙ₈—R^{8A}, where R^{8A} is hydrogen or substituted or unsubstituted alkyl (e.g. C₁-C₅ alkyl).

R^{8A} is hydrogen, halogen, oxo, —CF₃, —CN, —OR¹⁵, —N(R^{15.1})(R^{15.2}), —COOR¹⁵, —CON(R^{15.1})(R^{15.2}), —NO₂, —SR¹⁵, —S(O)₂R¹⁵, —S(O)₃R¹⁵, —S(O)₄R¹⁵, —S(O)₂N(R^{15.1})(R^{15.2}), —NHN(R^{15.1})(R^{15.2}), —ON(R^{15.1})(R^{15.2}), —NHC(O)NHN(R^{15.1})(R^{15.2}), —NHC(O)N(R^{15.1})(R^{15.2}), —NHS(O)₂R¹⁵, —NHC(O)R¹⁵, —NHC(O)—OR¹⁵, —NHOR¹⁵, —OCF₃, —OCHF₂, R¹⁵-substituted or unsubstituted alkyl, R¹⁵-substituted or unsubstituted heteroalkyl, R¹⁵-substituted or unsubstituted cycloalkyl, R¹⁵-substituted or unsubstituted heterocycloalkyl, R¹⁵-substituted or unsubstituted aryl, or R¹⁵-substituted or unsubstituted heteroaryl.

R¹⁵, R^{15.1}, and R^{15.2} are independently hydrogen, halogen, oxo, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCHF₂, R¹⁶-substituted or unsubstituted alkyl, R¹⁶-substituted or unsubstituted heteroalkyl, R¹⁶-substituted or unsubstituted cycloalkyl, R¹⁶-substituted or unsubstituted heterocycloalkyl, R¹⁶-substituted or unsubstituted aryl, or R¹⁶-substituted or unsubstituted heteroaryl.

R¹⁶ is hydrogen, halogen, oxo, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

R^{8C} may be hydrogen, halogen, oxo, —OH, —NH₂, —COOH, —CONH₂, —S(O)₂Cl, —S(O)₂NH₂, —NHNH₂, —ONH₂, —NHC(O)NHNH₂, —NHC(O)NH₂, —NHS(O)₂H, —NHC(O)H, —NHC(O)—OH, —NHOH, R¹⁵-substituted or unsubstituted alkyl, R¹⁵-substituted or unsubstituted heteroalkyl, R¹⁵-substituted or unsubstituted cycloalkyl, R¹⁵-substituted or unsubstituted heterocycloalkyl, R¹⁵-substituted or unsubstituted aryl, or R¹⁵-substituted or unsubstituted heteroaryl.

R⁸ may be hydrogen, halogen, —OR^{8A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ may be —OR^{8A}, where R^{8A} is hydrogen, substituted or unsubstituted alkyl, or substituted or unsubstituted heteroalkyl. R^{8A} may be substituted or unsubstituted alkyl, or substituted or unsubstituted heteroalkyl.

R^{8A} may be —CH₃, —C₂H₅, —CD₃, —CD₂CD₃, —(CH₂)₂OH, —(CH₂CH₂)₃OH, —CH₂C(CH₃)₂OH, —(CH₂)₂C(CH₃)₂OH, —(CH₂)₂F, —(CH₂)₃F, —CH₂C(CH₃)₂F, —(CH₂)₂C(CH₃)₂F,

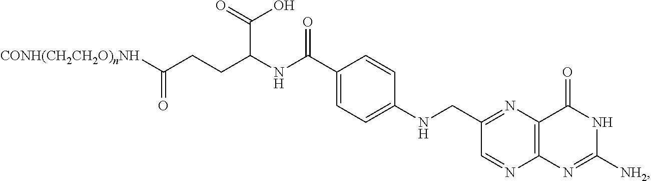

—(CH₂CH₂O)ₙCH₂CH₂-G^{8A}, or —CO(CH₂)₂COO(CH₂CH₂O)ₙCH₂CH₂-G^{8B}, where n is 2-20.
G^{8A} is H, —OH, —NH₂, —OCH₃, —OCF₃, F, Cl, N₃, —NHCH₂C₆H₄NO₂, —NHCH₂C₆H₄F,NHCH₂C₆H₄NO₂, —NHCH₂C₆H₄F,

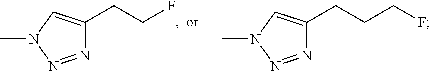

G^{8B} is H, —OH, —NH₂, —OCH₃, F, Cl,

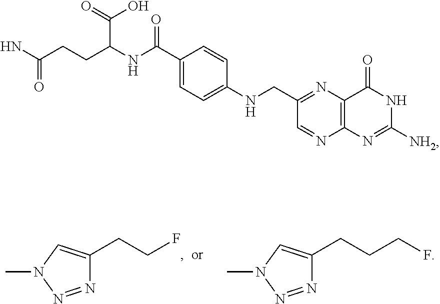

R^{8A} may be —(CH₂)₂NHSO₂CH₃, —(CH₂)₂F, —(CH₂)₃F, —(CH₂CH₂O)ₙF, or —(CH₂CH₂O)ₙCH₃, wherein n is 2 to 5.

R^{1A} and R^{8A} may independently be substituted or unsubstituted alkyl or substituted or unsubstituted heteroalkyl as described herein. R^{1A} may be —O-L^{1A}-R^{1A}, where L^{1A} is as described herein and R^{8A} may be —O-L^{8A}-R^{8A}, where L^{8A} is as described herein. L^{1A} may independently be —(CH₂)ₘ—R^{1A}, and L^{8A} may be —(CH₂)ₘ—R^{8A} where R^{1A}, R^{8A} and m are as described herein. L^{1A} may be —(CH₂CH₂O)ₘ₁—R^{1A}, and L^{8A} may be —(CH₂CH₂O)ₘ₁—R^{8A}, where R^{1A}, R^{8A}, and m are as described herein. The symbol m may independently be 1, 2, or 3. The symbol m1 may independently be 1, 2, 3, or 4.

R¹ may be —O-L^{1A}-N(R^{1C})—S(O)ₙ₁—R^{1A} as described herein and R^{8A} may be OR^{8A}, where R^{8A} is substituted or unsubstituted alkyl. R¹ may be —O-L^{1A}-N(R^{1C})—S(O)ₙ₁—R^{1A} as described herein and R^{8A} may be —OR^{8A}, where R^{8A} is unsubstituted C₁-C₃ alkyl.

R⁹ may be hydrogen, halogen, —N₃, —CF₃, —CCl₃, —CBr₃, —CI₃, —CN, —COR^{9A}, —OR^{9A}, —NR^{9A}R^{9B}, —C(O)OR^{9A}, —C(O)NR^{9A}R^{9B}, —NO₂, —SR^{9A}, —S(O)ₙ₉R^{9A}, —S(O)ₙ₉OR^{9A}, —S(O)ₙ₉NR^{9A}R^{9B}, —NHNR^{9A}R^{9B}, —ONR^{9A}R^{9B}, or —NHC(O)NHNR^{9A}R^{9B}. R⁹ may be hydrogen, halogen, —CF₃, —OR^{9A}, or —NR^{9A}R^{9B}. R⁹ may hydrogen. R⁹ may be halogen. R⁹ may be —CF₃. R⁹ may be —OR^{9A}. R⁹ may be —NR^{9A}R^{9B}.

R⁹ may be substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁹ may be substituted or unsubstituted alkyl. R⁹ may be unsubstituted alkyl R⁹ may be substituted alkyl. R⁹ may be substituted or unsubstituted C₁-C₂₀ alkyl. R⁹ may be substituted or unsubstituted C₁-C₁₀ alkyl. R⁹ may be substituted C₁-C₁₀ alkyl. R⁹ may be unsubstituted C₁-C₁₀ alkyl. R⁹ may be C₁-C₅ substituted or unsubstituted alkyl. R⁹ may be substituted C₁-C₅ alkyl. R⁹ may be unsubstituted C₁-C₅ alkyl. R⁹ may be substituted or unsubstituted C₁-C₃ alkyl. R⁹ may be unsubstituted C₁-C₃ alkyl. R⁹ may be saturated C₁-C₃ alkyl. R⁹ may be methyl. R⁹ may be ethyl. R⁹ may be propyl.

R⁹ may be substituted or unsubstituted heteroalkyl. R² may be substituted heteroalkyl. R⁹ may be unsubstituted alkyl. R⁹ may be substituted or unsubstituted 2 to 10 membered heteroalkyl. R⁹ may be substituted 2 to 10 membered heteroalkyl. R⁹ may be unsubstituted 2 to 10 membered heteroalkyl. R⁹ may be 2 to 6 membered heteroalkyl. R⁹ may be substituted 2 to 6 membered heteroalkyl. R⁹ may be unsubstituted 2 to 6 membered heteroalkyl.

R⁹ may be substituted or unsubstituted 3 to 8 membered cycloalkyl. R⁹ may be substituted 3 to 8 membered cycloalkyl. R⁹ may be unsubstituted 3 to 8 membered cycloalkyl. R⁹ may be substituted or unsubstituted 3 to 6 membered cycloalkyl. R⁹ may be substituted 3 to 6 membered cycloalkyl. R⁹ may be unsubstituted 3 to 6 membered cycloalkyl. R⁹ may be substituted or unsubstituted 3 membered cycloalkyl. R⁹ may be substituted or unsubstituted 4 membered cycloalkyl. R² may be 5 membered cycloalkyl. R⁹ may be 6 membered cycloalkyl.

R⁹ may be substituted or unsubstituted 3 to 8 membered heterocycloalkyl. R⁹ may be substituted 3 to 8 membered heterocycloalkyl. R⁹ may be unsubstituted 3 to 8 membered heterocycloalkyl. R⁹ may be substituted or unsubstituted 3 to 6 membered heterocycloalkyl. R⁹ may be substituted 3 to 6 membered heterocycloalkyl. R⁹ may be unsubstituted 3 to 6 membered heterocycloalkyl. R⁹ may be substituted or unsubstituted 3 membered heterocycloalkyl. R⁹ may be substituted or unsubstituted 4 membered heterocycloalkyl. R⁹ may be 5 membered heterocycloalkyl. R⁹ may be 6 membered heterocycloalkyl.

R⁹ may be substituted or unsubstituted 5 to 8 membered aryl. R⁹ may be substituted 5 to 8 membered aryl. R⁹ may be unsubstituted 5 to 8 membered aryl. R⁹ may be substituted or unsubstituted 5 membered aryl. R⁹ may be substituted 5 membered aryl. R⁹ may be unsubstituted 5 membered aryl. R⁹ may be substituted 6 membered aryl. R⁹ may be unsubstituted 6 membered aryl (e.g. phenyl).

R⁹ may be substituted or unsubstituted 5 to 8 membered heteroaryl. R⁹ may be substituted 5 to 8 membered heteroaryl. R⁹ may be unsubstituted 5 to 8 membered heteroaryl. R⁹ may be substituted or unsubstituted 5 membered heteroaryl. R⁹ may be substituted 5 membered aryl. R⁹ may be unsubstituted 5 membered heteroaryl. R⁹ may be substituted 6 membered aryl. R⁹ may be unsubstituted 6 membered heteroaryl.

R^{1B}, R^{2A}, R^{2B}, R^{3A}, R^{3B}, R^{4A}, R^{4B}, R^{5A}, R^{5B}, R^{7A}, R^{7B}, R^{8B}, R^{9A}, and R^{9B}, may independently be hydrogen, halogen, or substituted or unsubstituted alkyl.

The compound of formula (I) may have the formula:

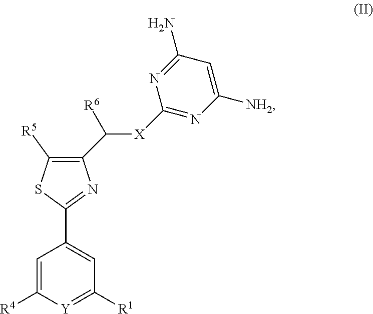

where R¹, R⁴, R⁵, R⁶, Y and X are as described herein.

In the compound of formula (II), R⁴ may be hydrogen or halogen. In the compound of formula (II), R⁵ may be substituted or unsubstituted alkyl. R⁵ may be C₁-C₅ unsubstituted alkyl. R⁵ may be methyl. R⁵ may be ethyl. R⁵ may be propyl. R⁶ may be C₁-C₄ unsubstituted alkyl. R⁶ may be methyl. R⁶ may be ethyl. R⁶ may be propyl.

The compound of formula (I) may have the formula:

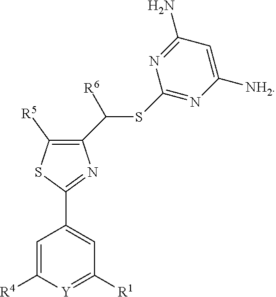

Y, R¹, R⁴, R⁵, and R⁶ are as described herein.

The compound of formula (I) may have the formula:

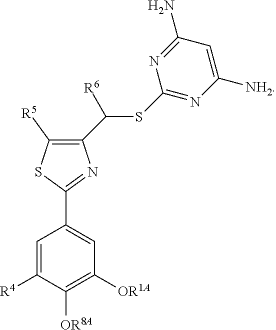

R^{1A}, R⁴, R⁵, R⁶ and R^{8A} are as described herein.

The compound of formula (I) may have the formula:

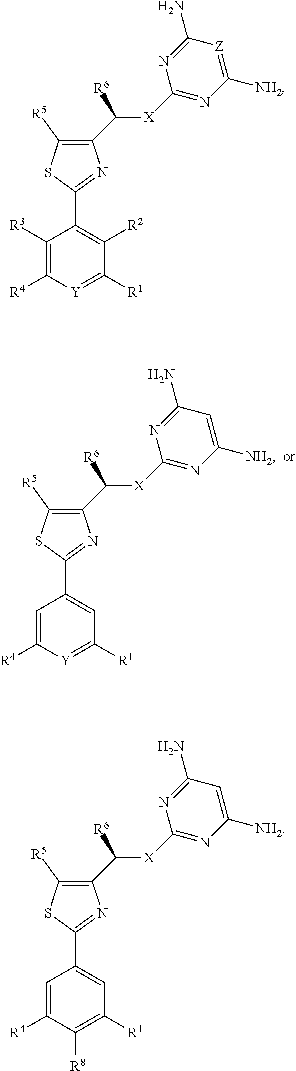

Y, R¹, R⁴, R⁵, and R⁶ are as described herein. R¹ may be —OR^{1A}, wherein R^{1A} is —OCH₃, —OCH₂CH₃, —O(CH₂)₂F, —(CH₂)₂NHSO₂CH₃, —(CH₂CH₂O)ₙF, —(CH₂CH₂O)ₙCH₃, and the symbol n is 2 to 5. R⁴ may be hydrogen or halogen. R⁵ may be methyl or propyl. R⁶ may be methyl. R⁸ may be —OR^{8A}, where R^{8A} may be —OCH₃, —(CH₂)₂NHSO₂CH₃, —(CH₂)₂F, (CH₂)₃F, —(CH₂CH₂O)ₙF, or —(CH₂CH₂O)ₙCH₃, wherein n is 2 to 5.

The compound of formula (I) may have the formula

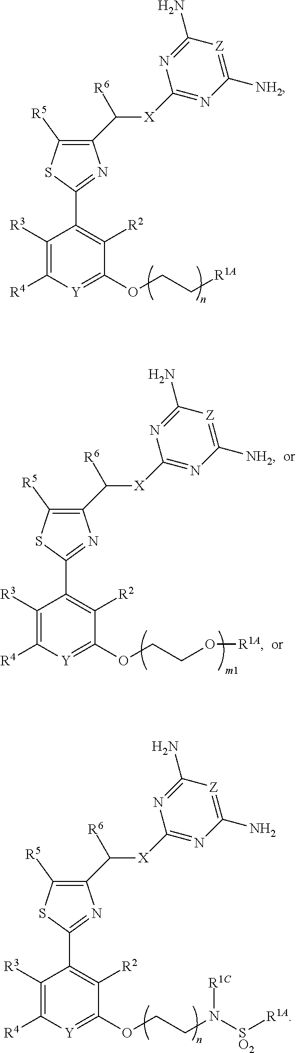

X, Y, Z, R¹, R^{1A}, R^{1C}, R², R³, R⁴, R⁵, R⁶ and R^{8A} are as described herein. The symbol n and m1 may independently be 1, 2, 3, or 4. R^{1A} may be unsubstituted alkyl. R^{1A} may be methyl. R^{1A} may be hydrogen. R⁵ may be methyl, ethyl, or propyl and R⁶ may be methyl.

The compound of formula (I) may have the formula

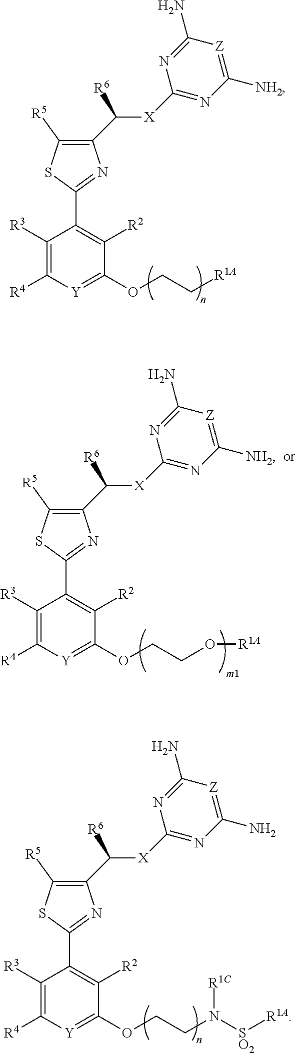

X, Y, Z, R¹, R^{1A}, R^{1C}, R², R³, R⁴, R⁵, R⁶ and R^{8A} are as described herein. The symbol n and m1 may independently be 1, 2, 3, or 4. R^{1A} may be unsubstituted alkyl. R^{1A} may be methyl. R^{1A} may be hydrogen. R⁵ may be methyl, ethyl, or propyl and R⁶ may be methyl.

The compound of formula (I) may have the formula:

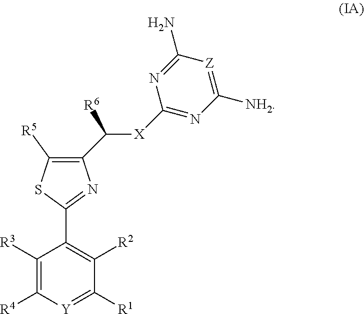

The compound of formula (I) may have the formula:

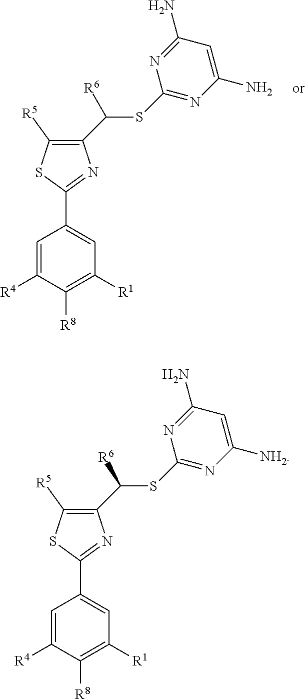

Also provided herein are pharmaceutical formulations. In one aspect is a pharmaceutical composition that includes a compound described herein and a pharmaceutically acceptable excipient.

The pharmaceutical composition may be prepared and administered in a wide variety of dosage formulations. Compounds described may be administered orally, rectally, or by injection (e.g. intravenously, intramuscularly, intracutaneously, subcutaneously, intraduodenally, or intraperitoneally).

The optimal dose of the combination of agents for treatment of disease can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages can be established using routine testing and procedures that are well known in the art.

The amount of combination of agents that can be combined with the carrier materials to produce a single dosage form will vary depending upon the individual treated and the particular mode of administration. In some embodiments the unit dosage forms containing the combination of agents as described herein will contain the amounts of each agent of the combination that are typically administered when the agents are administered alone.

Frequency of dosage can vary depending on the compound used and the particular condition to be treated or prevented. In general, the use of the minimum dosage that is sufficient to provide effective therapy is preferred. Patients can generally be monitored for therapeutic effectiveness using assays suitable for the condition being treated or prevented, which will be familiar to those of ordinary skill in the art.

The dosage form can be prepared by various conventional mixing, comminution and fabrication techniques readily apparent to those skilled in the chemistry of drug formulations.

The oral dosage form containing the combination of agents or individual agents of the combination of agents can be in the form of micro-tablets enclosed inside a capsule, e.g. a gelatin capsule.

Many of the oral dosage forms useful herein contain the combination of agents or individual agents of the combination of agents in the form of particles. Such particles can be compressed into a tablet, present in a core element of a coated dosage form, such as a taste-masked dosage form, a press coated dosage form, or an enteric coated dosage form, or can be contained in a capsule, osmotic pump dosage form, or other dosage form.

The drug compounds of the present invention are present in the combinations (fixed or non-fixed), dosage forms, pharmaceutical compositions and pharmaceutical formulations disclosed herein in a ratio in the range of 100:1 to 1:100.

The optimum ratios, individual and combined dosages, and concentrations of the drug compounds that yield efficacy without toxicity are based on the kinetics of the active ingredients' availability to target sites, and are determined using methods known to those of skill in the art.

The pharmaceutical compositions or combinations provided herein can be tested in clinical studies. Suitable clinical studies can be, for example, open label, dose escalation studies in patients with cancer. Such studies prove in particular the synergism of the active ingredients of the combination of the invention. The beneficial effects on cancer can be determined directly through the results of these studies which are known as such to a person skilled in the art. Such studies can be, in particular, suitable to compare the effects of a monotherapy using the active ingredients and a combination of the invention. Each patient can receive doses of the compounds either daily or intermittently. The efficacy of the treatment can be determined in such studies, e.g., after 12, 18 or 24 weeks by evaluation of symptom scores every 6 weeks.

The administration of a combination therapy of the invention can result not only in a beneficial effect, e.g. a synergistic therapeutic effect, e.g. with regard to alleviating, delaying progression of or inhibiting the symptoms, but also in further surprising beneficial effects, e.g. fewer side-effects, an improved quality of life or a decreased morbidity, compared with a monotherapy applying only one of the pharmaceutically active ingredients used in the combination of the invention.

A further benefit can be that lower doses of the active ingredients of the combination of the invention can be used, for example, that the dosages need not only often be smaller but can also be applied less frequently, which can diminish the incidence or severity of side-effects. This is in accordance with the desires and requirements of the patients to be treated.

It is one objective of this invention to provide a pharmaceutical combination comprising a quantity, which can be jointly therapeutically effective at targeting or preventing cancer. In this combination, two or three compounds disclosed herein can be administered together, one after the other, in one combined unit dosage form or separately in two or more separate unit dosage forms. The unit dosage form can also be a fixed combination.

The pharmaceutical compositions for separate administration (or non-fixed dose) of the compounds disclosed herein, or for the administration in a fixed combination, i.e. a single composition comprising at least two compounds according to the invention can be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g. as indicated above, or in combination with one or more pharmaceutically acceptable carriers or diluents, especially suitable for enteral or parenteral application.

The drug combinations provided herein can be formulated by a variety of methods apparent to those of skill in the art of pharmaceutical formulation. As discussed above, the compounds disclosed herein can be formulated into the same pharmaceutical composition or into separate pharmaceutical compositions for individual administration. Suitable formulations include, for example, tablets, capsules, press coat formulations, intravenous solutions or suspensions, and other easily administered formulations.

One or more combination partners can be administered in a pharmaceutical formulation comprising one or more pharmaceutically acceptable carriers. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

Suitable pharmaceutical formulations can contain, for example, from about 0.1% to about 99.9%, preferably from about 1% to about 60%, of the active ingredient(s). Pharmaceutical formulations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In accordance with the present invention, a therapeutically effective amount of each of the combination partners of the combination of the invention can be administered simultaneously or sequentially and in any order, and the components can be administered separately or as a fixed combination. Alternatively, an amount, which is jointly therapeutically effective for the treatment of cancer, of each combination partner of the combination of the invention can be administered simultaneously or sequentially and in any order, and the components can be administered separately or as a fixed combination.

For example, the method of treating a disease according to the invention can comprise (i) administration of the first agent in free or pharmaceutically acceptable salt form, (ii) administration of the second agent in free or pharmaceutically acceptable salt form and (iii) administration of the second agent in free or pharmaceutically acceptable salt form simultaneously or sequentially in any order, in jointly therapeutically effective amounts, preferably in synergistically effective amounts, e.g. in daily or intermittently dosages corresponding to the amounts described herein. The method of treating a disease according to the invention can comprise (i) administration of the first agent in free or pharmaceutically acceptable salt form and (ii) administration of the second agent in free or pharmaceutically acceptable salt form or sequentially in any order, in jointly therapeutically effective amounts, preferably in synergistically effective amounts, e.g. in daily or intermittently dosages corresponding to the amounts described herein. The individual combination partners of the combination of the invention can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention can vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition. A clinician or physician of ordinary skill can also readily determine the effective dosage using the Response Evaluation Criteria In Solid Tumors (RECIST) guidelines (see e.g., Therasse et al. 2000, JNCI 92:2, 205, which is hereby incorporated by reference in its entirety).

Suitable dosages for the compounds used in the methods described herein are on the order of about 0.1 mg to about 200 mg, (e.g., about 0.1, 0.3, 0.5, 0.7, 1, 3, 5, 10, 15, 20, 25, 30, 35, 40, 45, 50, 55, 60, 65, 70, 75, 80, 90, 95, 100, 120, 140, 160, 180, 200, or 220 mg).

Suitable administration frequencies for the compounds used in the methods described herein are on the order of about 10 times per day to about once per month (e.g., about 10, 9, 8, 7, 6, 5, 4, 3, 2, 1 times per day to about 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 times per month).

For preparing pharmaceutical compositions from compounds described herein, pharmaceutically acceptable carriers can be either solid or liquid. Solid form preparations include powders, tablets, pills, capsules, cachets, suppositories, and dispersible granules. A solid carrier may be one or more substance that may also act as diluents, flavoring agents, binders, preservatives, tablet disintegrating agents, or an encapsulating material.

In powders, the carrier may be a finely divided solid in a mixture with the finely divided active component. In tablets, the active component may be mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight. Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The pharmaceutical compositions may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

In one aspect, provided herein are compounds of Formula (I), or a pharmaceutically acceptable salt or solvate thereof:

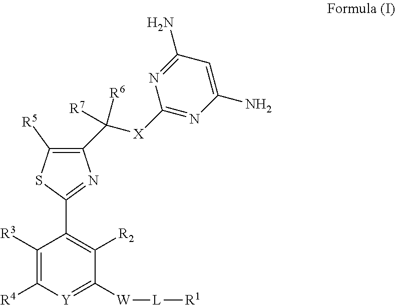

wherein

### W is —O—, —S—, or —N(R⁸)—;

L is optionally substituted alkylene, optionally substituted alkenylene, or optionally substituted alkynylene;
X is —CH₂—, —O—, —N(R⁸)—, —S—, —S(O)—, or —S(O)₂—;

### Y is N or C(R⁹);

R¹ is optionally substituted heterocycloalkyl;
R², R³, R⁴ are independently hydrogen, halogen, —CN, optionally substituted alkyl, optionally substituted alkoxy, or optionally substituted cycloalkyl;
R⁵ is hydrogen, halogen, optionally substituted alkyl, optionally substituted alkoxy, optionally substituted cycloalkyl, optionally substituted aryl, or optionally substituted heteroaryl;
R⁶ and R⁷ are independently hydrogen, halogen, or optionally substituted alkyl; or
R⁶ and R⁷ are taken together with the carbon to which they are attached to form a cycloalkyl;
R⁸ is hydrogen or optionally substituted alkyl; and
R⁹ is hydrogen, halogen, —CN, optionally substituted alkyl, optionally substituted alkoxy, or optionally substituted cycloalkyl.

In some embodiment is a compound of Formula (I), wherein R² and R³ are hydrogen. In some embodiments is a compound of Formula (I), wherein R² and R³ are independently hydrogen or halogen. In some embodiments is a compound of Formula (I), wherein R² and R³ are independently hydrogen or optionally substituted alkyl. In some embodiments is a compound of Formula (I), wherein R² and R³ are independently hydrogen or unsubstituted alkyl.

In some embodiments is a compound of Formula (I), wherein R⁴ is hydrogen or halogen. In some embodiments is a compound of Formula (I), wherein R⁴ is hydrogen.

In some embodiments is a compound of Formula (I), wherein R⁶ and R⁷ are independently hydrogen or optionally substituted alkyl. In some embodiments is a compound of Formula (I), wherein R⁶ and R⁷ are independently hydrogen or unsubstituted alkyl. In some embodiments is a compound of Formula (I), wherein R⁷ is hydrogen.

In some embodiments is a compound of Formula (I), wherein the compound of Formula (I) is a compound of Formula (Ia):

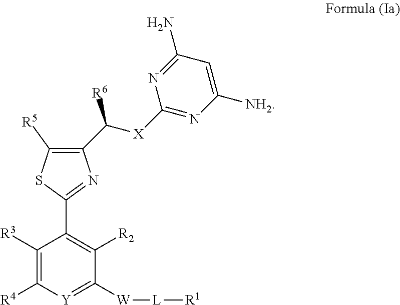

In some embodiments is a compound of Formula (I), wherein the compound of Formula (I) is a compound of Formula (Ib):

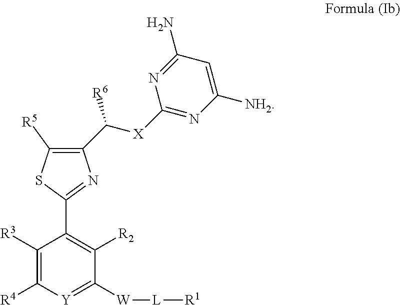

In some embodiments is a compound of Formula (I), wherein R⁶ is optionally substituted alkyl. In some embodiments is a compound of Formula (I), wherein R⁶ is unsubstituted alkyl. In some embodiments is a compound of Formula (I), wherein R⁶ is substituted alkyl. In some embodiments is a compound of Formula (I), wherein R⁶ is methyl, ethyl, or propyl. In some embodiments is a compound of Formula (I), wherein R⁶ is methyl. In some embodiments is a compound of Formula (I), wherein R⁶ and R⁷ are not both hydrogen. In some embodiments is a compound of Formula (I), wherein R⁶ and R⁷ are both optionally substituted alkyl. In some embodiments is a compound of Formula (I), wherein R⁶ and R⁷ are both unsubstituted alkyl. In some embodiments is a compound of Formula (I), wherein R⁶ and R⁷ are both methyl. In some embodiments is a compound of Formula (I), wherein R⁶ and R⁷ are both unsubstituted alkyl. In some embodiments is a compound of Formula (I), wherein R⁶ and R⁷ are taken together with the carbon to which they are attached to form a cycloalkyl. In some embodiments is a compound of Formula (I), wherein R⁶ and R⁷ are taken together with the carbon to which they are attached to form a cyclopropyl, cyclobutyl, cyclopentyl, or cyclohexyl. In some embodiments is a compound of Formula (I), wherein R⁶ and R⁷ are taken together with the carbon to which they are attached to form a cyclopropyl.

In some embodiments is a compound of Formula (I), wherein R⁵ is optionally substituted alkyl. In some embodiments is a compound of Formula (I), wherein R⁵ is substituted alkyl. In some embodiments is a compound of Formula (I), wherein R⁵ is unsubstituted alkyl. In some embodiments is a compound of Formula (I), wherein R⁵ is methyl, ethyl, propyl, or butyl. In some embodiments is a compound of Formula (I), wherein R⁵ is methyl. In some embodiments is a compound of Formula (I), wherein R⁵ is ethyl. In some embodiments is a compound of Formula (I), wherein R⁵ is propyl.

In some embodiments is a compound of Formula (I), wherein R⁵ is optionally substituted aryl. In some embodiments is a compound of Formula (I), wherein R⁵ is substituted aryl. In some embodiments is a compound of Formula (I), wherein R⁵ is unsubstituted aryl. In some embodiments is a compound of Formula (I), wherein R⁵ is phenyl.

In some embodiments is a compound of Formula (I), wherein X is —S—. In some embodiments is a compound of Formula (I), wherein X is —CH₂—. In some embodiments is a compound of Formula (I), wherein X is —N(R⁸)—. In some embodiments is a compound of Formula (I), wherein X is —N(R⁸)— and R⁸ is hydrogen. In some embodiments is a compound of Formula (I), wherein X is —N(R⁸)— and R⁸ is optionally substituted alkyl. In some embodiments is a compound of Formula (I), wherein X is —N(R⁸)— and R⁸ is substituted alkyl. In some embodiments is a compound of Formula (I), wherein X is —N(R⁸)— and R⁸ is unsubstituted alkyl. In some embodiments is a compound of Formula (I), wherein X is —S(O)—. In some embodiments is a compound of Formula (I), wherein X is —S(O)₂—. In some embodiments is a compound of Formula (I), wherein X is —O—.

In some embodiments is a compound of Formula (I), wherein Y is N. In some embodiments is a compound of Formula (I), wherein Y is C(R⁹). In some embodiments is a compound of Formula (I), wherein Y is C(R⁹) and R⁹ is hydrogen, optionally substituted alkyl, or optionally substituted alkoxy. In some embodiments is a compound of Formula (I), wherein Y is C(R⁹) and R⁹ is hydrogen. In some embodiments is a compound of Formula (I), wherein Y is C(R⁹) and R⁹ is optionally substituted alkyl. In some embodiments is a compound of Formula (I), wherein Y is C(R⁹) and R⁹ is substituted alkyl. In some embodiments is a compound of Formula (I), wherein Y is C(R⁹) and R⁹ is —CF₃. In some embodiments is a compound of Formula (I), wherein Y is C(R⁹) and R⁹ is unsubstituted alkyl. In some embodiments is a compound of Formula (I), wherein Y is C(R⁹) and R⁹ is methyl, ethyl, or propyl.

In some embodiments is a compound of Formula (I), wherein Y is C(R⁹) and R⁹ is optionally substituted alkoxy. In some embodiments is a compound of Formula (I), wherein Y is C(R⁹) and R⁹ is substituted alkoxy. In some embodiments is a compound of Formula (I), wherein Y is C(R⁹) and R⁹ is —O—(CH₂CH₂—O)ₙ—CH₃ wherein n is an integer between 0 and 6. In some embodiments is a compound of Formula (I), wherein Y is C(R⁹) and R⁹ is —O—(CH₂CH₂—O)ₙ—CH₃ wherein n is 2. In some embodiments is a compound of Formula (I), wherein Y is C(R⁹) and R⁹ is —O—(CH₂CH₂—O)ₙ—CH₃ wherein n is 3. In some embodiments is a compound of Formula (I), wherein Y is C(R⁹) and R⁹ is —O—(CH₂CH₂—O)ₙ—CH₃ wherein n is 4. In some embodiments is a compound of Formula (I), wherein Y is C(R⁹) and R⁹ is —O—(CH₂CH₂—O)ₙ—CH₃ wherein n is 5. In some embodiments is a compound of Formula (I), wherein Y is C(R⁹) and R⁹ is —O—(CH₂CH₂—O)ₙ—CH₃ wherein n is 6.

In some embodiments is a compound of Formula (I), wherein Y is C(R⁹) and R⁹ is unsubstituted alkoxy. In some embodiments is a compound of Formula (I), wherein Y is C(R⁹) and R⁹ is methoxy, ethoxy, or propoxy. In some embodiments is a compound of Formula (I), wherein Y is C(R⁹) and R⁹ is methoxy.

In some embodiments is a compound of Formula (I), wherein W is —O—. In some embodiments is a compound of Formula (I), wherein W is —S—. In some embodiments is a compound of Formula (I), wherein W is —N(R⁸)—. In some embodiments is a compound of Formula (I), wherein W is —N(R⁸)— and R⁸ is hydrogen. In some embodiments is a compound of Formula (I), wherein W is —N(R⁸)— and R⁸ is optionally substituted alkyl. In some embodiments is a compound of Formula (I), wherein W is —N(R⁸)— and R⁸ is substituted alkyl. In some embodiments is a compound of Formula (I), wherein W is —N(R⁸)— and R⁸ is unsubstituted alkyl. In some embodiments is a compound of Formula (I), wherein W is —N(R⁸)— and R⁸ is methyl, ethyl or propyl.

In some embodiments is a compound of Formula (I), wherein L is optionally substituted alkylene. In some embodiments is a compound of Formula (I), wherein L is substituted alkylene. In some embodiments is a compound of Formula (I), wherein L is unsubstituted alkylene. In some embodiments is a compound of Formula (I), wherein L is —CH₂CH₂—. In some embodiments is a compound of Formula (I), wherein L is —CH₂CH₂CH₂—. In some embodiments is a compound of Formula (I), wherein L is —CH₂CH₂CH₂CH₂—.

In some embodiments is a compound of Formula (I), wherein L is optionally substituted alkenylene. In some embodiments is a compound of Formula (I), wherein L is substituted alkenylene. In some embodiments is a compound of Formula (I), wherein L is unsubstituted alkenylene. In some embodiments is a compound of Formula (I), wherein L is —CH₂═CH₂—.

In some embodiments is a compound of Formula (I), wherein L is optionally substituted alkynylene. In some embodiments is a compound of Formula (I), wherein L is substituted alkynylene. In some embodiments is a compound of Formula (I), wherein L is unsubstituted alkynylene. In some embodiments is a compound of Formula (I), wherein L is —CH₂≡CH₂—.

In some embodiments is a compound of Formula (I), wherein R¹ is a 3-membered optionally substituted heterocycloalkyl. In some embodiments is a compound of Formula (I), wherein R¹ is a 3-membered substituted heterocycloalkyl. In some embodiments is a compound of Formula (I), wherein R¹ is a 3-membered unsubstituted heterocycloalkyl. In some embodiments is a compound of Formula (I), wherein R¹ is azyridinyl.

In some embodiments is a compound of Formula (I), wherein R¹ is a 4-membered optionally substituted heterocycloalkyl. In some embodiments is a compound of Formula (I), wherein R¹ is a 4-membered substituted heterocycloalkyl. In some embodiments is a compound of Formula (I), wherein R¹ is a 4-membered unsubstituted heterocycloalkyl. In some embodiments is a compound of Formula (I), wherein R¹ is azetidinyl.

In some embodiments is a compound of Formula (I), wherein R¹ is a 5-membered optionally substituted heterocycloalkyl. In some embodiments is a compound of Formula (I), wherein R¹ is a 5-membered substituted heterocycloalkyl. In some embodiments is a compound of Formula (I), wherein R¹ is a 5-membered unsubstituted heterocycloalkyl. In some embodiments is a compound of Formula (I), wherein R¹ is pyrrolidinyl.

In some embodiments is a compound of Formula (I), wherein R¹ is a 6-membered optionally substituted heterocycloalkyl. In some embodiments is a compound of Formula (I), wherein R¹ is a 6-membered substituted heterocycloalkyl. In some embodiments is a compound of Formula (I), wherein R¹ is a 6-membered unsubstituted heterocycloalkyl. In some embodiments is a compound of Formula (I), wherein R¹ is piperidinyl, piperizanyl, or morpholinyl. In some embodiments is a compound of Formula (I), wherein R¹ is morpholinyl. In some embodiments is a compound of Formula (I), wherein R¹ is piperidinyl. In some embodiments is a compound of Formula (I), wherein R¹ is piperazinyl. In some embodiments is a compound of Formula (I), wherein R¹ is 4-methyl piperazinyl. In some embodiments is a compound of Formula (I), wherein R¹ is thiomorpholinyl.

In some embodiments is a compound of Formula (I), wherein R¹ is a 7-membered optionally substituted heterocycloalkyl. In some embodiments is a compound of Formula (I), wherein R¹ is a 7-membered substituted heterocycloalkyl. In some embodiments is a compound of Formula (I), wherein R¹ is a 7-membered unsubstituted heterocycloalkyl. In some embodiments is a compound of Formula (I), wherein R¹ is azepanyl.

In some embodiments is a compound of Formula (I), wherein the compound of Formula (I) is a compound of Formula (Ic):

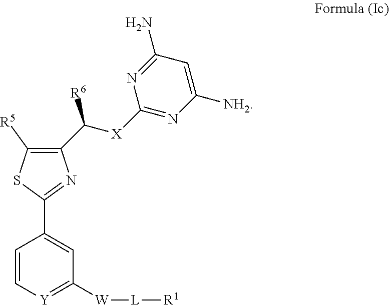

In some embodiments is a compound of Formula (I), wherein the compound of Formula (I) is a compound of Formula (Id):

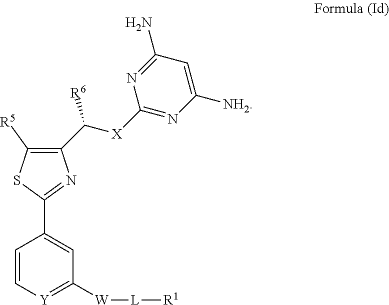

In some embodiments is a compound of Formula (Ic) or Formula (Id), wherein R⁵ is alkyl. In some embodiments is a compound of Formula (Ic) or Formula (Id), wherein R⁵ is methyl, ethyl, or propyl. In some embodiments is a compound of Formula (Ic) or Formula (Id), wherein R⁵ is methyl.

In some embodiments is a compound of Formula (Ic) or Formula (Id), wherein R⁶ is alkyl. In some embodiments is a compound of Formula (Ic) or Formula (Id), wherein R⁶ is methyl, ethyl, or propyl. In some embodiments is a compound of Formula (Ic) or Formula (Id), wherein R⁶ is methyl.

In some embodiments is a compound of Formula (Ic) or Formula (Id), wherein X is —S—.

In some embodiments is a compound of Formula (Ic) or Formula (Id), wherein Y is C(R⁹), R⁹ is optionally substituted alkoxy, W is —O—, L is optionally substituted alkylene, and R¹ is optionally substituted heterocycloalkyl. In some embodiments is a compound of Formula (Ic) or Formula (Id), wherein Y is C(R⁹), R⁹ is unsubstituted alkoxy, W is —O—, L is unsubstituted alkylene, and R¹ is optionally substituted heterocycloalkyl. In some embodiments is a compound of Formula (Ic) or Formula (Id), wherein Y is C(R⁹), R⁹ is unsubstituted alkoxy, W is —O—, L is unsubstituted alkylene, and R¹ is unsubstituted heterocycloalkyl. In some embodiments is a compound of Formula (Ic) or Formula (Id), wherein Y is C(R⁹), R⁹ is unsubstituted alkoxy, W is —O—, L is unsubstituted alkylene, and R¹ is piperidinyl, piperizanyl, or morpholinyl. In some embodiments is a compound of Formula (Ic) or Formula (Id), wherein Y is C(R⁹), R⁹ is methoxy, W is —O—, L is unsubstituted alkylene, and R¹ is piperidinyl, piperizanyl, or morpholinyl.

In some embodiments is a compound of Formula (I), wherein the compound of Formula (I) is selected from:

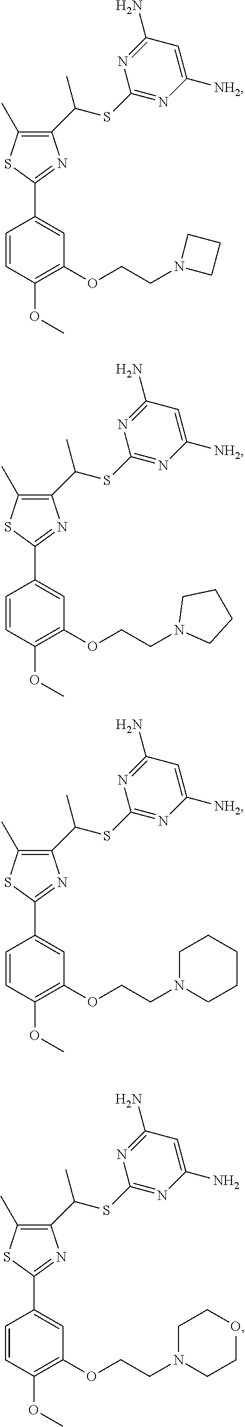 [SMILES: COc1ccc(-c2nc(C(C)Sc3nc(N)cc(N)n3)c(C)s2)cc1OCCN1CCC1 ; COc1ccc(-c2nc(C(C)Sc3nc(N)cc(N)n3)c(C)s2)cc1OCCN1CCCC1 ; COc1ccc(-c2nc(C(C)Sc3nc(N)cc(N)n3)c(C)s2)cc1OCCN1CCCCC1 ; COc1ccc(-c2nc(C(C)Sc3nc(N)cc(N)n3)c(C)s2)cc1OCCN1CCOCC1]

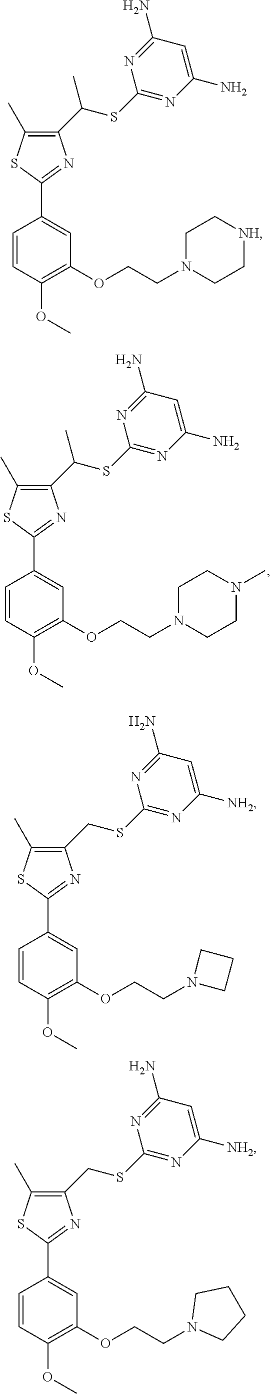

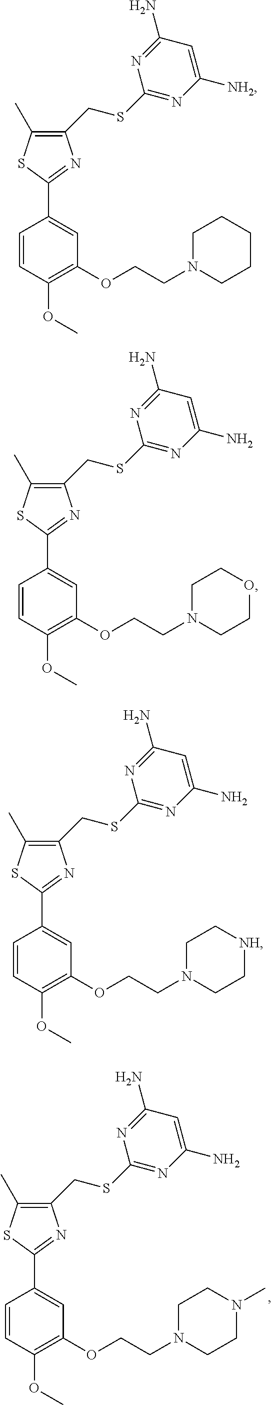

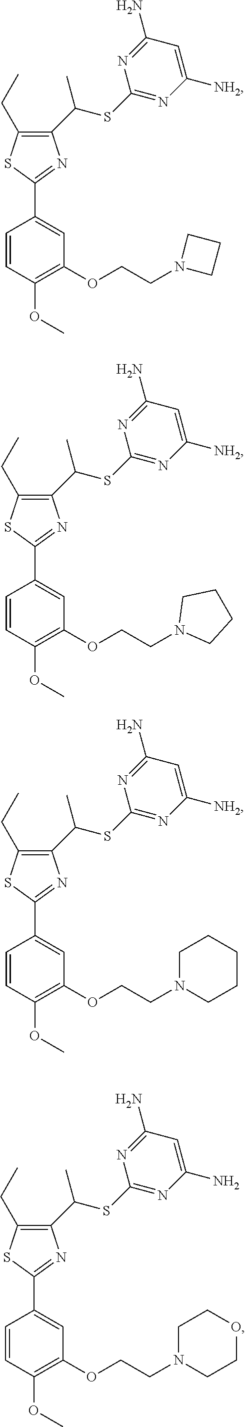 [SMILES: CCc1sc(-c2ccc(OC)c(OCCN3CCC3)c2)nc1C(C)Sc1nc(N)cc(N)n1 ; CCc1sc(-c2ccc(OC)c(OCCN3CCCC3)c2)nc1C(C)Sc1nc(N)cc(N)n1 ; CCc1sc(-c2ccc(OC)c(OCCN3CCCCC3)c2)nc1C(C)Sc1nc(N)cc(N)n1 ; CCc1sc(-c2ccc(OC)c(OCCN3CCOCC3)c2)nc1C(C)Sc1nc(N)cc(N)n1]

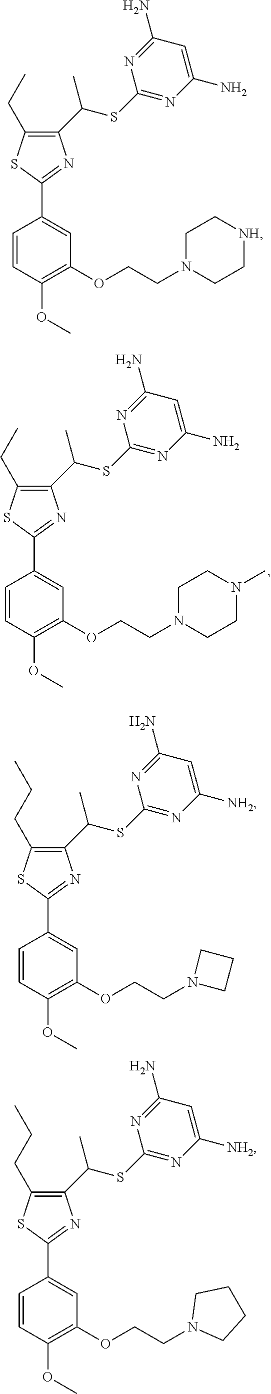 [SMILES: CCc1sc(-c2ccc(OC)c(OCCN3CCNCC3)c2)nc1C(C)Sc1nc(N)cc(N)n1 ; CCc1sc(-c2ccc(OC)c(OCCN3CCN(C)CC3)c2)nc1C(C)Sc1nc(N)cc(N)n1 ; CCCc1sc(-c2ccc(OC)c(OCCN3CCC3)c2)nc1C(C)Sc1nc(N)cc(N)n1 ; CCCc1sc(-c2ccc(OC)c(OCCN3CCCC3)c2)nc1C(C)Sc1nc(N)cc(N)n1]

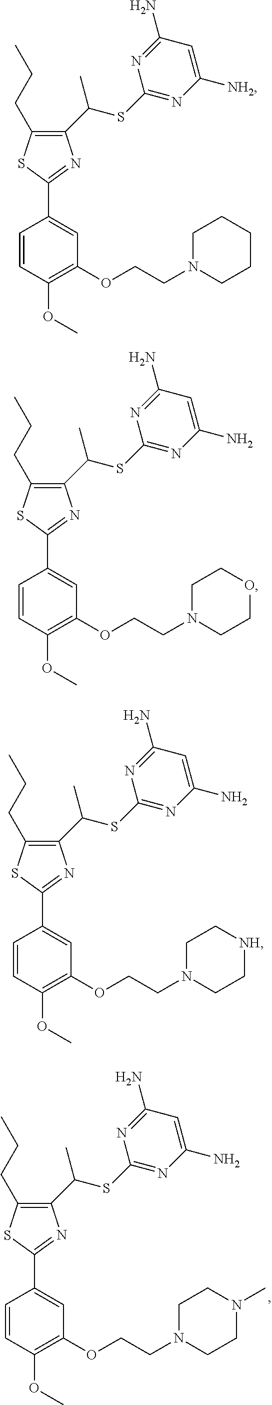 [SMILES: CCCc1sc(-c2ccc(OC)c(OCCN3CCCCC3)c2)nc1C(C)Sc1nc(N)cc(N)n1 ; CCCc1sc(-c2ccc(OC)c(OCCN3CCOCC3)c2)nc1C(C)Sc1nc(N)cc(N)n1 ; CCCc1sc(-c2ccc(OC)c(OCCN3CCNCC3)c2)nc1C(C)Sc1nc(N)cc(N)n1 ; CCCc1sc(-c2ccc(OC)c(OCCN3CCN(C)CC3)c2)nc1C(C)Sc1nc(N)cc(N)n1]

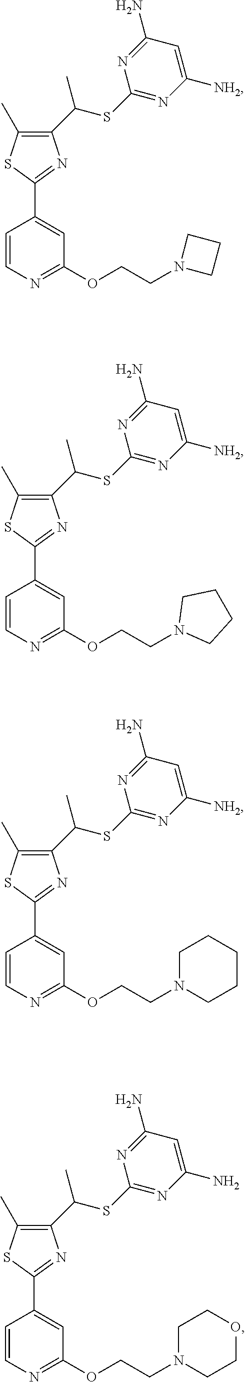 [SMILES: Cc1sc(-c2ccnc(OCCN3CCC3)c2)nc1C(C)Sc1nc(N)cc(N)n1 ; Cc1sc(-c2ccnc(OCCN3CCCC3)c2)nc1C(C)Sc1nc(N)cc(N)n1 ; Cc1sc(-c2ccnc(OCCN3CCCCC3)c2)nc1C(C)Sc1nc(N)cc(N)n1 ; Cc1sc(-c2ccnc(OCCN3CCOCC3)c2)nc1C(C)Sc1nc(N)cc(N)n1]

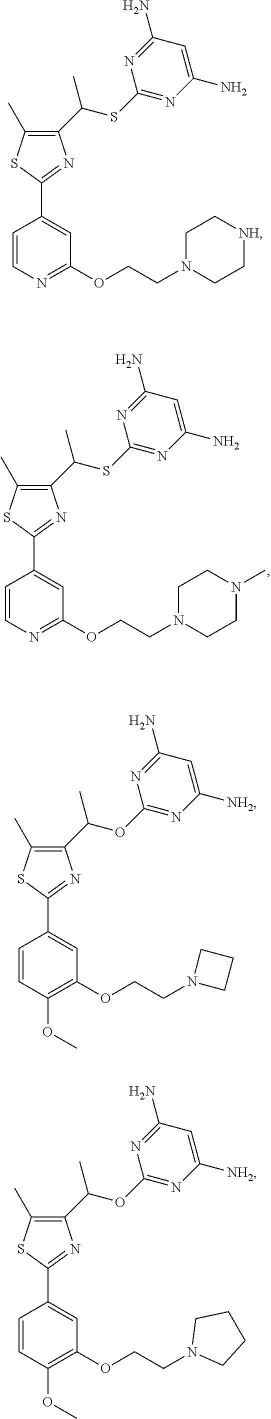

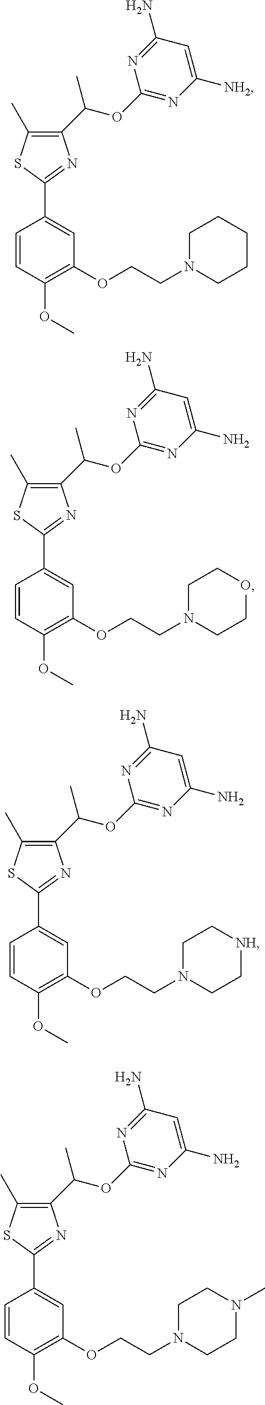

or a pharmaceutically acceptable salt or solvate thereof.

In some embodiments is a compound selected from:

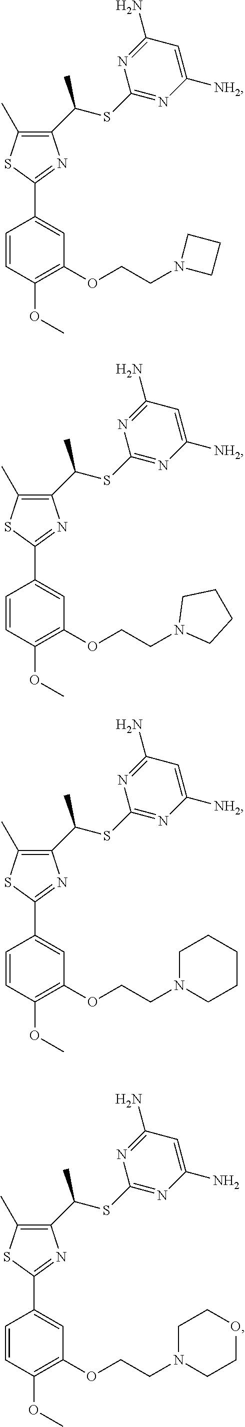 [SMILES: COc1ccc(-c2nc([C@@H](C)Sc3nc(N)cc(N)n3)c(C)s2)cc1OCCN1CCC1 ; COc1ccc(-c2nc([C@@H](C)Sc3nc(N)cc(N)n3)c(C)s2)cc1OCCN1CCCC1 ; COc1ccc(-c2nc([C@@H](C)Sc3nc(N)cc(N)n3)c(C)s2)cc1OCCN1CCCCC1 ; COc1ccc(-c2nc([C@@H](C)Sc3nc(N)cc(N)n3)c(C)s2)cc1OCCN1CCOCC1]

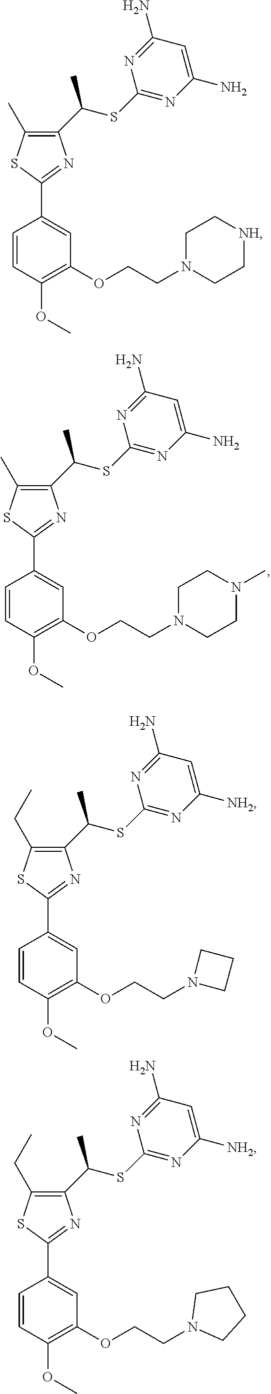

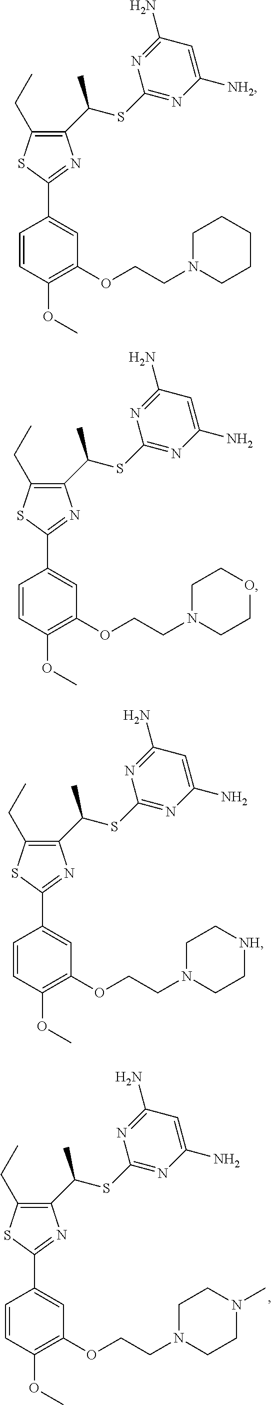 [SMILES: CCc1sc(-c2ccc(OC)c(OCCN3CCCCC3)c2)nc1[C@@H](C)Sc1nc(N)cc(N)n1 ; CCc1sc(-c2ccc(OC)c(OCCN3CCOCC3)c2)nc1[C@@H](C)Sc1nc(N)cc(N)n1 ; CCc1sc(-c2ccc(OC)c(OCCN3CCNCC3)c2)nc1[C@@H](C)Sc1nc(N)cc(N)n1 ; CCc1sc(-c2ccc(OC)c(OCCN3CCN(C)CC3)c2)nc1[C@@H](C)Sc1nc(N)cc(N)n1]

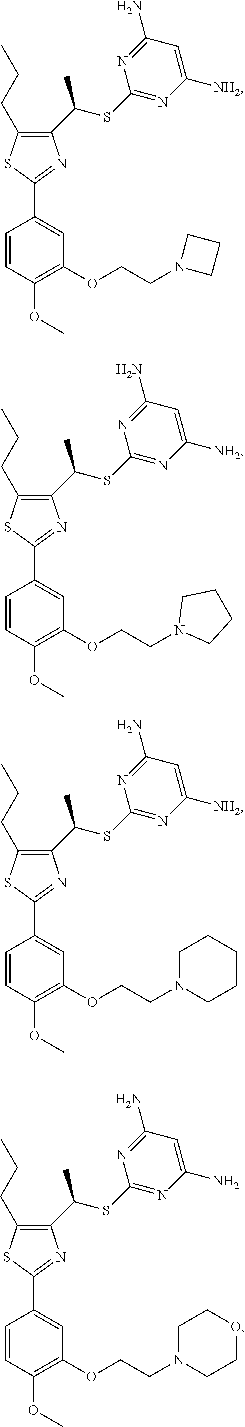 [SMILES: CCCc1sc(-c2ccc(OC)c(OCCN3CCC3)c2)nc1[C@@H](C)Sc1nc(N)cc(N)n1 ; CCCc1sc(-c2ccc(OC)c(OCCN3CCCC3)c2)nc1[C@@H](C)Sc1nc(N)cc(N)n1 ; CCCc1sc(-c2ccc(OC)c(OCCN3CCCCC3)c2)nc1[C@@H](C)Sc1nc(N)cc(N)n1 ; CCCc1sc(-c2ccc(OC)c(OCCN3CCOCC3)c2)nc1[C@@H](C)Sc1nc(N)cc(N)n1]

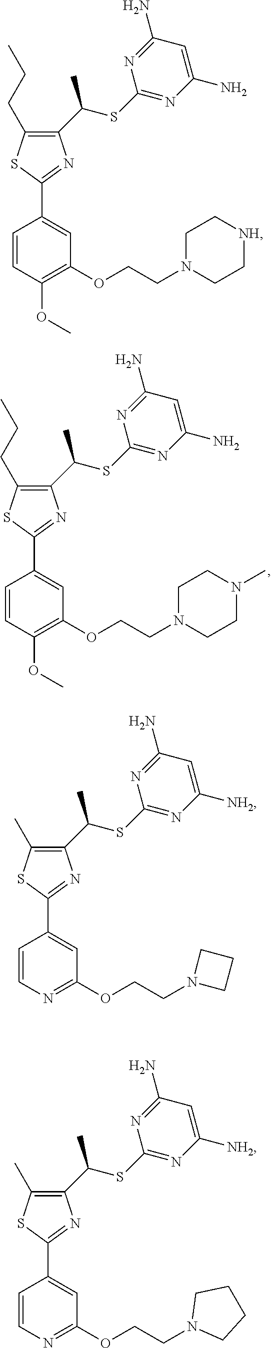

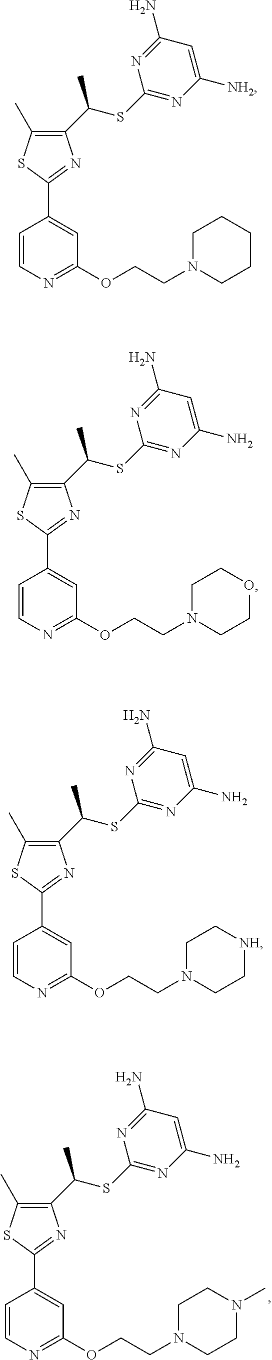

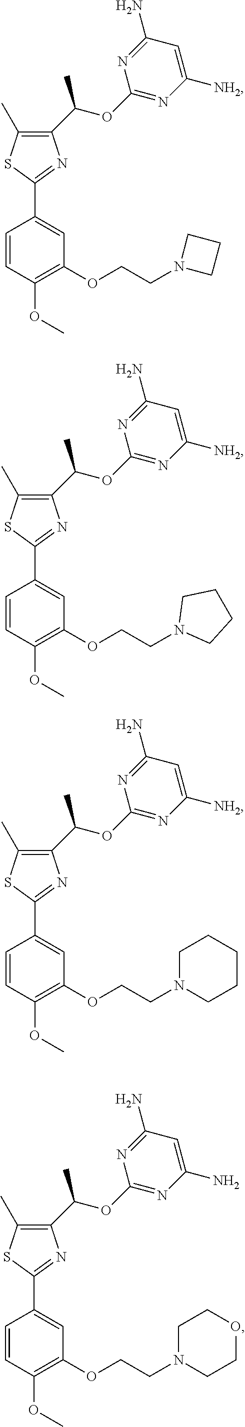 [SMILES: COc1ccc(-c2nc([C@@H](C)Oc3nc(N)cc(N)n3)c(C)s2)cc1OCCN1CCC1 ; COc1ccc(-c2nc([C@@H](C)Oc3nc(N)cc(N)n3)c(C)s2)cc1OCCN1CCCC1 ; COc1ccc(-c2nc([C@@H](C)Oc3nc(N)cc(N)n3)c(C)s2)cc1OCCN1CCCCC1 ; COc1ccc(-c2nc([C@@H](C)Oc3nc(N)cc(N)n3)c(C)s2)cc1OCCN1CCOCC1]

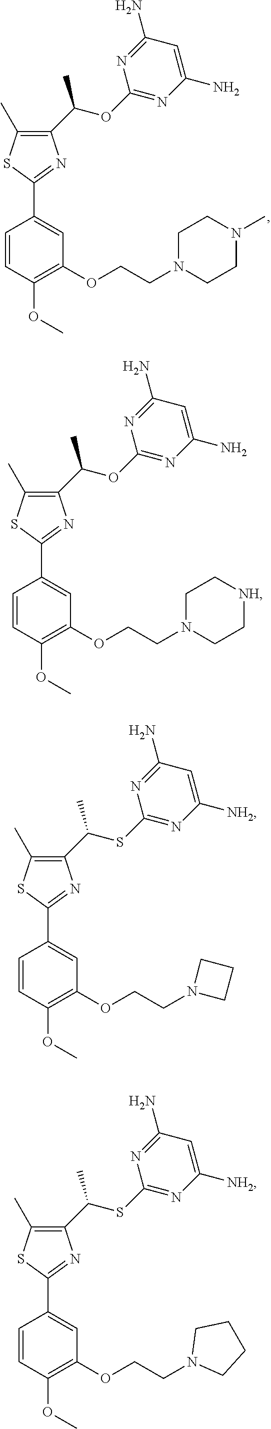

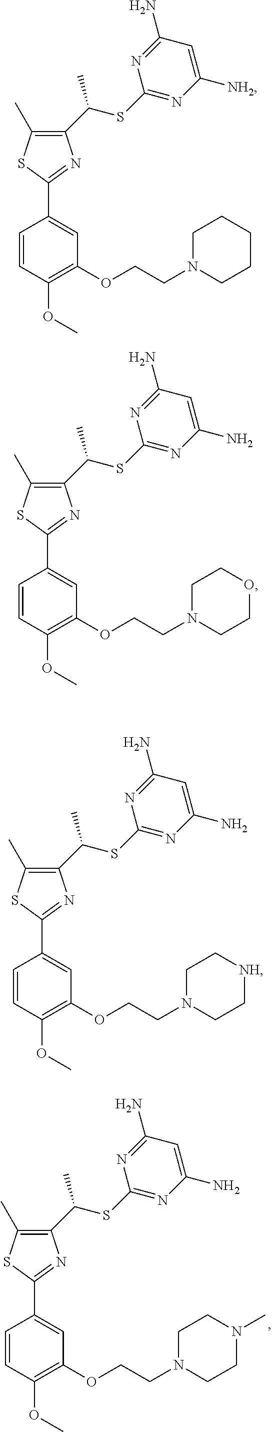

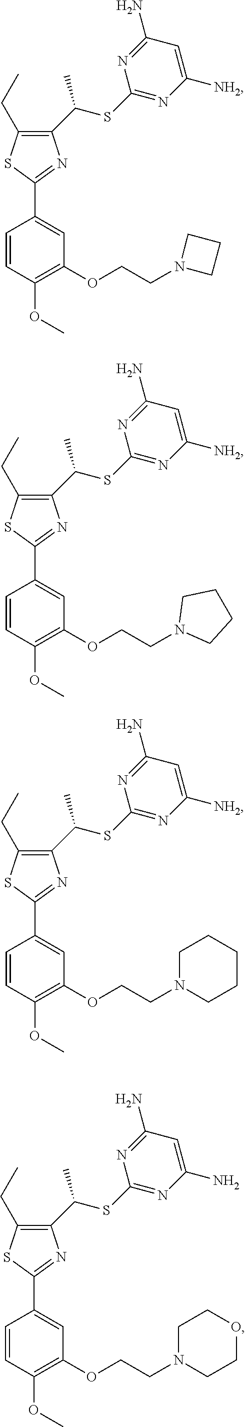 [SMILES: CCc1sc(-c2ccc(OC)c(OCCN3CCC3)c2)nc1[C@H](C)Sc1nc(N)cc(N)n1 ; CCc1sc(-c2ccc(OC)c(OCCN3CCCC3)c2)nc1[C@H](C)Sc1nc(N)cc(N)n1 ; CCc1sc(-c2ccc(OC)c(OCCN3CCCCC3)c2)nc1[C@H](C)Sc1nc(N)cc(N)n1 ; CCc1sc(-c2ccc(OC)c(OCCN3CCOCC3)c2)nc1[C@H](C)Sc1nc(N)cc(N)n1]

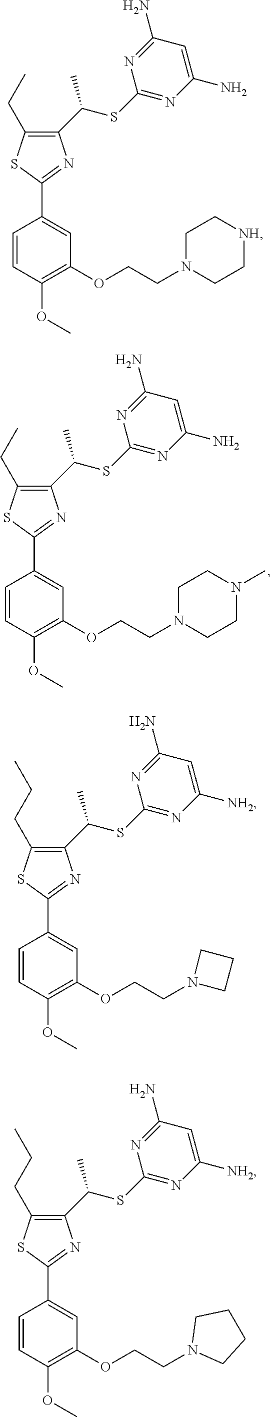 [SMILES: CCc1sc(-c2ccc(OC)c(OCCN3CCNCC3)c2)nc1[C@H](C)Sc1nc(N)cc(N)n1 ; CCc1sc(-c2ccc(OC)c(OCCN3CCN(C)CC3)c2)nc1[C@H](C)Sc1nc(N)cc(N)n1 ; CCCc1sc(-c2ccc(OC)c(OCCN3CCC3)c2)nc1[C@H](C)Sc1nc(N)cc(N)n1 ; CCCc1sc(-c2ccc(OC)c(OCCN3CCCC3)c2)nc1[C@H](C)Sc1nc(N)cc(N)n1]

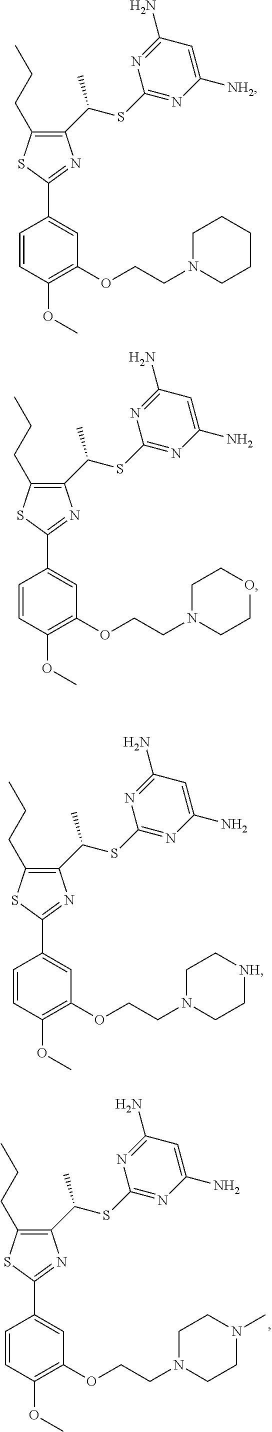 [SMILES: CCCc1sc(-c2ccc(OC)c(OCCN3CCCCC3)c2)nc1[C@H](C)Sc1nc(N)cc(N)n1 ; CCCc1sc(-c2ccc(OC)c(OCCN3CCOCC3)c2)nc1[C@H](C)Sc1nc(N)cc(N)n1 ; CCCc1sc(-c2ccc(OC)c(OCCN3CCNCC3)c2)nc1[C@H](C)Sc1nc(N)cc(N)n1 ; CCCc1sc(-c2ccc(OC)c(OCCN3CCN(C)CC3)c2)nc1[C@H](C)Sc1nc(N)cc(N)n1]

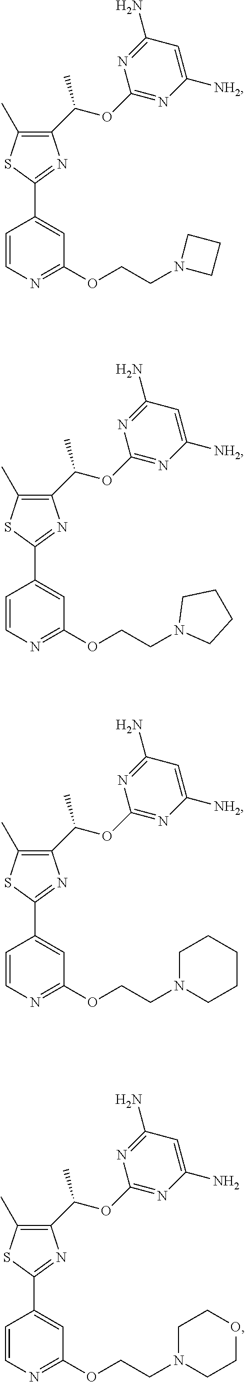 [SMILES: Cc1sc(-c2ccnc(OCCN3CCC3)c2)nc1[C@H](C)Oc1nc(N)cc(N)n1 ; Cc1sc(-c2ccnc(OCCN3CCCC3)c2)nc1[C@H](C)Oc1nc(N)cc(N)n1 ; Cc1sc(-c2ccnc(OCCN3CCCCC3)c2)nc1[C@H](C)Oc1nc(N)cc(N)n1 ; Cc1sc(-c2ccnc(OCCN3CCOCC3)c2)nc1[C@H](C)Oc1nc(N)cc(N)n1]

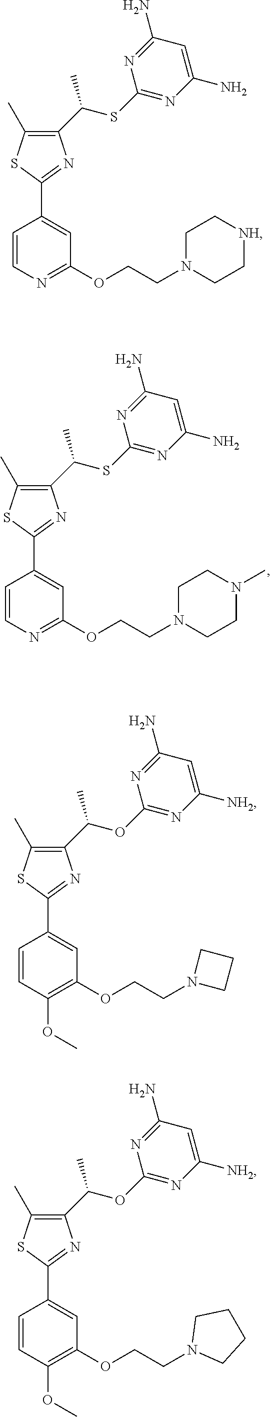

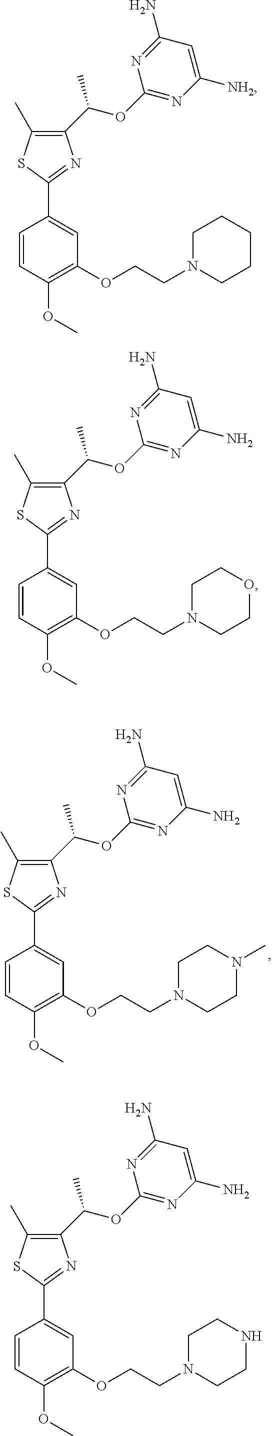

or a pharmaceutically acceptable salt or solvate thereof.

In embodiments, the replication stress response pathway inhibitor is an ATR inhibitor. In embodiments, the replication stress response pathway inhibitor is a Chk1 inhibitor. In embodiments, the replication stress response pathway inhibitor is a WEE1 inhibitor. In embodiments, the replication stress response pathway inhibitor is a compound listed in Table 3. In embodiments, the replication stress response pathway inhibitor is VE-822.

In embodiments of the pharmaceutical compositions, the one or more compounds, or pharmaceutically acceptable salts thereof, are included in a therapeutically effective amount.

In embodiments, the pharmaceutical composition further includes an additional agent (e.g. therapeutic agent). In embodiments, the further agent is an anti-cancer agent. In embodiments, the further agent is a chemotherapeutic. In embodiments of the pharmaceutical compositions, the pharmaceutical composition includes a further agent (e.g. therapeutic agent) in a therapeutically effective amount.

Methods

In an aspect is provided a method of treating cancer in a patient in need of the treatment, the method including administering pharmaceutical composition as described herein (including in an aspect, embodiment, table, figure, claim, sequence listing, or example).

In an aspect is provided a pharmaceutical composition as described herein for use in the manufacture of a medicament for treatment of a disease (e.g., cancer). The use includes administering to the subject a pharmaceutical composition described herein. The use may include administering to the subject a therapeutically effective amount of a pharmaceutical composition described herein.

In an aspect is provided a pharmaceutical composition as described herein for use in the treatment of a cancer in a subject in need of such treatment. The use includes administering to the subject a pharmaceutical composition described herein. The use may include administering to the subject a therapeutically effective amount of a pharmaceutical composition described herein.

In embodiments, the method or use includes administering a therapeutically effective amount of a pharmaceutical composition described herein (including in an aspect, embodiment, table, figure, claim, sequence listing, or example).

In embodiments, the method or use includes systemic administration of the pharmaceutical composition. In embodiments, the method or use includes parenteral administration of the pharmaceutical composition. In embodiments, the method or use includes intravenous administration of the pharmaceutical composition. In embodiments, the method or use includes administration directly to a tumor. In embodiments, the method or use includes local administration to the site of cancer.

In embodiments, the cancer is a hematopoietic cell cancer. In embodiments, the cancer is not a hematopoietic cell cancer. In embodiments, the cancer is prostate cancer, breast cancer, glioblastoma, ovarian cancer, lung cancer, head and neck cancer, esophageal cancer, skin cancer, melanoma, brain cancer, colorectal cancer, leukemia, lymphoma, or myeloma. In embodiments, the cancer is prostate cancer (e.g. castration-resistant). In embodiments, the cancer is breast cancer (e.g. triple negative). In embodiments, the cancer is glioblastoma. In embodiments, the cancer is ovarian cancer. In embodiments, the cancer is lung cancer. In embodiments, the cancer is head and neck cancer. In embodiments, the cancer is esophageal cancer. In embodiments, the cancer is skin cancer. In embodiments, the cancer is melanoma. In embodiments, the cancer is brain cancer. In embodiments, the cancer is colorectal cancer. In embodiments, the cancer is leukemia (e.g. AML, ALL, or CML). In embodiments, the cancer is lymphoma. In embodiments, the cancer is myeloma (e.g. multiple myeloma). In embodiments, the cancer is squamous cell carcinoma (e.g. head and neck cancer or esophageal cancer). In embodiments, the cancer is metastatic cancer. In embodiments, the cancer is acute myeloid leukemia. In embodiments, the cancer is B cell lymphoma. In embodiments, the cancer is multiple myeloma.

In embodiments, the cancer has an increased level of de novo nucleotide biosynthesis pathway activity relative to a control (e.g. non-cancerous cell of the same type as the cancer cell). In embodiments, the cancer has an increased level of nucleoside salvage pathway activity relative to a control (e.g. non-cancerous cell of the same type as the cancer cell). In embodiments, the cancer has an increased level of replication stress response pathway activity relative to a control (e.g. non-cancerous cell of the same type as the cancer cell).

In embodiments, the cancer includes an increased level of RNR relative to a control (e.g. non-cancerous cell of the same type as the cancer cell). In embodiments, the cancer includes an increased level of RNR activity relative to a control (e.g. non-cancerous cell of the same type as the cancer cell). In embodiments, the cancer includes an increased level of dCK relative to a control (e.g. non-cancerous cell of the same type as the cancer cell). In embodiments, the cancer includes an increased level of dCK activity relative to a control (e.g. non-cancerous cell of the same type as the cancer cell). In embodiments, the cancer includes an increased level of ATR relative to a control (e.g. non-cancerous cell of the same type as the cancer cell). In embodiments, the cancer includes an increased level of ATR activity relative to a control (e.g. non-cancerous cell of the same type as the cancer cell). In embodiments, the cancer includes an increased level of Chk1 relative to a control (e.g. non-cancerous cell of the same type as the cancer cell). In embodiments, the cancer includes an increased level of Chk1 activity relative to a control (e.g. non-cancerous cell of the same type as the cancer cell). In embodiments, the cancer includes an increased level of WEE1 relative to a control (e.g. non-cancerous cell of the same type as the cancer cell). In embodiments, the cancer includes an increased level of WEE1 activity relative to a control (e.g. non-cancerous cell of the same type as the cancer cell).

In an aspect is provided a method of inhibiting the growth of a cancer cell including contacting the cancer cell with a pharmaceutical composition described herein (including in an aspect, embodiment, table, figure, claim, sequence listing, or example).

In an aspect is provided a pharmaceutical composition as described herein for use in inhibiting the growth of a cancer cell. The use includes contacting the cancer cell with a pharmaceutical composition described herein. The use may include contacting the cancer cell with an effective amount of a pharmaceutical composition described herein.

In an aspect is provided a pharmaceutical composition as described herein for use in the manufacture of a medicament for inhibiting the growth of a cancer cell.

In embodiments, the cancer cell includes an increased level of RNR relative to a control (e.g. non-cancerous cell of the same type as the cancer cell). In embodiments, the cancer cell includes an increased level of RNR activity relative to a control (e.g. non-cancerous cell of the same type as the cancer cell). In embodiments, the cancer cell includes an increased level of dCK relative to a control (e.g. non-cancerous cell of the same type as the cancer cell). In embodiments, the cancer cell includes an increased level of dCK activity relative to a control (e.g. non-cancerous cell of the same type as the cancer cell). In embodiments, the cancer cell includes an increased level of ATR relative to a control (e.g. non-cancerous cell of the same type as the cancer cell). In embodiments, the cancer cell includes an increased level of ATR activity relative to a control (e.g. non-cancerous cell of the same type as the cancer cell). In embodiments, the cancer cell includes an increased level of Chk1 relative to a control (e.g. non-cancerous cell of the same type as the cancer cell). In embodiments, the cancer cell includes an increased level of Chk1 activity relative to a control (e.g. non-cancerous cell of the same type as the cancer cell). In embodiments, the cancer cell includes an increased level of WEE1 relative to a control (e.g. non-cancerous cell of the same type as the cancer cell). In embodiments, the cancer cell includes an increased level of WEE1 activity relative to a control (e.g. non-cancerous cell of the same type as the cancer cell).

In embodiments, the method or use includes inducing apoptosis of the cancer cell. In embodiments, the method or use includes inducing apoptosis in a cancer cell but not a non-cancer cell. In embodiments, the method or use includes inducing apoptosis in a cancer cell in a patient but not a non-cancer cell in the same patient. In embodiments, the method or use includes inducing apoptosis in a cancer cell but not a non-cancer cell of the same cell type as the cancer cell (e.g. lung cell, breast cell, pancreatic cell, colorectal cell, prostate cell, hematopoietic cell). In embodiments, the cancer cell is in an organ. In embodiments, the cancer cell is in a bone. In embodiments, the cancer cell is in bone.

### EMBODIMENTS

Embodiments includes embodiment P1 to P24 following.

### Embodiment P1

A pharmaceutical composition comprising a pharmaceutically acceptable excipient, a de novo nucleotide biosynthesis pathway inhibitor, a nucleoside salvage pathway inhibitor, and a replication stress response pathway inhibitor.

### Embodiment P2

The pharmaceutical composition of embodiment P1, wherein the de novo nucleotide biosynthesis pathway inhibitor is an RNR inhibitor.

### Embodiment P3

The pharmaceutical composition of embodiment P1, wherein the de novo nucleotide biosynthesis pathway inhibitor is selected from the compounds of Table 1.

### Embodiment P4

The pharmaceutical composition of embodiment P, wherein the de novo nucleotide biosynthesis pathway inhibitor is 3-AP.

### Embodiment P5

The pharmaceutical composition of one of embodiments P1 to P4, wherein the nucleoside salvage pathway inhibitor is a dCK inhibitor.

### Embodiment P6

The pharmaceutical composition of one of embodiments P1 to P4, wherein the nucleoside salvage pathway inhibitor is selected from the compounds of Table 2.

### Embodiment P7

The pharmaceutical composition of one of embodiments P1 to P4, wherein the nucleoside salvage pathway inhibitor is a racemic mixture of DI-82.

### Embodiment P8

The pharmaceutical composition of one of embodiments P1 to P4, wherein the nucleoside salvage pathway inhibitor is (R) DI-82.

### Embodiment P9

The pharmaceutical composition of one of embodiments P1 to P8, wherein the replication stress response pathway inhibitor is an ATR inhibitor.

### Embodiment P10

The pharmaceutical composition of one of embodiments P1 to P8, wherein the replication stress response pathway inhibitor is a Chk1 inhibitor.

### Embodiment P11

The pharmaceutical composition of one of embodiments P1 to P8, wherein the replication stress response pathway inhibitor is a WEE1 inhibitor.

### Embodiment P12

The pharmaceutical composition of one of embodiments P1 to P8, wherein the replication stress response pathway inhibitor is selected from the compounds of Table 3.

### Embodiment P13

The pharmaceutical composition of one of embodiments P1 to P12, wherein the replication stress response pathway inhibitor is VE-822.

### Embodiment P14

The pharmaceutical composition of one of embodiments P1 to P13 for use in treating cancer in a patient in need of such treatment, the use comprising administering an effective amount of the pharmaceutical composition to the patient.

### Embodiment P15

A pharmaceutical composition of one of embodiments P1 to P13 for use in inhibiting the growth of a cancer cell comprising contacting the cancer cell with the pharmaceutical composition.

### Embodiment P16

A pharmaceutical composition, comprising:
(i) a pharmaceutically acceptable excipient; and
(ii) a de novo nucleotide biosynthesis pathway inhibitor, a nucleoside salvage pathway inhibitor, a replication stress response pathway inhibitor, or any combination thereof.

### Embodiment P17

The pharmaceutical composition of embodiment P16, wherein the composition comprises a de novo nucleotide biosynthesis pathway inhibitor.

### Embodiment P18

The pharmaceutical composition of embodiment P16, wherein the composition comprises a nucleoside salvage pathway inhibitor.

### Embodiment P19

The pharmaceutical composition of embodiment P16, wherein the composition comprises a replication stress response pathway inhibitor.

### Embodiment P20

The pharmaceutical composition of embodiment P16, wherein the composition comprises a de novo nucleotide biosynthesis pathway inhibitor and a nucleoside salvage pathway inhibitor.

### Embodiment P21

The pharmaceutical composition of embodiment P16, wherein the composition comprises a de novo nucleotide biosynthesis pathway inhibitor and a a replication stress response pathway inhibitor.

### Embodiment P22

The pharmaceutical composition of embodiment P16, wherein the composition comprises a nucleoside salvage pathway inhibitor, a replication stress response pathway inhibitor.

### Embodiment P23

The pharmaceutical composition of one of embodiments P16 to P22 for use in treating cancer in a patient in need of such treatment, the use comprising administering an effective amount of the pharmaceutical composition to the patient.

### Embodiment P24

A pharmaceutical composition of one of embodiments P16 to P22 for use in inhibiting the growth of a cancer cell comprising contacting the cancer cell with the pharmaceutical composition.

Further embodiments include embodiments 1 to 24 following.

### Embodiment 1

A pharmaceutical composition comprising a pharmaceutically acceptable excipient, a de novo nucleotide biosynthesis pathway inhibitor, a nucleoside salvage pathway inhibitor, and a replication stress response pathway inhibitor.

### Embodiment 2

The pharmaceutical composition of embodiment 1, wherein the de novo nucleotide biosynthesis pathway inhibitor is an RNR inhibitor.

### Embodiment 3

The pharmaceutical composition of embodiment 1, wherein the de novo nucleotide biosynthesis pathway inhibitor is selected from the compounds of Table 1.

### Embodiment 4

The pharmaceutical composition of embodiment 1, wherein the de novo nucleotide biosynthesis pathway inhibitor is 3-AP.

### Embodiment 5

The pharmaceutical composition of one of embodiments 1 to 4, wherein the nucleoside salvage pathway inhibitor is a dCK inhibitor.

### Embodiment 6

The pharmaceutical composition of one of embodiments 1 to 4, wherein the nucleoside salvage pathway inhibitor is selected from the compounds of Table 2.

### Embodiment 7

The pharmaceutical composition of one of embodiments 1 to 4, wherein the nucleoside salvage pathway inhibitor is a racemic mixture of DI-82.

### Embodiment 8

The pharmaceutical composition of one of embodiments 1 to 4, wherein the nucleoside salvage pathway inhibitor is (R) DI-82.

### Embodiment 9

The pharmaceutical composition of one of embodiments 1 to 8, wherein the replication stress response pathway inhibitor is an ATR inhibitor.

### Embodiment 10

The pharmaceutical composition of one of embodiments 1 to 8, wherein the replication stress response pathway inhibitor is a Chk1 inhibitor.

### Embodiment 11

The pharmaceutical composition of one of embodiments 1 to 8, wherein the replication stress response pathway inhibitor is a WEE1 inhibitor.

### Embodiment 12

The pharmaceutical composition of one of embodiments 1 to 8, wherein the replication stress response pathway inhibitor is selected from the compounds of Table 3.

### Embodiment 13

The pharmaceutical composition of one of embodiments 1 to 12, wherein the replication stress response pathway inhibitor is VE-822.

### Embodiment 14

The pharmaceutical composition of one of embodiments 1 to 13 for use in treating cancer in a patient in need of such treatment, the use comprising administering an effective amount of the pharmaceutical composition to the patient.

### Embodiment 15

A pharmaceutical composition of one of embodiments 1 to 13 for use in inhibiting the growth of a cancer cell comprising contacting the cancer cell with the pharmaceutical composition.

### Embodiment 16

A pharmaceutical composition, comprising:
(i) a pharmaceutically acceptable excipient; and
(ii) a de novo nucleotide biosynthesis pathway inhibitor, a nucleoside salvage pathway inhibitor, a replication stress response pathway inhibitor, or any combination thereof.

### Embodiment 17

The pharmaceutical composition of embodiment 16, wherein the composition comprises a de novo nucleotide biosynthesis pathway inhibitor.

### Embodiment 18

The pharmaceutical composition of embodiment 16, wherein the composition comprises a nucleoside salvage pathway inhibitor.

### Embodiment 19

The pharmaceutical composition of embodiment 16, wherein the composition comprises a replication stress response pathway inhibitor.

### Embodiment 20

The pharmaceutical composition of embodiment 16, wherein the composition comprises a de novo nucleotide biosynthesis pathway inhibitor and a nucleoside salvage pathway inhibitor.

### Embodiment 21

The pharmaceutical composition of embodiment 16, wherein the composition comprises a de novo nucleotide biosynthesis pathway inhibitor and a a replication stress response pathway inhibitor.

### Embodiment 22

The pharmaceutical composition of embodiment 16, wherein the composition comprises a nucleoside salvage pathway inhibitor, a replication stress response pathway inhibitor.

### Embodiment 23

The pharmaceutical composition of one of embodiments 16 to 22 for use in treating cancer in a patient in need of such treatment, the use comprising administering an effective amount of the pharmaceutical composition to the patient.

### Embodiment 24

A pharmaceutical composition of one of embodiments 16 to 22 for use in inhibiting the growth of a cancer cell comprising contacting the cancer cell with the pharmaceutical composition.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Replication stress has emerged as a targetable hallmark of cancer. Inhibitors of the replication stress response (RSRi) pathways represent a novel class of anti-cancer drugs. RSRi impacts both signaling network involved in monitoring genomic integrity and the metabolic pathways involved in nucleotide biosynthesis, such as the expression level of a rate limiting step in the de novo pathway, RNR. For example, VE-821, an ATR inhibitor, was shown to reduce RNR expression through a CDK1 dependent post-translational mechanism. Resistance to RSRi is traditionally thought to involve only signaling mechanisms—for instance, re-routing of the replication stress response through alternate pathways (in the case of ATR inhibition, related kinases ATM and DNA-PK mediate compensatory mechanisms).

Many aggressive cancers are characterized by high levels of replication stress because of elevated oncogenic signaling and/or defects in DNA damage response/repair pathways. This phenomenon renders such cancers significantly more susceptible than normal cells to therapies that constrain dNTP biosynthesis, but such therapies are often ineffective because of two resistance mechanisms: 1) redundancy in nucleotide biosynthesis and 2) control mechanisms mediated by the replication stress response and DNA damage response pathways that enable adaptation to conditions of dNTP pool insufficiencies.

We hypothesize that a hitherto unappreciated but important resistance mechanism to ATR inhibition involves its target in nucleotide biosynthesis, in particular dCK, the rate-limiting enzyme in the salvage pathway. Thus, dCK activity could compensate for ATRi-induced downregulation of RNR. A better understanding of the metabolic shifts following ATR inhibition can lead to the development of effective combination treatment. We tested and validated this hypothesis and found that dCK is a resistance mechanism to ATR inhibition in leukemia. However, we also observed that residual RNR levels may meditate an additional resistance mechanism to ATR inhibition. We hypothesized that this additional resistance mechanism could be targeted using low doses of RNR inhibitors. To this end, we profiled existing RNR inhibitors to develop a novel combination therapy to treat leukemia.

Here, we describe an integrated analytical platform to study the interplay between replication stress response, nucleotide biosynthetic pathway activity and cell cycle progression. This strategy enabled the development of a new synthetic lethal approach shown to be effective and well tolerated in a representative model of preB-ALL.

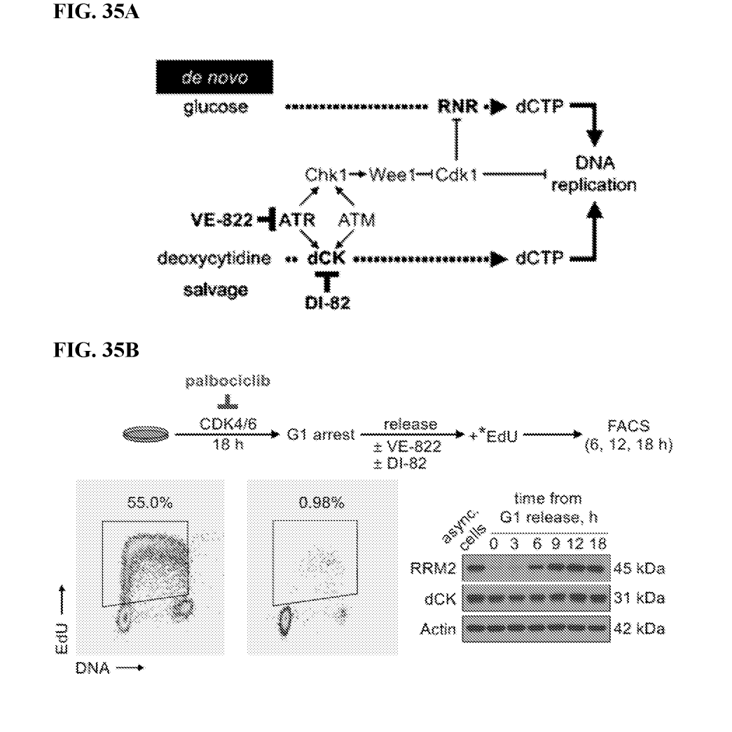

Cancer cells in G1 phase of the cell cycle do not possess adequate dNTPs to complete DNA replication. In addition, many components of nucleotide biosynthesis (e.g. RRM2 and TK1) are not constitutively expressed and are S-phase specific. Thus, cells do not possess maximal nucleotide biosynthetic capacity at the G1/S transition. An inadequate supply of dNTP during S-phase results in intrinsic replication stress. ATR, a master regulator of the replication stress response pathway, mitigates this intrinsic replication stress by promoting RRM2 transcription, activating dCK and suppressing DNA replication (FIG. 35A).

While the transcriptional regulation of RRM2 by ATR is known, the connection between ATR and dCK is largely unexplored. This is particularly relevant at the G1/S transition where RRM2 activity is rate-limiting but dCK is constitutively expressed. This suggests that dCK provides a resistance mechanism to ATR inhibition by VE-822. DI-82, a dCK specific inhibitor, is used to investigate this possibility.

CCRF-CEM T-ALL cells were synchronized by treatment with the CDK4/6 inhibitor, palbociclib, released in drug free or media containing VE-822 with and without DI-82 and cell cycle progression was subsequently monitored by an EdU pulse assay (FIG. 35B). Following release from G1, cells progress to early, mid and late S phase (defined as S1, S2 and S3, respectively) and G2/M. By 18 h following release in drug-free media, 42.5% of cells progressed to S3. VE-822 addition resulted in a 16% decrease in the number of cells in S2 at 12 h, highlighting the importance of ATR in cell cycle progression (FIG. 35C). Whereas dCK inhibition alone minimally affected cell cycle progression the addition of DI-82 exacerbated the G1-S transition in VE-822 treated cells. Thus, dCK represents a resistance mechanism to ATR inhibition.

We reasoned that the cell cycle progression defect in VE-822+DI-82 treated cells could be explained by decreased nucleotide biosynthetic capacity resulting from impaired expression of rate-limiting de novo pathways enzymes.

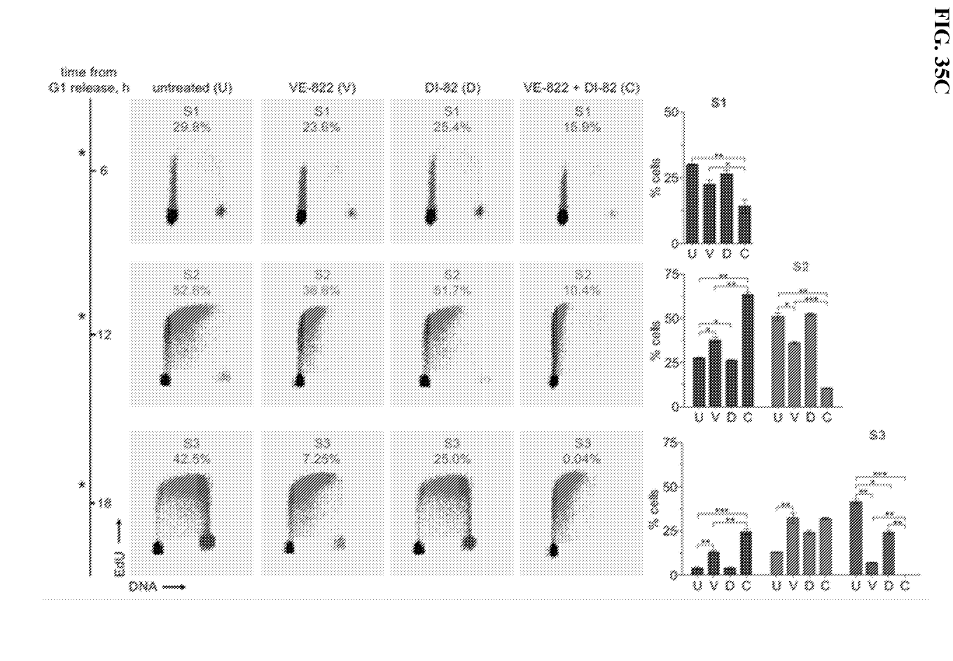

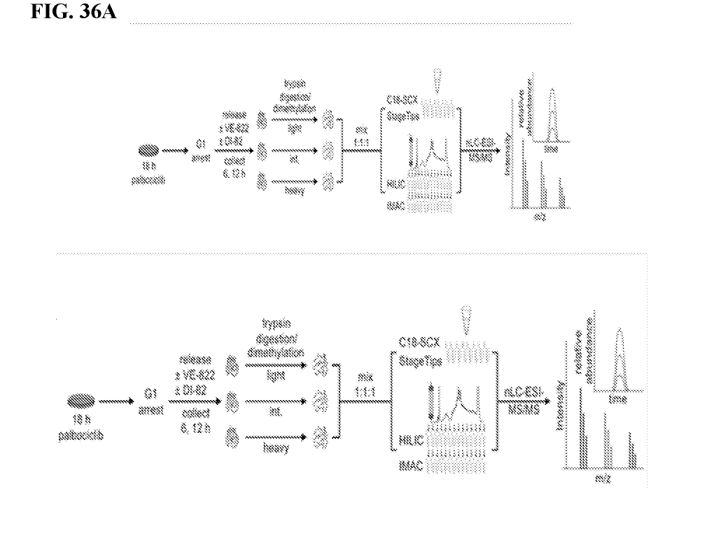

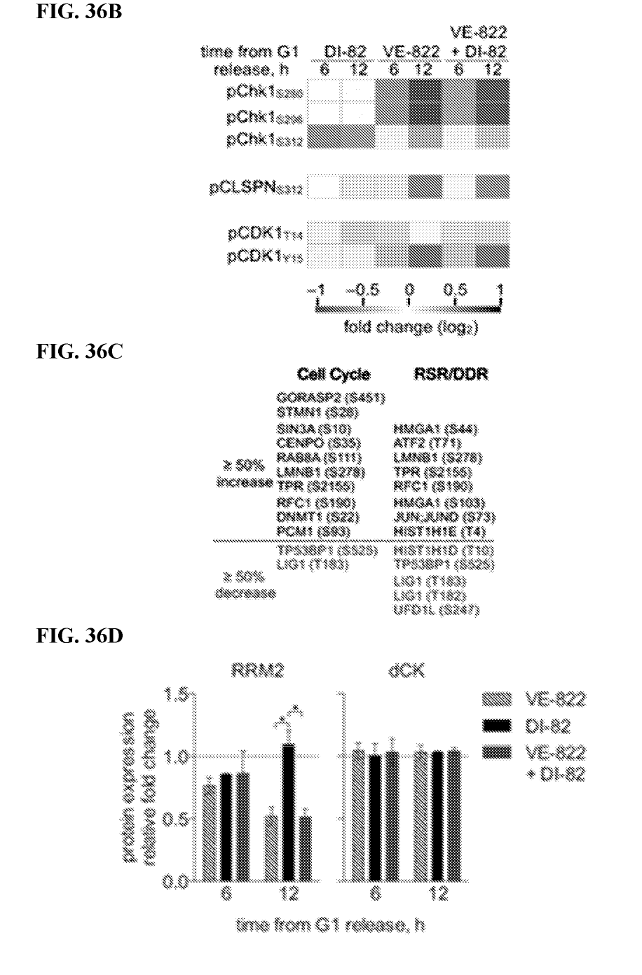

To characterize this mechanism, we performed global proteomics and phosphoproteomics analysis of treated cells following G1 release. This approach enables accurate and unbiased quantification of protein expression and signaling changes in our treatment conditions. We generated a phosphoproteomic dataset from four treatment groups and two time points and identified about 15,000 unique phosphopeptides and 4300 protein groups (FIG. 36A).

VE-822 treatment with and without DI-82 resulted in decreased phosphorylation of Chk1 and Claspin, proteins located downstream of ATR in the RSR/DDR signaling pathway, compared to untreated cells (FIG. 36B). Signaling from RSR and DDR ultimately funnels to CDK1, a master regulator of cell cycle progression. We observed a transient decrease in CDK1 phosphorylation at Thr-14; Tyr-15, sites known to inhibit CDK1 kinase activity and cell cycle progression, following ATR inhibition (FIG. 36B).

The addition of DI-82 to VE-822 treated cells altered the phosphorylation status of only a small subset of proteins involved in cell cycle regulation and the RSR/DDR signaling pathways even though there was a significant impendence on the cell cycle progression (FIG. 36C). ATR inhibition with and without dCK inhibition blunted 2-fold increase in RRM2 expression at 12 h. In contrast, dCK expression was unaltered in all treatment groups (FIG. 36D).

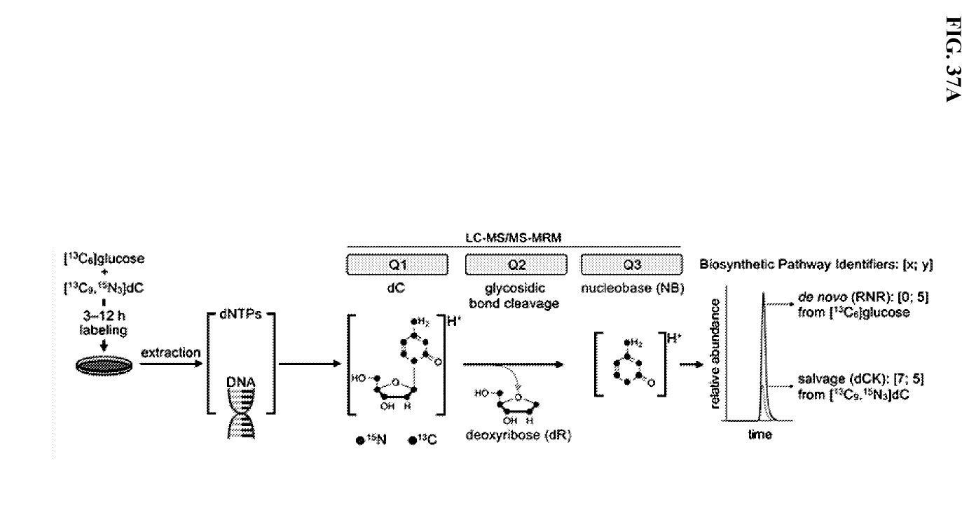

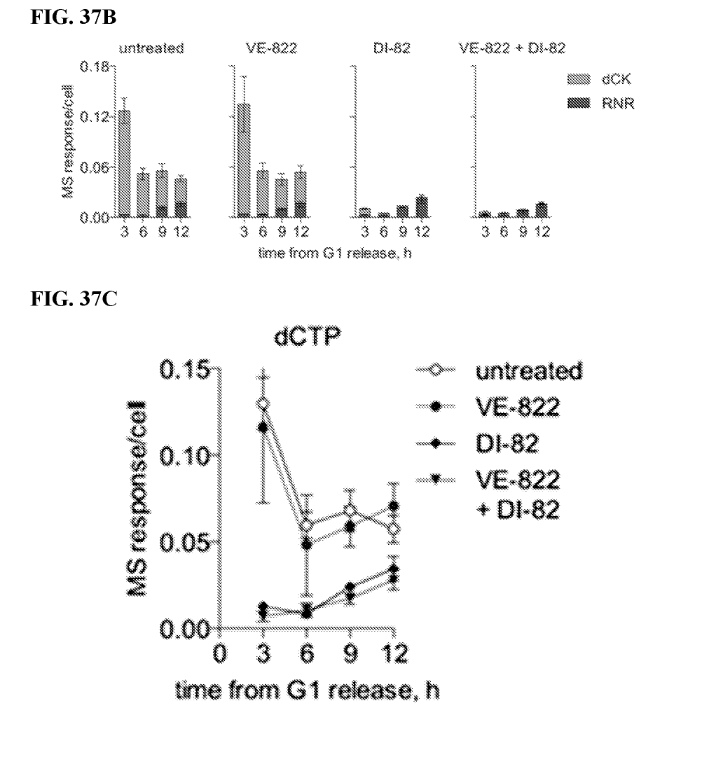

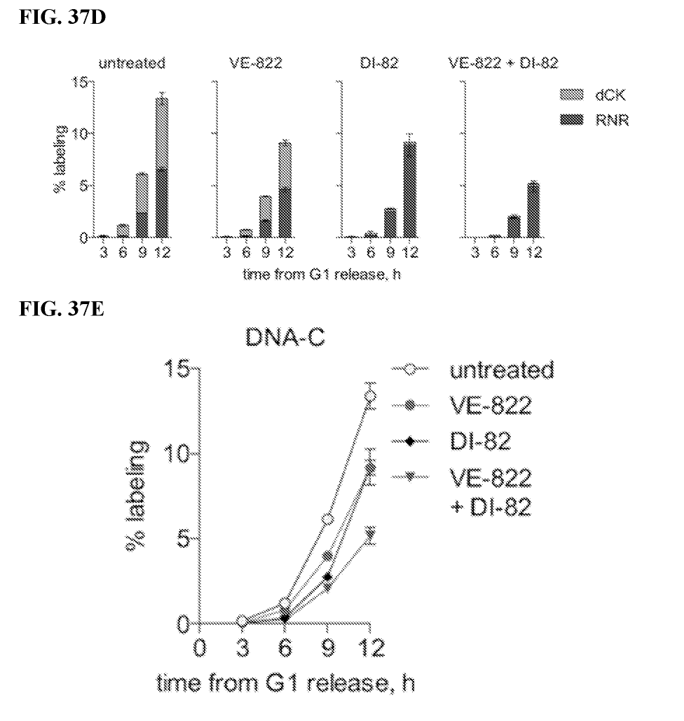

A mass spectrometric assay was used to simultaneously measure the differential contribution of de novo and salvage pathways to newly replicated DNA. Although, VE-822 inhibited the expression of RRM2, the activity of residual RNR was unclear. Thus, we developed a new mass spectrometric assay to enable routine measurements of the differential contributions of de novo and salvage pathways to dNTP pools and newly replicated DNA in cancer cells (FIG. 37A).

To analyze the differential utilization of de novo and nucleotide biosynthetic salvage pathways, cultured cells were incubated with stable isotope labeled DNA precursors. In the example shown in FIG. 37A, [U-¹³C₆]glucose labels the DNA synthesized from de novo pathway while [U-¹³C₉,¹⁵N₃]deoxycytidine (dC) labels the DNA synthesized from the salvage pathways. Extracted dNTP and DNA were analyzed in a triple quadrupole mass spectrometer (QQQ) using multiple reaction monitoring (MRM). The first (Q1) and third (Q3) quadrupoles function as mass filters while the second (Q2) quadrupole serves as a collision chamber. In this example, Q1 selects an intact protonated dC ion with a defined mass-to-charge ratio (m/z). In Q2, the glycosidic bond of the selected dC is cleaved, releasing a protonated nucleobase (NB) fragment and a neutral deoxyribose (dR) molecule. The m/z specific NB fragments from dC is separated in Q3 and detected to generate ion chromatograms. Quantifications of mass increases in both NB and dR generated a set of biosynthetic pathway identifiers (FIG. 37A). Each identifier corresponded to a specific biosynthetic pathway and is defined as [x;y], where “x” is the number of heavy isotope-labeled atoms in NB and “y” is the number of heavy isotope atoms in dR. For example, dC with a [7;5] identifier contains seven heavy isotope-labeled atoms in its NB and five heavy isotope-labeled atoms in its dR component. According to current biochemical maps of nucleotide metabolism, the [7;5] identifier can only arise from salvaging [U-¹³C₉,¹⁵N₃]dC into newly synthesized DNA. In contrast, a dC with a [0,5] identifier is a product of the de novo pathway. The ratio of the peak areas in the ion chromatograms of salvage [7;5] and de novo [0;5] identifiers describes the relative contributions of the two biosynthetic routes to the dCTP pool used for DNA replication.

We then utilized the developed assay to assess the differential contribution of de novo (RNR) and salvage (dCK) pathways to dCTP and DNA-C biosynthesis following G1 release (FIG. 37B-E). In the untreated group dCTP biosynthesis is predominantly dCK dependent following G1 release. This result is consistent with the cell cycle dependent expression of RNR and dCK. The contribution of RNR to dCTP is less than dCK at all timepoints. However, the relative contribution of RNR and dCK to DNA-C at 12 h following release is equal indicating differential utilization of dCTP produced by RNR over dCK. The addition of VE-822 did not significantly impact the dCTP pool levels in G1 released cells compared to the untreated group (FIG. 37B-C). Total DNA-C labeling was 30% less in the VE-822 group compared to untreated controls which is consistent with the reported decreased rate of DNA replication (FIG. 37D-E).

Because of the substantial contribution of dCK to dCTP and DNA-C in VE-822 treated cells, we sought to determine if inhibition of dCK would decreased the rate of DNA replication induced by ATR inhibition. DI-82 completely abrogates the contribution of dCK to dCTP and DNA-C but does not significantly impair cell cycle progression. This can be explained by a compensatory increase in the contribution of RNR to DNA-C in DI-82 treated cells.

Addition of DI-82 to VE-822 decreased the total DNA-C labeling by more than two-fold compared to the untreated at group 12 h and the dCTP pool size was reduced to a level similar to the DI-82 group. (FIG. 37B-C).

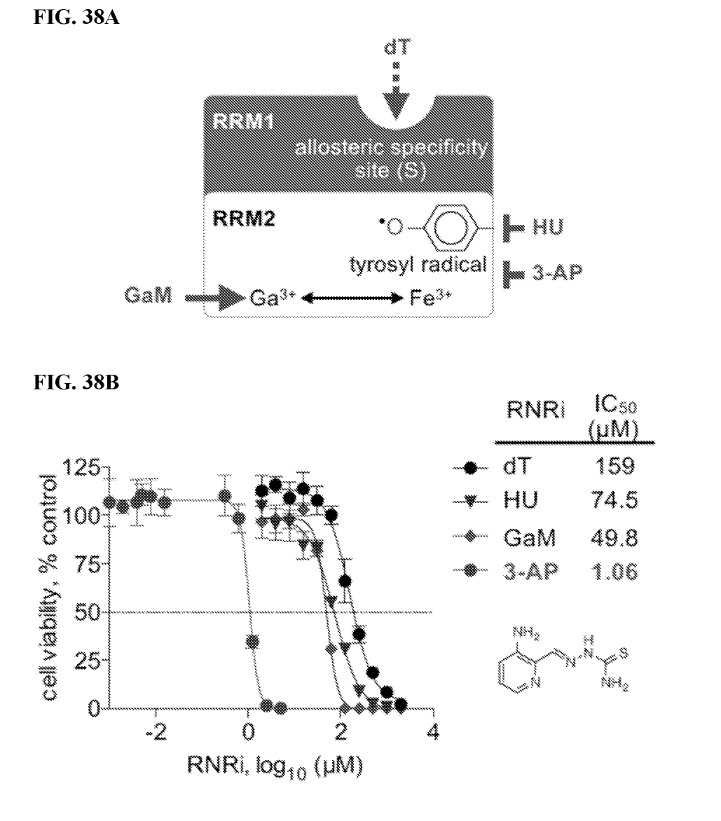

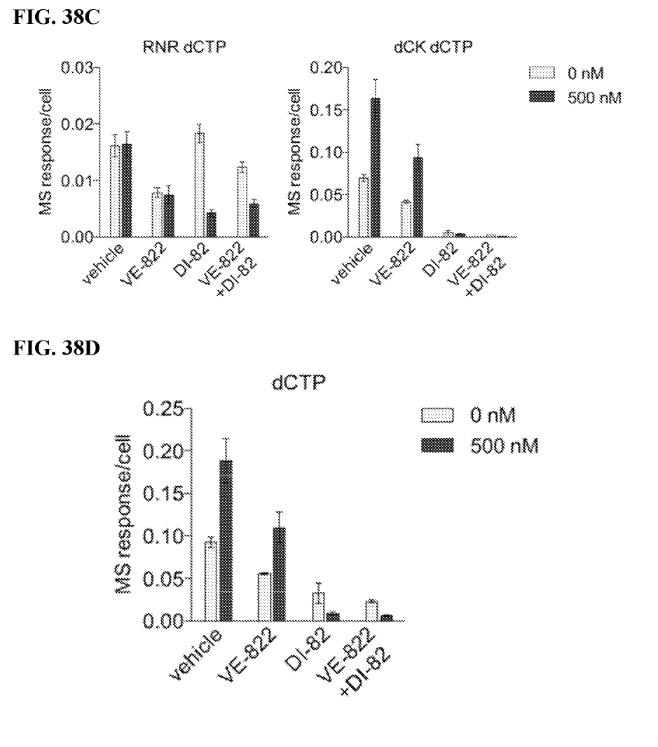

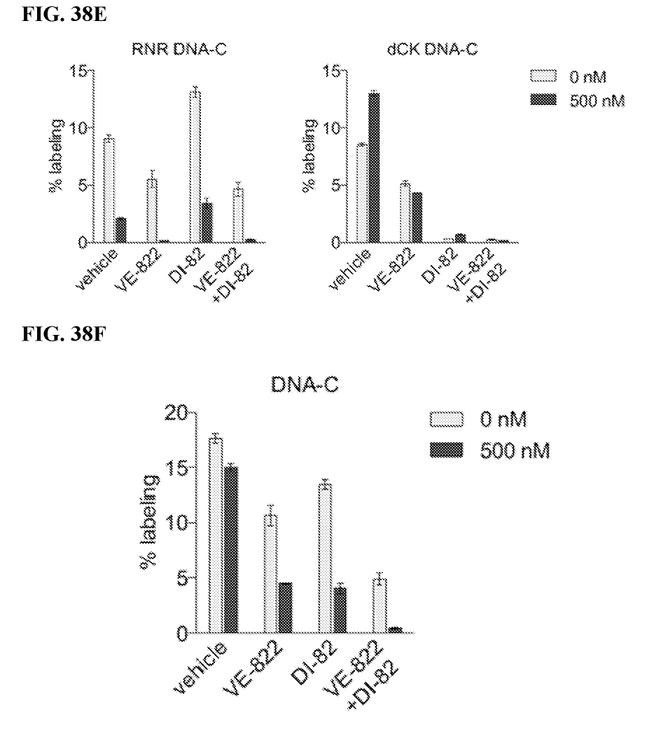

We reasoned that residual RNR activity enables cancer cell survival and that directly targeting RNR would increase replication stress to an intolerable level, replication stress overload, and result in cell death. We assessed four RNR inhibitors—thymidine (dT), gallium maltolate (GaM), hydroxyurea (HU) and triapine (3-AP), each with a distinct mechanism of action, for their ability to inhibit cell growth (FIG. 38A). Amongst the four candidate RNR inhibitors, 3-AP demonstrated an IC₅₀ value of 50 to 150-fold lower in inhibiting CEM cell growth (FIG. 38B).

3-AP at 500 nM did not affect the de novo dCTP pool, but did induced a 2-fold increase in salvage dCTP pool that contributed significantly to the overall dCTP pool expansion (FIG. 38C-D). VE-822 alone reduced both dCTP pools and that the addition of 3-AP increased only the salvage dCTP pool. This is consistent with dCK role as an alternative dCTP biosynthesis to RNR. The addition of DI-82 transformed the dCTP pool dynamic by abolishing the salvage dCTP pool.

At the DNA level, 3-AP decreased the RNR contribution in all treatment conditions and DI-82 severely limited dCK contribution to DNA-C biosynthesis (FIG. 38E). The combined targeting of ATR, dCK and RNR nearly abrogates DNA-C biosynthesis completely (FIG. 38F).

We next investigated the effect of this drug combination on biomarkers of replication stress and DNA damage. Combining VE-822 and DI-82 resulted in a ˜4-fold increase in the levels of ssDNA accumulation after 0.5 h. This damage progressed to DSB at 4 h as evidenced by a ˜10-fold increase in pH2A.X signaling compared to untreated cells.

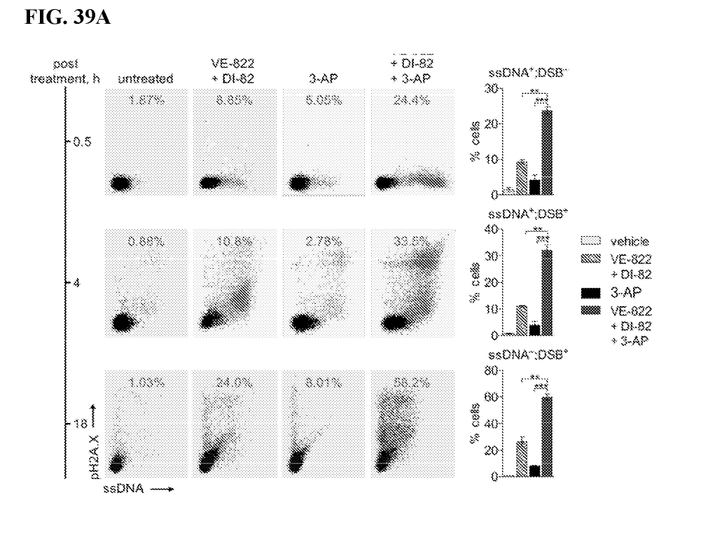

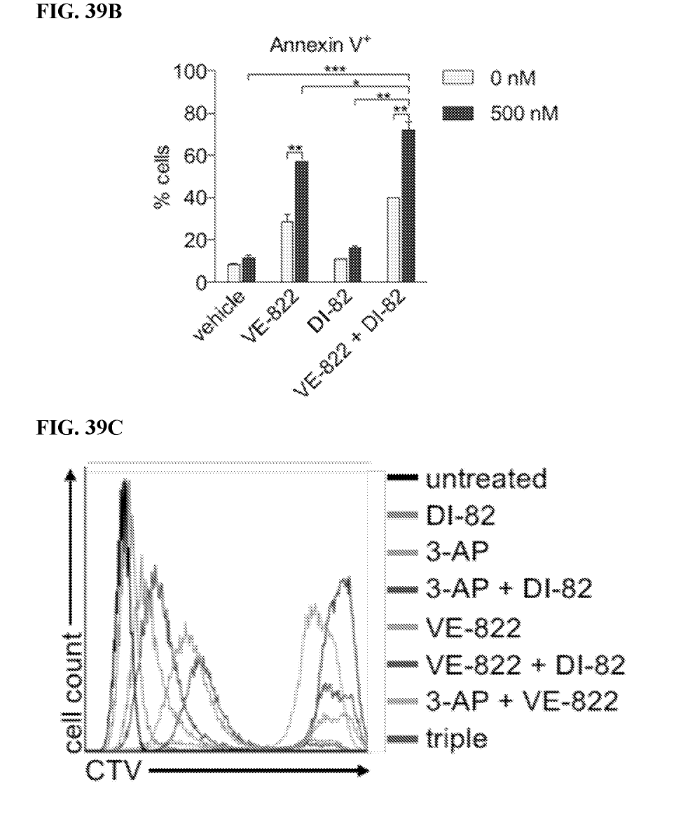

3-AP minimally induced RS and DNA damage biomarker expression alone. When combined with VE-822 and DI-82, 3-AP synergistically increased ssDNA exposure at 0.5 h, ssDNA/pH2A.X levels at 4 h and pH2A.X levels at later time point 18 h (FIG. 39A). Combining VE-822, DI-82 and 3-AP induced replication stress overload that is manifested in intolerable level of ssDNA and DSBs. This induction is consistent with increasing apoptosis as measured by Annexin V staining 72 h after treatment (FIG. 39B). Moreover, when cell proliferation is compared after 5 days of treatment, among all treatment combinations, the cells treated with all three drugs showed minimal dye dilution, indicating negligible cell proliferation with treatment (FIG. 39C). These findings indicate that low dose 3-AP (500 nM), which induces minimal replication stress alone, exhibits synthetic lethality and induces replication stress overload when combined with ATR and dCK inhibition.

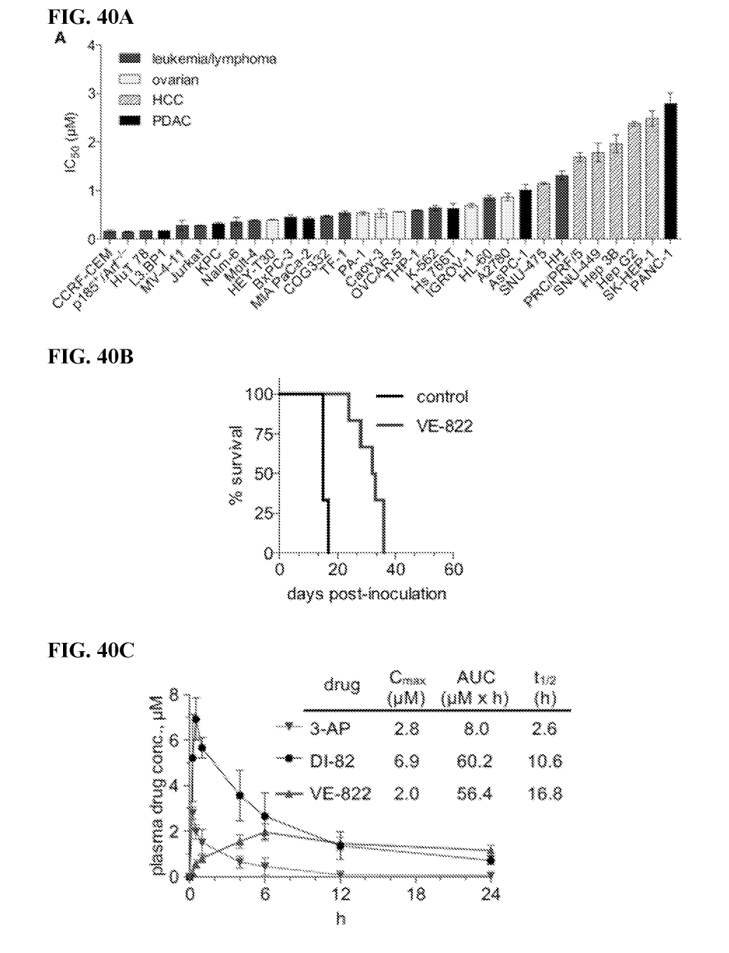

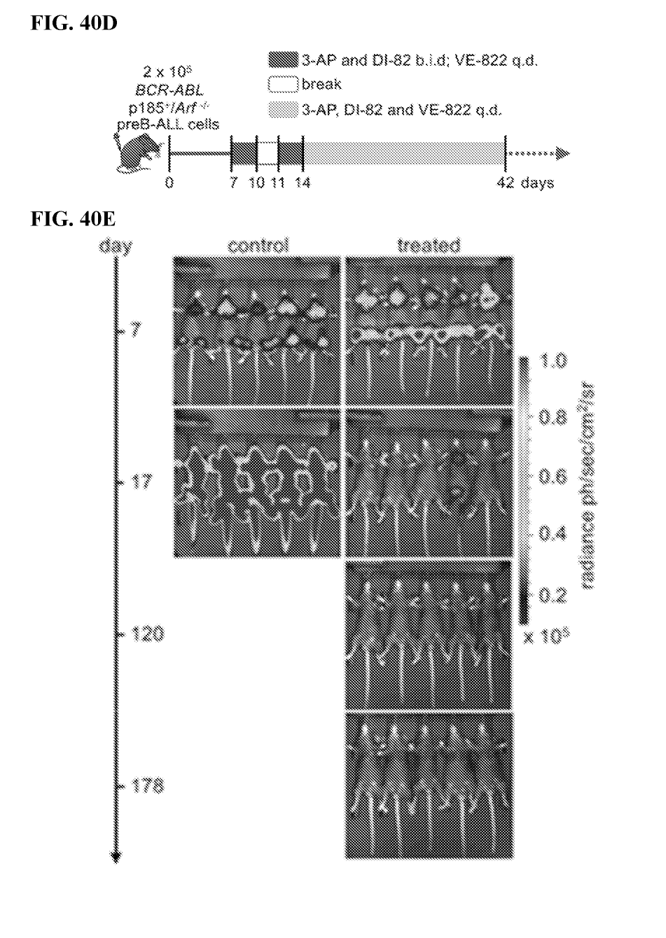

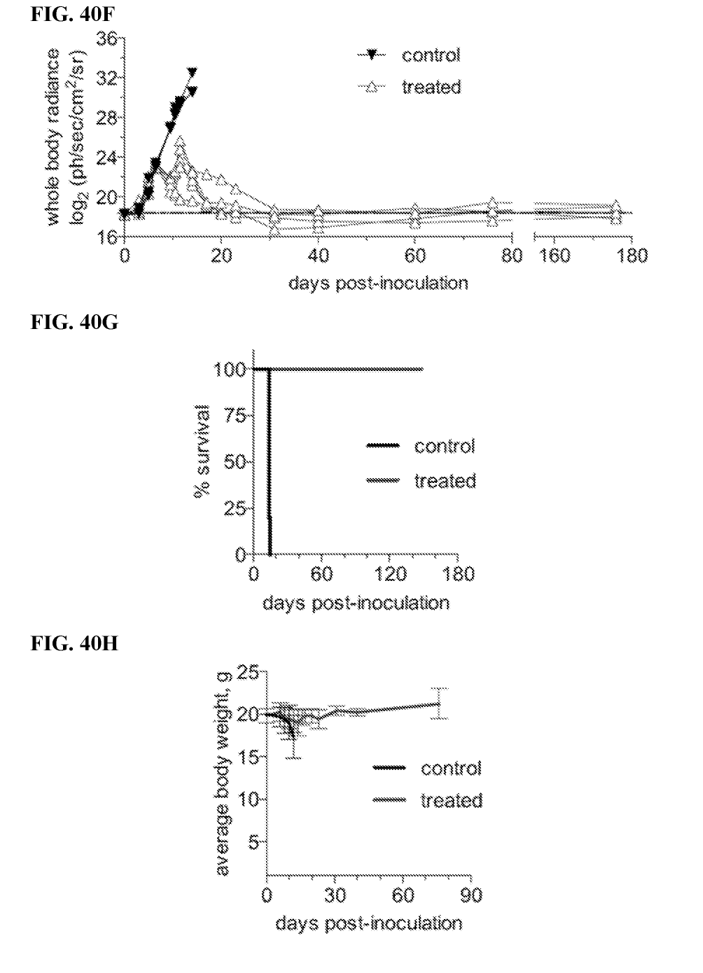

Cancer cells that exhibit high intrinsic replication stress have a decreased tolerance to replication stress overload. We profiled a panel of cancer cell lines for sensitivity to VE-822, which we used as a surrogate biomarker for intrinsic replication stress. Sensitivity to VE-822 in our cancer cell line panel showed a 10-fold range in IC₅₀ values from ˜300 nM in CEM T-ALL to 3 μM in PANC-1 PDAC cells (FIG. 40A).

We next determined if our observed in vitro sensitivity would translate in vivo. For validation studies, we selected BCR ABL p185⁺/Arf^{-/-} cell line which is VE-822 sensitive and is a clinically relevant, aggressive and difficult-to-treat model for leukemia. Luciferase expressing p185 cells were inoculated in syngeneic mice which quickly develop systemic leukemia as monitored by BLI. Despite the potency of VE-822 in p185 cell culture, single agent therapy was not capable of eradicating p185 cells in vivo (FIG. 40B). Based on our profiling of cellular responses to VE-822, the lack of efficacy in vivo may result from residual nucleotide biosynthetic activity (FIGS. 36-39). We reasoned that this innate resistance mechanism can be blocked by the addition of 3-AP and DI-82.

A drug formulation was developed to enable oral administration of 3-AP, DI-82 and VE-822 and to achieve therapeutic plasma concentrations (FIG. 40C). To maintain therapeutic plasma concentrations in treated mice group, 3-AP and DI-82 were administered twice/day, while VE-822 once/day. BCR-ABL p185⁺/Arf^{-/-} preB-ALL bearing mice were treated as shown in FIG. 40D, and the disease progression was monitored by BLI to evaluate therapeutic efficacy (FIG. 40E). The control group succumbed to disease within 20 days. In contrast, the treated group showed reduced tumor burden as evidenced by BLI quantification (FIGS. 40E and F) and continued to survive with undetectable disease for 120 days following treatment withdrawal (FIG. 40G). In addition, the combination therapy was well-tolerated as evidenced by no significant weight loss in the treated mice group (FIG. 40H).

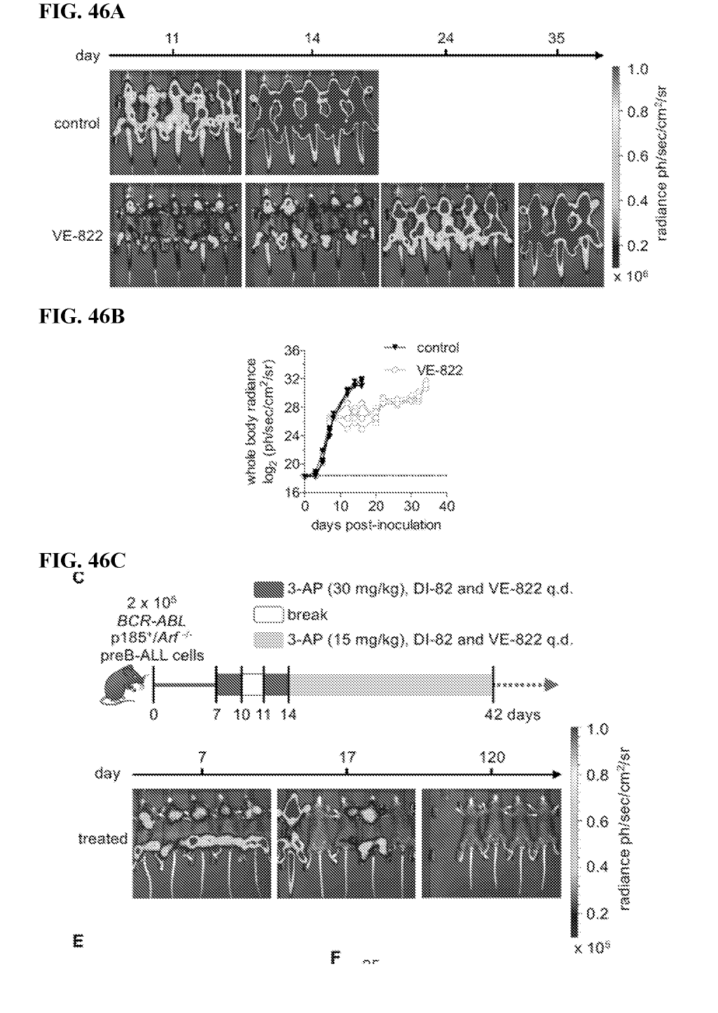

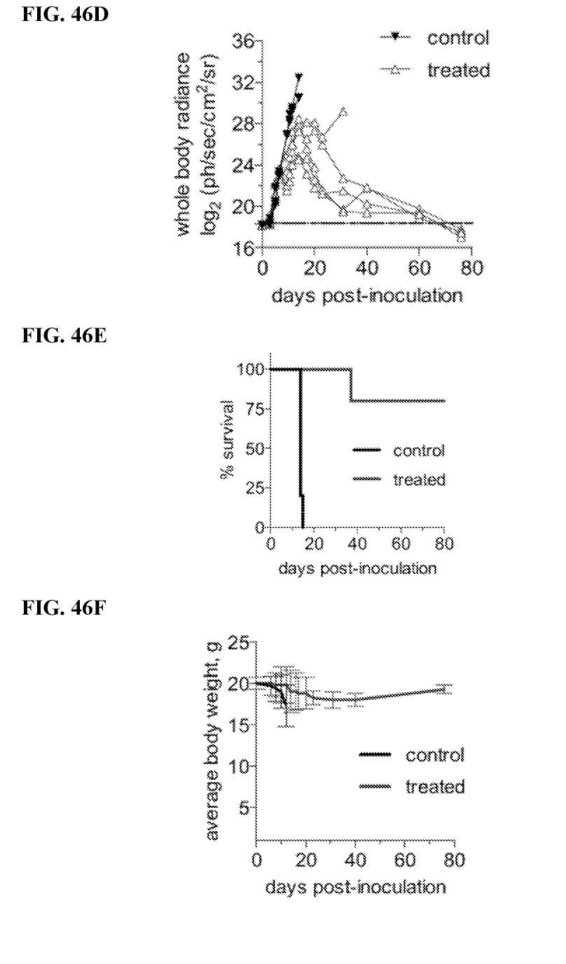

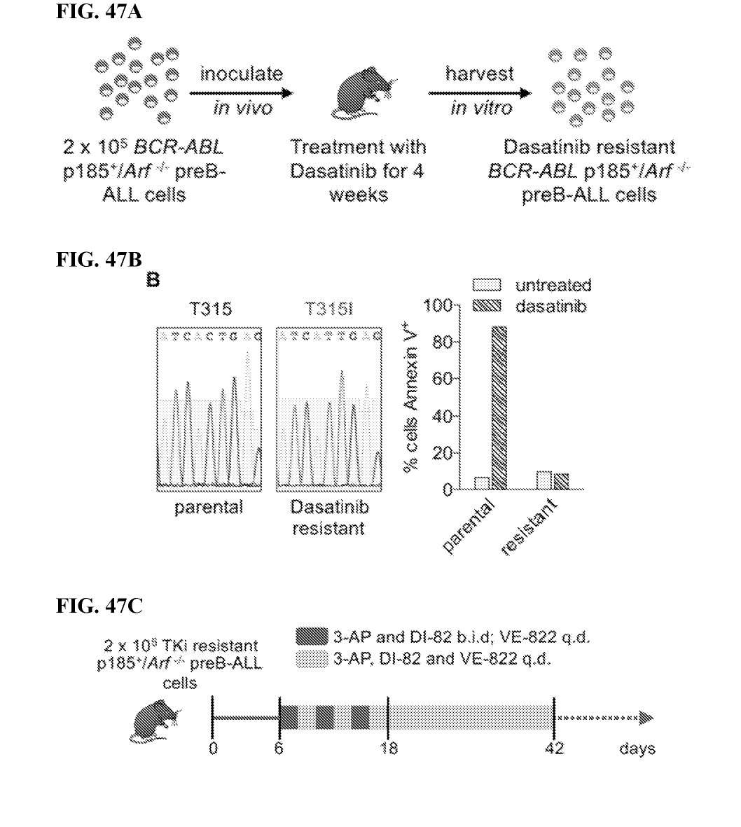

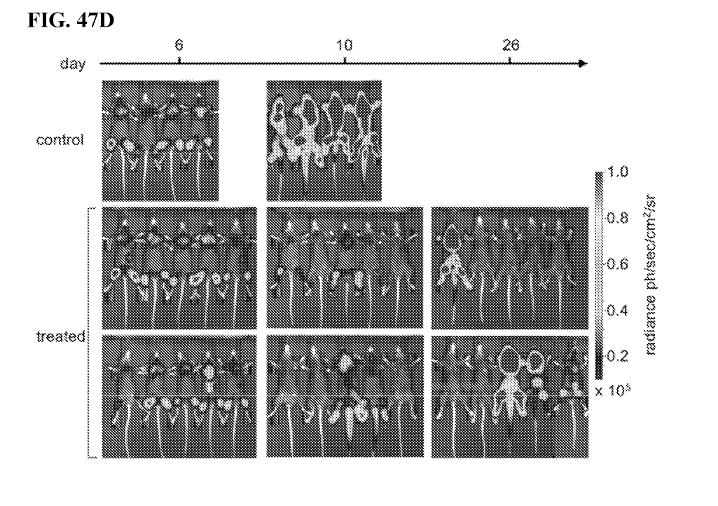

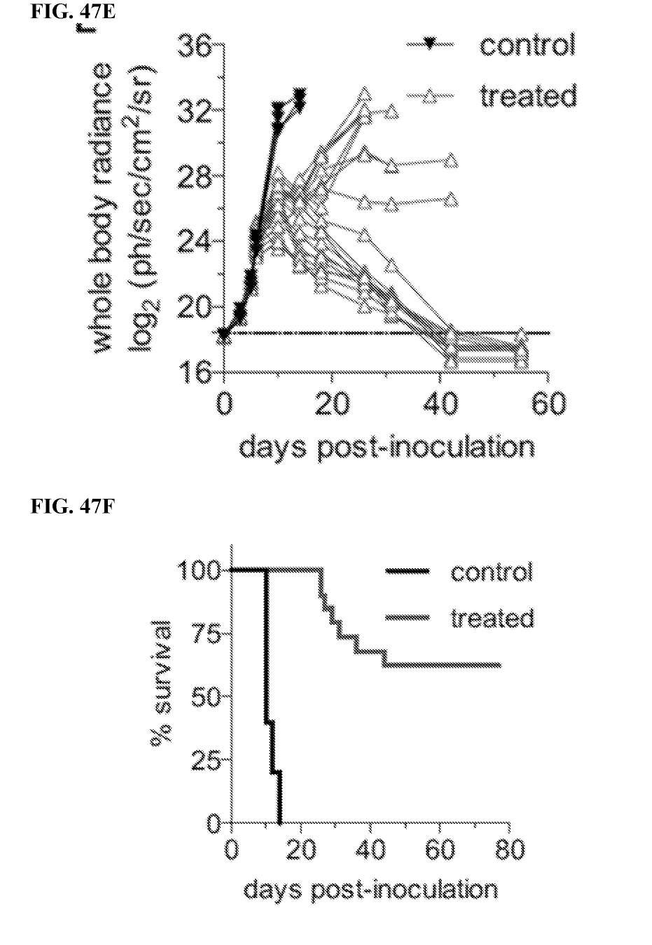

We assessed the efficacy of combination therapy administered once daily in an independent cohort, which eradicated leukemia in four out of five mice and was well-tolerated (FIG. 46C-F). To determine if our therapy can be applied to cancers without an actionable driver mutation, we derived a dasatinib-resistant model of the p185⁺/Arf^{-/-} preB-ALL cells harboring BCR-ABL gatekeeper mutation T315I (FIG. 47A-B). No detectable disease burden was found in 13 of 20 mice after 30 days of treatment (FIG. 47D-F), demonstrating that pharmacological replication stress overload can eradicate leukemia and is well-tolerated in vivo.

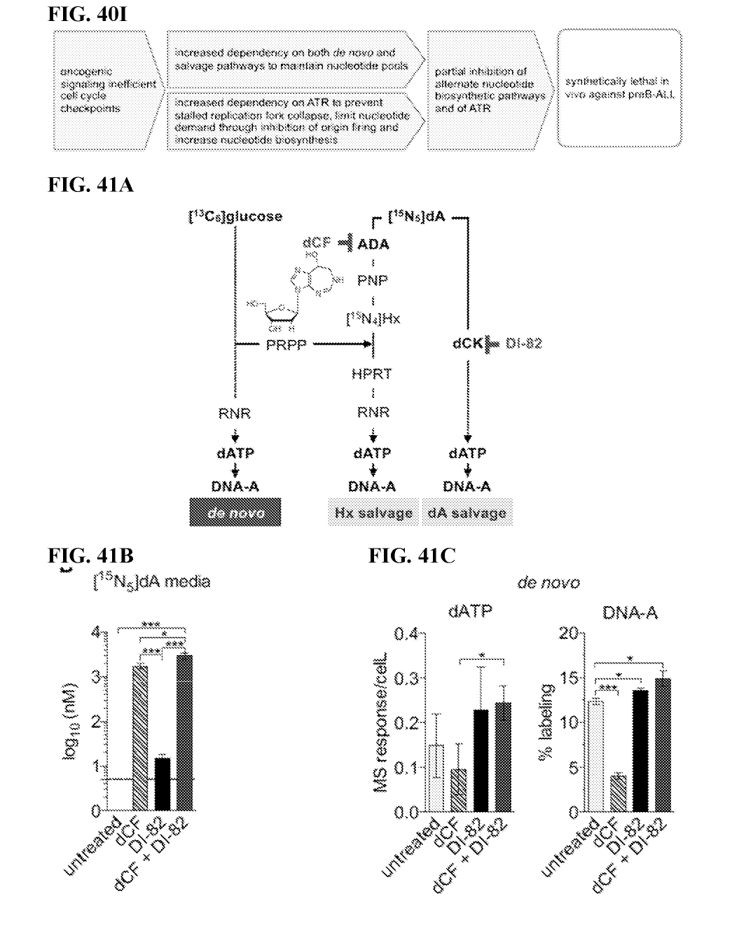

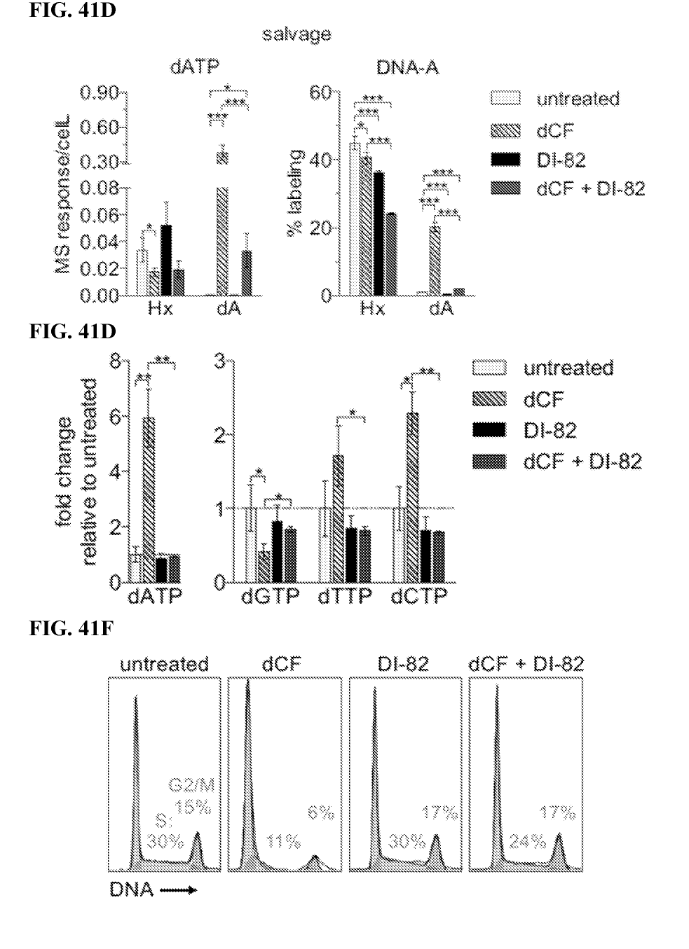

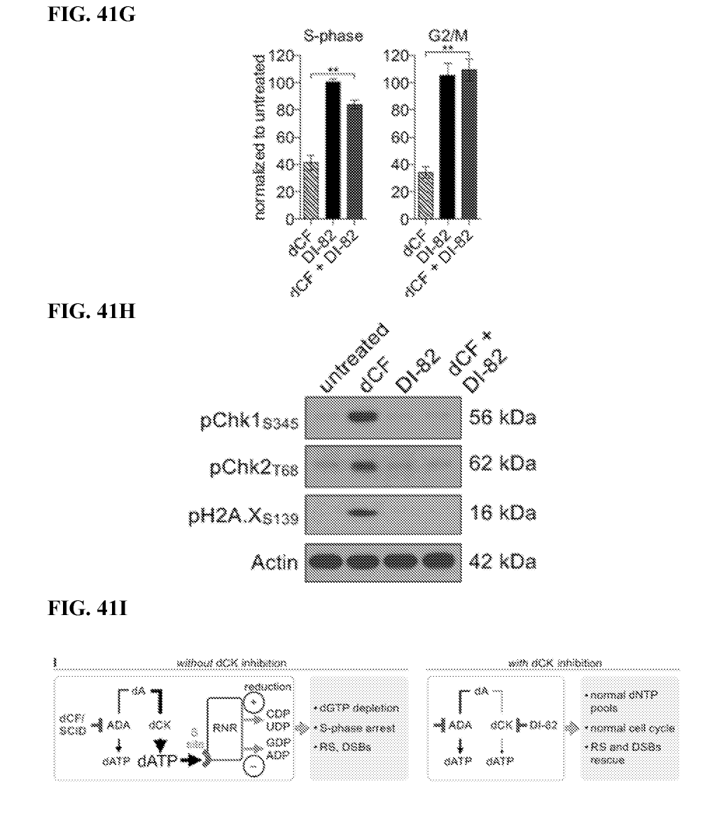

Co-targeting the replication stress response and nucleotide metabolism may be an effective therapeutic strategy for cancers with high intrinsic replication stress. Our data (FIGS. 41 and 40) suggest that dCK inhibition may be effective in the ADA and PNP SCID syndromes. Reducing the dose of 3-AP has potential therapeutic implications: 1) an effective 3-AP concentration in plasma is more readily achieved given the unfavorable pharmacokinetic (PK) properties of this drug and 2) a lower 3-AP dose is expected to reduce or even abolish off-target effects which limit clinical utility of this drug, such as methemoglobinemia.

The precursor for DNA-A, dATP, can be produced from glucose via the de novo pathway and from salvage of extracellular dA either as nucleobase and/or intact nucleoside (FIG. 41A). As shown in FIG. 35A, dA can be salvaged via three pathways: (i) an ADA dependent pathway eventually generates hypoxanthine (Hx), a substrate for HPRT; (ii) an APRT dependent pathway uses adenine generated from dA via PNP, and (iii) a dCK dependent pathway phosphorylates intact dA. Pharmacological perturbations were used to investigate the contributions of each dA salvage pathway (FIG. 41A). Jurkat cells were labeled for 18 h in the following conditions: untreated, ADA inhibitor Pentostatin (dCF) and DI-82, a specific dCK inhibitor. The labeled media contained 11 μM [U-¹³C₆]glucose to represent the de novo contribution and 5 μM [¹⁵N₅]dA to represent salvage pathways. At the end of the incubation time, [¹⁵N₅]dA was undetectable in the culture media from the untreated Jurkat cells, reflecting the high ADA activity in these leukemia cells (FIG. 41B). In contrast, the culture media from dCF treated cells contained 1.5 μM [¹⁵N₅]dA, indicative of reduced dA catabolism.

We then monitored the individual contributions to the dATP pool and newly replicated DNA-A of the de novo (FIG. 41C) and salvage pathways (FIG. 41D). In each experimental group, the APRT pathway contribution was less than 1%. In untreated Jurkat cells, the ADA pathway (or Hx salvage) accounted for over 90% of the salvaged dA. Despite the well-documented ability of dCK to phosphorylate dA in cell free systems (Sabini 2008), this kinase play a minimal role in salvaging dA unless dA catabolism was inhibited by dCF treatment. Upon ADA inhibition, the dATP pools increased 6-fold compared to untreated cells (FIG. 41E). This increased dATP level was attributed to dCK-dependent biosynthesis where there was over 190-fold increase in dA salvage into dATP (FIG. 41D). The effects of dCF and the increased dATP pool reduced the proportion of cells in S-phase by 3-fold and G2/M by more than 2-fold (FIG. 41F,G). The presence of DI-82 completely reversed the increased dATP pool level caused by dCF and restored the cell cycle profile. These findings were consistent with analyses of replication stress/DNA damage induction (FIG. 41H). A summary of these findings is shown in FIG. 41I.

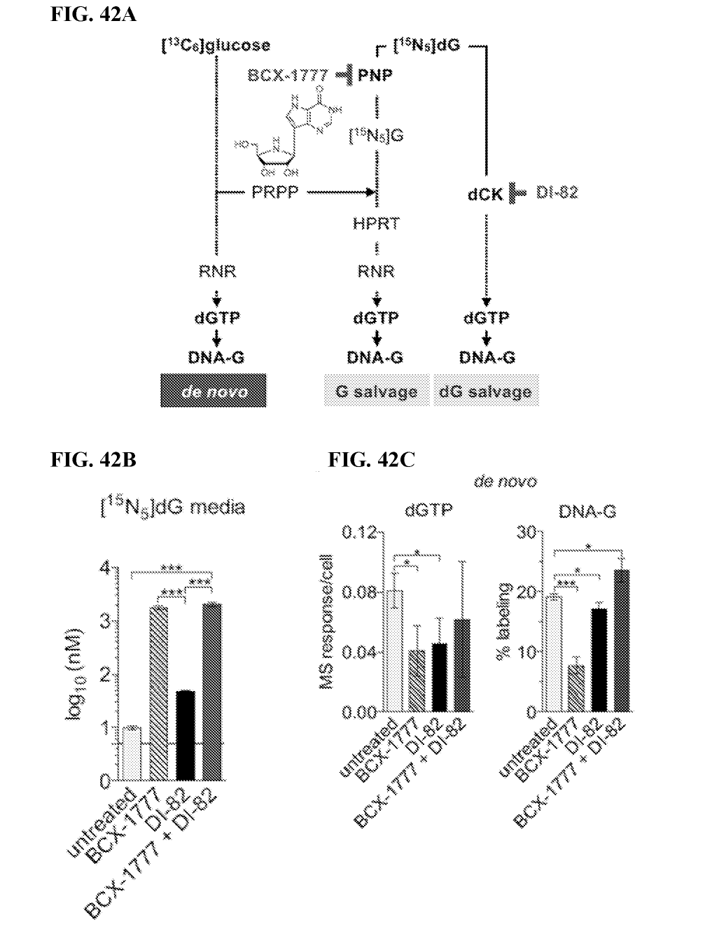

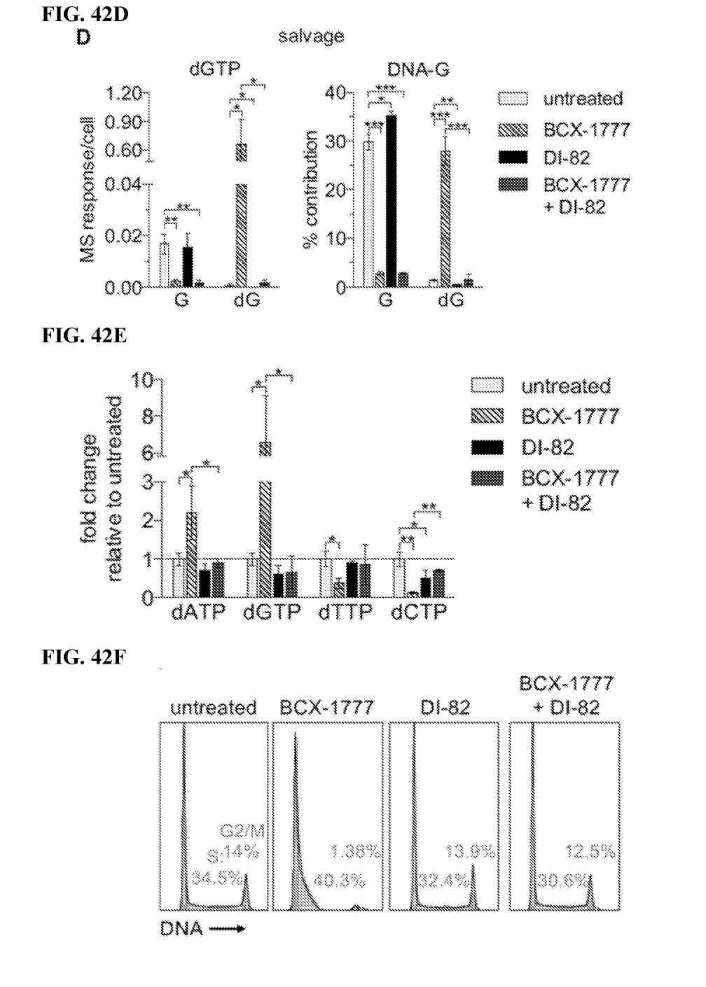

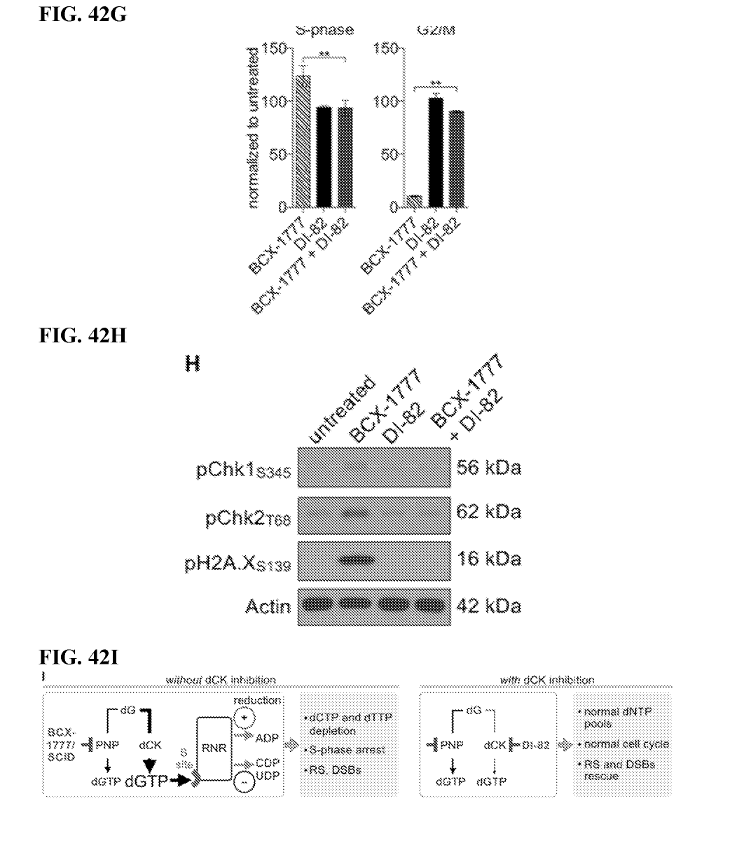

We utilized an isogenic cell line, CEM-R (Owen 1992), which lacks the enzymes dCK and HPRT that are necessary for the salvage pathways for DNA-G biosynthesis. CEM-R cells were engineered to express enhanced yellow fluorescent protein (CEM-R-EYFP, a negative control) and human HPRT (CEM-R-HPRT) and were confirmed by WB. The labeled media contained 11 μM [U-¹³C₆]glucose to represent the de novo contribution and 5 μM [¹⁵N₅]dG to represent salvage pathways, particularly HPRT-dependent nucleobase salvaging. CEM-R-EYFP relied exclusively on the de novo pathway for the DNA-G biosynthesis. In contrast, CEM-R-HPRT utilized both de novo and nucleobase salvage pathways nearly equally. Upon 6-thioguanine (6-TG) treatment, an HPRT-dependent nucleobase prodrug (FIG. 42A), CEM-R-HPRT decreased both de novo and salvage pathway labeling by nearly 10-fold while CEM-R-EYFP DNA-G biosynthesis remained unaffected.

Another approach to study dGTP/DNA-G biosynthetic pathways is through anticancer therapy Forodesine (BCX-1777). The precursor for DNA-G, dGTP, can be produced from glucose via the de novo pathway and from salvage of extracellular dG either as nucleobase and/or intact nucleoside (FIG. 42A). As shown in FIG. 35A, dG can be salvaged via two pathways: (i) a PNP dependent pathway eventually generates guanine (G), a substrate for HPRT; and (ii) a dCK dependent pathway phosphorylates intact dG. Jurkat cells were labeled for 18 h in the following conditions: untreated, PNP inhibitor (BCX-1777) and DI-82, a specific dCK inhibitor. The labeled media contained 11 μM [U-¹³C₆]glucose to represent the de novo contribution and 5 μM [¹⁵N₅]dG to represent salvage pathways. At the end of the incubation time, [¹⁵N₅]dG decreased nearly three orders of magnitude in the culture media from the untreated Jurkat cells, reflecting the high PNP activity in these leukemia cells (FIG. 42B). In contrast, the culture media from BCX-1777 treated cells contained 1.8 μM [¹⁵N₅]dG, indicative of reduced dG catabolism.

We then monitored the individual contributions to the dGTP pool and newly replicated DNA-G of the de novo (FIG. 42C) and salvage pathways (FIG. 42D). In untreated Jurkat cells, the PNP pathway (or G salvage) accounted for about 95% of the salvaged dG. Despite the documented ability of dCK to phosphorylate dG in cell free systems, this kinase play a minimal role in salvaging dG unless dG catabolism was inhibited by BCX-1777 treatment. Upon PNP inhibition, the dGTP pools increased 6-fold compared to untreated cells (FIG. 42E). This increased dGTP level was attributed to dCK-dependent biosynthesis where there was over 300-fold increase in dG salvage into dGTP (FIG. 42D). The effects of BCX-1777 and the increased dGTP pool altered the cell cycle profile in which cells are piled up in G1/S transition and have reduced G2/M population (FIG. 42F,G). The presence of DI-82 completely reversed the increased dGTP pool level caused by BCX-1777 and restored the cell cycle profile. These findings were consistent with analyses of replication stress/DNA damage induction (FIG. 42H). A summary of these findings is shown in FIG. 42I.

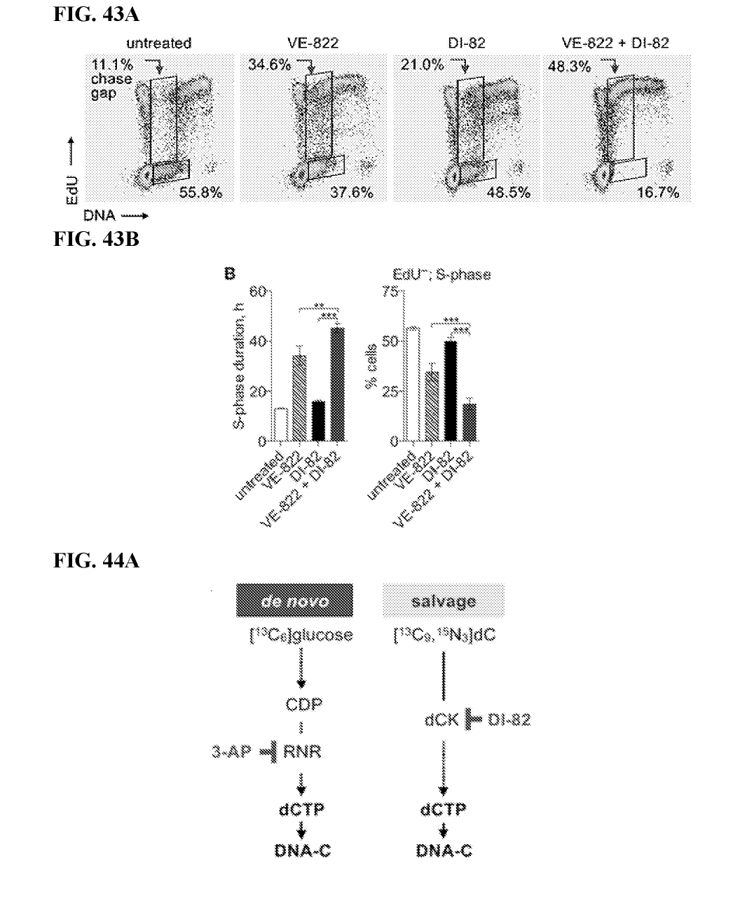

Asynchronous CEM cells undergoing DNA replication were pulse labeled with EdU, and the progression of EdU positive cells in different treatments was monitored at 4, 8 and 18 h by FACS. Similar slowdown in cell cycle progression in VE-822+DI-82 was noticed (1.25 and 2.25 times VE-822 and DI-82 treatments respectively), as shown in the bivariate EdU/DNA plots, calculated S-phase duration, and progression of EdU negative (EdU) cells under treatments (FIG. 43A).

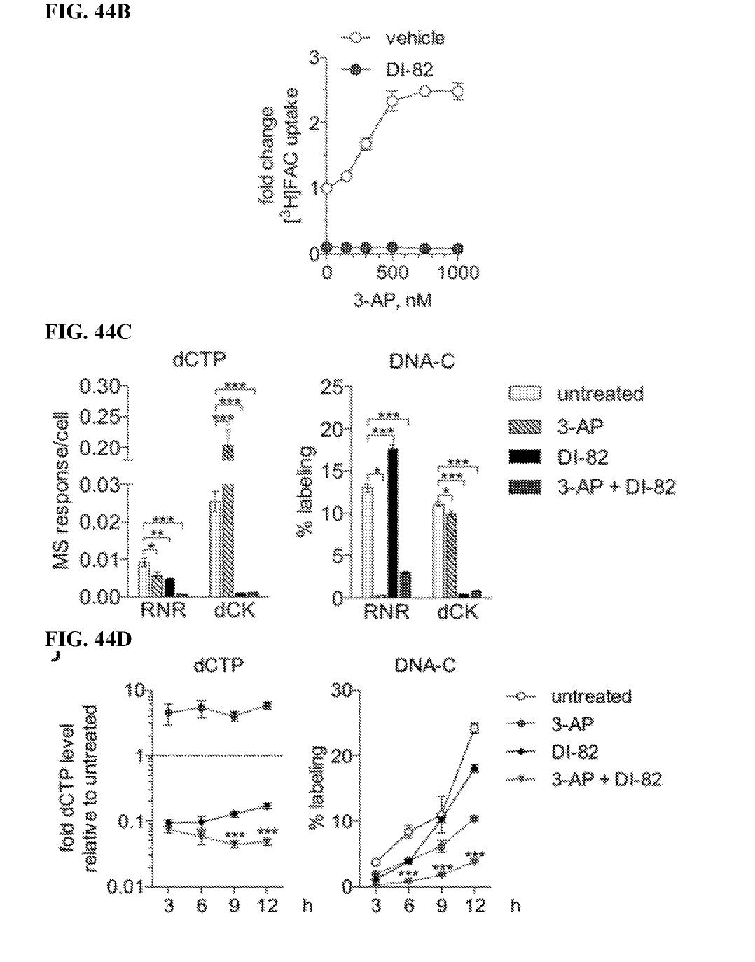

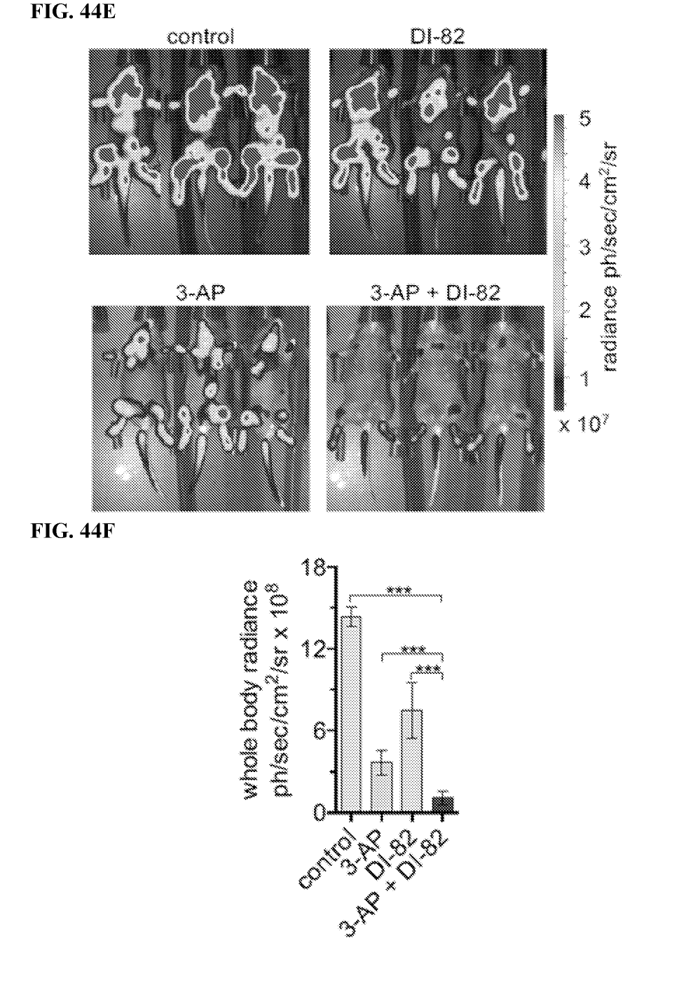

To evaluate changes in dCK activity with 3-AP treatment, ³H-FAC (labeled dC analog) uptake was performed, and a significant increase (˜2.5 fold) in uptake after 1 h with 3-AP treatment was observed in p185^{BCR-ABL}Arf^{-/-} pre-B cells (FIG. 44A). We further investigated if dCK is a resistance mechanism of 3-AP treatment (FIG. 44B). Therefore, p185^{BCR-ABL}Arf^{-/-} pre-B cells were labeled with [U-¹³C₉,¹⁵N₃]dC and [U-¹³C₆]glucose to measure the contribution of salvage pathway via dCK and de novo pathway via RNR, respectively. Labeled cells were treated with ±3-AP±DI-82, for 3, 6, 9 and 12 h. dCTP pool and DNA-C measurements are summarized in FIG. 44C,D. Cells treated with DI-82 maintained DNA replication primarily through the de novo pathway. However, the dCTP pool size was nearly depleted, indicating that de novo-derived dCTP was quickly utilized after synthesis. Cells treated with 3-AP increased their dCTP pool from higher dCK activity and the dCTP produced by dCK was predominantly used for DNA-C. These findings revealed the plasticity of nucleotide metabolism pathways for DNA-C, where upregulation of either pathway compensates for the other. The combined targeting of the two dCTP biosynthetic pathways, de novo (RNR) and salvage (dCK) with 3-AP and DI-82, respectively, nearly abolished the dCTP pool severely limiting DNA replication as indicated by low % enrichment from either dCK and RNR.

To determine whether co-targeting with 3-AP and DI-82 has a therapeutic effect, we performed an in vivo treatment study where C57BL/6 mice were inoculated i.v. with p185^{BCR-ABL}Arf^{-/-} pre-B cells (leukemia initiating cells or LICs) to induce systemic leukemia. Cohorts of mice (N=5/group) were administered with 3-AP, DI-82 alone or in combination by intraperitoneal injection, starting seven days after inoculation. The tumor burden was monitored by bioluminescence imaging (BLI) of firefly luciferase-expressing p185^{BCR-ABL}Arf^{-/-} pre-B cells in mice throughout the 20-day treatment. The double combination group showed significantly lower leukemic tumor burden at the end of the treatment compared to any of the single treatment groups (FIG. 44E). FIG. 44F shows the quantification of bioluminescence for all treatment groups.

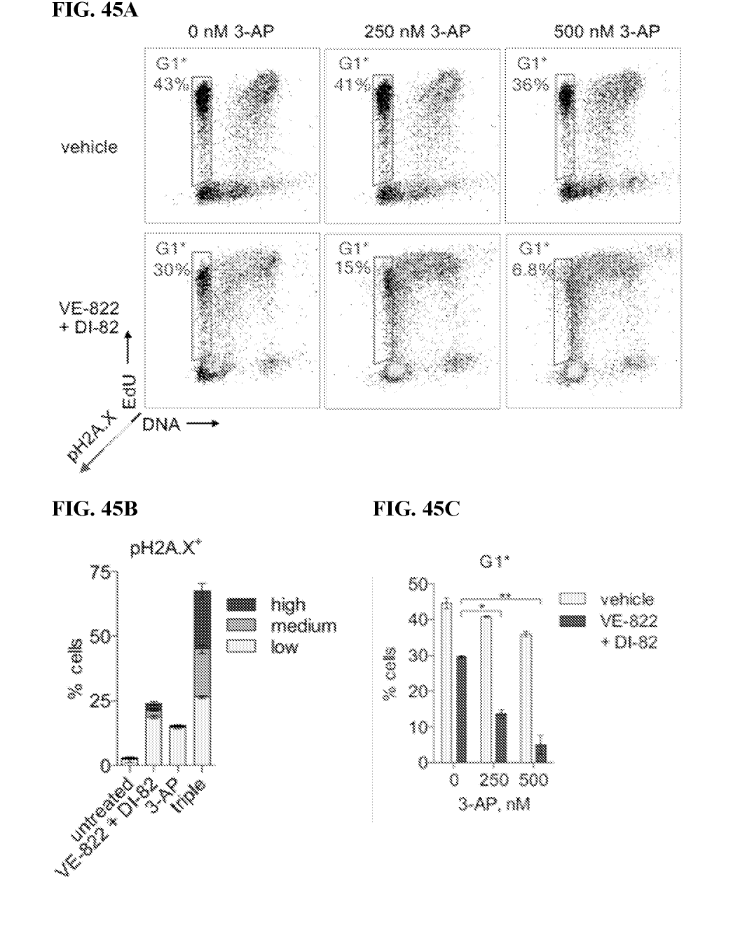

The need for an RNR inhibitor in addition to ATR and dCK inhibition is rationalized based on residual RNR upon VE-822 and DI-82 treatment. Therefore, we investigated the biosynthesis and utilization of dCTP pool with various treatment groups of the triple combination. The triple combination of RNR, dCK and ATR inhibition abolished the dCTP pools both at 250 nM 3-AP and 500 nM 3-AP, and restricted DNA synthesis, in contrast to single agents and double combinations (FIG. 45A, B). In order to follow a defined population throughout the cell cycle, CEM cells were pulse labeled with EdU and the progression of EdU positive cells together with double stranded biomarker, pH2A.X was monitored at various time points (5, 10 h chase) by FACS under different treatments. FIG. 45C shows the cell cycle progression of EdU labeled cells at 10 h following indicated treatments. G1* population indicates the percentage of cells that have completed one cell cycle and entered a new one. When the treatment groups are compared, a significant percentage of cells are arrested in the triple combination treatment group, shown by 50% and 80% decrease in G1* population with 250 and 500 nM respectively, compared to VE-822+DI-82 double combination treatment (FIG. 45E). In addition, triple combination treatment has high levels of pH2A.X induction (three times compared to VE-822+DI-82 combination) following 10 hours of treatment (FIG. 45D). Measurement of cell proliferation by dye dilution assay also confirmed that triple combination treatment is necessary to halt cell proliferation, in contrast to single and double combinations (FIG. 45F).

Single agent VE-822 treatment is marginally efficacious in vivo. The efficacy of triple combination therapy against p185 preB-ALL model cohort #2 is demonstrated in FIG. 46. ATR inhibition was shown to be effective in cell culture against p185 cells, so was assessed in vivo. C57BL/6 mice were injected i.v. with luciferase expressing p185 preB-ALL cells for leukemia induction. Treatment was started seven days post-inoculation and treated with vehicle or VE-822 (40 mg/kg) orally once daily for two weeks. Disease burden was subsequently compared between vehicle and treated groups (FIG. 46A) and whole body bioluminescence was quantified (FIG. 46B). Additionally, the therapeutic efficacy of triple combination therapy was evaluated in a second cohort of mice bearing preB-ALL. FIG. 46C shows the treatment regimen, which comprises of all three drugs—3-AP, DI-82 and VE-822 administered q.d. for a duration of 35 days, with a break of 3 days in between. The efficacy was shown to be reproducible in a second cohort of mice, summarized in FIG. 46D-F.

The therapeutic efficacy of triple combination therapy against an aggressive Dasatinib resistant preB-ALL model is demonstrated in FIG. 47. A more stringent experiment of the efficacy of triple combination therapy was performed by generating tyrosine kinase inhibitor resistant leukemia model. p185 pre-B ALL recipient mice were treated with Dasatinib (standard of care for ALL) for 4 weeks. Cells were harvested from the mice when the disease relapsed, and cultured to generate the Dasatinib resistant p185 cells (FIG. 47A). These resistant cells were treated with Dasatinib to confirm resistance, and sequenced to validate T315I gatekeeper mutation (FIG. 47B). The resistant cells harboring T315I gatekeeper mutation were inoculated in C57BL/6 mice for more aggressive leukemia induction, and treated following the regimen shown in FIG. 47C. Vehicle treated mice were moribund in 10 days, in contrast to 15-17 days in case of non-resistant model. Treatment was carried out for 42 days, and a significant therapeutic efficacy was observed as shown by the bioluminescence images of vehicle and triple combination treated mice (FIG. 47D). Of 20 treated mice, 13 were disease-free after 42 days of treatment, with no relapse until 120 days (FIGS. 47E and F).

### General Methods

### Cell Culture and Culture Conditions

Cell lines CCRF-CEM, Nalm-6, EL4, Jurkat, Molt-4, CEM-R, THP-1, HL-60, TF-1, MV-4-11, HH, HuT 78, HCT 116, MIA PaCa-2 were obtained from American Type Culture Collection (ATCC). All leukemic cell lines were cultured in RPMI-1640 (Corning) containing 10% fetal bovine serum (FBS, Omega Scientific) and were grown at 37° C., 20% O₂ and 5% CO₂; with the exception of a few cell lines, HK374 was cultured in DMEM-F12 (Invitrogen) containing B27 supplement (Life Technologies), 20 ng/mL basic fibroblast growth factor (bFGF; Peprotech), 50 ng/mL epidermal growth factor (EGF; Life Technologies), penicillin/streptomycin (Invitrogen), Glutamax (Invitrogen), and 5 μg/mL heparin (Sigma-Aldrich); and p185^{BCR-ABL}Arf^{-/-} pre-B cells was maintained in RPMI-1640 containing 10% FBS and 0.1% ß-mercaptoethanol.

### Plasmids and EL-4 and CEM-R Isogenic Cell Lines.

The pMSCV-HPRT-IRES-EYFP and pMSCV-TK1-IRES-EYFP plasmids were generated by inserting the human HPRT and TK1 coding sequence into the multiple cloning sites of the MSCV-IRES-EYFP plasmid, respectively. The pMSCV-hdCK-IRES-EYFP plasmid was generated as previously described (Laing RE 2010). Amphotropic retroviruses were generated by transient co-transfection of the MSCV retroviral plasmid and pCL-10A1 packaging plasmid into Phoenix-Ampho packaging cells. To generate EL4-EYFP, EL4-TK1, CEM-R-EYFP, CEM-R-dCK and CEM-R-HPRT lines, parental cells underwent spin-fection with the respective amphotropic retrovirus and were then sorted by flow cytometry to isolate pure populations of transduced cells.

### Isotopic Labeling

Cells were transferred into RPMI-1640 without glucose and supplemented with 10% dialyzed FBS (Gibco) containing any of the following labeled substrates (Cambridge Isotopes): precursors for de novo [U-¹³C₆]glucose (Sigma-Aldrich) at 11 mM; precursors for purine salvage [U-¹³C₁₀,¹⁵N₅]dA (Cambridge Isotopes), [¹⁵N₅]dA (Cambridge Isotopes), [¹⁵N₅]dG (Cambridge Isotopes), [U-¹³C₅,¹⁵N₄]Hx (Cambridge Isotopes) at 5 μM or as indicated in the text; and precursors for pyrimidine salvage: [U-¹³C₉,¹⁵N₃]dC (Silantes) and [U-¹³C₁₀,¹⁵N₂]dT (Cambridge Isotopes) at 5 μM. The cells were incubated for 18 h or as indicated in the text before samples collection and processing.

### DNA Sample Processing

Genomic DNA was extracted using the Quick-gDNA MiniPrep kit (Zymo Research, D3021) and hydrolyzed to nucleosides using the DNA Degradase Plus kit (Zymo Research, E2021) following manufacturer-supplied instructions. In the final step of DNA extraction, 50 μL of water was used to elute the DNA. A nuclease premix solution was prepared by mixing 10× buffer, DNA degradase Plus™ and water in the ratio of 2.5/1/1.5 (v/v/v). DNA hydrolysis reaction was prepared in mass spec vials by mixing 5 μL of the nuclease premix solution (5 U of enzyme) and 20 μL of eluted genomic DNA, the total volume becomes 25 μL. The samples were tapped and flicked downward prior to overnight before incubation at 37° C.

### Media Sample Processing

Culture medium was collected 20 μL at the indicated time points. Stock solutions (10 mM) of [U-¹³C₁₀,¹⁵N₅]dA, and [¹⁵N₃]dC (Cambridge Isotope Laboratories) were prepared individually in dimethyl sulfoxide (DMSO), and stored at −20° C. before use as internal standards. The internal standards were diluted to 20 nM in methanol to generate internal standard solution. Calibration standards were prepared by spiking working stock solutions of [U-¹³C₁₀,¹⁵N₅]dA and [U-¹³C₉,¹⁵N₃]dC blank media to give concentrations in the 10 nM-10 μM range. Each 20 μL calibration standard sample was mixed with 60 μL of internal standard solution, mixed (30 s) and centrifuged (15,000 g, 10 min, 4° C.) and 60 μL of supernatant were transferred into a clean mass spec vial for LC-MS/MS-MRM analysis. Media samples were processed similarly and in parallel to the calibration standard samples.

dNTP Sample Processing

Cells were collected into microcentrifuge tube and centrifuged (450×g, 4 min, 4° C.). The supernatant was carefully aspirated. The cells were washed twice by the addition of 1 mL of cold PBS and centrifugation (450×g, 4 min 4° C.). PBS washes were aspirated. Thereafter, cells were lysed in 40 μL of 70% methanol/water (v/v), vortexed (30 s) and incubated on ice for 10 min. Then 40 μL of chloroform (Fisher) was added, vortexed (30 s) and incubated on ice for another 10 min. The samples were then centrifuged at max speed for 10 min at 4° C. Finally, the supernatant was transferred (≈25 μL) into a mass spec vial.

### LC-MS/MS-MRM Analysis

For genomic DNA and media analysis, an aliquot of DNA hydrolysis or media samples (20 μL) was injected directly onto a porous graphitic carbon column (Thermo Fisher Scientific Hypercarb, 100×2.1 mm, 5 μm particle size) equilibrated in solvent A (water/acetonitrile/formic acid, 95/5/0.2, v/v/v) and eluted (200 μL/min) with an increasing concentration of solvent B (acetonitrile/water/formic acid, 90/10/0.2, v/v/v): min/% B/flow rate (μL/min); 0/0/200, 5/0/200, 10/15/200, 20/15/200, 21/40/200, 25/50/200, 26/100/700, 30/100/700, 3I/O/700, 34/0/700, 35/0/200).

For free dNTP analysis, a modified version of the same previously reported method (Cohen et al., 2009) was used in which dNTP lysate sample (20 μL) was injected directly onto Hypercarb column equilibrated in solvent C (5 mM hexylamine and 0.5% diethylamine v/v, pH 10.0 with about 2.35 mL of glacial acetic acid for every 1 L solvent made). The dNTPs were eluted (250 μL/min) with an increasing concentration of solvent D (acetonitrile/water, 50/50): min/% D/flow rate (μL/min); 0/0/200, 5/0/200, 10/15/200, 20/15/200, 21/40/200, 25/50/200, 26/100/700, 30/100/700, 31/0/700, 34/0/700, 35/0/200).

The effluent from the Hypercarb column was directed to Agilent Jet Stream ion source connected to the triple quadrupole mass spectrometer (Agilent 6460) operating in the multiple reaction monitoring mode. The peak areas for each of the nucleosides and nucleotides (precursor→fragment ion transitions) were recorded with instrument manufacturer-supplied software (Agilent MassHunter).

### % Labeling and % Contribution Calculation

Percent labeling was determined by dividing the MS response of labeled nucleosides enriched from each biosynthetic pathway to the sum of the MS response from all labeled and unlabeled ion transitions. Enrichment (% contribution) was determined by dividing the MS response of labeled nucleosides enriched from each biosynthetic pathway to the sum of the MS response from only labeled ion transitions.

### FACS and Flow Cytometry Analysis

### Cell Synchronization

Cells were treated with a CDK4/6 inhibitor, PD-0332991 (Selleckchem) for 18 h to arrest in G1 phase. Subsequently, cells were washed and released into fresh media containing 10% FBS to monitor the progression of synchronized cells throughout the cell cycle by flow cytometry analysis.

### Cell Cycle Histograms (DNA Content)

Cells were plated at the density of 0.5 million cells per ml per well in respective media/treatment. After 24 h incubation, cells were harvested and washed with PBS twice before staining with 0.5 ml of propidium iodide (Calbiochem, 1 μg/ml) solution containing Ribonuclease A and 0.3% Triton-X 100. The samples were protected from light before acquisition by flow cytometry.

Intracellular detection of DNA-incorporated 5-ethynyl-2-deoxyuridine (EdU) with DNA content to measure cell cycle kinetics (pulse chase analysis) and cell cycle progression (synchronous chase pulse analysis)

CEM T-ALL cells plated at a density of 0.5×10⁶ cells/mL. The cells were pulsed with 10 μM EdU (Invitrogen) for 1 h, washed in PBS twice, and released in fresh warm media containing 5 μM deoxyribonucleosides, with and without drugs. Cells were harvested at various time points after release in fresh media, and then fixed with 4% Paraformaldehyde and permeabilized using saponin perm/wash reagent (Invitrogen). Cells were then stained with azide-Alexa Fluor 647 (Invitrogen; Click-iT EdU Flow cytometry kit) by Click reaction according to manufacturer's protocol. Total DNA content was assessed by staining samples with FxCycle-Violet (Invitrogen) at 1 μg/mL final concentration in PBS containing 2% FBS.

For measuring cell cycle progression of synchronous population of cells, cells were first arrested in G1 phase, then washed and released in treated media. Before collecting and fixing cells at various time points, cells were pulse-labeled with 10 μM EdU for 1 h, and then click-labeled with azide-Alexa Fluor 647 (Invitrogen; Click-iT EdU Flow cytometry kit) according to manufacturer's protocol. Total DNA content was assessed by staining with FxCycle-Violet (Invitrogen) at 1 μg/mL final concentration in PBS containing 2% FBS.

pH2A.X/DNA Staining

Cells were harvested and fixed and permeabilized with cytofix/cytoperm (BD biosciences) for 15 min in dark on ice. Cells were washed and subsequently resuspended in 100 μL 1× Perm/wash buffer (BD Sciences) for 15 min on ice. Cells were washed and subsequently resuspended in 50 μL with phospho-Histone H2A.X (Ser139) antibody conjugated to fluorochrome FITC (EMD Milipore, 1:800 dilutions in perm/wash) for 20 min in dark at ambient temperature. Subsequently, cells were washed and stained with 0.5 ml of DAPI for DNA content (1 μg/ml in 2% FBS in PBS) before data acquisition.

ssDNA Measured with F7-26 Antibody

Cells were harvested and fixed with ice-cold methanol:PBS (6:1) for 24 h. Staining with F7-26 monoclonal antibody (Mab) was performed according to manufacturer's instructions (EMD Milipore).

Fixed cells were resuspended in 250 μL of formamide and heated in a water bath at 75° C. for 10 min. Cells were then allowed to return to room temperature and then washed with 2 ml of 1% non-fat dry milk in PBS for 15 min. Subsequently, cells were resuspended in 100 μL of anti-ssDNA Mab F7-26 (EMD Milipore, 1:10 in 5% FBS in PBS) and incubated for 45 min at room temperature. Cells were washed with PBS and stained with 100 μL of fluorescence-conjugated goat anti-mouse IgM antibody (Santa Cruz Biotechnology, 1:50) for 45 min at room temperature. Cells were then washed with PBS stained with 0.5 ml of DAPI for DNA content (1 μg/ml in 2% FBS in PBS) before data acquisition.

### RRM2/DNA Staining

Cells were harvested and fixed with 100 μL Cytofix/Cytoperm solution (BD Sciences) for 15 min. Cells were then permeabilized with 100 μL of Perm/Wash (BD Sciences, 1:10) for 15 min. Subsequently, cells were resuspended in 50 μL with RRM2 antibody (Santa Cruz, 1:200) for 30 min, washed and then incubated with 100 μLof anti-goat secondary antibody conjugated to fluorochrome. Subsequently, cells were washed with perm wash and stained for DNA content with DAPI (1 μg/ml in 2% FBS in PBS).

### Apoptosis by Annexin V Staining

Cells were treated with respective treatments for 24 or, 72 h, then collected and washed twice with 1 ml of FACS buffer (2% FBS in PBS). Induction of apoptosis and cell death were assayed by staining cells with Annexin V-FITC and PI according to manufacturer's instructions (FITC Annexin V Apoptosis Detection Kit, BD Sciences).

Dye Dilution Assay Using cellTrace Violet to Measure Cell Cycle Divisions

CEM T-ALL cells were loaded with 5 μM cellTrace violet (Molecular probes, Invitrogen) dye by incubating the cells with dye for 20 min. The cells were then washed and resuspended in fresh media with and without treatments. An aliquot of cells of all treatment groups were analyzed by flow cytometry everyday till 5 days for dye dilution. The decrease in dye intensity of the loaded dye was interpreted to be proportional to cell proliferation.

### FACS Analysis:

All flow cytometry data were acquired on five-laser LSRII cytometers (BD) for analysis, and analyzed using FlowJo software (Tree Star). The cell cycle durations were calculated using equations for multiple time-point measurements according to Terry et al., Nat. Prot. 2006.

### Cell Viability Assay

Cells were plated in a 384-well plate (1,000 cells/well for suspension cell lines and 500 cells/well for adherent cell lines in 30 μL). For suspension cell lines, the plate was incubated in 37° C. for 1 h to let the cells settle. For adherent cell lines, the plate was incubated overnight to allow the cells to seed. During incubation, drugs were diluted to desired concentration by serial dilution, vehicle condition was created by adding equivalent amount of DMSO. After incubation, 10 μL of diluted drugs were added to each well, and incubated for 72 h. After incubation, Cell TiterGlo reagent was added to each well according to manufacturer's instructions (Promega, CellTiter-Glo® Luminescent Cell Viability Assay). The plates were shaken for 2 min and incubated in dark for 8 min. Luminescence readings were obtained with luminometer (Molecular devices, SpectraMax).

### Western Blot

To prepare cell lysates for western blotting, the cells were lysed on the dish using RIPA buffer supplemented with protease and phosphatase inhibitors, scraped and placed into microcentrifuge tubes, sonicated, and centrifuged at 20,000 g at 4° C. to remove insoluble material. Protein concentration was determined using the Micro BCA Protein Assay kit (Thermo) and equal amounts of protein were resolved on Bis-Tris polyacrylamide gels. The primary and secondary antibodies that were used in this study are as follows: anti-dCK (described in reference (Bunimovich et al., 2014, 1:2000), HPRT (Santa Cruz Biotechnology, SC200A5, 1:1000), TK1 (1:1000), anti-Actin (Cell Signaling Technology, 9470, 1:10,000) and anti-CDA (Sigma-Aldrich, SAB1300716, 1:1000), anti-rabbit IgG HRP-linked (Cell Signaling Technology, 7074) and anti-mouse IgG HRP-linked (Cell Signaling Technology, 7076). Primary antibodies were stored in 5% BSA (Sigma-Aldrich) and 0.1% NaN3 in 1×TBST. Chemiluminescent substrates (ThermoFisher Scientific, 34077 and 34095) and autoradiography film (Denville) were used for detection.

### Proteomics

Cells were treated with a CDK4/6 inhibitor, PD-0332991 (Selleckchem) for 18 h to arrest in G1 phase. Cells were then washed twice with PBS and released in fresh media with treatment at a density of 0.5×10⁶ cells/ml. Cells were collected at time point 6 and 12 h and solubilized using 0.5% sodium deoxycholate, 12 mM sodium lauryl sarcosine, and 50 mM triethylammonium bicarbonate pH 8.0 with 1 mL per 5×10⁷ cells with trituration and vortexing. Cell lysates were heated at 95° C. for 5 min and water bath sonicated at RT for 5 min. Bicinchoninic acid protein assay (Pierce) was performed to determine protein concentration. Disulfide bridges were reduced with 5 mM tris(2-carboxyethyl)phosphine (final concentration) at RT for 30 min and subsequently alkylated with 10 mM iodoacetamide (final concentration) at RT in the dark for 30 min. Cell lysates were diluted 1:5 (v:v) with 50 mM triethylammonium bicarbonate. Proteins were cleaved with sequencing grade trypsin (Promega) 1:100 (enzyme:protein) for 4 hr at 37° C. followed by a second aliquot of trypsin 1:100 (enzyme:protein) overnight at 37° C. Samples were acidified with 0.5% trifluoroacetic acid (final concentration), vortexed rapidly for 5 min, and centrifuged at 16,000×g for 5 min at RT to pellet sodium deoxycholate. Supernatants were desalted using 200 mg tC18 Sep-Pak cartridges (Waters) and dimethyl labeled following previously described methods (1). Briefly, Sep-Paks were equilibrated with 1 mL of 250 mM 2-(N-morpholino)ethanesulfonic acid (MES) pH 5.5. Tryptic peptides were dimethyl labeled using 60 mM sodium cyanoborohydride, 0.4% formaldehyde, and 250 mM MES pH 5.5 for 10 min. Dimethyl labeled peptides were eluted from Sep-Paks using 1.5 mL of 80% acetonitrile with 0.1% trifluoroacetic acid and lyophilized to dryness. Labeled peptides were reconstituted with 80% acetonitrile and 0.1% trifluoroacetic acid. Light, medium, and heavylabeled peptides were mixed 1:1:1, diluted with 0.1% formic acid to a final concentration of 3% acetonitrile and analyzed using 180 min data-dependent nLC-MS/MS on Thermo Orbitrap XL as later discussed. Light, medium, and heavy labeled samples were mixed using the protein median ratios as normalization from the “trial” analysis. 100 ug of mixed light, medium, and heavy labeled peptides were sub-fractionated using strong cation exchange (SCX) STAGETips (Millipore) as previously described (2). Briefly, 8 fractions were made using 25, 35, 50, 70, 90, 150, 350, and 750 mM ammonium acetate in 30% acetonitrile and 0.5% acetic acid. Each SCX fraction was desalted using C18 STAGETips, speed-vac concentrated to 1 uL, and resuspended with 10 uL of 3% acetonitrile and 0.1% formic acid. 5 uL of each SCX fraction was analyzed using 180 min data-dependent reverse-phase nLC-MS/MS on Thermo Orbitrap XL equipped with Eksigent Spark autosampler, Eksigent 2D nanoLC, and Phoenix ST Nimbus dual column source. Briefly, samples were loaded onto laser-pulled reverse-phase nanocapillary (150 um I.D., 360 um O.D.×25 cm length) with C18 (300A, 3 um particle size) (AcuTech Scientific) for 30 min with mobile phase A (3% acetonitrile, 3% dimethylsulfoxide, and 0.1% formic acid) at 600 nL/min. Peptides were analyzed over 180 min linear gradient of 0-40% mobile phase B (97% acetonitrile, 3% dimethylsulfoxide, and 0.1% formic acid) at 300 nL/min. Electrospray ionization and source parameters were as follows: spray voltage of 2.2 kV, capillary temperature of 200° C., capillary voltage at 35V, and tube lens at 90V, Data-dependent MS/MS was operated using the following parameters: full MS from 400-1700 m/z with 60,000 resolution at 400 m/z and target ion count of 3×10⁵ or fill time of 700 ms with lock-mass at 401.922718 m/z, and twelve MS/MS with charge-state screening excluding +1 and unassigned charge states for ions surpassing 6000 counts, target ion count of 5,000 or fill time of 50 ms, CID collision energy of 35, and dynamic exclusion of 30 sec. Raw data was searched against Uniprot human database using MaxQuant 1.5.3.30 with standard preset search parameters. Briefly, the search parameters were as follows: 3-plex dimethyl labeling to lysine and peptide N-terminus, trypsin cleavage allowing up to 2 missed cleavages, fixed modification of carbamidomethyl to cysteines, variable modifications of acetylation to protein N-terminus and methionine oxidation, 10 ppm and 0.5 Da mass errors for Full MS and MS/MS, respectively, 1% false-discovery rate on peptide and protein identifications, and peptide match between run feature with 1.5 min time window.

### Phosphoproteomics

Phosphopeptide enrichment was performed using HILIC/IMAC as previously described (3). Briefly, 6 mg of mixed light, medium, and heavy dimethyl labeled samples was injected onto HILIC TSK gel Amide-80 column (4.6×25 cm, 100 A pore size, 5 um particle size) (TOSOH Biosciences) using an Agilent 1090 HPLC equipped with a rheodyne 6-way port rotor with 1 mL sample loop. Fourty-one 1 min fractions were collected from 16-56 min and pooled into 28 fractions for subsequent IMAC enrichment using the previously described pooling strategy (3). IMAC enrichment was performed using 20 uL PHOS-Select Agarose beads(Sigma) on each of the 28 pooled HILIC fractions with AcroPrep Advances 96-well Filter Plates (0.45 um PTFE, PALL Corporation). Eluted phosphopeptides were further pooled into 14 fractions, speed-vac concentrated, and desalted using C18 STAGETips as previously described (2). Desalted fractions were handled same as SCX fractions and subjected to the same nLC-MS/MS conditions on Thermo Orbitrap XL.

### Animal Studies:

### Animals

Mice were housed under specific pathogen-free conditions and were treated in accordance with UCLA Animal Research protocol guidelines. All C57BL/6 female mice were purchased from the UCLA Radiation Oncology breeding colony.

### Preclinical Therapeutics

VE-822 (ApeXBio), 3-AP (ApeXBio) and DI-82 (Sundia Pharmaceuticals) were administered by intraperitoneal (i.p.) injections or oral gavage to recipient animals. All drugs for oral administration were solubilized in Prototype 9′ (PEG-200:Transcutol:Labrasol:Tween-80 mixed in 5:3:1:1 ratio) as single agents or in combination. For i.p. administration, drugs were solubilized in PEG-400 and 1 mM Tris-HCl at a 1:1 (v:v) ratio. Dasatinib (LC Laboratories) was solubilized in 80 mM citric acid (pH 3.1, Boulos et al.) and was administered at a dose of 10 mg/kg by oral gavage. 2×10⁵ luciferase expressing p185 cells were injected intravenously into C57BL/6 female mice for leukemia induction. All drugs were administered starting 6 or, 7 days after intravenous cell inoculation, when animals had developed a significant leukemic burden as monitored by bioluminescence imaging (IVIS Bioluminescence Imaging scanner). Dosing schedules are indicated in the text and figure legends. Mice were observed daily; those that became moribund during the trials (paralysis of hind limbs, significant body weight loss) were sacrificed immediately. Kaplan-Meier curves and Bioluminescence quantifications were generated using GraphPad Prism (v6.0f for Mac).

### Bioluminescence Imaging (BLI)

Mice were anesthetized with 2% isoflurane followed by an intraperitoneal injection of 50 μL (50 mg/ml) substrate luciferin. Mice were imaged with IVIS Bioluminescence Imaging scanner after 10 minutes after luciferin administration. All mice were imaged in groups of five with 1 minute exposure time and images were acquired at low binning.

### Sequencing of Dasatinib Resistant Clones

Bone marrow cells were harvested from dasatinib-relapsed mice at sacrifice and cultured in standard culture conditions. Genomic DNA from was collected from resistant cell populations and a 2-step nested PCR strategy was utilized to amplify the human ABL kinase domain. PCR products were sequenced and assessed for presence of T315I mutation.

### Pharmacokinetic Assays

### Treatment and Plasma Collection

Groups of C57BL/6 female mice were treated with therapeutic dosages of three drugs 3-AP (15 mg/kg), DI-82 (50 mg/kg) and VE-822 (40 mg/kg) as a single combination dosage solubilized in prototype 9′, by oral gavage. Blood was collected in heparin-EDTA tubes by retro-orbital technique at time points 0.5, 4 and 12 h from first set of mice and 1, 8 and 24 h from second set of mice. The blood samples were spun at 2000 g for 15 min and supernatant was collected for plasma. All plasma samples were frozen down at −20° C. before sample processing.

### Standard and Plasma Preparation

The stock solutions of 3-AP, VE-822, DI-82, 3-AP(NSC 266749), VE-821, DI-39 were prepared individually by dissolving appropriate amount of each chemical in a known volume of dimethyl sulfoxide (DMSO) to make 10 mM concentration and were kept at −20° C. before use. 3-AP analog and VE-821 (internal standards) were diluted to 200 nM concentrations with methanol to make the internal solution. The calibration standards were prepared by spiking working stock solutions of 3-AP, VE-822 and DI-82 in plasma from untreated mice to give the following range: 0.01-10 pmol/μL. Each 20 μL calibration standard sample was mixed with 60 μL of internal solution (methanol with 200 nM internal standards) and vortexed for 30 s. Following centrifugation at 15,000×g for 10 min, about 60 μL, was carefully transferred into mass spec vials for LC-MS/MS analysis. Plasma samples were process the same way as the calibration standard samples.

### Instrumentation

20 μL samples were injected onto a reverse phase column (Thermo Scientific Aquasil RP18 column 3.0 μm; 2.1×50 mm) equilibrated in water/formic acid, 100/0.1, and eluted (200 μL/min) with an increasing concentration of solvent B (acetonitrile/formic acid, 100/0.1, vol/vol: min/% acetonitrile; 0/0, 5/0, 15/60, 16/100, 19/100, 20/0, and 25/0). Another 20 μL samples were injected onto a reverse phase column (Thermo Scientific Aquasil RPC8 column 3.0 μm; 2.1×50 mm) equilibrated in water/formic acid, 100/0.1, and eluted (200 μL/min) with an increasing concentration of solvent B (acetonitrile/formic acid, 100/0.1, vol/vol: min/% acetonitrile; 0/0, 5/0, 15/60, 16/100, 19/100, 20/0, and 24/0). The effluent from the column was directed to an electrospray ion source (Jet Stream; Agilent Technologies) connected to a triple quadrupole mass spectrometer (6460 QQQ; Agilent Technologies) operating in the positive ion multiple-reaction-monitoring mode. The ion transitions used were the following:

| Drug | Ion transitions (m/z) |
|---|---|
| 3-AP | 196 > 179 |
| DI-82 | 511 > 369 |
| VE-822 | 464 > 433 |
| VE-821 | 369 > 276 |
| SAP-analog | 199 > 182 |
| DI-39 | 525−>383 |

### Inhibitors

The inhibitors that were used in this study are as follows: Pentostatin (Santa Cruz Biotechnology, 10 μM), DI-82 (refer to Nomme et al., 2014, 1 μM), 6-thioguanine (Sigma-Aldrich, as indicated), Gemcitabine (Sigma-Aldrich, as indicated), Pemetrexed (Selleckchem, 100 nM and as indicated), Dasatinib (1 nM, LC Laboratories) and 3-AP (ApeXBio, 500 nM and as indicated), VE-822 (ApeXBio, 1 μM and as indicated), Pablociclib (Selleckchem, 1 μM).

### Statistical Analyses

Data are presented as means±SD of at least three biological replicates. Statistical significance is determined by two-tailed t-test and values less than 0.05 were considered significant. Prism 6.0 h (Graphpad Software) was used to calculate statistics and generate graphs.

1. A pharmaceutical composition comprising a pharmaceutically acceptable excipient, a de novo nucleotide biosynthesis pathway inhibitor, a nucleoside salvage pathway inhibitor, and a replication stress response pathway inhibitor.

2. The pharmaceutical composition of claim 1, wherein the de novo nucleotide biosynthesis pathway inhibitor is an RNR inhibitor.

3. The pharmaceutical composition of claim 1, wherein the de novo nucleotide biosynthesis pathway inhibitor is selected from hydroxyurea, gallium maltolate, triapine (3-AP), thymidine, or clofarabine.

4. (canceled)

5. The pharmaceutical composition of claim 1, wherein the nucleoside salvage pathway inhibitor is a dCK inhibitor.

6. The pharmaceutical composition of claim 1, wherein the nucleoside salvage pathway inhibitor is selected from LP-661438, DI-39, or (R)-D1-82.

7. The pharmaceutical composition of claim 1, wherein the nucleoside salvage pathway inhibitor is a racemic mixture of DI-82.

8. (canceled)

9. The pharmaceutical composition of claim 1, wherein the replication stress response pathway inhibitor is an ATR inhibitor.

10. The pharmaceutical composition of claim 1, wherein the replication stress response pathway inhibitor is a Chk1 inhibitor.

11. The pharmaceutical composition of claim 1, wherein the replication stress response pathway inhibitor is a WEE1 inhibitor.

12. The pharmaceutical composition of claim 1, wherein the replication stress response pathway inhibitor is selected from VE-821, Ve-822, AZ20, AZD6738, torin-2, NVP-BEZ235, CHIR-124, SB-218078, SAR-020106, SCH900776, LY2603618, GNE-783, GNE-900, PD-321852, CCT244747, UCN-01, PF 00477736, XL844, AZD7762, AR458323, GDC0425, GDC0575, AR323, AR678, TCS2312, V158411, CEP-3891, MK-1775, or PD-407824.

13. (canceled)

14. The pharmaceutical composition of claim 1 for use in treating cancer in a patient in need of such treatment, the use comprising administering an effective amount of the pharmaceutical composition to the patient.

15. The pharmaceutical composition of claim 1 for use in inhibiting the growth of a cancer cell comprising contacting the cancer cell with the pharmaceutical composition.

16. A pharmaceutical composition, comprising:
(i) a pharmaceutically acceptable excipient; and
(ii) a de novo nucleotide biosynthesis pathway inhibitor, a nucleoside salvage pathway inhibitor, a replication stress response pathway inhibitor, or any combination thereof.

17. The pharmaceutical composition of claim 16, wherein the composition comprises a de novo nucleotide biosynthesis pathway inhibitor.

18. The pharmaceutical composition of claim 16, wherein the composition comprises a nucleoside salvage pathway inhibitor.

19. The pharmaceutical composition of claim 16, wherein the composition comprises a replication stress response pathway inhibitor.

20. The pharmaceutical composition of claim 16, wherein the composition comprises a de novo nucleotide biosynthesis pathway inhibitor and a nucleoside salvage pathway inhibitor.

21. The pharmaceutical composition of claim 16, wherein the composition comprises a de novo nucleotide biosynthesis pathway inhibitor and a replication stress response pathway inhibitor.

22. The pharmaceutical composition of claim 16, wherein the composition comprises a nucleoside salvage pathway inhibitor and a replication stress response pathway inhibitor.

23. The pharmaceutical composition of claim 16 for use in treating cancer in a patient in need of such treatment, the use comprising administering an effective amount of the pharmaceutical composition to the patient.

24. The pharmaceutical composition of claim 16 for use in inhibiting the growth of a cancer cell comprising contacting the cancer cell with the pharmaceutical composition.
